SEQUENCE LISTING (425 sequences)

SEQ ID NO: 1 - RNA, 36 nt, Artificial Sequence
  Synthetic
guugcaaaacccaagaaauccgucuuucauugacgg

SEQ ID NO: 2 - RNA, 36 nt, Artificial Sequence
  Synthetic
aauagcggcccuaagaaauccgucuuucauugacgg

SEQ ID NO: 3 - RNA, 36 nt, Artificial Sequence
  Synthetic
auuggaacuggcgagaaauccgucuuucauugacgg

SEQ ID NO: 4 - RNA, 36 nt, Artificial Sequence
  Synthetic
ccagcaacaccuaagaaauccgucuuucauugacgg

SEQ ID NO: 5 - RNA, 36 nt, Artificial Sequence
  Synthetic
cggcgcucgaauaggaaauccgucuuucauugacgg

SEQ ID NO: 6 - RNA, 36 nt, Artificial Sequence
  Synthetic
guggcaacaccuaagaaauccgucuuucauugacgg

SEQ ID NO: 7 - RNA, 36 nt, Artificial Sequence
  Synthetic
guugcaacaccuaagaaauccgucuuucauugacgg

SEQ ID NO: 8 - RNA, 36 nt, Artificial Sequence
  Synthetic
guugcaaugccuaagaaauccgucuuucauugacgg

SEQ ID NO: 9 - RNA, 34 nt, Artificial Sequence
  Synthetic
gcaacaccuaagaaauccgucuuucauugacggg

SEQ ID NO: 10 - RNA, 23 nt, Artificial Sequence
  Synthetic
agaaauccgucuuucauugacgg

SEQ ID NO: 11 - DNA, 30 bp, Artificial Sequence
  Synthetic
ttggcaggatggcttctcatcgtctgctcc

SEQ ID NO: 12 - DNA, 30 bp, Artificial Sequence
  Synthetic
tcatcgtctgctcctcctctgccttgctgg

SEQ ID NO: 13 - DNA, 30 bp, Artificial Sequence
  Synthetic
ctggactggtatttgtgtctgaggctggcc

SEQ ID NO: 14 - DNA, 30 bp, Artificial Sequence
  Synthetic
gtgtctgaggctggccctacggtgagtgtt

SEQ ID NO: 15 - DNA, 30 bp, Artificial Sequence
  Synthetic
tgtctgaggctggccctacggtgagtgttt

SEQ ID NO: 16 - DNA, 30 bp, Artificial Sequence
  Synthetic
ctgtgacatcccattcctacatttaagatt

SEQ ID NO: 17 - DNA, 30 bp, Artificial Sequence
  Synthetic
tgtgacatcccattcctacatttaagattc

SEQ ID NO: 18 - DNA, 30 bp, Artificial Sequence
  Synthetic
agcgtgaatcttaaatgtaggaatgggatg

SEQ ID NO: 19 - DNA, 30 bp, Artificial Sequence
  Synthetic
gcgtgaatcttaaatgtaggaatgggatgt

SEQ ID NO: 20 - DNA, 30 bp, Artificial Sequence
  Synthetic
aatgtaggaatgggatgtcacagaaacact

SEQ ID NO: 21 - DNA, 30 bp, Artificial Sequence
  Synthetic
gcaggatggcttctcatcgtctgctcctcc

SEQ ID NO: 22 - DNA, 24 bp, Artificial Sequence
  Synthetic
ctacatttaagattcacgctaaat

SEQ ID NO: 23 - DNA, 30 bp, Artificial Sequence
  Synthetic
gattcaccggtgccctgggtgtagagaaga

SEQ ID NO: 24 - DNA, 30 bp, Artificial Sequence
  Synthetic
tgttaacttctcacgtgtcttctctacacc

SEQ ID NO: 25 - DNA, 30 bp, Artificial Sequence
  Synthetic
gttaacttctcacgtgtcttctctacaccc

SEQ ID NO: 26 - DNA, 30 bp, Artificial Sequence
  Synthetic
ttaacttctcacgtgtcttctctacaccca

SEQ ID NO: 27 - DNA, 30 bp, Artificial Sequence
  Synthetic
tcacgtgtcttctctacacccagggcaccg

SEQ ID NO: 28 - DNA, 30 bp, Artificial Sequence
  Synthetic
accggtgccctgggtgtagagaagacacgt

SEQ ID NO: 29 - DNA, 30 bp, Artificial Sequence
  Synthetic
tagatgctgtccgaggcagtcctgccatca

SEQ ID NO: 30 - DNA, 30 bp, Artificial Sequence
  Synthetic
accatcagaggacacttggattcaccggtg

SEQ ID NO: 31 - DNA, 30 bp, Artificial Sequence
  Synthetic
gaccatcagaggacacttggattcaccggt

SEQ ID NO: 32 - DNA, 30 bp, Artificial Sequence
  Synthetic
atggcaggactgcctcggacagcatctaga

SEQ ID NO: 33 - DNA, 30 bp, Artificial Sequence
  Synthetic
tgaacacatgcacggccacattgatggcag

SEQ ID NO: 34 - DNA, 30 bp, Artificial Sequence
  Synthetic
ctgaacacatgcacggccacattgatggca

SEQ ID NO: 35 - DNA, 30 bp, Artificial Sequence
  Synthetic
cccagaggcaaatggctcccaggtgtcatc

SEQ ID NO: 36 - DNA, 30 bp, Artificial Sequence
  Synthetic
gcaacttacccagaggcaaatggctcccag

SEQ ID NO: 37 - DNA, 30 bp, Artificial Sequence
  Synthetic
tctacacccagggcaccggtgaatccaagt

SEQ ID NO: 38 - DNA, 30 bp, Artificial Sequence
  Synthetic
ggcaacttacccagaggcaaatggctccca

SEQ ID NO: 39 - DNA, 30 bp, Artificial Sequence
  Synthetic
ccaaagaaccctcccacaggacttggtttt

SEQ ID NO: 40 - DNA, 30 bp, Artificial Sequence
  Synthetic
cctctgggtaagttgccaaagaaccctccc

SEQ ID NO: 41 - DNA, 30 bp, Artificial Sequence
  Synthetic
gcctctgggtaagttgccaaagaaccctcc

SEQ ID NO: 42 - DNA, 30 bp, Artificial Sequence
  Synthetic
agaaaggctgctgatgacacctgggagcca

SEQ ID NO: 43 - DNA, 30 bp, Artificial Sequence
  Synthetic
tttggcaacttacccagaggcaaatggctc

SEQ ID NO: 44 - DNA, 30 bp, Artificial Sequence
  Synthetic
acttctcacgtgtcttctctacacccaggg

SEQ ID NO: 45 - DNA, 20 bp, Artificial Sequence
  Synthetic
acaaaattgatcagagtgaa

SEQ ID NO: 46 - DNA, 30 bp, Artificial Sequence
  Synthetic
gcatctccccattccatgagcatgcagagg

SEQ ID NO: 47 - DNA, 30 bp, Artificial Sequence
  Synthetic
ctggaaggcacttggcatctccccattcca

SEQ ID NO: 48 - DNA, 30 bp, Artificial Sequence
  Synthetic
tagaagggatatacaaagtggaaatagaca

SEQ ID NO: 49 - DNA, 30 bp, Artificial Sequence
  Synthetic
tacaaattcctcctcagttgtgagcccatg

SEQ ID NO: 50 - DNA, 30 bp, Artificial Sequence
  Synthetic
gagggttgttttggttttggtttttgcttt

SEQ ID NO: 51 - DNA, 30 bp, Artificial Sequence
  Synthetic
cagtaagatttggtgtctatttccactttg

SEQ ID NO: 52 - DNA, 30 bp, Artificial Sequence
  Synthetic
ggtgtctatttccactttgtatatcccttc

SEQ ID NO: 53 - DNA, 30 bp, Artificial Sequence
  Synthetic
gtgtctatttccactttgtatatcccttct

SEQ ID NO: 54 - DNA, 30 bp, Artificial Sequence
  Synthetic
ccactttgtatatcccttctacaaattcct

SEQ ID NO: 55 - DNA, 30 bp, Artificial Sequence
  Synthetic
cactttgtatatcccttctacaaattcctc

SEQ ID NO: 56 - DNA, 30 bp, Artificial Sequence
  Synthetic
gtatatcccttctacaaattcctcctcagt

SEQ ID NO: 57 - DNA, 30 bp, Artificial Sequence
  Synthetic
tatatcccttctacaaattcctcctcagtt

SEQ ID NO: 58 - DNA, 30 bp, Artificial Sequence
  Synthetic
gtagaagggatatacaaagtggaaatagac

SEQ ID NO: 59 - DNA, 30 bp, Artificial Sequence
  Synthetic
ctcctcagttgtgagcccatgcagctctcc

SEQ ID NO: 60 - DNA, 30 bp, Artificial Sequence
  Synthetic
tgagcccatgcagctctccagactcactgg

SEQ ID NO: 61 - DNA, 30 bp, Artificial Sequence
  Synthetic
tcctataaggtgtgaaagtctggattaagt

SEQ ID NO: 62 - DNA, 30 bp, Artificial Sequence
  Synthetic
cctataaggtgtgaaagtctggattaagtt

SEQ ID NO: 63 - DNA, 30 bp, Artificial Sequence
  Synthetic
ctataaggtgtgaaagtctggattaagtta

SEQ ID NO: 64 - DNA, 30 bp, Artificial Sequence
  Synthetic
catgagcatgcagaggtgagtatacagacc

SEQ ID NO: 65 - DNA, 30 bp, Artificial Sequence
  Synthetic
taggaaaaccagtgagtctggagagctgca

SEQ ID NO: 66 - DNA, 30 bp, Artificial Sequence
  Synthetic
gtttcctccatgcgtaacttaatccagact

SEQ ID NO: 67 - DNA, 30 bp, Artificial Sequence
  Synthetic
cacaccttataggaaaaccagtgagtctgg

SEQ ID NO: 68 - DNA, 30 bp, Artificial Sequence
  Synthetic
atccagactttcacaccttataggaaaacc

SEQ ID NO: 69 - DNA, 30 bp, Artificial Sequence
  Synthetic
ctccatgcgtaacttaatccagactttcac

SEQ ID NO: 70 - DNA, 30 bp, Artificial Sequence
  Synthetic
cctccatgcgtaacttaatccagactttca

SEQ ID NO: 71 - DNA, 30 bp, Artificial Sequence
  Synthetic
tttcctccatgcgtaacttaatccagactt

SEQ ID NO: 72 - DNA, 30 bp, Artificial Sequence
  Synthetic
acaccttataggaaaaccagtgagtctgga

SEQ ID NO: 73 - DNA, 24 bp, Artificial Sequence
  Synthetic
ttttggttttggtttttgcttttg

SEQ ID NO: 74 - DNA, 21 bp, Artificial Sequence
  Synthetic
tggttttggtttttgcttttg

SEQ ID NO: 75 - DNA, 20 bp, Artificial Sequence
  Synthetic
ggttttggtttttgcttttg

SEQ ID NO: 76 - DNA, 25 bp, Artificial Sequence
  Synthetic
agttacgcatggaggaaacaaatgg

SEQ ID NO: 77 - DNA, 20 bp, Artificial Sequence
  Synthetic
cgcatggaggaaacaaatgg

SEQ ID NO: 78 - DNA, 30 bp, Artificial Sequence
  Synthetic
ctaaagcagtgttttcacctcatatgctat

SEQ ID NO: 79 - DNA, 30 bp, Artificial Sequence
  Synthetic
actaaagcagtgttttcacctcatatgcta

SEQ ID NO: 80 - DNA, 30 bp, Artificial Sequence
  Synthetic
tactaaagcagtgttttcacctcatatgct

SEQ ID NO: 81 - DNA, 30 bp, Artificial Sequence
  Synthetic
ttactaaagcagtgttttcacctcatatgc

SEQ ID NO: 82 - DNA, 30 bp, Artificial Sequence
  Synthetic
catttttactaaagcagtgttttcacctca

SEQ ID NO: 83 - DNA, 30 bp, Artificial Sequence
  Synthetic
atgtaaccaagagtattccatttttactaa

SEQ ID NO: 84 - DNA, 30 bp, Artificial Sequence
  Synthetic
catgtaaccaagagtattccatttttacta

SEQ ID NO: 85 - DNA, 30 bp, Artificial Sequence
  Synthetic
caccacggctgtcgtcaccaatcccaagga

SEQ ID NO: 86 - DNA, 30 bp, Artificial Sequence
  Synthetic
tcacctcatatgctatgttagaagtccagg

SEQ ID NO: 87 - DNA, 30 bp, Artificial Sequence
  Synthetic
ccgccctgctgagcccctactcctattcca

SEQ ID NO: 88 - DNA, 30 bp, Artificial Sequence
  Synthetic
acagccaacgactccggcccccgccgctac

SEQ ID NO: 89 - DNA, 30 bp, Artificial Sequence
  Synthetic
tctcataggtggtattcacagccaacgact

SEQ ID NO: 90 - DNA, 30 bp, Artificial Sequence
  Synthetic
gggctctggtggaaatggatctgtctgtct

SEQ ID NO: 91 - DNA, 30 bp, Artificial Sequence
  Synthetic
cgggctctggtggaaatggatctgtctgtc

SEQ ID NO: 92 - DNA, 30 bp, Artificial Sequence
  Synthetic
tcgggctctggtggaaatggatctgtctgt

SEQ ID NO: 93 - DNA, 30 bp, Artificial Sequence
  Synthetic
tcctccagtggacctgaaggacgagggatg

SEQ ID NO: 94 - DNA, 30 bp, Artificial Sequence
  Synthetic
cacctcatatgctatgttagaagtccaggc

SEQ ID NO: 95 - DNA, 30 bp, Artificial Sequence
  Synthetic
cacaggaatgttttattgtctctgcctgga

SEQ ID NO: 96 - DNA, 30 bp, Artificial Sequence
  Synthetic
gaagtccaggcagagacaataaaacattcc

SEQ ID NO: 97 - DNA, 30 bp, Artificial Sequence
  Synthetic
gctgtgaataccacctatgagagaagacag

SEQ ID NO: 98 - DNA, 30 bp, Artificial Sequence
  Synthetic
gtgacgacagccgtggtggaataggagtag

SEQ ID NO: 99 - DNA, 30 bp, Artificial Sequence
  Synthetic
ggattggtgacgacagccgtggtggaatag

SEQ ID NO: 100 - DNA, 30 bp, Artificial Sequence
  Synthetic
cttgggattggtgacgacagccgtggtgga

SEQ ID NO: 101 - DNA, 30 bp, Artificial Sequence
  Synthetic
aggtccactggaggagaagtccctcattcc

SEQ ID NO: 102 - DNA, 30 bp, Artificial Sequence
  Synthetic
catgaaatcccatccctcgtccttcaggtc

SEQ ID NO: 103 - DNA, 30 bp, Artificial Sequence
  Synthetic
gttacatgaaatcccatccctcgtccttca

SEQ ID NO: 104 - DNA, 30 bp, Artificial Sequence
  Synthetic
gtaaaaatggaatactcttggttacatgaa

SEQ ID NO: 105 - DNA, 30 bp, Artificial Sequence
  Synthetic
agtaaaaatggaatactcttggttacatga

SEQ ID NO: 106 - DNA, 30 bp, Artificial Sequence
  Synthetic
taacatagcatatgaggtgaaaacactgct

SEQ ID NO: 107 - DNA, 30 bp, Artificial Sequence
  Synthetic
tctctgcctggacttctaacatagcatatg

SEQ ID NO: 108 - DNA, 30 bp, Artificial Sequence
  Synthetic
ttgtctctgcctggacttctaacatagcat

SEQ ID NO: 109 - DNA, 30 bp, Artificial Sequence
  Synthetic
attgtctctgcctggacttctaacatagca

SEQ ID NO: 110 - DNA, 30 bp, Artificial Sequence
  Synthetic
tattgtctctgcctggacttctaacatagc

SEQ ID NO: 111 - DNA, 30 bp, Artificial Sequence
  Synthetic
acaggaatgttttattgtctctgcctggac

SEQ ID NO: 112 - DNA, 30 bp, Artificial Sequence
  Synthetic
acctcatatgctatgttagaagtccaggca

SEQ ID NO: 113 - DNA, 30 bp, Artificial Sequence
  Synthetic
ttcgggctctggtggaaatggatctgtctg

SEQ ID NO: 114 - DNA, 22 bp, Artificial Sequence
  Synthetic
ccaccagagcccgaaaaacgtg

SEQ ID NO: 115 - DNA, 21 bp, Artificial Sequence
  Synthetic
caccagagcccgaaaaacgtg

SEQ ID NO: 116 - RNA, 30 nt, Artificial Sequence
  Synthetic
uuggcaggauggcuucucaucgucugcucc

SEQ ID NO: 117 - RNA, 30 nt, Artificial Sequence
  Synthetic
ucaucgucugcuccuccucugccuugcugg

SEQ ID NO: 118 - RNA, 30 nt, Artificial Sequence
  Synthetic
cuggacugguauuugugucugaggcuggcc

SEQ ID NO: 119 - RNA, 30 nt, Artificial Sequence
  Synthetic
gugucugaggcuggcccuacggugaguguu

SEQ ID NO: 120 - RNA, 30 nt, Artificial Sequence
  Synthetic
ugucugaggcuggcccuacggugaguguuu

SEQ ID NO: 121 - RNA, 30 nt, Artificial Sequence
  Synthetic
cugugacaucccauuccuacauuuaagauu

SEQ ID NO: 122 - RNA, 30 nt, Artificial Sequence
  Synthetic
ugugacaucccauuccuacauuuaagauuc

SEQ ID NO: 123 - RNA, 30 nt, Artificial Sequence
  Synthetic
agcgugaaucuuaaauguaggaaugggaug

SEQ ID NO: 124 - RNA, 30 nt, Artificial Sequence
  Synthetic
gcgugaaucuuaaauguaggaaugggaugu

SEQ ID NO: 125 - RNA, 30 nt, Artificial Sequence
  Synthetic
aauguaggaaugggaugucacagaaacacu

SEQ ID NO: 126 - RNA, 30 nt, Artificial Sequence
  Synthetic
gcaggauggcuucucaucgucugcuccucc

SEQ ID NO: 127 - RNA, 24 nt, Artificial Sequence
  Synthetic
cuacauuuaagauucacgcuaaau

SEQ ID NO: 128 - RNA, 30 nt, Artificial Sequence
  Synthetic
gauucaccggugcccuggguguagagaaga

SEQ ID NO: 129 - RNA, 30 nt, Artificial Sequence
  Synthetic
uguuaacuucucacgugucuucucuacacc

SEQ ID NO: 130 - RNA, 30 nt, Artificial Sequence
  Synthetic
guuaacuucucacgugucuucucuacaccc

SEQ ID NO: 131 - RNA, 30 nt, Artificial Sequence
  Synthetic
uuaacuucucacgugucuucucuacaccca

SEQ ID NO: 132 - RNA, 30 nt, Artificial Sequence
  Synthetic
ucacgugucuucucuacacccagggcaccg

SEQ ID NO: 133 - RNA, 30 nt, Artificial Sequence
  Synthetic
accggugcccuggguguagagaagacacgu

SEQ ID NO: 134 - RNA, 30 nt, Artificial Sequence
  Synthetic
uagaugcuguccgaggcaguccugccauca

SEQ ID NO: 135 - RNA, 30 nt, Artificial Sequence
  Synthetic
accaucagaggacacuuggauucaccggug

SEQ ID NO: 136 - RNA, 30 nt, Artificial Sequence
  Synthetic
gaccaucagaggacacuuggauucaccggu

SEQ ID NO: 137 - RNA, 30 nt, Artificial Sequence
  Synthetic
auggcaggacugccucggacagcaucuaga

SEQ ID NO: 138 - RNA, 30 nt, Artificial Sequence
  Synthetic
ugaacacaugcacggccacauugauggcag

SEQ ID NO: 139 - RNA, 30 nt, Artificial Sequence
  Synthetic
cugaacacaugcacggccacauugauggca

SEQ ID NO: 140 - RNA, 30 nt, Artificial Sequence
  Synthetic
cccagaggcaaauggcucccaggugucauc

SEQ ID NO: 141 - RNA, 30 nt, Artificial Sequence
  Synthetic
gcaacuuacccagaggcaaauggcucccag

SEQ ID NO: 142 - RNA, 30 nt, Artificial Sequence
  Synthetic
ucuacacccagggcaccggugaauccaagu

SEQ ID NO: 143 - RNA, 30 nt, Artificial Sequence
  Synthetic
ggcaacuuacccagaggcaaauggcuccca

SEQ ID NO: 144 - RNA, 30 nt, Artificial Sequence
  Synthetic
ccaaagaacccucccacaggacuugguuuu

SEQ ID NO: 145 - RNA, 30 nt, Artificial Sequence
  Synthetic
ccucuggguaaguugccaaagaacccuccc

SEQ ID NO: 146 - RNA, 30 nt, Artificial Sequence
  Synthetic
gccucuggguaaguugccaaagaacccucc

SEQ ID NO: 147 - RNA, 30 nt, Artificial Sequence
  Synthetic
agaaaggcugcugaugacaccugggagcca

SEQ ID NO: 148 - RNA, 30 nt, Artificial Sequence
  Synthetic
uuuggcaacuuacccagaggcaaauggcuc

SEQ ID NO: 149 - RNA, 30 nt, Artificial Sequence
  Synthetic
acuucucacgugucuucucuacacccaggg

SEQ ID NO: 150 - RNA, 20 nt, Artificial Sequence
  Synthetic
acaaaauugaucagagugaa

SEQ ID NO: 151 - RNA, 30 nt, Artificial Sequence
  Synthetic
gcaucuccccauuccaugagcaugcagagg

SEQ ID NO: 152 - RNA, 30 nt, Artificial Sequence
  Synthetic
cuggaaggcacuuggcaucuccccauucca

SEQ ID NO: 153 - RNA, 30 nt, Artificial Sequence
  Synthetic
uagaagggauauacaaaguggaaauagaca

SEQ ID NO: 154 - RNA, 30 nt, Artificial Sequence
  Synthetic
uacaaauuccuccucaguugugagcccaug

SEQ ID NO: 155 - RNA, 30 nt, Artificial Sequence
  Synthetic
gaggguuguuuugguuuugguuuuugcuuu

SEQ ID NO: 156 - RNA, 30 nt, Artificial Sequence
  Synthetic
caguaagauuuggugucuauuuccacuuug

SEQ ID NO: 157 - RNA, 30 nt, Artificial Sequence
  Synthetic
ggugucuauuuccacuuuguauaucccuuc

SEQ ID NO: 158 - RNA, 30 nt, Artificial Sequence
  Synthetic
gugucuauuuccacuuuguauaucccuucu

SEQ ID NO: 159 - RNA, 30 nt, Artificial Sequence
  Synthetic
ccacuuuguauaucccuucuacaaauuccu

SEQ ID NO: 160 - RNA, 30 nt, Artificial Sequence
  Synthetic
cacuuuguauaucccuucuacaaauuccuc

SEQ ID NO: 161 - RNA, 30 nt, Artificial Sequence
  Synthetic
guauaucccuucuacaaauuccuccucagu

SEQ ID NO: 162 - RNA, 30 nt, Artificial Sequence
  Synthetic
uauaucccuucuacaaauuccuccucaguu

SEQ ID NO: 163 - RNA, 30 nt, Artificial Sequence
  Synthetic
guagaagggauauacaaaguggaaauagac

SEQ ID NO: 164 - RNA, 30 nt, Artificial Sequence
  Synthetic
cuccucaguugugagcccaugcagcucucc

SEQ ID NO: 165 - RNA, 30 nt, Artificial Sequence
  Synthetic
ugagcccaugcagcucuccagacucacugg

SEQ ID NO: 166 - RNA, 30 nt, Artificial Sequence
  Synthetic
uccuauaaggugugaaagucuggauuaagu

SEQ ID NO: 167 - RNA, 30 nt, Artificial Sequence
  Synthetic
ccuauaaggugugaaagucuggauuaaguu

SEQ ID NO: 168 - RNA, 30 nt, Artificial Sequence
  Synthetic
cuauaaggugugaaagucuggauuaaguua

SEQ ID NO: 169 - RNA, 30 nt, Artificial Sequence
  Synthetic
caugagcaugcagaggugaguauacagacc

SEQ ID NO: 170 - RNA, 30 nt, Artificial Sequence
  Synthetic
uaggaaaaccagugagucuggagagcugca

SEQ ID NO: 171 - RNA, 30 nt, Artificial Sequence
  Synthetic
guuuccuccaugcguaacuuaauccagacu

SEQ ID NO: 172 - RNA, 30 nt, Artificial Sequence
  Synthetic
cacaccuuauaggaaaaccagugagucugg

SEQ ID NO: 173 - RNA, 30 nt, Artificial Sequence
  Synthetic
auccagacuuucacaccuuauaggaaaacc

SEQ ID NO: 174 - RNA, 30 nt, Artificial Sequence
  Synthetic
cuccaugcguaacuuaauccagacuuucac

SEQ ID NO: 175 - RNA, 30 nt, Artificial Sequence
  Synthetic
ccuccaugcguaacuuaauccagacuuuca

SEQ ID NO: 176 - RNA, 30 nt, Artificial Sequence
  Synthetic
uuuccuccaugcguaacuuaauccagacuu

SEQ ID NO: 177 - RNA, 30 nt, Artificial Sequence
  Synthetic
acaccuuauaggaaaaccagugagucugga

SEQ ID NO: 178 - RNA, 24 nt, Artificial Sequence
  Synthetic
uuuugguuuugguuuuugcuuuug

SEQ ID NO: 179 - RNA, 21 nt, Artificial Sequence
  Synthetic
ugguuuugguuuuugcuuuug

SEQ ID NO: 180 - RNA, 20 nt, Artificial Sequence
  Synthetic
gguuuugguuuuugcuuuug

SEQ ID NO: 181 - RNA, 25 nt, Artificial Sequence
  Synthetic
aguuacgcauggaggaaacaaaugg

SEQ ID NO: 182 - RNA, 20 nt, Artificial Sequence
  Synthetic
cgcauggaggaaacaaaugg

SEQ ID NO: 183 - RNA, 30 nt, Artificial Sequence
  Synthetic
cuaaagcaguguuuucaccucauaugcuau

SEQ ID NO: 184 - RNA, 30 nt, Artificial Sequence
  Synthetic
acuaaagcaguguuuucaccucauaugcua

SEQ ID NO: 185 - RNA, 30 nt, Artificial Sequence
  Synthetic
uacuaaagcaguguuuucaccucauaugcu

SEQ ID NO: 186 - RNA, 30 nt, Artificial Sequence
  Synthetic
uuacuaaagcaguguuuucaccucauaugc

SEQ ID NO: 187 - RNA, 30 nt, Artificial Sequence
  Synthetic
cauuuuuacuaaagcaguguuuucaccuca

SEQ ID NO: 188 - RNA, 30 nt, Artificial Sequence
  Synthetic
auguaaccaagaguauuccauuuuuacuaa

SEQ ID NO: 189 - RNA, 30 nt, Artificial Sequence
  Synthetic
cauguaaccaagaguauuccauuuuuacua

SEQ ID NO: 190 - RNA, 30 nt, Artificial Sequence
  Synthetic
caccacggcugucgucaccaaucccaagga

SEQ ID NO: 191 - RNA, 30 nt, Artificial Sequence
  Synthetic
ucaccucauaugcuauguuagaaguccagg

SEQ ID NO: 192 - RNA, 30 nt, Artificial Sequence
  Synthetic
ccgcccugcugagccccuacuccuauucca

SEQ ID NO: 193 - RNA, 30 nt, Artificial Sequence
  Synthetic
acagccaacgacuccggcccccgccgcuac

SEQ ID NO: 194 - RNA, 30 nt, Artificial Sequence
  Synthetic
ucucauaggugguauucacagccaacgacu

SEQ ID NO: 195 - RNA, 30 nt, Artificial Sequence
  Synthetic
gggcucugguggaaauggaucugucugucu

SEQ ID NO: 196 - RNA, 30 nt, Artificial Sequence
  Synthetic
cgggcucugguggaaauggaucugucuguc

SEQ ID NO: 197 - RNA, 30 nt, Artificial Sequence
  Synthetic
ucgggcucugguggaaauggaucugucugu

SEQ ID NO: 198 - RNA, 30 nt, Artificial Sequence
  Synthetic
uccuccaguggaccugaaggacgagggaug

SEQ ID NO: 199 - RNA, 30 nt, Artificial Sequence
  Synthetic
caccucauaugcuauguuagaaguccaggc

SEQ ID NO: 200 - RNA, 30 nt, Artificial Sequence
  Synthetic
cacaggaauguuuuauugucucugccugga

SEQ ID NO: 201 - RNA, 30 nt, Artificial Sequence
  Synthetic
gaaguccaggcagagacaauaaaacauucc

SEQ ID NO: 202 - RNA, 30 nt, Artificial Sequence
  Synthetic
gcugugaauaccaccuaugagagaagacag

SEQ ID NO: 203 - RNA, 30 nt, Artificial Sequence
  Synthetic
gugacgacagccgugguggaauaggaguag

SEQ ID NO: 204 - RNA, 30 nt, Artificial Sequence
  Synthetic
ggauuggugacgacagccgugguggaauag

SEQ ID NO: 205 - RNA, 30 nt, Artificial Sequence
  Synthetic
cuugggauuggugacgacagccguggugga

SEQ ID NO: 206 - RNA, 30 nt, Artificial Sequence
  Synthetic
agguccacuggaggagaagucccucauucc

SEQ ID NO: 207 - RNA, 30 nt, Artificial Sequence
  Synthetic
caugaaaucccaucccucguccuucagguc

SEQ ID NO: 208 - RNA, 30 nt, Artificial Sequence
  Synthetic
guuacaugaaaucccaucccucguccuuca

SEQ ID NO: 209 - RNA, 30 nt, Artificial Sequence
  Synthetic
guaaaaauggaauacucuugguuacaugaa

SEQ ID NO: 210 - RNA, 30 nt, Artificial Sequence
  Synthetic
aguaaaaauggaauacucuugguuacauga

SEQ ID NO: 211 - RNA, 30 nt, Artificial Sequence
  Synthetic
uaacauagcauaugaggugaaaacacugcu

SEQ ID NO: 212 - RNA, 30 nt, Artificial Sequence
  Synthetic
ucucugccuggacuucuaacauagcauaug

SEQ ID NO: 213 - RNA, 30 nt, Artificial Sequence
  Synthetic
uugucucugccuggacuucuaacauagcau

SEQ ID NO: 214 - RNA, 30 nt, Artificial Sequence
  Synthetic
auugucucugccuggacuucuaacauagca

SEQ ID NO: 215 - RNA, 30 nt, Artificial Sequence
  Synthetic
uauugucucugccuggacuucuaacauagc

SEQ ID NO: 216 - RNA, 30 nt, Artificial Sequence
  Synthetic
acaggaauguuuuauugucucugccuggac

SEQ ID NO: 217 - RNA, 30 nt, Artificial Sequence
  Synthetic
accucauaugcuauguuagaaguccaggca

SEQ ID NO: 218 - RNA, 30 nt, Artificial Sequence
  Synthetic
uucgggcucugguggaaauggaucugucug

SEQ ID NO: 219 - RNA, 22 nt, Artificial Sequence
  Synthetic
ccaccagagcccgaaaaacgug

SEQ ID NO: 220 - RNA, 21 nt, Artificial Sequence
  Synthetic
caccagagcccgaaaaacgug

SEQ ID NO: 221 - DNA, 3162 bp, Artificial Sequence
  Synthetic
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 222 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 223 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 224 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 225 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 226 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 227 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 228 - DNA, 6945 bp, Artificial Sequence
  Synthetic
acagaagtccactcattcttggcaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 229 - DNA, 145 bp, Artificial Sequence
  Synthetic
acagaagtccactcattcttggcaggatggcttctcatcgtctgctcctcctctgccttgctggactggtatttgtgtctgaggctggccctacggtgagtgtttctgtgacatcccattcctacatttaagattcacgctaaat

SEQ ID NO: 230 - DNA, 231 bp, Artificial Sequence
  Synthetic
ttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgttt

SEQ ID NO: 231 - DNA, 236 bp, Artificial Sequence
  Synthetic
ccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttg

SEQ ID NO: 232 - DNA, 304 bp, Artificial Sequence
  Synthetic
cacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 233 - RNA, 36 nt, Artificial Sequence
  Synthetic
ucucaacgauagucagacauguguccucagugacac

SEQ ID NO: 234 - RNA, 36 nt, Artificial Sequence
  Synthetic
uuuuaacaacacucaggcauguguccacagugacac

SEQ ID NO: 235 - RNA, 36 nt, Artificial Sequence
  Synthetic
uugaacggauacucagacauguguuuccagugacac

SEQ ID NO: 236 - RNA, 36 nt, Artificial Sequence
  Synthetic
ugcccucaauagucagauguguguccacagugacac

SEQ ID NO: 237 - RNA, 36 nt, Artificial Sequence
  Synthetic
ucucaaugauacuuagauacguguccucagugacac

SEQ ID NO: 238 - RNA, 36 nt, Artificial Sequence
  Synthetic
ucucaaugauacucagacauguguccccagugacac

SEQ ID NO: 239 - RNA, 36 nt, Artificial Sequence
  Synthetic
ucucaaugauacuaagacauguguccucagugacac

SEQ ID NO: 240 - RNA, 36 nt, Artificial Sequence
  Synthetic
ucucaacuauacucagacauguguccucagugacac

SEQ ID NO: 241 - RNA, 36 nt, Artificial Sequence
  Synthetic
ucucaacgauacucagacauguguccucagugacac

SEQ ID NO: 242 - RNA, 36 nt, Artificial Sequence
  Synthetic
ucucaacgauacuaagauauguguccucagcgacac

SEQ ID NO: 243 - RNA, 36 nt, Artificial Sequence
  Synthetic
ucucaacgauacuaagauauguguccccagugacac

SEQ ID NO: 244 - RNA, 36 nt, Artificial Sequence
  Synthetic
ucucaacgauacuaagauauguguccacagugacac

SEQ ID NO: 245 - RNA, 36 nt, Artificial Sequence
  Synthetic
ucucaacaauacucagacauguguccccagugacac

SEQ ID NO: 246 - RNA, 36 nt, Artificial Sequence
  Synthetic
ucucaacaauacuaaggcauguguccccagugaccc

SEQ ID NO: 247 - RNA, 36 nt, Artificial Sequence
  Synthetic
ucucaaagauacucagacacguguccccagugacac

SEQ ID NO: 248 - RNA, 36 nt, Artificial Sequence
  Synthetic
ucucaaaaauacucagacauguguccucagugacac

SEQ ID NO: 249 - RNA, 36 nt, Artificial Sequence
  Synthetic
gcgaaacaacagucagacauguguccccagugacac

SEQ ID NO: 250 - RNA, 36 nt, Artificial Sequence
  Synthetic
ccucaacgauauuaagacauguguccgcagugacac

SEQ ID NO: 251 - RNA, 22 nt, Artificial Sequence
  Synthetic
agacauguguccucagugacac

SEQ ID NO: 252 - DNA, 3222 bp, Artificial Sequence
  Synthetic
atggcttccatctctaggccatacggcaccaagctgcgaccggacgcacggaagaaggagatgctcgataagttctttaatacactgactaagggtcagcgcgtgttcgcagacctggccctgtgcatctatggctccctgaccctggagatggccaagtctctggagccagaaagtgattcagaactggtgtgcgctattgggtggtttcggctggtggacaagaccatctggtccaaggatggcatcaagcaggagaatctggtgaaacagtacgaagcctattccggaaaggaggcttctgaagtggtcaaaacatacctgaacagccccagctccgacaagtacgtgtggatcgattgcaggcagaaattcctgaggtttcagcgcgagctcggcactcgcaacctgtccgaggacttcgaatgtatgctctttgaacagtacattagactgaccaagggcgagatcgaagggtatgccgctatttcaaatatgttcggaaacggcgagaaggaagaccggagcaagaaaagaatgtacgctacacggatgaaagattggctggaggcaaacgaaaatatcacttgggagcagtatagagaggccctgaagaaccagctgaatgctaaaaacctggagcaggttgtggccaattacaaggggaacgctggcggggcagaccccttctttaagtatagcttctccaaagagggaatggtgagcaagaaagaacatgcacagcagctcgacaagttcaaaaccgtcctgaagaacaaagcccgggacctgaattttccaaacaaggagaagctgaagcagtacctggaggccgaaatcggcattccggtcgacgctaacgtgtactcccagatgttctctaacggggtgagtgaggtccagcctaagaccacacggaatatgtcttttagtaacgagaaactggatctgctcactgaactgaaggacctgaacaagggcgatgggttcgagtacgccagagaagtgctgaacgggttctttgactccgagctccacactaccgaggataagtttaatatcacctctaggtacctgggaggcgacaaatcaaaccgcctgagcaaactctataagatctggaagaaagagggtgtggactgcgaggaaggcattcagcagttctgtgaagccgtcaaagataagatgggccagatccccattcgaaatgtgctgaagtacctgtggcagttccgggagacagtcagtgccgaggattttgaagcagccgctaaggctaaccatctggaggaaaagatcagccgggtgaaagcccacccaatcgtgattagcaataggtactgggcttttgggacttccgcactggtgggaaacattatgcccgcagacaagaggcatcagggagagtatgccggtcagaatttcaaaatgtggctggaggctgaactgcactacgatggcaagaaagcaaagcaccatctgcctttttataacgcccgcttctttgaggaagtgtactgctatcacccctctgtcgccgagatcactcctttcaaaaccaagcagtttggctgtgaaatcgggaaggacattccagattacgtgagcgtcgctctgaaggacaatccgtataagaaagcaaccaaacgaatcctgcgtgcaatctacaatcccgtcgccaacacaactggcgttgataagaccacaaactgcagcttcatgatcaaacgcgagaatgacgaatataagctggtcatcaaccgaaaaatttccgtggatcggcctaagagaatcgaagtgggcaggacaattatggggtacgaccgcaatcagacagctagcgatacttattggattggccggctggtgccacctggaacccggggcgcataccgcatcggagagtggagcgtccagtatattaagtccgggcctgtcctgtctagtactcagggagttaacaattccactaccgaccagctggtgtacaacggcatgccatcaagctccgagcggttcaaggcctggaagaaagccagaatggcttttatccgaaaactcattcgtcagctgaatgacgagggactggaatctaagggtcaggattatatccccgagaacccttctagtttcgatgtgcggggcgaaaccctgtacgtctttaacagtaattatctgaaggccctggtgagcaaacacagaaaggccaagaaacctgttgaggggatcctggacgagattgaagcctggacatctaaagacaaggattcatgcagcctgatgcggctgagcagcctgagcgatgcttccatgcagggaatcgccagcctgaagagtctgattaacagctacttcaacaagaatggctgtaaaaccatcgaggacaaagaaaagtttaatcccgtgctgtatgccaagctggttgaggtggaacagcggagaacaaacaagcggtctgagaaagtgggaagaatcgcaggtagtctggagcagctggccctgctgaacggggttgaggtggtcatcggcgaagctgacctgggggaggtcgaaaaaggaaagagtaagaaacagaattcacggaacatggattggtgcgcaaagcaggtggcacagcggctggagtacaaactggccttccatggaatcggttactttggagtgaaccccatgtataccagccaccaggaccctttcgaacataggcgcgtggctgatcacatcgtcatgcgagcacgttttgaggaagtcaacgtggagaacattgccgaatggcacgtgcgaaatttctcaaactacctgcgtgcagacagcggcactgggctgtactataagcaggccaccatggacttcctgaaacattacggtctggaggaacacgctgagggcctggaaaataagaaaatcaagttctatgactttagaaagatcctggaggataaaaacctgacaagcgtgatcattccaaagaggggcgggcgcatctacatggccaccaacccagtgacatccgactctaccccgattacatacgccggcaagacttataataggtgtaacgctgatgaggtggcagccgctaatatcgttatttctgtgctggctccccgcagtaagaaaaacgaggaacaggacgatatccctctgattaccaagaaagccgagagtaagtcaccaccgaaagaccggaagagatcaaaaacaagccagctgcctcagaaa

SEQ ID NO: 253 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 254 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 255 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 256 - protein, 147 aa, Artificial Sequence
  Synthetic
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 257 - protein, 127 aa, Artificial Sequence
  Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 258 - DNA, 444 bp, Artificial Sequence
  Synthetic
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 259 - RNA, 36 nt, Artificial Sequence
  Synthetic
guuggaaugacuaauuuuugugcccaccguuggcac

SEQ ID NO: 260 - RNA, 24 nt, Artificial Sequence
  Synthetic
aauuuuugugcccaucguuggcac

SEQ ID NO: 261 - RNA, 23 nt, Artificial Sequence
  Synthetic
auuuuugugcccaucguuggcac

SEQ ID NO: 262 - RNA, 36 nt, Artificial Sequence
  Synthetic
cuagcaaugaccuaauaguguguccuuaguugacau

SEQ ID NO: 263 - RNA, 36 nt, Artificial Sequence
  Synthetic
ccuacaauaccuaagaaauccguccuaaguugacgg

SEQ ID NO: 264 - RNA, 22 nt, Artificial Sequence
  Synthetic
auaguguguccuuaguugacau

SEQ ID NO: 265 - protein, 1093 aa, Artificial Sequence
  Synthetic
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 266 - protein, 1098 aa, Artificial Sequence
  Synthetic
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 267 - DNA, 20 bp, Artificial Sequence
  Synthetic
tgaacacatgcacggccaca

SEQ ID NO: 268 - DNA, 20 bp, Artificial Sequence
  Synthetic
ctgaacacatgcacggccac

SEQ ID NO: 269 - DNA, 20 bp, Artificial Sequence
  Synthetic
caccacggctgtcgtcacca

SEQ ID NO: 270 - DNA, 20 bp, Artificial Sequence
  Synthetic
ggattggtgacgacagccgt

SEQ ID NO: 271 - DNA, 20 bp, Artificial Sequence
  Synthetic
caccacggctgtcgtcacca

SEQ ID NO: 272 - DNA, 20 bp, Artificial Sequence
  Synthetic
ggattggtgacgacagccgt

SEQ ID NO: 273 - RNA, 43 nt, Artificial Sequence
  Synthetic
agaaauccgucuuucauugacggugaacacaugcacggccaca

SEQ ID NO: 274 - RNA, 43 nt, Artificial Sequence
  Synthetic
agaaauccgucuuucauugacggcugaacacaugcacggccac

SEQ ID NO: 275 - RNA, 43 nt, Artificial Sequence
  Synthetic
agaaauccgucuuucauugacggcaccacggcugucgucacca

SEQ ID NO: 276 - RNA, 43 nt, Artificial Sequence
  Synthetic
agaaauccgucuuucauugacggggauuggugacgacagccgu

SEQ ID NO: 277 - RNA, 43 nt, Artificial Sequence; identical to SEQ ID NO: 275
  Synthetic

SEQ ID NO: 278 - RNA, 43 nt, Artificial Sequence; identical to SEQ ID NO: 276
  Synthetic

SEQ ID NO: 279 - DNA, 100 bp, Artificial Sequence
  Synthetic
ccagaggcaaatggctcccaggtgtcatcagcagcctttctgaacacatgcatagctacattgatggcaggactgcctcggacagcatctagaactttga

SEQ ID NO: 280 - DNA, 100 bp, Artificial Sequence
  Synthetic
ccagaggcaaatggctcccaggtgtcatcagcagcctttctgaacacatgcatagctacgttgatggcaggactgcctcggacagcatctagaactttga

SEQ ID NO: 281 - DNA, 100 bp, Artificial Sequence
  Synthetic
ccagaggcaaatggctcccaggtgtcatcagcagcctttctgaacacatgcatggccacattgatggcaggactgcctcggacagcatctagaactttga

SEQ ID NO: 282 - DNA, 100 bp, Artificial Sequence
  Synthetic
ccagaggcaaatggctcccaggtgtcatcagcagcctttcgaaatacgtgcatagctacgttgatggcaggactgcctcggacagcatctagaactttga

SEQ ID NO: 283 - DNA, 100 bp, Artificial Sequence
  Synthetic
cccagaggcaaatggctcccaggtgtcatcagcagcctttcgaaatacgtgcatagctacattgatggcaggactgcctcggacagcatctagaactttg

SEQ ID NO: 284 - DNA, 100 bp, Artificial Sequence
  Synthetic
cccagaggcaaatggctcccaggtgtcatcagcagcctttctgaacacatgcatagctacattgatggcaggactgcctcggacagcatctagaactttg

SEQ ID NO: 285 - DNA, 100 bp, Artificial Sequence
  Synthetic
cccagaggcaaatggctcccaggtgtcatcagcagcctttctgaacacatgcatggccacattgatggcaggactgcctcggacagcatctagaactttg

SEQ ID NO: 286 - DNA, 100 bp, Artificial Sequence
  Synthetic
cccagaggcaaatggctcccaggtgtcatcagcagccttccgaaatacgtgcatagctacattgatggcaggactgcctcggacagcatctagaactttg

SEQ ID NO: 287 - DNA, 100 bp, Artificial Sequence
  Synthetic
gctacaccattgccgccctgctgagcccctactcctatagtactatggctgtcgtcaccaatcccaaggaatgagggacttctcctccagtggacctgaa

SEQ ID NO: 288 - DNA, 100 bp, Artificial Sequence
  Synthetic
gctacaccattgccgccctgctgagcccctactcctattccaccatggctgttgttactaatcccaaggaatgagggacttctcctccagtggacctgaa

SEQ ID NO: 289 - DNA, 100 bp, Artificial Sequence
  Synthetic
gctacaccattgccgccctgctgagcccctactcctattccaccatggctgtcgtcaccaatcccaaggaatgagggacttctcctccagtggacctgaa

SEQ ID NO: 290 - DNA, 100 bp, Artificial Sequence
  Synthetic
gctacaccattgccgccctgctgagcccctactcctatagtactatggccgttgttactaatcccaaggaatgagggacttctcctccagtggacctgaa

SEQ ID NO: 291 - DNA, 100 bp, Artificial Sequence
  Synthetic
gtccttcaggtccactggaggagaagtccctcattccttggggttagtgacgacagccatggtggaataggagtaggggctcagcagggcggcaatggtg

SEQ ID NO: 292 - DNA, 100 bp, Artificial Sequence
  Synthetic
gtccttcaggtccactggaggagaagtccctcattccttgggattggtgacaacggccatggtggaataggagtaggggctcagcagggcggcaatggtg

SEQ ID NO: 293 - DNA, 100 bp, Artificial Sequence
  Synthetic
gtccttcaggtccactggaggagaagtccctcattccttgggattggtgacgacagccatggtggaataggagtaggggctcagcagggcggcaatggtg

SEQ ID NO: 294 - DNA, 100 bp, Artificial Sequence
  Synthetic
gtccttcaggtccactggaggagaagtccctcattccttggggttagtaacaacggccatggtggaataggagtaggggctcagcagggcggcaatggtg

SEQ ID NO: 295 - DNA, 100 bp, Artificial Sequence
  Synthetic
gctacaccattgccgccctgctgagcccctactcctattctactacagccgttatcactaatcccaaggaatgagggacttctcctccagtggacctgaa

SEQ ID NO: 296 - DNA, 100 bp, Artificial Sequence
  Synthetic
gctacaccattgccgccctgctgagcccctactcctattccaccacggctgttatcactaatcccaaggaatgagggacttctcctccagtggacctgaa

SEQ ID NO: 297 - DNA, 100 bp, Artificial Sequence
  Synthetic
gctacaccattgccgccctgctgagcccctactcctattccaccacggctgtcatcaccaatcccaaggaatgagggacttctcctccagtggacctgaa

SEQ ID NO: 298 - DNA, 100 bp, Artificial Sequence
  Synthetic
gctacaccattgccgccctgctgagcccctactcctatagtactacagccgttatcactaatcccaaggaatgagggacttctcctccagtggacctgaa

SEQ ID NO: 299 - DNA, 100 bp, Artificial Sequence
  Synthetic
gtccttcaggtccactggaggagaagtccctcattccttgggattggtgataacggccgtggtggaataggagtaggggctcagcagggcggcaatggtg

SEQ ID NO: 300 - DNA, 100 bp, Artificial Sequence
  Synthetic
gtccttcaggtccactggaggagaagtccctcattccttgggattggtgataacggctgtggtggaataggagtaggggctcagcagggcggcaatggtg

SEQ ID NO: 301 - DNA, 100 bp, Artificial Sequence
  Synthetic
gtccttcaggtccactggaggagaagtccctcattccttgggattggtgatgacagccgtggtggaataggagtaggggctcagcagggcggcaatggtg

SEQ ID NO: 302 - DNA, 100 bp, Artificial Sequence
  Synthetic
gtccttcaggtccactggaggagaagtccctcattccttggggttagtgataacggctgtggtggaataggagtaggggctcagcagggcggcaatggtg

SEQ ID NO: 303 - DNA, 120 bp, Artificial Sequence
  Synthetic
ggcccccgccgctacaccattgccgccctgctgagcccctactcctatagtactatggctgtcgtcaccaatcccaaggaatgagggacttctcctccagtggacctgaaggacgaggga

SEQ ID NO: 304 - DNA, 120 bp, Artificial Sequence
  Synthetic
ggcccccgccgctacaccattgccgccctgctgagcccctactcctattccaccatggctgttgttactaatcccaaggaatgagggacttctcctccagtggacctgaaggacgaggga

SEQ ID NO: 305 - DNA, 120 bp, Artificial Sequence
  Synthetic
ggcccccgccgctacaccattgccgccctgctgagcccctactcctattccaccatggctgtcgtcaccaatcccaaggaatgagggacttctcctccagtggacctgaaggacgaggga

SEQ ID NO: 306 - DNA, 120 bp, Artificial Sequence
  Synthetic
ggcccccgccgctacaccattgccgccctgctgagcccctactcctatagtactatggccgttgttactaatcccaaggaatgagggacttctcctccagtggacctgaaggacgaggga

SEQ ID NO: 307 - DNA, 120 bp, Artificial Sequence
  Synthetic
cccatccctcgtccttcaggtccactggaggagaagtccctcattccttggggttagtgacgacagccatggtggaataggagtaggggctcagcagggcggcaatggtgtagcggcggg

SEQ ID NO: 308 - DNA, 120 bp, Artificial Sequence
  Synthetic
cccatccctcgtccttcaggtccactggaggagaagtccctcattccttgggattggtgacaacggccatggtggaataggagtaggggctcagcagggcggcaatggtgtagcggcggg

SEQ ID NO: 309 - DNA, 120 bp, Artificial Sequence
  Synthetic
cccatccctcgtccttcaggtccactggaggagaagtccctcattccttgggattggtgacgacagccatggtggaataggagtaggggctcagcagggcggcaatggtgtagcggcggg

SEQ ID NO: 310 - DNA, 120 bp, Artificial Sequence
  Synthetic
cccatccctcgtccttcaggtccactggaggagaagtccctcattccttggggttagtaacaacggccatggtggaataggagtaggggctcagcagggcggcaatggtgtagcggcggg

SEQ ID NO: 311 - DNA, 120 bp, Artificial Sequence
  Synthetic
ggcaacttacccagaggcaaatggctcccaggtgtcatcagcagcctttctgaacacatgcatagctacattgatggcaggactgcctcggacagcatctagaactttgaccatcagagg

SEQ ID NO: 312 - DNA, 120 bp, Artificial Sequence
  Synthetic
ggcaacttacccagaggcaaatggctcccaggtgtcatcagcagcctttctgaacacatgcatagctacgttgatggcaggactgcctcggacagcatctagaactttgaccatcagagg

SEQ ID NO: 313 - DNA, 120 bp, Artificial Sequence
  Synthetic
ggcaacttacccagaggcaaatggctcccaggtgtcatcagcagcctttctgaacacatgcatggccacattgatggcaggactgcctcggacagcatctagaactttgaccatcagagg

SEQ ID NO: 314 - DNA, 120 bp, Artificial Sequence
  Synthetic
ggcaacttacccagaggcaaatggctcccaggtgtcatcagcagcctttcgaaatacgtgcatagctacgttgatggcaggactgcctcggacagcatctagaactttgaccatcagagg

SEQ ID NO: 315 - DNA, 120 bp, Artificial Sequence
  Synthetic
tggcaacttacccagaggcaaatggctcccaggtgtcatcagcagcctttcgaaatacgtgcatagctacattgatggcaggactgcctcggacagcatctagaactttgaccatcagag

SEQ ID NO: 316 - DNA, 120 bp, Artificial Sequence
  Synthetic
tggcaacttacccagaggcaaatggctcccaggtgtcatcagcagcctttctgaacacatgcatagctacattgatggcaggactgcctcggacagcatctagaactttgaccatcagag

SEQ ID NO: 317 - DNA, 120 bp, Artificial Sequence
  Synthetic
tggcaacttacccagaggcaaatggctcccaggtgtcatcagcagcctttctgaacacatgcatggccacattgatggcaggactgcctcggacagcatctagaactttgaccatcagag

SEQ ID NO: 318 - DNA, 120 bp, Artificial Sequence
  Synthetic
tggcaacttacccagaggcaaatggctcccaggtgtcatcagcagccttccgaaatacgtgcatagctacattgatggcaggactgcctcggacagcatctagaactttgaccatcagag

SEQ ID NO: 319 - DNA, 120 bp, Artificial Sequence
  Synthetic
ggcccccgccgctacaccattgccgccctgctgagcccctactcctattctactacagccgttatcactaatcccaaggaatgagggacttctcctccagtggacctgaaggacgaggga

SEQ ID NO: 320 - DNA, 120 bp, Artificial Sequence
  Synthetic
ggcccccgccgctacaccattgccgccctgctgagcccctactcctattccaccacggctgttatcactaatcccaaggaatgagggacttctcctccagtggacctgaaggacgaggga

SEQ ID NO: 321 - DNA, 120 bp, Artificial Sequence
  Synthetic
ggcccccgccgctacaccattgccgccctgctgagcccctactcctattccaccacggctgtcatcaccaatcccaaggaatgagggacttctcctccagtggacctgaaggacgaggga

SEQ ID NO: 322 - DNA, 120 bp, Artificial Sequence
  Synthetic
ggcccccgccgctacaccattgccgccctgctgagcccctactcctatagtactacagccgttatcactaatcccaaggaatgagggacttctcctccagtggacctgaaggacgaggga

SEQ ID NO: 323 - DNA, 120 bp, Artificial Sequence
  Synthetic
cccatccctcgtccttcaggtccactggaggagaagtccctcattccttgggattggtgataacggccgtggtggaataggagtaggggctcagcagggcggcaatggtgtagcggcggg

SEQ ID NO: 324 - DNA, 120 bp, Artificial Sequence
  Synthetic
cccatccctcgtccttcaggtccactggaggagaagtccctcattccttgggattggtgataacggctgtggtggaataggagtaggggctcagcagggcggcaatggtgtagcggcggg

SEQ ID NO: 325 - DNA, 120 bp, Artificial Sequence
  Synthetic
cccatccctcgtccttcaggtccactggaggagaagtccctcattccttgggattggtgatgacagccgtggtggaataggagtaggggctcagcagggcggcaatggtgtagcggcggg

SEQ ID NO: 326 - DNA, 120 bp, Artificial Sequence
  Synthetic
cccatccctcgtccttcaggtccactggaggagaagtccctcattccttggggttagtgataacggctgtggtggaataggagtaggggctcagcagggcggcaatggtgtagcggcggg

SEQ ID NO: 327 - DNA, 20 bp, Artificial Sequence
  Synthetic
tcatcgtctgctcctcctct

SEQ ID NO: 328 - DNA, 20 bp, Artificial Sequence
  Synthetic
tctacacccagggcaccggt

SEQ ID NO: 329 - DNA, 20 bp, Artificial Sequence
  Synthetic
gaccatcagaggacacttgg

SEQ ID NO: 330 - DNA, 20 bp, Artificial Sequence
  Synthetic
tagatgctgtccgaggcagt

SEQ ID NO: 331 - DNA, 20 bp, Artificial Sequence
  Synthetic
agaaaggctgctgatgacac

SEQ ID NO: 332 - DNA, 20 bp, Artificial Sequence
  Synthetic
ctgaacacatgcacggccac

SEQ ID NO: 333 - DNA, 20 bp, Artificial Sequence
  Synthetic
ggcaacttacccagaggcaa

SEQ ID NO: 334 - DNA, 20 bp, Artificial Sequence
  Synthetic
tttggcaacttacccagagg

SEQ ID NO: 335 - DNA, 20 bp, Artificial Sequence
  Synthetic
cacaccttataggaaaacca

SEQ ID NO: 336 - DNA, 20 bp, Artificial Sequence
  Synthetic
tacaaattcctcctcagttg

SEQ ID NO: 337 - DNA, 20 bp, Artificial Sequence
  Synthetic
gtatatcccttctacaaatt

SEQ ID NO: 338 - DNA, 20 bp, Artificial Sequence
  Synthetic
cagtaagatttggtgtctat

SEQ ID NO: 339 - DNA, 20 bp, Artificial Sequence
  Synthetic
ccgccctgctgagcccctac

SEQ ID NO: 340 - DNA, 20 bp, Artificial Sequence
  Synthetic
caccacggctgtcgtcacca

SEQ ID NO: 341 - DNA, 20 bp, Artificial Sequence
  Synthetic
gtgacgacagccgtggtgga

SEQ ID NO: 342 - DNA, 20 bp, Artificial Sequence
  Synthetic
ggattggtgacgacagccgt

SEQ ID NO: 343 - DNA, 20 bp, Artificial Sequence
  Synthetic
cttgggattggtgacgacag

SEQ ID NO: 344 - DNA, 20 bp, Artificial Sequence
  Synthetic
aggtccactggaggagaagt

SEQ ID NO: 345 - RNA, 43 nt, Artificial Sequence
  Synthetic
agaaauccgucuuucauugacggucaucgucugcuccuccucu

SEQ ID NO: 346 - RNA, 43 nt, Artificial Sequence
  Synthetic
agaaauccgucuuucauugacggucuacacccagggcaccggu

SEQ ID NO: 347 - RNA, 43 nt, Artificial Sequence
  Synthetic
agaaauccgucuuucauugacgggaccaucagaggacacuugg

SEQ ID NO: 348 - RNA, 43 nt, Artificial Sequence
  Synthetic
agaaauccgucuuucauugacgguagaugcuguccgaggcagu

SEQ ID NO: 349 - RNA, 43 nt, Artificial Sequence
  Synthetic
agaaauccgucuuucauugacggagaaaggcugcugaugacac

SEQ ID NO: 350 - RNA, 43 nt, Artificial Sequence; identical to SEQ ID NO: 274
  Synthetic

SEQ ID NO: 351 - RNA, 43 nt, Artificial Sequence
  Synthetic
agaaauccgucuuucauugacggggcaacuuacccagaggcaa

SEQ ID NO: 352 - RNA, 43 nt, Artificial Sequence
  Synthetic
agaaauccgucuuucauugacgguuuggcaacuuacccagagg

SEQ ID NO: 353 - RNA, 43 nt, Artificial Sequence
  Synthetic
agaaauccgucuuucauugacggcacaccuuauaggaaaacca

SEQ ID NO: 354 - RNA, 43 nt, Artificial Sequence
  Synthetic
agaaauccgucuuucauugacgguacaaauuccuccucaguug

SEQ ID NO: 355 - RNA, 43 nt, Artificial Sequence
  Synthetic
agaaauccgucuuucauugacggguauaucccuucuacaaauu

SEQ ID NO: 356 - RNA, 43 nt, Artificial Sequence
  Synthetic
agaaauccgucuuucauugacggcaguaagauuuggugucuau

SEQ ID NO: 357 - RNA, 43 nt, Artificial Sequence
  Synthetic
agaaauccgucuuucauugacggccgcccugcugagccccuac

SEQ ID NO: 358 - RNA, 43 nt, Artificial Sequence; identical to SEQ ID NO: 275
  Synthetic

SEQ ID NO: 359 - RNA, 43 nt, Artificial Sequence
  Synthetic
agaaauccgucuuucauugacgggugacgacagccguggugga

SEQ ID NO: 360 - RNA, 43 nt, Artificial Sequence; identical to SEQ ID NO: 276
  Synthetic

SEQ ID NO: 361 - RNA, 43 nt, Artificial Sequence
  Synthetic
agaaauccgucuuucauugacggcuugggauuggugacgacag

SEQ ID NO: 362 - RNA, 43 nt, Artificial Sequence
  Synthetic
agaaauccgucuuucauugacggagguccacuggaggagaagu

SEQ ID NO: 363 - DNA, 20 bp, Artificial Sequence
  Synthetic
ttggcaggatggcttctcat

SEQ ID NO: 364 - DNA, 20 bp, Artificial Sequence
  Synthetic
gcaggatggcttctcatcgt

SEQ ID NO: 365 - DNA, 20 bp, Artificial Sequence
  Synthetic
accatcagaggacacttgga

SEQ ID NO: 366 - DNA, 20 bp, Artificial Sequence
  Synthetic
acaccttataggaaaaccag

SEQ ID NO: 367 - DNA, 20 bp, Artificial Sequence
  Synthetic
taggaaaaccagtgagtctg

SEQ ID NO: 368 - DNA, 20 bp, Artificial Sequence
  Synthetic
gtagaagggatatacaaagt

SEQ ID NO: 369 - DNA, 20 bp, Artificial Sequence
  Synthetic
tagaagggatatacaaagtg

SEQ ID NO: 370 - DNA, 20 bp, Artificial Sequence
  Synthetic
cactttgtatatcccttcta

SEQ ID NO: 371 - DNA, 20 bp, Artificial Sequence
  Synthetic
ccactttgtatatcccttct

SEQ ID NO: 372 - DNA, 20 bp, Artificial Sequence
  Synthetic
gtgtctatttccactttgta

SEQ ID NO: 373 - DNA, 20 bp, Artificial Sequence
  Synthetic
ggtgtctatttccactttgt

SEQ ID NO: 374 - DNA, 20 bp, Artificial Sequence
  Synthetic
tctcataggtggtattcaca

SEQ ID NO: 375 - RNA, 43 nt, Artificial Sequence
  Synthetic
agaaauccgucuuucauugacgguuggcaggauggcuucucau

SEQ ID NO: 376 - RNA, 43 nt, Artificial Sequence
  Synthetic
agaaauccgucuuucauugacgggcaggauggcuucucaucgu

SEQ ID NO: 377 - RNA, 43 nt, Artificial Sequence
  Synthetic
agaaauccgucuuucauugacggaccaucagaggacacuugga

SEQ ID NO: 378 - RNA, 43 nt, Artificial Sequence
  Synthetic
agaaauccgucuuucauugacggacaccuuauaggaaaaccag

SEQ ID NO: 379 - RNA, 43 nt, Artificial Sequence
  Synthetic
agaaauccgucuuucauugacgguaggaaaaccagugagucug

SEQ ID NO: 380 - RNA, 43 nt, Artificial Sequence
  Synthetic
agaaauccgucuuucauugacggguagaagggauauacaaagu

SEQ ID NO: 381 - RNA, 43 nt, Artificial Sequence
  Synthetic
agaaauccgucuuucauugacggcacuuuguauaucccuucua

SEQ ID NO: 382 - RNA, 43 nt, Artificial Sequence
  Synthetic
agaaauccgucuuucauugacggccacuuuguauaucccuucu

SEQ ID NO: 383 - RNA, 43 nt, Artificial Sequence
  Synthetic
agaaauccgucuuucauugacgggugucuauuuccacuuugua

SEQ ID NO: 384 - RNA, 43 nt, Artificial Sequence
  Synthetic
agaaauccgucuuucauugacggggugucuauuuccacuuugu

SEQ ID NO: 385 - RNA, 43 nt, Artificial Sequence
  Synthetic
agaaauccgucuuucauugacggucucauaggugguauucaca

SEQ ID NO: 386 - RNA, 42 nt, Artificial Sequence
  Synthetic
agacauguguccucagugacacucaucgucugcuccuccucu

SEQ ID NO: 387 - RNA, 42 nt, Artificial Sequence
  Synthetic
agacauguguccucagugacacuuggcaggauggcuucucau

SEQ ID NO: 388 - RNA, 42 nt, Artificial Sequence
  Synthetic
agacauguguccucagugacacgcaggauggcuucucaucgu

SEQ ID NO: 389 - RNA, 42 nt, Artificial Sequence
  Synthetic
agacauguguccucagugacacucuacacccagggcaccggu

SEQ ID NO: 390 - RNA, 42 nt, Artificial Sequence
  Synthetic
agacauguguccucagugacacgaccaucagaggacacuugg

SEQ ID NO: 391 - RNA, 42 nt, Artificial Sequence
  Synthetic
agacauguguccucagugacacuagaugcuguccgaggcagu

SEQ ID NO: 392 - RNA, 42 nt, Artificial Sequence
  Synthetic
agacauguguccucagugacacaccaucagaggacacuugga

SEQ ID NO: 393 - RNA, 42 nt, Artificial Sequence
  Synthetic
agacauguguccucagugacaccacaccuuauaggaaaacca

SEQ ID NO: 394 - RNA, 42 nt, Artificial Sequence
  Synthetic
agacauguguccucagugacacuacaaauuccuccucaguug

SEQ ID NO: 395 - RNA, 42 nt, Artificial Sequence
  Synthetic
agacauguguccucagugacacguauaucccuucuacaaauu

SEQ ID NO: 396 - RNA, 42 nt, Artificial Sequence
  Synthetic
agacauguguccucagugacaccaguaagauuuggugucuau

SEQ ID NO: 397 - RNA, 42 nt, Artificial Sequence
  Synthetic
agacauguguccucagugacacacaccuuauaggaaaaccag

SEQ ID NO: 398 - RNA, 42 nt, Artificial Sequence
  Synthetic
agacauguguccucagugacacuaggaaaaccagugagucug

SEQ ID NO: 399 - RNA, 42 nt, Artificial Sequence
  Synthetic
agacauguguccucagugacacguagaagggauauacaaagu

SEQ ID NO: 400 - RNA, 42 nt, Artificial Sequence
  Synthetic
agacauguguccucagugacacuagaagggauauacaaagug

SEQ ID NO: 401 - RNA, 42 nt, Artificial Sequence
  Synthetic
agacauguguccucagugacaccacuuuguauaucccuucua

SEQ ID NO: 402 - RNA, 42 nt, Artificial Sequence
  Synthetic
agacauguguccucagugacacccacuuuguauaucccuucu

SEQ ID NO: 403 - RNA, 42 nt, Artificial Sequence
  Synthetic
agacauguguccucagugacacgugucuauuuccacuuugua

SEQ ID NO: 404 - RNA, 42 nt, Artificial Sequence
  Synthetic
agacauguguccucagugacacggugucuauuuccacuuugu

SEQ ID NO: 405 - RNA, 42 nt, Artificial Sequence
  Synthetic
agacauguguccucagugacaccaccacggcugucgucacca

SEQ ID NO: 406 - RNA, 42 nt, Artificial Sequence
  Synthetic
agacauguguccucagugacacgugacgacagccguggugga

SEQ ID NO: 407 - RNA, 42 nt, Artificial Sequence
  Synthetic
agacauguguccucagugacacggauuggugacgacagccgu

SEQ ID NO: 408 - RNA, 42 nt, Artificial Sequence
  Synthetic
agacauguguccucagugacaccuugggauuggugacgacag

SEQ ID NO: 409 - RNA, 42 nt, Artificial Sequence
  Synthetic
agacauguguccucagugacacucucauaggugguauucaca

SEQ ID NO: 410 - RNA, 20 nt, Artificial Sequence
  Synthetic
gaccaucagaggacacuugg

SEQ ID NO: 411 - RNA, 20 nt, Artificial Sequence
  Synthetic
uagaugcuguccgaggcagu

SEQ ID NO: 412 - RNA, 20 nt, Artificial Sequence
  Synthetic
cugaacacaugcacggccac

SEQ ID NO: 413 - RNA, 20 nt, Artificial Sequence
  Synthetic
ggcaacuuacccagaggcaa

SEQ ID NO: 414 - RNA, 20 nt, Artificial Sequence
  Synthetic
uuuggcaacuuacccagagg

SEQ ID NO: 415 - RNA, 20 nt, Artificial Sequence
  Synthetic
cacaccuuauaggaaaacca

SEQ ID NO: 416 - RNA, 20 nt, Artificial Sequence
  Synthetic
guauaucccuucuacaaauu

SEQ ID NO: 417 - RNA, 20 nt, Artificial Sequence
  Synthetic
caguaagauuuggugucuau

SEQ ID NO: 418 - RNA, 20 nt, Artificial Sequence
  Synthetic
caccacggcugucgucacca

SEQ ID NO: 419 - DNA, 109 bp, Artificial Sequence
  Synthetic
ccgctacaccattgccgccctgctgagcccctactcctattccaccacggctgtcgtcaccaatcccaaggaatgagggacttctcctccagtggacctgaaggacgag

SEQ ID NO: 420 - DNA, 109 bp, Artificial Sequence
  Synthetic
ggcgatgtggtaacggcgggacgactcggggatgaggataaggtggtgccgacagcagtggttagggttccttactccctgaagaggaggtcacctggacttcctgctc

SEQ ID NO: 421 - protein, 35 aa, Artificial Sequence
  Synthetic
RYTIAALLSPYSYSTTAVVTNPKEGTSPPVDLKDE

SEQ ID NO: 422 - DNA, 100 bp, Artificial Sequence
  Synthetic
gtggtaacggcgggacgactcggggatgaggataaggtggtaccgacagcagtggttagggttccttactccctgaagaggaggtcacctggacttcctg

SEQ ID NO: 423 - DNA, 100 bp, Artificial Sequence
  Synthetic
gtggtaacggcgggacgactcggggatgaggataaggtggtaccggcaacaatgattggggttccttactccctgaagaggaggtcacctggacttcctg

SEQ ID NO: 424 - DNA, 100 bp, Artificial Sequence
  Synthetic
gtggtaacggcgggacgactcggggatgaggataaggtggtaccggcaacagtggttagggttccttactccctgaagaggaggtcacctggacttcctg

SEQ ID NO: 425 - DNA, 100 bp, Artificial Sequence
  Synthetic
gtggtaacggcgggacgactcggggatgaggataaggtggtaccgacagcagtgattggggttccttactccctgaagaggaggtcacctggacttcctg

(19) United States
(12) Patent Application Publication
(10) Pub. No.: US 2022/0396782 A1
(43) Pub. Date: Dec. 15, 2022

(54) GENE EDITING SYSTEMS COMPRISING AN RNA GUIDE TARGETING TRANSTHYRETIN (TTR) AND USES THEREOF

(71) Applicant: Arbor Biotechnologies, Inc., Cambridge, MA (US)

(72) Inventors: Quinton Norman WESSELLS, Cambridge, MA (US); Jeffrey Raymond HASWELL, Needham, MA (US); Tia Marie DITOMMASO, Newton, MA (US); Noah Michael JAKIMO, San Francisco, CA (US); Sejuti SENGUPTA, Ashland, MA (US)

(21) Appl. No.: 17/831,852

(22) Filed: Jun. 3, 2022

Related U.S. Application Data
(60) Provisional application No. 63/197,036, filed on Jun. 4, 2021.
(60) Provisional application No. 63/247,080, filed on Sep. 22, 2021.
(60) Provisional application No. 63/321,960, filed on Mar. 21, 2022.

(51) Int. Cl. C12N 9/22; C12N 15/11; C12N 15/86; A61K 38/46; A61K 31/7105; C12N 15/90

(52) U.S. Cl. CPC C12N 9/22; C12N 15/11; C12N 15/86; A61K 38/465; A61K 31/7105; C12N 15/907; C12N 2310/20; C12N 2800/80; C12N 2750/14343; A61K 48/00

(57) ABSTRACT

A system for genetic editing of a transthyretin (TTR) gene, comprising (i) a Cas12i2 polypeptide or a first nucleic acid encoding the Cas12i2 polypeptide, and (ii) an RNA guide or a second nucleic acid encoding the RNA guide, wherein the RNA guide comprises a spacer sequence specific to a target sequence within an TTR gene. Also provided herein are methods for editing a TTR gene using the gene editing system disclosed herein and/or for treating diseases associated with the TTR gene.

### CROSS REFERENCE TO RELATED APPLICATIONS

This application claims the benefit under 35 U.S.C. § 119(e) of U.S. Provisional Application No. 63/197,036, filed Jun. 4, 2021, U.S. Provisional Application No. 63/247,080, filed Sep. 22, 2021, and U.S. Provisional Application No. 63/321,960, filed Mar. 21, 2022, the contents of each of which are incorporated by reference herein in their entirety.

### SEQUENCE LISTING

The instant application contains a Sequence Listing which has been filed electronically in ASCII format and is hereby incorporated by reference in its entirety. Said ASCII copy, created on Jun. 3, 2022, is named 116928-0033-0002US00_SEQ.txt and is 203,161 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE PRESENT DISCLOSURE

The present disclosure is based, at least in part, on the development of a system for genetic editing of a transthyretin (TTR) gene. The system involves a Cas12i CRISPR nuclease polypeptide (e.g., a Cas12i2 polypeptide or a Cas12i4 polypeptide) and a guide RNA (gRNA) mediating cleavage at a genetic site within a TTR gene by the Cas12i CRISPR nuclease polypeptide. The system may further involve a template nucleic acid, which may be incorporated at a genetic site within the TTR gene via homologous recombination, leading to introduction of one or more modifications into the TTR gene at the genetic site. As reported herein, the gene editing system disclosed herein has achieved successful editing of the TTR gene with high editing efficiency and accuracy.

Without being bound by theory, the gene editing system disclosed herein may further exhibit one or more of the following advantageous features. Compared to SpCas9 and Cas12a, Cas12i effectors are smaller (1033 to 1093aa), which, in conjunction with their short mature crRNA (40-43 nt), is preferable in terms of delivery and cost of synthesis. Cas12i cleavage results in larger deletions compared to the small deletions and +1 insertions induced by Cas9 cleavage. Cas12i PAM sequences also differ from those of Cas9. Therefore, larger and different portions of genetic sites of interest can be disrupted with a Cas12i polypeptide and RNA guide compared to Cas9. Using an unbiased approach of tagmentation-based tag integration site sequencing (TTISS), more potential off-target sites with a higher number of unique integration events were identified for SpCas9 compared to Cas12i2. See WO/2021/202800. Therefore, Cas12i2 may be more specific than Cas9.

Accordingly, provided herein are gene editing systems for editing a TTR gene, pharmaceutical compositions or kits comprising such, methods of using the gene editing systems to produce genetically modified cells, and the resultant cells thus produced. Also provided herein are uses of the gene editing systems disclosed herein, the pharmaceutical compositions and kits comprising such, and/or the genetically modified cells thus produced for treating amyloidogenic transthyretin (ATTR) in a subject.

In some aspects, the present disclosure features a gene editing system for genetic editing of a transthyretin (TTR) gene, comprising (i) a Cas12i polypeptide or a first nucleic acid encoding the Cas1212 polypeptide, and (ii) an RNA guide or a second nucleic acid encoding the RNA guide. The RNA guide comprises a spacer sequence specific to a target sequence within a TTR gene, the target sequence being adjacent to a protospacer adjacent motif (PAM) comprising the motif of 5′-TIN-3′, which is located 5′ to the target sequence.

In some embodiments, the Cas12i polypeptide can be a Cas12i2 polypeptide. In other embodiments, the Cas12i polypeptide can be a Cas12i4 polypeptide.

In some embodiments, the Cas12i polypeptide is a Cas12i2 polypeptide, which comprises an amino acid sequence at least 95% identical to SEQ ID NO: 222 and comprises one or more mutations relative to SEQ ID NO: 222. In some embodiments, the one or more mutations in the Cas1212 polypeptide are at positions D581, G624, F626, P868, I926, V1030, E1035, and/or S1046 of SEQ ID NO: 222. For example, the one or more mutations are amino acid substitutions, e.g., D581R, G624R, F626R, P868T, I926R, V1030G, E1035R, S10460, or a combination thereof.

In one example, the Cas12i2 polypeptide comprises mutations at positions D581, D911, I926, and V1030 (e.g., amino acid substitutions of D581R, D91 IR, I926R, and V1030G). In another example, the Cas12i2 polypeptide comprises mutations at positions D581, I926, and V1030 (e.g., amino acid substitutions of D581R, I926R, and V1030G). In yet another example, the Cas12i2 polypeptide comprises mutations at positions D581, I926, V1030, and S1046 (e.g., amino acid substitutions of D581R, I926R, V1030G, and S1046G). In still another example, the Cas12i2 polypeptide comprises mutations at positions D581, G624, F626, I926, V1030, E1035, and S1046 (e.g., amino acid substitutions of D581R, G624R, F626R, I926R, V10300, E1035R, and S1046G). In another example, the Cas12i2 polypeptide comprises mutations at positions D581, G624, F626, P868, I926, V1030, E1035, and S1046 (e.g., amino acid substitutions of D581R. G624R, F626R, P868T, I926R, V10300, E1035R, and S1046G).

Exemplary Cas12i2 polypeptides for use in any of the gene editing systems disclosed herein may comprise the amino acid sequence of any one of SEQ ID NOs: 223-227. In one example, the exemplary Cas12i2 polypeptide for use in any of the gene editing systems disclosed herein comprises the amino acid sequence of SEQ ID NO: 224. In another example, the exemplary Cas12i2 polypeptide for use in any of the gene editing systems disclosed herein comprises the amino acid sequence of SEQ ID NO: 227.

In some embodiments, the gene editing system may comprise the first nucleic acid encoding the Cas12i polypeptide (e.g., the Cas12i2 polypeptide). In some instances, the first nucleic acid is located in a first vector (e.g., a viral vector such as an adeno-associated viral vector or AAV vector). In some instances, the first nucleic acid is a messenger RNA (mRNA). In some instances, the coding sequence for the Cas12i polypeptide is codon optimized.

In some embodiments, the target sequence is within exon 2, exon 3, or exon 4 of the TTR gene. In some examples, the target sequence comprises: (i) GACCATCAGAGGACACTTGG (SEQ ID NO: 329), (ii) TAGATGCTGTCCGAGGCAGT (SEQ ID NO: 330), (iii) CTGAACACATGCACGGCCAC (SEQ ID NO: 332), (iv) GGCAACTTACCCAGAGGCAA (SEQ ID NO: 333), (v) TITGGCAACTTACCCAGAGG (SEQ ID NO: 334), (vi) CACACCTTATAGGAAAACCA (SEQ ID NO: 335), (vii) GTATATCCCTTCTACAAATT (SEQ ID NO: 337), (viii) CAGTAAGATITGGTGTCTAT (SEQ ID NO: 338), or (ix) CACCACGGCTGTCGTCACCA (SEQ ID NO: 271).

In some embodiments, the spacer sequence may be 20-30-nucleotide in length. In some examples, the spacer sequence is 20-nucleotide in length. In some examples, the spacer sequence comprises: (i) GACCAUCAGAGGACACUUGG (SEQ ID NO: 410), (ii) UAGAUGCUGUCCGAGGCAGU (SEQ ID NO: 411), (iii) CUGAACACAUGCACGGCCAC (SEQ ID NO: 412), (iv) GGCAACUUACCCAGAGGCAA (SEQ ID NO: 413), (v) UUUGGCAACUUACCCAGAGG (SEQ ID NO: 414), (vi) CACACCUUAUAGGAAAACCA (SEQ ID NO: 415), (vii) GUAUAUCCCUUCUACAAAUU (SEQ ID NO: 416), (viii) CAGUAAGAUUUGGUGUCUAU (SEQ ID NO: 417), or (ix) CACCACGGCUGUCGUCACCA (SEQ ID NO: 418).

In some embodiments, the RNA guide (gRNA) comprises the spacer and a direct repeat sequence. In some examples, the direct repeat sequence is 23-36-nucleotide in length. In one example, the direct repeat sequence is at least 90% identical to any one of SEQ ID NOs: 1-10 or a fragment thereof that is at least 23-nucleotide in length. In some specific examples, the direct repeat sequence is any one of SEQ ID NOs: 1-10, or a fragment thereof that is at least 23-nucleotide in length. By way of non-limiting example, the direct repeat sequence is 5′-AGAAAUCCGUCUUUCAUUGACGG-3′ (SEQ ID NO: 10).

In specific examples, the RNA guide comprises the nucleotide sequence of: (i) AGAAAUCCGUCUUUCAUUGACGGGACCAUCAGAGGACACUUGG (SEQ ID NO: 347), (ii) AGAAAUCCGUCUUUCAUUGACGGUAGAUGCUGUCCGAGGCAGU (SEQ ID NO: 348), (iii) AGAAAUCCGUCUUUCAUUGACGGCUGAACACAUGCACGGCCAC (SEQ ID NO: 350), (iv) AGAAAUCCGUCUUUCAUUGACGGGGCAACUUACCCAGAGGCAA (SEQ ID NO: 351), (v) AGAAAUCCGUCUUUCAUUGACGGUUUGGCAACUUACCCAGAGG (SEQ ID NO: 352), (vi) AGAAAUCCGUCUUUCAUUGACGGCACACCUUAUAGGAAAAC CA (SEQ ID NO: 353), (vii) AGAAAUCCGUCUUUCAUUGACGGGUAUAUCCCUUCUAC AAAULU (SEQ ID NO: 355), (viii) AGAAAUCCGUCUUUCAUUGACGGCAGUAAGAUUU GGUGUCUAU (SEQ ID NO: 356), or (ix) AGAAAUCCGUCUUUCAUUGACGGCACCACG GCUGUCGUCACCA (SEQ ID NO: 358). In some examples, the RNA guide may contain one or more modifications, for example, comprising the modified nucleotide sequence of SEQ ID NO: 277.

In some embodiments, the system may comprise the second nucleic acid encoding the RNA guide. In some examples, the nucleic acid encoding the RNA guide may be located in a viral vector. In some examples, the viral vector comprises the both the first nucleic acid encoding the Cas12i polypeptide (e.g., the Cas12i2 polypeptide) and the second nucleic acid encoding the RNA guide.

In some embodiments, any of the systems described herein may comprise the first nucleic acid encoding the Cas12i polypeptide (e.g., the Cas12i2 polypeptide), which is located in a first vector, and the second nucleic acid encoding the RNA guide, which is located on a second vector. In some examples, the first and/or second vector is a viral vector. In some specific examples, the first and second vectors are the same vector. In other examples, the first and second vectors are different vectors.

Any of the gene editing systems disclosed herein may further comprise (iii) a template DNA, which comprising (a) a first segment homologous to a first site in the TTR gene that is upstream to a TTR gene target site for genetic editing, (b) a second segment homologous to a second site that is downstream to the TTR gene target site for genetic editing, and (c) a donor region, which is homologous to the TTR gene target site for genetic editing and comprises at least one nucleotide variation relative to the TTR gene target site for genetic editing; and wherein the donor region is flanked by the first and second segments. In some embodiments, the TTR gene target site for genetic editing comprises the target sequence, the PAM, or a combination thereof. The templated DNA is located in a viral vector, for example, an AAV vector.

In some embodiments, the TTR gene target site for genetic editing comprises a mutation associated with a disease, and wherein the donor region comprises a sequence that fixes the mutation. Exemplary mutations associated with the disease may lead to the amino acid residue substitution of V30M, V122I, T60A, L58H, or 184S relative to the TTR sequence of SEQ ID NO: 257.

In other embodiments, the donor region comprises a protective mutation relative to the TTR gene target site for genetic editing. For example, the protective mutation leads to the amino acid residue substitution of T119M relative to the TTR sequence of SEQ ID NO: 257.

In some embodiments, the gene editing system may comprise one or more lipid nanoparticles (LNPs), which encompass (i), (ii), or both, and optionally (iii). In some examples, the gene editing system may comprise the LNP, which encompass (i), and wherein the system comprises a viral vector comprising the second nucleic acid encoding the RNA guide; optionally wherein the viral vector is an AAV vector. In other examples, the gene editing system may comprise the LNP, which encompass (ii), and wherein the system comprises a viral vector comprising the first nucleic acid encoding Cas12i polypeptide (e.g., the Cas12i2 polypeptide); optionally wherein the viral vector is an AAV vector.

In other aspects, the present disclosure provides a gene editing system for genetic editing of a transthyretin (TTR) gene, comprising (i) a Cas12i polypeptide (e.g., a Cas12i2 or a Cas12i4 polypeptide) or a first nucleic acid encoding the Cas12i polypeptide, (ii) an RNA guide or a second nucleic acid encoding the RNA guide. The RNA guide may comprise a spacer sequence specific to a target sequence within exon 2, exon 3, or exon 4 of a TTR gene.

The target sequence being adjacent to a protospacer adjacent motif (PAM) comprising the motif of 5′-TTN-3′, which is located 5′ to the target sequence. Any of the target sequences and/or spacer sequences can be used in this gene editing system.

In some embodiments, provided herein is a gene editing system for genetic editing of a transthyretin (TTR) gene, comprising (i) a Cas12i polypeptide (e.g., a Cas12i2 polypeptide or a Cas12i4 polypeptide) or a first nucleic acid encoding the Cas12i polypeptide as disclosed herein, (ii) an RNA guide or a second nucleic acid encoding the RNA guide as disclosed herein, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence; and (iii) a template DNA as disclosed herein, which comprising (a) a first segment homologous to a first site in the TTR gene that is upstream to a TTR gene target site for genetic editing, (b) a second segment homologous to a second site that is downstream to the TTR gene target site for genetic editing, and (c) a donor region, which is homologous to the TTR gene target site for genetic editing and comprises at least one nucleotide variation relative to the TTR gene target site for genetic editing; and wherein the donor region is flanked by the first and second segments. In some examples, the donor region in the template DNA comprises a sequence that fixes a mutation in the TTR gene target site associated with a disease, comprises a protective mutation, or a combination thereof.

In some aspects, the present disclosure also provides a pharmaceutical composition comprising any of the gene editing systems disclosed herein, or a kit comprising the components of the gene editing system.

In some aspects, the present disclosure provides a method for editing a transthyretin (TTR) gene in a cell, the method comprising contacting a host cell with any of the gene editing systems disclosed herein for editing the TTR gene to genetically edit the TTR gene in the host cell. In some instances, the host cell is cultured in vitro. In other instances, the contacting step is performed by administering the system for editing the TTR gene to a subject comprising the host cell.

Further, provided herein is a cell comprising a mutated transthyretin (TTR) gene, wherein the cell optionally is produced by contacting a host cell with the gene editing system disclosed herein to genetically edit the TTR gene in the host cell, thereby mutating the TTR gene. In some instances, the cell comprises a disrupted TTR gene. In other instances, the cell comprises a modified TTR gene, which expresses a mutated TTR relative to a wild-type counterpart cell.

Further, the present disclosure features a method for treating amyloidogenic transthyretin (ATTR) in a subject, comprising administering to a subject in need thereof any of the gene editing systems disclosed herein for editing a transthyretin (TTR) gene or the modified cell produced by the gene editing system. In some embodiments, the subject is a human patient having hereditary ATTR (hATTR) or wild-type ATTR amyloidosis.

Also provided herein are any of the gene editing systems disclosed herein, pharmaceutical compositions or kits comprising such, or genetically modified cells generated by the gene editing system for use in treating ATTR in a subject, as well as uses of the gene editing systems disclosed herein, pharmaceutical compositions or kits comprising such, or genetically modified cells generated by the gene editing system for manufacturing a medicament for treatment of ATTR in a subject.

In addition, the present disclosure provides an RNA guide, comprising (i) a spacer sequence that is specific to a target sequence in a transthyretin (TTR) gene, wherein the target sequence is adjacent to a protospacer adjacent motif (PAM) comprising the motif of 5′-TTN-3′, which is located 5′ to the target sequence; and (ii) a direct repeat sequence. In some embodiments, the spacer sequence is 20-30-nucleotide in length, optionally 20-nucleotide in length. Alternatively or in addition, the direct repeat sequence is 23-36-nucleotide in length, optionally 23-nucleotide in length.

In some embodiments, the target sequence is within exon 2, exon 3, or exon 4 of the TTR gene. In some examples, the target sequence comprises the nucleotide sequence of SEQ ID NO: 329, 330, 332-335, 337, 338, or 271. In some examples, the spacer sequence is set forth as any one of SEQ ID NOs: 410-418.

In some embodiments, the direct repeat sequence in any of the RNA guides disclosed herein is at least 90% identical to any one of SEQ ID NOs: 1-10 or a fragment thereof that is at least 23-nucleotide in length. In some examples, the direct repeat sequence is any one of SEQ ID NOs: 1-10, or a fragment thereof that is at least 23-nucleotide in length. In one example, the direct repeat sequence is SEQ ID NO: 10. In specific examples, the RNA guide may comprise a nucleotide sequence of any one of SEQ ID NOs: 347, 348, 350-353, 355, 356, or 358. In some instances, the RNA guide may comprise one or more modifications. In one example, the RNA guide is a modified RNA molecule comprising SEQ ID NO: 277.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 shows % total edits observed within and/or adjacent to TTR target sequences using variant Cas12i2, as described in Example 1.

FIG. 2 is a schematic showing a Cas12i TTR target sequence and exemplary template DNA sequences. The template DNA sequences comprise 40-nucleotide homology arms and a donor region to introduce the TI 19M mutation into the target sequence. Sequences, from top to bottom, are SEQ ID NOs: 419-425.

FIG. 3 shows total edits (medium greys), partial integration of the V30M SNP (light grey), and full integration of the insert region of template DNAs (black) into TTR target sites. Template DNAs comprising 40-nucleotide homology arms and 50-nucleotide homology arms were tested and editing efficiency of variant Cas12i2 was measured.

FIG. 4 shows total edits (medium greys), partial integration of the V122I SNP (light grey), and full integration of the insert region of template DNAs (black) into TTR target sites. Template DNAs comprising 40-nucleotide homology arms and 50-nucleotide homology arms were tested and editing efficiency of variant Cas12i2 was measured.

FIG. 5 shows total edits (medium greys), partial integration of the T119M SNP (light grey), and full integration of the insert region of template DNAs (black) into TTR target sites. Template DNAs comprising 40-nucleotide homology arms and 50-nucleotide homology arms were tested and editing efficiency of variant Cas12i2 was measured.

FIG. 6 is a graph showing indels in TTR target sites following variant Cas12i2 RNP delivery to HEK293T cells.

FIG. 7 is a graph showing indels in TTR target sites following variant Cas12i2 RNP delivery to HepG2 cells.

FIGS. 8A and 8B include diagrams showing gene editing efficiency at TTR target sites using variant Cas12i2/guide RNA RNPs. FIG. 8A is a graph showing indels in TTR target sites following variant Cas12i2 RNP delivery to primary human hepatocytes after 3 days (darker bars) and after 7 days (lighter bars). FIG. 8B is a plot showing indels in TTR target sites on the x-axis and TTR protein knockdown on the y-axis following variant Cas12i2 RNP delivery to primary human hepatocytes. The black datapoints represent target sequences with perfect homology to non-human primate TTR sequences. The grey datapoints represent target sequences without perfect homology to non-human primate TTR sequences.

FIGS. 9A and 9B include diagrams showing editing efficiency at TTR targeting sites using TTR-targeting crRNAs and the Cas12i2 variant as indicated. FIG. 9A is a graph showing % indels induced by TTR-targeting crRNAs and the Cas12i2 variant of SEQ ID NO: 224, the Cas12i2 variant of SEQ ID NO: 227, or the Cas12i4 variant of SEQ ID NO: 255. FIG. 9B shows the size (left) and start position (right) of indels induced in HepG2 cells by the variant Cas12i2 of SEQ ID NO: 224 and the TTR-targeting RNA guide of E2T3 (SEQ ID NO: 348).

FIGS. 10A and 10B show plots depicting tagmentation-based tag integration site sequencing (TTISS) reads for variant Cas12i2 of SEQ ID NO: 224 and TTR-targeting RNA guides. FIG. 10A shows TTISS plots for E3T1 (SEQ ID NO: 353), E2T2 (SEQ ID NO: 347), E3T4 (SEQ ID NO: 356), E3T3 (SEQ ID NO: 355), and E4T2 (SEQ ID NO: 358). FIG. 10B shows TTISS plots for E1T2 (SEQ ID NO: 363) and E1T1 (SEQ ID NO: 345). The black wedge and centered number represent the fraction of on-target TTISS reads. Each gray wedge represents a unique off-target site identified by TTISS. The size of each gray wedge represents the fraction of TTISS reads mapping to a given off-target.

### DETAILED DESCRIPTION

The present disclosure relates to a system for genetic editing of a transthyretin (TTR) gene, which comprises (i) a Cas12i polypeptide or a first nucleic acid encoding the Cas12i2 polypeptide; and (ii) an RNA guide or a second nucleic acid encoding the RNA guide, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence. Also provided in the present disclosure are a pharmaceutical composition or a kit comprising such system as well as uses thereof. Further disclosed herein are a method for editing a TTR gene in a cell, a cell so produced that comprises a disrupted a TTR gene, a method of treating amyloidogenic transthyretin (ATTR) in a subject, and an RNA guide that comprises (i) a spacer sequence that is specific to a target sequence in a TTR gene, wherein the target sequence is adjacent to a potospacer adjacent motif (PAM) comprising the motif of 5′-TTN-3′, which is located 5′ to the target sequence; and (ii) a direct repeat sequence, as well as uses thereof.

The Cas12i polypeptide for use in the gene editing system disclosed herein may be a Cas12i2 polypeptide, e.g., a wild-type Cas12i polypeptide or a variant thereof as those disclosed herein. In some examples, the Cas12i2 polypeptide comprises an amino acid sequence at least 95% identical to SEQ ID NO: 222 and comprises one or more mutations relative to SEQ ID NO: 222. In other examples, the Cas12i polypeptide may be a Cas12i4 polypeptide, which is also disclosed herein.

### Definitions

The present disclosure will be described with respect to particular embodiments and with reference to certain Figures, but the present disclosure is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of a Cas12i polypeptide. For example, activity can include nuclease activity.

As used herein the term “TTR” refers to “transthyretin.” TTR is a transport protein in the serum and cerebrospinal fluid that carries the thyroid hormone thyroxin (T4) and retinol-binding protein bound to retinol (vitamin A). TTR misfolding and aggregation is associated with amyloid disease, senile systemic amyloidosis, familial amyloid polyneuropathy, and familial amyloid cardiomyopathy. SEQ ID NO: 228 as set forth herein provides an example of a TTR gene sequence. SEQ ID NO: 258 set forth herein provides an example of a TTR coding sequence.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference for the subject matter and purpose referenced herein. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 8, 2, 11, and 9 of the present application. In some embodiments, a Cas12i polypeptide of the disclosure is a Cas12i2 polypeptide as described in WO/2021/202800, the relevant disclosures of which are incorporated by reference for the subject matter and purpose referenced herein. In some embodiments, the Cas12i polypeptide cleaves a target nucleic acid (e.g., as a nick or a double strand break).

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides. 6-12 nucleotides. 8-10 nucleotides. 8-12 nucleotides, 10-12 nucleotides, 10-15 nucleotides, or 12-15 nucleotides.

As used herein, the term “complex” refers to a grouping of two or more molecules. In some embodiments, the complex comprises a polypeptide and a nucleic acid molecule interacting with (e.g., binding to, coming into contact with, adhering to) one another. For example, the term “complex” can refer to a grouping of an RNA guide and a polypeptide (e.g., a Cas12i polypeptide). Alternatively, the term “complex” can refer to a grouping of an RNA guide, a polypeptide, and the complementary region of a target sequence. As used herein, the term “complex” can refer to a grouping of a TTR-targeting RNA guide and a Cas12i polypeptide.

As used herein, the term “homology arm” refers to a portion of a template nucleic acid that has homology to a target nucleic acid. A template nucleic acid, in some embodiments, may comprise one or two homology arms. In some embodiments, the homology arm has substantial identity or perfect identity to the corresponding region of the target nucleic acid. In some embodiments, the homology arm is single stranded DNA or double stranded DNA. In some embodiments, use of a template nucleic acid comprising a homology arm results in a greater level of incorporation of a sequence difference into a target nucleic acid compared to use of an otherwise similar template nucleic acid that lacks the homology arm.

As used herein, the term “left homology arm” refers to a homology arm situated such that: i) a plurality of nucleotides of the homology arm are homologous to a portion of the target site that is between the cut site and the PAM-proximal end of the target site, ii) a plurality of nucleotides of the homology arm are homologous to a portion of the target nucleic acid that is on the opposite side of the PAM-distal end of the target site from the cut site, or both of i) and ii).

As used herein, the term “right homology arm” refers to a homology arm situated such that: i) a plurality of nucleotides of the homology arm are homologous to portion of the target site that is between the cut site and the PAM-distal end of the target site, ii) a plurality of nucleotides of the homology arm are homologous to a portion of the target nucleic acid that is on the opposite side of the cut site from the PAM-proximal end of the target site, or both of i) and ii).

As used herein, the term “protospacer adjacent motif” or “PAM” refers to a DNA sequence adjacent to a target sequence (e.g., a TTR target sequence) to which a complex comprising an RNA guide (e.g., a TTR-targeting RNA guide) and a Cas12i polypeptide binds. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein.

In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein). In some embodiments, the target sequence is a sequence within a TTR gene sequence, including, but not limited, to the sequence set forth in SEQ ID NO: 228 or SEQ ID NO: 258.

As used herein, the term “spacer” or “spacer sequence” is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “RNA guide” or “RNA guide sequence” refers to any RNA molecule or a modified RNA molecule that facilitates the targeting of a polypeptide (e.g., a Cas12i polypeptide) described herein to a target sequence (e.g., a sequence of a TTR gene). For example, an RNA guide can be a molecule that is designed to be complementary to a specific nucleic acid sequence (a target sequence such as a target sequence with a TTR gene). An RNA guide may comprise a spacer sequence and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA and mature crRNA are also used herein to refer to an RNA guide.

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=l00, wordlength-12 to obtain nucleotide sequences homologous to the nucleic acid molecules of the present disclosure. BLAST protein searches can be performed with the XBLAST program, score=50, word length=3 to obtain amino acid sequences homologous to the protein molecules of the present disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the terms “template nucleic acid,” “template DNA,” “donor,” “donor nucleic acid,” refer to a nucleic acid molecule comprising a nucleic acid sequence which can be used in conjunction with a Cas12i polypeptide and an RNA guide to modify a target nucleic acid (e.g., a TTR gene target site) to have the some or all of the sequence of the template nucleic acid. In some embodiments, the modification of the target nucleic acid sequence is at or near a cleavage site(s). In some embodiments, the template nucleic acid is a single-stranded nucleic acid. In some embodiments, the template nucleic acid is a double-stranded nucleic acid. In some embodiments, the template DNA directs modification of the target nucleic acid using HDR.

As used herein, the term “edit” refers to one or more modifications introduced into a target nucleic acid, e.g., within the HAO1 gene. The edit can be one or more substitutions, one or more insertions, one or more deletions, or a combination thereof. As used herein, the term “substitution” refers to a replacement of a nucleotide or nucleotides with a different nucleotide or nucleotides, relative to a reference sequence. As used herein, the term “insertion” refers to a gain of a nucleotide or nucleotides in a nucleic acid sequence, relative to a reference sequence. As used herein, the term “deletion” refers to a loss of a nucleotide or nucleotides in a nucleic acid sequence, relative to a reference sequence.

No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to a deletion of up to several kilobases.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ or 5′-TTN-3′ sequence is upstream of an indel described herein, and a Cas12i-induced indel is downstream of the 5′-NTTN-3′ or 5′-TTN-3′ sequence.

### I. Gene Editing Systems

In some aspects, the present disclosure provides gene editing systems comprising an RNA guide targeting a TTR gene. Such a gene editing system can be used to edit the TTR target gene, e.g., to disrupt the TTR gene.

Transthyretin “TTR” is a transport protein in the serum and cerebrospinal fluid that carries the thyroid hormone thyroxin (T4) and vitamin A. TTR misfolding and aggregation is associated with amyloid disease, senile systemic amyloidosis, familial amyloid polyneuropathy, and familial amyloid cardiomyopathy. Accordingly, the gene editing systems disclosed here, targeting the TTR gene, could be used to treat amyloid diseases in a subject in need of the treatment.

In some embodiments, the RNA guide is comprised of a direct repeat component and a spacer sequence. In some embodiments, the RNA guide binds a Cas12i polypeptide. In some embodiments, the spacer sequence is specific to a TTR target sequence, wherein the TTR target sequence is adjacent to a 5′-NTTN-3′ or 5′-T′N-3′ PAM sequence as described herein. In the case of a double-stranded target, the RNA guide binds to a first strand of the target (i.e., the non-PAM strand) and a PAM sequence as described herein is present in the second, complementary strand (i.e., the PAM strand).

In some embodiments, the present disclosure provides compositions comprising a complex, wherein the complex comprises an RNA guide targeting a TTR. In some embodiments, the present disclosure comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to the complementary region of a target sequence within a TTR gene. In some embodiments, a complex comprising an RNA guide targeting a TTR and a Cas12i polypeptide binds to the complementary region of a target sequence within the TTR gene at a molar ratio of about 1:1. In some embodiments, the complex comprises enzymatic activity, such as nuclease activity, that can cleave the TTR target sequence and/or the complementary sequence. The RNA guide, the Cas12i polypeptide, and the complementary region of the TTR target sequence, either alone or together, do not naturally occur. In some embodiments, the RNA guide in the complex comprises a direct repeat and/or a spacer sequence described herein. In some embodiments, the sequence of the RNA guide has at least 90% identity (e.g., at least 90%, 91%, 92%, 93%, 94%, 95%, 96%, 97%, 98%, or 99% identity) to a sequence of any one of SEQ ID NOs: 345-362. In some embodiments, the RNA guide has a sequence of any one of SEQ ID NOs: 345-362.

In some embodiments, the present disclosure comprises compositions comprising an RNA guide as described herein and/or an RNA encoding a Cas12i polypeptide as described herein. In some embodiments, the RNA guide and the RNA encoding a Cas12i polypeptide are comprised together within the same composition. In some embodiments, the RNA guide and the RNA encoding a Cas12i polypeptide are comprised within separate compositions. In some embodiments, the RNA guide comprises a direct repeat and/or a spacer sequence described herein. In some embodiments, the sequence of the RNA guide has at least 90% identity (e.g., at least 90%, 91%, 92%, 93%, 94%, 95%, 96%, 97%, 98%, or 99% identity) to a sequence of any one of SEQ ID NOs: 345-362. In some embodiments, the RNA guide has a sequence of any one of SEQ ID NOs: 345-362.

In some embodiments, the compositions of the present disclosure further comprise a template DNA. In some embodiments, the compositions comprise a Cas12i polypeptide, an RNA guide, and a template DNA. In some embodiments, the template DNA comprises a homology arm (e.g., a left homology arm and/or a right homology arm). In some embodiments, the homology arm has at least 80%, 85%, 90%, 95%, 99%, or 100% identity to the corresponding region of the target nucleic acid (e.g., the target strand or non-target strand of the target nucleic acid). In some embodiments, the template DNA comprises a sequence difference relative to a target nucleic acid sequence comprising a TlR target sequence. In some embodiments, the sequence difference is a polymorphism. In some embodiments, the polymorphism is a mutation associated with a disease (e.g., hereditary ATIR amyloidosis (hATTR), familial amyloid polyneuropathy (FAP), or senile systemic amyloidosis (SSA)). In some embodiments, the sequence difference is a protective mutation. In some embodiments, through homology directed repair (HDR) using the template DNA, the mutation associated with disease is corrected (e.g., the mutant TTR target sequence is corrected to be the wild-type TTR sequence). In some embodiments, through HDR and using template DNA, a protective mutation is introduced into the mutant TTR target sequence. In some embodiments, through HDR using the template DNA, a mutation-associated disease is corrected, and a protective mutation is introduced into the TTR target sequence.

Use of the gene editing systems disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1,368 amino acids in length, S. thermophilus Cas9 (StCas9) is 1,128 amino acids in length. FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See, WO/2021/202800, the relevant disclosures of which are incorporated by reference for the subject matter and purpose referenced herein. Furthermore, indels induced by compositions comprising a Cas12i polypeptide differ from indels induced by compositions comprising an SpCas9 polypeptide. For example, SpCas9 polypeptides primarily induce insertions and deletions of 1 nucleotide in length. However, Cas12i polypeptides induce larger deletions, which can be beneficial in disrupting a larger portion of a gene such as TTR.

Also provided herein is a system for genetic editing of a TTR gene, which comprises (i) a Cas12i polypeptide (e.g., a Cas12i2 polypeptide) or a first nucleic acid encoding the Cas12i polypeptide (e.g., a Cas12i2 polypeptide comprises an amino acid sequence at least 95% identical to SEQ ID NO: 222, which may and comprises one or more mutations relative to SEQ ID NO: 222); and (ii) an RNA guide or a second nucleic acid encoding the RNA guide, wherein the RNA guide comprises a spacer sequence specific to a target sequence within the TTR gene (e.g., within exon 2, exon 3, or exon 4 of the TTR gene), the target sequence being adjacent to a protospacer adjacent motif (PAM) comprising the motif of 5′-TTN-3′ (5′-NTTN-3′), which is located 5′ to the target sequence.

A. RNA Guides

In some embodiments, the gene editing system described herein comprises an RNA guide targeting a TTR gene, for example, targeting exon 2, exon 3, or exon 4 of the TTR gene. In some embodiments, the gene editing system described herein may comprise two or more (e.g., 23, 4, 5, 6, 7, 8, 9, or more) RNA guides targeting TTR.

The RNA guide may direct the Cas12i polypeptide contained in the gene editing system as described herein to an HAO1 target sequence. Two or more RNA guides may direct two or more separate Cas12i polypeptides (e.g., Cas12i polypeptides having the same or different sequence) as described herein to two or more (e.g., 2, 3, 4, 5, 6, 7, 8, 9, or more) TTR target sequences. Those skilled in the art reading the below examples of particular kinds of RNA guides will understand that, in some embodiments, an RNA guide is TTR target-specific. That is, in some embodiments, an RNA guide binds specifically to one or more TTR target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

(i). Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some embodiments, the direct repeat sequence is a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8. The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or 8. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8. The direct repeat sequence can comprise nucleotide 1 through nucleotide 34 of SEQ ID NO: 9. The direct repeat sequence can comprise nucleotide 2 through nucleotide 34 of SEQ 1D NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ 1D NO: 10.

In some embodiments, the direct repeat sequence has at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have at least 90% identity to a sequence comprising nucleotide 1 through nucleotide 36 of any one of SEQ ID NOs: 1, 2, 3, 4, 5, 6, 7, or 8. The direct repeat sequence can have at least 90% identity to a sequence comprising 2 through nucleotide 36 of any one of SEQ ID NOs: 1, 2, 3, 4, 5, 6, 7, or 8. The direct repeat sequence can have at least 90% identity to a sequence comprising 3 through nucleotide 36 of any one of SEQ ID NOs: 1, 2, 3, 4, 5, 6, 7, or 8. The direct repeat sequence can have at least 90% identity to a sequence comprising 4 through nucleotide 36 of any one of SEQ ID NOs: 1, 2, 3, 4, 5, 6, 7, or 8. The direct repeat sequence can have at least 90% identity to a sequence comprising 5 through nucleotide 36 of any one of SEQ ID NOs: 1, 2, 3, 4, 5, 6, 7, or 8. The direct repeat sequence can have at least 90% identity to a sequence comprising 6 through nucleotide 36 of any one of SEQ ID NOs: 1, 2, 3, 4, 5, 6, 7, or 8. The direct repeat sequence can have at least 90% identity to a sequence comprising 7 through nucleotide 36 of any one of SEQ ID NOs: 1, 2, 3, 4, 5, 6, 7, or 8. The direct repeat sequence can have at least 90% identity to a sequence comprising 8 through nucleotide 36 of any one of SEQ ID NOs: 1, 2, 3, 4, 5, 6, 7, or 8. The direct repeat sequence can have at least 90% identity to a sequence comprising 9 through nucleotide 36 of any one of SEQ ID NOs: 1, 2, 3, 4, 5, 6, 7, or 8. The direct repeat sequence can have at least 90% identity to a sequence comprising 10 through nucleotide 36 of any one of SEQ ID NOs: 1, 2, 3, 4, 5, 6, 7, or 8. The direct repeat sequence can have at least 90% identity to a sequence comprising 11 through nucleotide 36 of any one of SEQ ID NOs: 1, 2, 3, 4, 5, 6, 7, or 8. The direct repeat sequence can have at least 90% identity to a sequence comprising 12 through nucleotide 36 of any one of SEQ ID NOs: 1, 2, 3, 4, 5, 6, 7, or 8. The direct repeat sequence can have at least 90% identity to a sequence comprising 13 through nucleotide 36 of any one of SEQ ID NOs: 1, 2, 3, 4, 5, 6, 7, or 8. The direct repeat sequence can have at least 90% identity to a sequence comprising 14 through nucleotide 36 of any one of SEQ ID NOs: 1, 2, 3, 4, 5, 6, 7, or 8. The direct repeat sequence can have at least 90% identity to a sequence comprising I through nucleotide 34 of SEQ ID NO: 9. The direct repeat sequence can have at least 90% identity to a sequence comprising 2 through nucleotide 34 of SEQ ID NO: 9. The direct repeat sequence can have at least 90% identity to a sequence comprising 3 through nucleotide 34 of SEQ ID NO: 9. The direct repeat sequence can have at least 90% identity to a sequence comprising 4 through nucleotide 34 of SEQ ID NO: 9. The direct repeat sequence can have at least 90% identity to a sequence comprising 5 through nucleotide 34 of SEQ ID NO: 9. The direct repeat sequence can have at least 90% identity to a sequence comprising 6 through nucleotide 34 of SEQ ID NO: 9. The direct repeat sequence can have at least 90% identity to a sequence comprising 7 through nucleotide 34 of SEQ ID NO: 9. The direct repeat sequence can have at least 90% identity to a sequence comprising 8 through nucleotide 34 of SEQ ID NO: 9. The direct repeat sequence can have at least 90% identity to a sequence comprising 9 through nucleotide 34 of SEQ ID NO: 9. The direct repeat sequence can have at least 90% identity to a sequence comprising 10 through nucleotide 34 of SEQ ID NO: 9. The direct repeat sequence can have at least 90% identity to a sequence comprising 11 through nucleotide 34 of SEQ ID NO: 9. The direct repeat sequence can have at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10. In some embodiments, the direct repeat sequence has at least 90% identity to a portion of the sequence set forth in SEQ ID NO: 10.

In some embodiments, compositions comprising a Cas12i2 polypeptide and an RNA guide described herein are capable of introducing indels into a TTR target sequence. See, e.g., Example 1, where indels were measured at six TTR target sequences following delivery of an RNA guide and a Cas12i2 polypeptide to HEK293T cells by RNP; Example 2, where substitutions were introduced within TTR target sites in HEK293T cells via HDR and use of an RNP comprising an RNA guide and a Cas12i2 polypeptide; Example 3, where genomic editing of the TTR gene was done using Cas12i2 introduced into HEK293 cells by RNP; Example 4, where genomic editing of the TTR gene was done using Cas12i2 introduced into HepG2 cells by RNP; and Example 5, where genomic editing of the TTR gene was done using Cas12i2 introduced into primary hepatocytes cells by RNP.

In some embodiments, the direct repeat sequence is at least 90% identical to the reverse complement of any one of SEQ ID NOs: 1-10 (see, Table 1). In some embodiments, the direct repeat sequence is the reverse complement of any one of SEQ ID NOs: 1-10.

TABLE 1
Cas12i2 Direct Repeat Sequences

| Sequence | |
|---|---|
| identifier | Direct Repeat Sequence |
| SEQ ID NO: 1 | GUUGCAAAACCCAAGAAAUCCGU |
| | CUUUCAUUGACGG |
| SEQ ID NO: 2 | AAUAGCGGCCCUAAGAAAUCCGU |
| | CUUUCAUUGACGG |
| SEQ ID NO: 3 | AUUGGAACUGGCGAGAAAUCCGU |
| | CUUUCAUUGACGG |
| SEQ ID NO: 4 | CCAGCAACACCUAAGAAAUCCGU |
| | CUUUCAUUGACGG |
| SEQ ID NO: 5 | CGGCGCUCGAAUAGGAAAUCCGU |
| | CUUUCAUUGACGG |
| SEQ ID NO: 6 | GUGGCAACACCUAAGAAAUCCGU |
| | CUUUCAUUGACGG |
| SEQ ID NO: 7 | GUUGCAACACCUAAGAAAUCCGU |
| | CUUUCAUUGACGG |
| SEQ ID NO: 8 | GUUGCAAUGCCUAAGAAAUCCGU |
| | CUUUCAUUGACGG |
| SEQ ID NO: 9 | GCAACACCUAAGAAAUCCGUCUU |
| | UCAUUGACGGG |
| SEQ ID NO: 10 | AGAAAUCCGUCUUUCAUUGACGG |

In some embodiments, the direct repeat sequence is a sequence of Table 2 or a portion of a sequence of Table 2. The direct repeat sequence can comprise nucleotide 1 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 2 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 3 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 4 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 5 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 6 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 7 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 8 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 9 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 10 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 11 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 12 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 13 through nucleotide 36 of any one of SEQ ID NOs: 233, 234, 235, 236, 237, 238, 239, 240, 241, 242, 243, 244, 245, 246, 247, 248, 249, or 250. The direct repeat sequence can comprise nucleotide 14 through nucleotide 36 of any one of SEQ ID NOs: 233, 234, 235, 236, 237, 238, 239, 240, 241, 242, 243, 244, 245, 246, 247, 248, 249, or 250.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 2 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 3 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 4 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 5 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 6 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 7 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 8 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 9 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 10 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 11 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 12 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 95% identity to a sequence comprising 13 through nucleotide 36 of any one of SEQ ID NOs: 233, 234, 235, 236, 237, 238, 239, 240, 241, 242, 243, 244, 245, 246, 247, 248, 249, or 250.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 2 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 3 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 4 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 5 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 6 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 7 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 8 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 9 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 10 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 11 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 12 through nucleotide 36 of any one of SEQ ID NOs: 233, 234, 235, 236, 237, 238, 239, 240, 241, 242, 243, 244, 245, 246, 247, 248, 249, or 250. The direct repeat sequence can have at least 90% identity to a sequence comprising 13 through nucleotide 36 of any one of SEQ ID NOs: 233, 234, 235, 236, 237, 238, 239, 240, 241, 242, 243, 244, 245, 246, 247, 248, 249, or 250.

In some embodiments, the direct repeat sequence is at least 90% identical to the reverse complement of any one of SEQ ID NOs: 233, 234, 235, 236, 237, 238, 239, 240, 241, 242, 243, 244, 245, 246, 247, 248, 249, or 250. In some embodiments, the direct repeat sequence is at least 95% identical to the reverse complement of any one of SEQ ID NOs: 233, 234, 235, 236, 237, 238, 239, 240, 241, 242, 243, 244, 245, 246, 247, 248, 249, or 250. In some embodiments, the direct repeat sequence is the reverse complement of any one of SEQ ID NOs: 233, 234, 235, 236, 237, 238, 239, 240, 241, 242, 243, 244, 245, 246, 247, 248, 249, or 250.

In some embodiments, the direct repeat sequence is at least 90% identical to SEQ ID NO: 251 or a portion of SEQ ID NO: 251. In some embodiments, the direct repeat sequence is at least 95% identical to SEQ ID NO: 251 or a portion of SEQ ID NO: 251. In some embodiments, the direct repeat sequence is 100% identical to SEQ ID NO: 251 or a portion of SEQ ID NO: 251.

TABLE 2
Cas12i4 Direct Repeat Sequences

| Sequence identifier | 1 Direct Repeat Sequence |
|---|---|
| SEQ ID NO: 233 | UCUCAACGAUAGUCAGACAUGUC |
| | UCCUCAGUGACAC |
| SEQ ID NO: 234 | UUUUAACAACACUCAGGCAUGUC |
| | UCCACAGUGACAC |
| SEQ ID NO: 235 | UUGAACGGAUACUCAGACAUGUC |
| | UUUCCAGUGACAC |
| SEQ ID NO: 236 | UGCCCUCAAUAGUCAGAUGUGUC |
| | UCCACAGUGACAC |
| SEQ ID NO: 237 | UCUCAAUGAUACUUAGAUACGUC |
| | UCCUCAGUGACAC |
| SEQ ID NO: 238 | UCUCAAUGAUACUCAGACAUGUC |
| | UCCCCAGUGACAC |
| SEQ ID NO: 239 | UCUCAAUGAUACUAAGACAUGUG |
| | UCCUCAGUGACAC |
| SEQ ID NO: 240 | UCUCAACUAUACUCAGACAUGUC |
| | UCCUCAGUGACAC |
| SEQ ID NO: 241 | UCUCAACGAUACUCAGACAUGUG |
| | UCCUCAGUGACAC |
| SEQ ID NO: 242 | UCUCAACGAUACUAAGAUAUGUC |
| | UCCUCAGCGACAC |
| SEQ ID NO: 243 | UCUCAACGAUACUAAGAUAUGUG |
| | UCCCCAGUGACAC |
| SEQ ID NO: 244 | UCUCAACGAUACUAAGAUAUGUG |
| | UCCACAGUGACAC |
| SEQ ID NO: 245 | UCUCAACAAUACUCAGACAUGUG |
| | UCCCCAGUGACAC |
| SEQ ID NO: 246 | UCUCAACAAUACUAAGGCAUGUG |
| | UCCCCAGUGACCC |
| SEQ ID NO: 247 | UCUCAAAGAUACUCAGACACGUG |
| | UCCCCAGUGACAC |
| SEQ ID NO: 248 | UCUCAAAAAUACUCAGACAUGUG |
| | UCCUCAGUGACAC |
| SEQ ID NO: 249 | GCGAAACAACAGUCAGACAUGUG |
| | UCCCCAGUGACAC |
| SEQ ID NO: 250 | CCUCAACGAUAUUAAGACAUGUG |
| | UCCCCAGUGACAC |
| SEQ ID NO: 251 | AGACAUGUGUCCUCAGUGACAC |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOs: 259-261. In some embodiments, the direct repeat sequence is at least 95% identical to the reverse complement of any one of SEQ ID NOs: 259-261. In some embodiments, the direct repeat sequence is the reverse complement of any one of SEQ ID NOs: 259-261.

TABLE 3
Cas12i1 Direct Repeat Sequences

| Sequence identifier | Direct Repeat Sequence |
|---|---|
| SEQ ID NO: 259 | GUUGGAAUGACUAAUUUU |
| | UGUGCCCACCGUUGGCAC |
| SEQ ID NO: 260 | AAUUUUUGUGCCCAUC |
| | GUUGGCAC |
| SEQ ID NO: 261 | AUUUUUGUGCCCAUCGU |
| | UGGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, 95%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 262-264. In some embodiments, the direct repeat sequence is at least 95% identical to the reverse complement of any one of SEQ ID NOs: 262-264. In some embodiments, the direct repeat sequence is the reverse complement of any one of SEQ ID NOs: 262-264.

TABLE 4
Cas12i3 Direct Repeat Sequences.

| Sequence identifier | Direct Repeat Sequence |
|---|---|
| SEQ ID NO: 262 | CUAGCAAUGACCUAAUAG |
| | UGUGUCCUUAGUUGACAU |
| SEQ ID NO: 263 | CCUACAAUACCUAAGAAA |
| | UCCGUCCUAAGUUGACGG |
| SEQ ID NO: 264 | AUAGUGUGUCCUUAGUUGACAU |

In some embodiments, a direct repeat sequence described herein comprises a uracil (U). In some embodiments, a direct repeat sequence described herein comprises a thymine (T). In some embodiments, a direct repeat sequence according to Tables 1-4 comprises a sequence comprising a thymine in one or more places indicated as uracil in Tables 1-4.

(ii). Spacer Sequences

In some embodiments, the RNA guide comprises a DNA targeting or spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary to a non-PAM strand sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is a sequence of Table 5 or a portion of a sequence of Table 5. It should be understood that an indication of SEQ ID NOs: 116-220 should be considered as equivalent to a listing of SEQ ID NOs: 116-220, with each of the intervening numbers present in the listing, i.e., 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, and 220.

The spacer sequence can comprise nucleotide 1 through nucleotide 16 of any one of SEQ ID NOs: 116-220. The spacer sequence can comprise nucleotide 1 through nucleotide 17 of any one of SEQ ID NOs: 116-220. The spacer sequence can comprise nucleotide 1 through nucleotide 18 of any one of SEQ ID NOs: 116-220. The spacer sequence can comprise nucleotide 1 through nucleotide 19 of any one of SEQ ID NOs: 116-220. The spacer sequence can comprise nucleotide 1 through nucleotide 20 of any one of SEQ ID NOs: 116-220. The spacer sequence can comprise nucleotide 1 through nucleotide 21 of any one of SEQ ID NOs: 116-220. The spacer sequence can comprise nucleotide 1 through nucleotide 22 of any one of SEQ ID NOs: 116-220. The spacer sequence can comprise nucleotide 1 through nucleotide 23 of any one of SEQ ID NOs: 116-220. The spacer sequence can comprise nucleotide 1 through nucleotide 24 of any one of SEQ ID NOs: 116-220. The spacer sequence can comprise nucleotide 1 through nucleotide 25 of any one of SEQ ID NOs: 116-220. The spacer sequence can comprise nucleotide 1 through nucleotide 26 of any one of SEQ ID NOs: 116-220. The spacer sequence can comprise nucleotide 1 through nucleotide 27 of any one of SEQ ID NOs: 116-220. The spacer sequence can comprise nucleotide 1 through nucleotide 28 of any one of SEQ ID NOs: 116-220. The spacer sequence can comprise nucleotide 1 through nucleotide 29 of any one of SEQ ID NOs: 116-220. The spacer sequence can comprise nucleotide 1 through nucleotide 30 of any one of SEQ ID NOs: 116-220.

In some embodiments, the spacer sequence has at least 90% identity (e.g., at least 90%, 91%, 92%, 93%, 94%, 95%, 96%, 97%, 98% or 99% identity) to a sequence of Table 5 or a portion of a sequence of Table 5. The spacer sequence can have at least 90% identity to a sequence comprising nucleotide 1 through nucleotide 16 of any one of SEQ ID NOs: 116-220. The spacer sequence can have at least 90% identity to a sequence comprising nucleotide 1 through nucleotide 17 of any one of SEQ ID NOs: 116-220. The spacer sequence can have at least 90% identity to a sequence comprising nucleotide 1 through nucleotide 18 of any one of SEQ ID NOs: 116-220. The spacer sequence can have at least 90% identity to a sequence comprising nucleotide 1 through nucleotide 19 of any one of SEQ ID NOs: 116-220. The spacer sequence can have at least 90% identity to a sequence comprising nucleotide 1 through nucleotide 20 of any one of SEQ ID NOs: 116-220. The spacer sequence can have at least 90% identity to a sequence comprising nucleotide 1 through nucleotide 21 of any one of SEQ ID NOs: 116-220. The spacer sequence can have at least 90% identity to a sequence comprising nucleotide 1 through nucleotide 22 of any one of SEQ ID NOs: 116-220. The spacer sequence can have at least 90% identity to a sequence comprising nucleotide 1 through nucleotide 23 of any one of SEQ ID NOs: 116-220. The spacer sequence can have at least 90% identity to a sequence comprising nucleotide 1 through nucleotide 24 of any one of SEQ 1D NOs: 116-220. The spacer sequence can have at least 90% identity to a sequence comprising nucleotide 1 through nucleotide 25 of any one of SEQ ID NOs: 116-220. The spacer sequence can have at least 90% identity to a sequence comprising nucleotide 1 through nucleotide 26 of any one of SEQ ID NOs: 116-220. The spacer sequence can have at least 90% identity to a sequence comprising nucleotide 1 through nucleotide 27 of any one of SEQ ID NOs: 116-220. The spacer sequence can have at least 90% identity to a sequence comprising nucleotide 1 through nucleotide 28 of any one of SEQ ID NOs: 116-220. The spacer sequence can have at least 90% identity to a sequence comprising nucleotide 1 through nucleotide 29 of any one of SEQ ID NOs: 116-220. The spacer sequence can have at least 90% identity to a sequence comprising nucleotide 1 through nucleotide 30 of any one of 116-220.

TABLE 5
Target and Spacer Sequences

| | | | SEQ | | SEQ | |
|---|---|---|---|---|---|---|
| | | | ID | | ID | |
| TTR | Strand | PAM* | NO | Target Sequence | NO | Spacer Sequence |
| TTR_exon1 | + | ATTC | 11 | TTGGCAGGATGGCTTCT | 116 | UUGGCAGGAUGGCUUCUCA |
| | | | | CATCGTCTGCTCC | | UCGUCUGCUCC |
| TTR_exon1 | + | CTTC | 12 | TCATCGTCTGCTCCTCC | 117 | UCAUCGUCUGCUCCUCCUC |
| | | | | TCTGCCTTGCTGG | | UGCCUUGCUGG |
| TTR_exon1 | + | CTTG | 13 | CTGGACTGGTATTTGTG | | CUGGACUGGUAUUUGUGUC |
| | | | | TCTGAGGCTGGCC | | UGAGGCUGGCC |
| TTR_exon1 | + | ATTT | 14 | GTGTCTGAGGCTGGCCC | 119 | GUGUCUGAGGCUGGCCCUA |
| | | | | TACGGTGAGTGTT | | CGGUGAGUGUU |
| TTR_exon1 | + | TTTG | 15 | TGTCTGAGGCTGGCCCT | 120 | UGUCUGAGGCUGGCCCUAC |
| | | | | ACGGTGAGTGTTT | | GGUGAGUGUUU |
| TTR_exon1 | + | GTTT | 16 | CTGTGACATCCCATTCC | 121 | CUGUGACAUCCCAUUCCUA |
| | | | | TACATTTAAGATT | | CAUUUAAGAUU |
| TTR_exon1 | + | TTTC | 17 | TGTGACATCCCATTCCT | 122 | UGUGACAUCCCAUUCCUAC |
| | | | | ACATTTAAGATTC | | AUUUAAGAUUC |
| TTR_exon1 | − | ATTT | 18 | AGCGTGAATCTTAAATG | 123 | AGCGUGAAUCUUAAAUGUA |
| | | | | TAGGAATGGGATG | | GGAAUGGGAUG |
| TTR_exon1 | − | TTTA | 19 | GCGTGAATCTTAAATGT | 124 | GCGUGAAUCUUAAAUGUAG |
| | | | | AGGAATGGGATGT | | GAAUGGGAUGU |
| TTR_exon1 | − | CTTA | 20 | AATGTAGGAATGGGATG | 125 | AAUGUAGGAAUGGGAUGUC |
| | | | | TCACAGAAACACT | | ACAGAAACACU |
| TTR_exon1 | + | CTTG | 21 | GCAGGATGGCTTCTCAT | 126 | GCAGGAUGGCUUCUCAUCG |
| | | | | CGTCTGCTCCTCC | | UCUGCUCCUCC |
| TTR_exon1 | + | ATTC | 22 | CTACATTTAAGATTCAC | 127 | CUACAUUUAAGAUUCACGC |
| | | | | GCTAAAT | | UAAAU |
| TTR_exon2 | − | CTTG | 23 | GATTCACCGGTGCCCTG | 128 | GAUUCACCGGUGCCCUGGG |
| | | | | GGTGTAGAGAAGA | | UGUAGAGAAGA |
| TTR_exon2 | + | ATTT | 24 | TGTTAACTTCTCACGTG | 129 | UGUUAACUUCUCACGUGUC |
| | | | | TCTTCTCTACACC | | UUCUCUACACC |
| TTR_exon2 | + | TTTT | 25 | GTTAACTTCTCACGTGT | 130 | GUUAACUUCUCACGUGUCU |
| | | | | CTTCTCTACACCC | | UCUCUACACCC |
| TTR_exon2 | + | TTTG | 26 | TTAACTTCTCACGTGTC | 131 | UUAACUUCUCACGUGUCUU |
| | | | | TTCTCTACACCCA | | CUCUACACCCA |
| TTR_exon2 | + | CTTC | 27 | TCACGTGTCTTCTCTAC | 132 | UCACGUGUCUUCUCUACAC |
| | | | | ACCCAGGGCACCG | | CCAGGGCACCG |
| TTR_exon2 | − | ATTC | 28 | ACCGGTGCCCTGGGTGT | 133 | ACCGGUGCCCUGGGUGUAG |
| | | | | AGAGAAGACACGT | | AGAAGACACGU |
| TTR_exon2 | + | GTTC | 29 | TAGATGCTGTCCGAGGC | 134 | UAGAUGCUGUCCGAGGCAG |
| | | | | AGTCCTGCCATCA | | UCCUGCCAUCA |
| TTR_exon2 | − | TTTG | 30 | ACCATCAGAGGACACTT | 135 | ACCAUCAGAGGACACUUGG |
| | | | | GGATTCACCGGTG | | AUUCACCGGUG |
| TTR_exon2 | − | CTTT | 31 | GACCATCAGAGGACACT | 136 | GACCAUCAGAGGACACUUG |
| | | | | TGGATTCACCGGT | | GAUUCACCGGU |
| TTR_exon2 | − | ATTG | 32 | ATGGCAGGACTGCCTCG | 137 | AUGGCAGGACUGCCUCGGA |
| | | | | GACAGCATCTAGA | | CAGCAUCUAGA |
| TTR_exon2 | − | TTTC | 33 | TGAACACATGCACGGCC | 138 | UGAACACAUGCACGGCCAC |
| | | | | ACATTGATGGCAG | | AUUGAUGGCAG |
| TTR_exon2 | − | CTTT | 34 | CTGAACACATGCACGGC | 139 | CUGAACACAUGCACGGCCA |
| | | | | CACATTGATGGCA | | CAUUGAUGGCA |
| TTR_exon2 | − | CTTA | 35 | CCCAGAGGCAAATGGCT | 140 | CCCAGAGGCAAAUGGCUCC |
| | | | | CCCAGGTGTCATC | | CAGGUGUCAUC |
| TTR_exon2 | − | TTTG | 36 | GCAACTTACCCAGAGGC | 141 | GCAACUUACCCAGAGGCAA |
| | | | | AAATGGCTCCCAG | | AUGGCUCCCAG |
| TTR_exon2 | + | CTTC | 37 | TCTACACCCAGGGCACC | 142 | UCUACACCCAGGGCACCGG |
| | | | | GGTGAATCCAAGT | | UGAAUCCAAGU |
| TTR_exon2 | − | CTTT | 38 | GGCAACTTACCCAGAGG | 143 | GGCAACUUACCCAGAGGCA |
| | | | | CAAATGGCTCCCA | | AAUGGCUCCCA |
| TTR_exon2 | + | GTTG | 39 | CCAAAGAACCCTCCCAC | 144 | CCAAAGAACCCUCCCAGAG |
| | | | | AGGACTTGGTTTT | | GACUUGGUUUU |
| TTR_exon2 | + | TTTG | 40 | CCTCTGGGTAAGTTGCC | 145 | CCUCUGGGUAAGUUGCCAA |
| | | | | AAAGAACCCTCCC | | AGAACCCUCCC |
| TTR_exon2 | + | ATTT | 41 | GCCTCTGGGTAAGTTGC | 146 | GCCUCUGGGUAAGUUGCCA |
| | | | | CAAAGAACCCTCC | | AAGAACCCUCC |
| TTR_exon2 | + | GTTC | 42 | AGAAAGGCTGCTGATGA | 147 | AGAAAGGCUGCUGAUGACA |
| | | | | CACCTGGGAGCCA | | CCUGGGAGCCA |
| TTR_exon2 | − | GTTC | 43 | TTTGGCAACTTACCCAG | 148 | UUUGGCAACUUACCCAGAG |
| | | | | AGGCAAATGGCTC | | GCAAAUGGCUC |
| TTR_exon2 | + | GTTA | 44 | ACTTCTCACGTGTCTTC | 149 | ACUUCUCACGUGUCUUCUC |
| | | | | TCTACACCCAGGG | | UACACCCAGGG |
| TTR_exon2 | − | GTTA | 45 | ACAAAATTGATCAGAGT | 150 | ACAAAAUUGAUCAGAGUGA |
| | | | | GAA | | A |
| TTR_exon3 | + | CTTG | 46 | GCATCTCCCCATTCCAT | 151 | GCAUCUCCCCAUUCCAUGA |
| | | | | GAGCATGCAGAGG | | GCAUGCAGAGG |
| TTR_exon3 | + | CTTA | 47 | CTGGAAGGCACTTGGCA | 152 | CUGGAAGGCACUUGGCAUC |
| | | | | TCTCCCCATTCCA | | UCCCCAUUCCA |
| TTR_exon3 | + | TTTG | 48 | TAGAAGGGATATACAAA | 153 | UAGAAGGGAUAUACAAAGU |
| | | | | GTGGAAATAGACA | | GGAAAUAGACA |
| TTR_exon3 | − | CTTC | 49 | TACAAATTCCTCCTCAG | 154 | UACAAAUUCCUCCUCAGUU |
| | | | | TTGTGAGCCCATG | | GUGAGCCCAUG |
| TTR_exon3 | + | CTTC | 50 | GAGGGTTGTTTTGGTTT | 155 | GAGGGUUGUUUUGGUUUUG |
| | | | | TGGTTTTTGCTTT | | GUUUUUGCUUU |
| TTR_exon3 | − | CTTC | 51 | CAGTAAGATTTGGTGTC | 156 | CAGUAAGAUUUGGUGUCUA |
| | | | | TATTTCCACTTTG | | UUUCCACUUUG |
| TTR_exon3 | − | ATTT | 52 | GGTGTCTATTTCCACTT | 157 | GGUGUCUAUUUCCACUUUG |
| | | | | TGTATATCCCTTC | | UAUAUCCCUUC |
| TTR_exon3 | − | TTTG | 53 | GTGTCTATTTCCACTTT | 158 | GUGUCUAUUUCCACUUUGU |
| | | | | GTATATCCCTTCT | | AUAUCCCUUCU |
| TTR_exon3 | − | ATTT | 54 | CCACTTTGTATATCCCT | 159 | CCACUUUGUAUAUCCCUUC |
| | | | | TCTACAAATTCCT | | UACAAAUUCCU |
| TTR_exon3 | − | TTTC | 55 | CACTTTGTATATCCCTT | 160 | CACUUUGUAUAUCCCUUCU |
| | | | | CTACAAATTCCTC | | ACAAAUUCCUC |
| TTR_exon3 | − | CTTT | 56 | GTATATCCCTTCTACAA | 161 | GUAUAUCCCUUCUACAAAU |
| | | | | ATTCCTCCTCAGT | | UCCUCCUCAGU |
| TTR_exon3 | − | TTTG | 57 | TATATCCCTTCTACAAA | 162 | UAUAUCCCUUC-UACAAAUU |
| | | | | TTCCTCCTCAGTT | | CCUCCUCAGUU |
| TTR_exon3 | + | ATTT | 58 | GTAGAAGGGATATACAA | 163 | GUAGAAGGGAUAUACAAAG |
| | | | | AGTGGAAATAGAC | | UGGAAAUAGAC |
| TTR_exon3 | − | ATTC | 59 | CTCCTCAGTTGTGAGCC | 164 | CUCCUCAGUUGUGAGCCCA |
| | | | | CATGCAGCTCTCC | | UGCAGCUCUCC |
| TTR_exon3 | − | GTTG | 60 | TGAGCCCATGCAGCTCT | 165 | UGAGCCCAUGCAGCUCUCC |
| | | | | CCAGACTCACTGG | | AGACUCACUGG |
| TTR_exon3 | − | GTTT | 61 | TCCTATAAGGTGTGAAA | 166 | UCCUAUAAGGUGUGAAAGU |
| | | | | GTCTGGATTAAGT | | CUGGAUUAAGU |
| TTR_exon3 | − | TTTT | 62 | COTATAAGGTGTGAAAG | 167 | CCUAUAAGGUGUGAAAGUC |
| | | | | TCTGGATTAAGTT | | UGGAUUAAGUU |
| TTR_exon3 | − | TTTC | 63 | CTATAAGGTGTGAAAGT | 168 | CUAUAAGGUGUGAAAGUCU |
| | | | | CTGGATTAAGTTA | | GGAUUAAGUUA |
| TTR_exon3 | + | ATTC | 64 | CATGAGCATGCAGAGGT | 169 | CAUGAGCAUGCAGAGGUGA |
| | | | | GAGTATACAGACC | | GUAUACAGACC |
| TTR_exon3 | + | CTTA | 65 | TAGGAAAACCAGTGAGT | 170 | UAGGAAAACCAGUGAGUCU |
| | | | | CTGGAGAGCTGCA | | GGAGAGCUGCA |
| TTR_exon3 | + | ATTT | 66 | GTTTCCTCCATGCGTAA | 171 | GUUUCCUCCAUGCGUAACU |
| | | | | CTTAATCCAGACT | | UAAUCCAGACU |
| TTR_exon3 | + | CTTT | 67 | CACACCTTATAGGAAAA | 172 | CACACCUUAUAGGAAAACC |
| | | | | CCAGTGAGTCTGG | | AGUGAGUCUGG |
| TTR_exon3 | + | CTTA | 68 | ATCCAGACTTTCACACC | 173 | AUCCAGACUUUCACACCUU |
| | | | | TTATAGGAAAACC | | AUAGGAAAACC |
| TTR_exon3 | + | TTTC | 69 | CTCCATGCGTAACTTAA | 174 | CUCCAUGCGUAACUUAAUC |
| | | | | TCCAGACTTTCAC | | CAGACUUUCAC |
| TTR_exon3 | + | GTTT | 70 | CCTCCATGCGTAACTTA | 175 | CCUCCAUGCGUAACUUAAU |
| | | | | ATCCAGACTTTCA | | CCAGACUUUCA |
| TTR_exon3 | + | TTTG | 71 | TTTCCTCCATGCGTAAC | 176 | UUUCCUCCAUGCGUAACUU |
| | | | | TTAATCCAGACTT | | AAUCCAGACUU |
| TTR_exon3 | + | TTTC | 72 | ACACCTTATAGGAAAAC | 177 | ACACCUUAUAGGAAAACCA |
| | | | | CAGTGAGTCTGGA | | GUGAGUCUGGA |
| TTR_exon3 | + | GTTG | 73 | TTTTGGTTTTGGTTTTT | 178 | UUUUGGUUUUGGUUGUGGC |
| | | | | GCTTTTG | | UUOOG |
| TTR_exon3 | + | GTTT | 74 | TGGTTTTGGTTTTTGCT | 179 | UGGUUUUGGUUUUUGCUUU |
| | | | | TTTG | | UG |
| TTR_exon3 | + | TTTT | 75 | GGTTTTGGTTTTTGCTT | 180 | GGUUUUGGUUUUUGCUUUU |
| | | | | TIG | | G |
| TTR_exon3 | − | ATTA | 76 | AGTTACGCATGGAGGAA | 181 | AGUUACGCAUGGAGGAAAC |
| | | | | ACAAATGG | | AAAUGG |
| TTR_exon3 | − | GTTA | 77 | CGCATGGAGGAAACAAA | 182 | CGCAGGGAGGAAACAAAUG |
| | | | | TGG | | G |
| TTR_exon4 | + | TTTA | 78 | CTAAAGCAGTGTTTTCA | 183 | CUAAAGCAGUGUUUUCACC |
| | | | | CCTCATATGCTAT | | UCAUAUGCUAU |
| TTR_exon4 | + | TTTT | 79 | ACIAAAGCAGTGTTITC | 184 | ACUAAAGCAGUGUUUUCAC |
| | | | | ACCTCATATGCTA | | CUCAUAUGCUA |
| TTR_exon4 | + | TTTT | 80 | TACTAAAGCAGTGTTTT | 185 | UACUAAAGCAGUGUUUUCA |
| | | | | CACCTCATATGCT | | CCUCAUAUGCU |
| TTR_exon4 | + | ATTT | 81 | TTACTAAAGCAGTGTTT | 186 | UUACUAAAGCAGUGUUUUC |
| | | | | TCACCTCATATGC | | ACCUCAUAUGC |
| TTR_exon4 | + | ATTC | 82 | CATTTTTACTAAAGCAG | 187 | CAUUUUUACUAAAGCAGUG |
| | | | | TGTTTTCACCTCA | | UUUUCACCUCA |
| TTR_exon4 | + | TTTC | 83 | ATGTAACCAAGAGTATT | 188 | AUGUAACCAAGAGUAUUCC |
| | | | | CCATTTTTACTAA | | AUUUUUACUAA |
| TTR_exon4 | + | ATTT | 84 | CATGTAACCAAGAGTAT | 189 | CAUGUAACCAAGAGUAUUC |
| | | | | TCCATTTTTACTA | | CAUUUUUACUA |
| TTR_exon4 | + | ATTC | 85 | CACCACGGCTGTCGTCA | 190 | CACCACGGCUGUCGUCACC |
| | | | | CCAATCCCAAGGA | | AAUCCCAAGGA |
| TTR_exon4 | + | GTTT | 86 | TCACCTCATATGCTATG | 191 | UCACCUCAUAUGCUAUGUU |
| | | | | TTAGAAGTCCAGG | | AGAAGUCCAGG |
| TTR_exon4 | + | ATTG | 87 | CCGCCCTGCTGAGCCCC | 192 | CCGCCCUGCUGAGCCCCUA |
| | | | | TACTCCTATTCCA | | CUCCUAUUCCA |
| TTR_exon4 | + | ATTC | 88 | ACAGCCAACGACTCCGG | 193 | ACAGCCAACGACUCCGGCC |
| | | | | CCCCCGCCGCTAC | | CCCGCCGCUAC |
| TTR_exon4 | + | CTTC | 89 | TCTCATAGGTGGTATTC | 194 | UCUCAUAGGUGGUAUUCAC |
| | | | | ACAGCCAACGACT | | AGCCAACGACU |
| TTR_exon4 | + | TTTC | 90 | GGGCTCTGGTGGAAATG | 195 | GGGCUCUGGUGGAAAUGGA |
| | | | | GATCTGTCTGTCT | | UCUGUCUGUCU |
| TTR_exon4 | + | TTTT | 91 | CGGGCTCTGGTGGAAAT | 196 | CGGGCUCUGGUGGAAAUGG |
| | | | | GGATCTGTCTGTC | | AUCUGUCUGUC |
| TTR_exon4 | + | TTTT | 92 | TCGGGCTCTGGTGGAAA | 197 | UCGGGCUCUGGUGGAAAUG |
| | | | | TGGATCTGTCTGT | | GAUCUGUCUGU |
| TTR_exon4 | + | CTTC | 93 | TCCTCCAGTGGACCTGA | 198 | UCCUCCAGUGGACCUGAAG |
| | | | | AGGACGAGGGATG | | GACGAGGGAUG |
| TTR_exon4 | + | TTTT | 94 | CACCTCATATGCTATGT | 199 | CACCUCAUAUGCUAUGUUA |
| | | | | TAGAAGTCCAGGC | | GAAGUCCAGGC |
| TTR_exon4 | − | CTTT | 95 | CACAGGAATGTTTTATT | 200 | CACAGGAAUGUUUUAUUGU |
| | | | | GTCTCTGCCTGGA | | CUCUGCCUGGA |
| TTR_exon4 | + | GTTA | 96 | GAAGTCCAGGCAGAGAC | 201 | GAAGUCCAGGCAGAGACAA |
| | | | | AATAAAACATTCC | | UAAAACAUUCC |
| TTR_exon4 | − | GTTC | 97 | GCTGTGAATACCACCTA | 202 | GCUGUGAAUACCACCUAUG |
| | | | | TGAGAGAAGACAG | | AGAGAAGACAG |
| TTR_exon4 | − | ATTG | 98 | GTGACGACAGCCGTGGT | 203 | GUGACGACAGCCGUGGUGG |
| | | | | GGAATAGGAGTAG | | AAUAGGAGUAG |
| TTR_exon4 | − | CTTG | 99 | GGATTGGTGACGACAGC | 204 | GGAUUGGUGACGACAGCCG |
| | | | | CGTGGTGGAATAG | | UGGUGGAAUAG |
| TTR_exon4 | − | ATTC | 100 | CTTGGGATTGGTGACGA | 205 | CUUGGGAUUGGUGACGACA |
| | | | | CAGCCGTGGTGGA | | GCCGUGGUGGA |
| TTR_exon4 | − | CTTC | 101 | AGGTCCACTGGAGGAGA | 206 | AGGUCCACUGGAGGAGAAG |
| | | | | AGTCCCTCATTCC | | UCCCUCAUUCC |
| TTR_exon4 | − | GTTA | 102 | CATGAAATCCCATCCCT | 207 | CAUGAAAUCCCAUCCCUCG |
| | | | | CGTCCTTCAGGTC | | UCCUUCAGGUC |
| TTR_exon4 | − | CTTG | 103 | GTTACATGAAATCCCAT | 208 | GUUACAUGAAAUCCCAUCC |
| | | | | CCCTCGTCCTTCA | | CUCGUCCUUCA |
| TTR_exon4 | − | TTTA | 104 | GTAAAAATGGAATACTC | 209 | GUAAAAAUGGAAUACUCUU |
| | | | | TTGGTTACATGAA | | GGUUACAUGAA |
| TTR_exon4 | − | CTTT | 105 | AGTAAAAATGGAATACT | 210 | AGUAAAAAUGGAACACUCU |
| | | | | CTTGGTTACATGA | | UGGUUACAUGA |
| TTR_exon4 | − | CTTC | 106 | TAACATAGCATATGAGG | 211 | UAACAUAGCAUAUGAGGUG |
| | | | | TGAAAACACTGCT | | AAAACACUGCU |
| TTR_exon4 | − | ATTG | 107 | TCTCTGCCTGGACTTCT | 212 | UCUCUGGCUGGACUUCUAA |
| | | | | AACATAGCATATG | | CAUAGCAUAUG |
| TTR_exon4 | − | TTTA | 108 | TTGTCTCTGCCTGGACT | 213 | UUGUCUCUGCCUGGACUUC |
| | | | | TCTAACATAGCAT | | UAACAUAGCAU |
| TTR_exon4 | − | TTTT | 109 | ATTGTCTCTGCCTGGAC | 214 | AUUGUCUCUGCCUGGACUU |
| | | | | TTCTAACATAGCA | | CUAACAUAGCA |
| TTR_exon4 | − | GTTT | 110 | TATTGTCTCTGCCTGGA | 215 | UAUUGUCUCUGCCUGGACU |
| | | | | CTTCTAACATAGC | | UCUAACAUAGC |
| TTR_exon4 | − | TTTC | 111 | ACAGGAATGTTTTATTG | 216 | ACAGGAAUGUUUUAUUGUC |
| | | | | TCTCTGCCTGGAC | | UCUGCCUGGAC |
| TTR_exon4 | + | TTTC | 112 | ACCTCATATGCTATGTT | 217 | ACCUCAUAUGCUAUGUUAG |
| | | | | AGAAGTCCAGGCA | | AAGUCCAGGCA |
| TTR_exon4 | + | GTTT | 113 | TTCGGGCTCTGGTGGAA | 218 | UUCGGGCUCUGGUGGAAAU |
| | | | | ATGGATCTGTCTG | | GGAUCUGUCUG |
| TTR_exon4 | − | ATTT | 114 | CCACCAGAGCCCGAAAA | 219 | CCACCAGAGCCCGAAAAAC |
| | | | | ACGTG | | GUG |
| TTR_exon4 | − | TTTC | 115 | CACCAGAGCCCGAAAAA | 220 | CACCAGAGCCCGAAAAACG |
| | | | | CGTG | | UG |

*The three 3′ nucleotides represent the 5′-TTN-3′ motif.

The present disclosure includes all combinations of the direct repeat sequences and spacer sequences listed above, consistent with the present disclosure herein.

In some embodiments, a spacer sequence described herein comprises a uracil (U). In some embodiments, a spacer sequence described herein comprises a thymine (T). In some embodiments, a spacer sequence according to Table 5 comprises a sequence comprising a thymine in one or more (e.g., all) places indicated as uracil in Table 5.

The present disclosure includes RNA guides that comprise any and all combinations of the direct repeats and spacers described herein (e.g., as set forth in Table 5, above). In some embodiments, the RNA guide has at least 90% identity (e.g., at least 90%, 91%, 92%. 93%, 94%, 95%, 96%, 97%, 98% or 99% identity to any one of SEQ ID NOs: 273-278 or 345-362. In some embodiments, the RNA guide has at least 95% identity (e.g., at least 95%, 96%, 97%, 98% or 99% identity to any one of SEQ ID NOs: 273-278 and 345-362. In some embodiments, the RNA guide has a sequence set forth in any one of SEQ ID NOs: 273-278 and 345-362.

B. Template DNA

The gene editing system as disclosed herein may further comprise a template DNA, a portion of which can be incorporated into a genetic site within a TTR gene via, e.g., homologous recombination, leading to genetic editing as desired at the TTR genetic site, e.g., correction of a mutation in the target TTR gene or introduction of a protective mutation. In some instances, the portion of the template DNA to be incorporated into the TTR gene may be codon-optimized.

In some embodiments, the template DNA is a single-stranded nucleic acid. In some embodiments, the template DNA is a double-stranded nucleic acid. In some embodiments, the template DNA is a DNA, RNA, or DNA/RNA hybrid molecule. In some embodiments, the template DNA is a single-stranded oligo DNA nucleotide (ssODN) template DNA or comprises ssODNs. In some embodiments, the template DNA is a double-stranded oligo DNA nucleotide (dsODN) template DNA or comprises dsODNs. In some embodiments, the template DNA is linear. In some embodiments, the template DNA is circular (e.g., a plasmid). In some embodiments, the template DNA is an exogenous nucleic acid molecule, e.g., exogenous to the target cell. In some embodiments, the template DNA is a chromatid (e.g., a sister chromatid). In some embodiments, the template DNA is delivered by a virus.

In some embodiments, the template DNA and the TTR target nucleic acid are not identical in sequence. In some embodiments, a template DNA comprises one or more nucleotides that are heterologous (e.g., not homologous) to the target nucleic acid. In some embodiments, the template DNA comprises one or more (e.g., one, two, three, four, five, or more sequence differences relative to the target nucleic acid). In some embodiments, the template DNA comprises an insertion, a deletion, a polymorphism, an inversion, or a rearrangement relative to the target nucleic acid. The insertion may comprise a restriction site or a selectable marker. In some embodiments, a break in the target nucleic acid (e.g., a break induced by a Cas12i polypeptide) is repaired by HDR using the template DNA. As such, use of a template DNA can result in an insertion, deletion, or substitution in the target nucleic acid by way of HDR. In some embodiments, the insertion may comprise a gene, e.g., a wild-type gene, or a portion thereof. In some embodiments, the insertion may comprise a deletion of a gene or portion thereof as compared to a target nucleic acid (e.g., the target genome of the cell).

It is of course understood that incorporation of a sequence difference from a template DNA into a target nucleic acid does not require physical insertion of one or more nucleotides from the template DNA into a target nucleic acid. Rather, as an example, the template DNA can be used as a template for DNA synthesis, such that informational content of the template DNA is incorporated into the target nucleic acid, without the physical incorporation of nucleotides of the template DNA into the target nucleic acid. For instance, without wishing to be bound by theory, the template DNA may be used for homology-directed repair (HDR) of the target nucleic acid.

In some embodiments, a template DNA comprises a donor region comprising one or more nucleotides (e.g., an insert sequence) between a 5′ homology arm and a 3′ homology arm. In some embodiments, a template DNA comprises a donor region (e.g., an insert sequence) downstream of a 5′ homology arm. In some embodiments, a template DNA comprises a donor region (e.g., an insert sequence) upstream of a 3′ homology arm. In some embodiments, a template DNA comprises sufficient homology to allow for HDR. In some embodiments, 5′ homology arm and/or 3′ homology arm of a template DNA will have at least 50% sequence identity the target nucleic acid. In certain embodiments, at least 60%, 70%, 80%, 90%, 95%, 98%, 99%, or 100% sequence identity is present.

In some embodiments, the 5′ homology arm and/or the 5′ homology arm can be homologous to upstream and/or downstream to a TTR gene target site where genetic editing is desired. The donor region, which is flanked by the homology arm(s), can be incorporated into the TTR gene target site via, e.g., homologous recombination, thereby introducing the edits encoded by the donor region into the TTR gene target site.

In some embodiments, a single-stranded template DNA comprises a donor region (e.g., an insert sequence) that is from 1 nucleotide to about 200 nucleotides in length, e.g., 1 nucleotide to 5 nucleotides, from 5 nucleotides to 10 nucleotides, from 10 nucleotides to 15 nucleotides, from 15 nucleotides to 20 nucleotides, from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides.

In some embodiments, a double-stranded template DNA comprises a donor region (e.g., an insert sequence) that is up to about 10,000 base pairs (10 kb) in length. For example, in some embodiments, a double-stranded template DNA comprises a donor region (e.g., an insert) that is I base pair, about 10 base pairs, about 20 base pairs, about 30 base pairs, about 40 base pairs, about 50 base pairs, about 60 base pairs, about 70 base pairs, about 80 base pairs, about 90 base pairs, about 100 base pairs, about 200 base pairs, about 300 base pairs, about 400 base pairs, about 500 base pairs, about 600 base pairs, about 700 base pairs, about 800 base pairs, about 900 base pairs, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, 3 kb, about 3.1 kb, about 3.2 kb, about 3.3 kb, about 3.4 kb, about 3.5 kb, about 3.6 kb, about 3.7 kb, about 3.8 kb, about 3.9 kb, about 4 kb, about 4.1 kb, about 4.2 kb, about 4.3 kb, about 4.4 kb, about 4.5 kb, about 4.6 kb, about 4.7 kb, about 4.8 kb, about 4.9 kb, about 5 kb, about 5.1 kb, about 5.2 kb, about 5.3 kb, about 5.4 kb, about 5.5 kb, about 5.6 kb, about 5.7 kb, about 5.8 kb, about 5.9 kb, about 6 kb, about 6.1 kb, about 6.2 kb, about 6.3 kb, about 6.4 kb, about 6.5 kb, about 6.6 kb, about 6.7 kb, about 6.8 kb, about 6.9 kb, about 7 kb, about 7.1 kb, about 7.2 kb, about 7.3 kb, about 7.4 kb, about 7.5 kb, about 7.6 kb, about 7.7 kb, about 7.8 kb, about 7.9 kb, about 8 kb, about 8.1 kb, about 8.2 kb, about 8.3 kb, about 8.4 kb, about 8.5 kb, about 8.6 kb, about 8.7 kb, about 8.8 kb, about 8.9 kb, about 9 kb, about 9.1 kb, about 9.2 kb, about 9.3 kb, about 9.4 kb, about 9.5 kb, about 9.6 kb, about 9.7 kb, about 9.8 kb, about 9.9 kb, or about 10 kb in length.

In some embodiments, the template DNA comprises one or two flanking homology arms. In some embodiments, the template DNA comprises a 5′ homology arm (e.g., a left homology arm). In some embodiments, the template DNA comprises a 3′ homology arm (e.g., a right homology arm). In some embodiments, the template DNA comprises a 5′ homology arm and a 3′ homology arm.

In some embodiment, a single-stranded template DNA comprises a 5′ homology arm (e.g., a left homology arm) that has a length of from about 20 nucleotides to about 200 nucleotides e.g., from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides. In some embodiments, a single-stranded template DNA comprises a 5′ homology arm (e.g., a left homology arm) that has a length of about 200 nucleotides to about 500 nucleotides, e.g., from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, from 290 nucleotides to 300 nucleotides, from 300 nucleotides to 310 nucleotides, from 310 nucleotides to 320 nucleotides, from 320 nucleotides to 330 nucleotides, from 330 nucleotides to 340 nucleotides, from 340 nucleotides to 350 nucleotides, from 350 nucleotides to 360 nucleotides, from 360 nucleotides to 370 nucleotides, from 370 nucleotides to 380 nucleotides, from 380 nucleotides to 390 nucleotides, from 390 nucleotides to 400 nucleotides, from 400 nucleotides to 410 nucleotides, from 410 nucleotides to 420 nucleotides, from 420 nucleotides to 430 nucleotides, from 430 nucleotides to 440 nucleotides, from 440 nucleotides to 450 nucleotides, from 450 nucleotides to 460 nucleotides, from 460 nucleotides to 470 nucleotides, from 470 nucleotides to 480 nucleotides, from 480 nucleotides to 490 nucleotides, or from 490 nucleotides to 500 nucleotides.

In some embodiment, a single-stranded template DNA comprises a 3′ homology arm (e.g., a right homology arm) that has a length of from about 20 nucleotides to about 200 nucleotides e.g., from 20 nucleotides to 25 nucleotides, from 25 nucleotides to 30 nucleotides, from 30 nucleotides to 35 nucleotides, from 35 nucleotides to 40 nucleotides, from 40 nucleotides to 45 nucleotides, from 45 nucleotides to 50 nucleotides, from 50 nucleotides to 55 nucleotides, from 55 nucleotides to 60 nucleotides, from 60 nucleotides to 65 nucleotides, from 65 nucleotides to 70 nucleotides, from 70 nucleotides to 75 nucleotides, from 75 nucleotides to 80 nucleotides, from 80 nucleotides to 85 nucleotides, from 85 nucleotides to 90 nucleotides, from 90 nucleotides to 95 nucleotides, from 95 nucleotides to 100 nucleotides, from 100 nucleotides to 105 nucleotides, from 105 nucleotides to 110 nucleotides, from 110 nucleotides to 115 nucleotides, from 115 nucleotides to 120 nucleotides, from 120 nucleotides to 125 nucleotides, from 125 nucleotides to 130 nucleotides, from 130 nucleotides to 135 nucleotides, from 135 nucleotides to 140 nucleotides, from 140 nucleotides to 145 nucleotides, from 145 nucleotides to 150 nucleotides, from 150 nucleotides to 155 nucleotides, from 155 nucleotides to 160 nucleotides, from 160 nucleotides to 165 nucleotides, from 165 nucleotides to 170 nucleotides, from 170 nucleotides to 175 nucleotides, from 175 nucleotides to 180 nucleotides, from 180 nucleotides to 185 nucleotides, from 185 nucleotides to 190 nucleotides, from 190 nucleotides to 195 nucleotides, or from 195 nucleotides to 200 nucleotides. In some embodiments, a single-stranded template DNA comprises a 3′ homology arm (e.g., a right homology arm) that has a length of about 200 nucleotides to about 500 nucleotides, e.g., from 200 nucleotides to 210 nucleotides, from 210 nucleotides to 220 nucleotides, from 220 nucleotides to 230 nucleotides, from 230 nucleotides to 240 nucleotides, from 240 nucleotides to 250 nucleotides, from 250 nucleotides to 260 nucleotides, from 260 nucleotides to 270 nucleotides, from 270 nucleotides to 280 nucleotides, from 280 nucleotides to 290 nucleotides, from 290 nucleotides to 300 nucleotides, from 300 nucleotides to 310 nucleotides, from 310 nucleotides to 320 nucleotides, from 320 nucleotides to 330 nucleotides, from 330 nucleotides to 340 nucleotides, from 340 nucleotides to 350 nucleotides, from 350 nucleotides to 360 nucleotides, from 360 nucleotides to 370 nucleotides, from 370 nucleotides to 380 nucleotides, from 380 nucleotides to 390 nucleotides, from 390 nucleotides to 400 nucleotides, from 400 nucleotides to 410 nucleotides, from 410 nucleotides to 420 nucleotides, from 420 nucleotides to 430 nucleotides, from 430 nucleotides to 440 nucleotides, from 440 nucleotides to 450 nucleotides, from 450 nucleotides to 460 nucleotides, from 460 nucleotides to 470 nucleotides, from 470 nucleotides to 480 nucleotides, from 480 nucleotides to 490 nucleotides, or from 490 nucleotides to 500 nucleotides.

In some embodiment, a double-stranded template DNA comprises a 5′ homology arm (e.g., a left homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs. In some embodiments, a double-stranded template DNA comprises a 5′ homology arm (e.g., a left homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs. In some embodiments, a double-stranded template DNA comprises a 5′ homology arm that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb).

In some embodiment, a double-stranded template DNA comprises a 3′ homology arm (e.g., a right homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs. In some embodiments, a double-stranded template DNA comprises a 3′ homology arm (e.g., a right homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs. In some embodiments, a double-stranded template DNA comprises a 3′ homology arm (e.g., a right homology arm) that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb).

In some embodiment, a double-stranded template DNA comprises a sense strand 5′ homology arm (e.g., a left homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs. In some embodiments, a double-stranded template DNA comprises a sense strand 5′ homology arm (e.g., a left homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs. In some embodiments, a double-stranded template DNA comprises a sense strand 5′ homology arm that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb).

In some embodiment, a double-stranded template DNA comprises a sense strand 3′ homology arm (e.g., a right homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs. In some embodiments, a double-stranded template DNA comprises a sense strand 3′ homology arm (e.g., a right homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs. In some embodiments, a double-stranded template DNA comprises a sense strand 3′ homology arm that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb).

In some embodiment a double-stranded template DNA comprises a sense strand 5′ homology arm (e.g., a left homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs and a sense strand 3′ homology arm (e.g., a right homology arm) that has a length of from about 20 base pairs to about 200 base pairs e.g., from 20 base pairs to 25 base pairs, from 25 base pairs to 30 base pairs, from 30 base pairs to 35 base pairs, from 35 base pairs to 40 base pairs, from 40 base pairs to 45 base pairs, from 45 base pairs to 50 base pairs, from 50 base pairs to 55 base pairs, from 55 base pairs to 60 base pairs, from 60 base pairs to 65 base pairs, from 65 base pairs to 70 base pairs, from 70 base pairs to 75 base pairs, from 75 base pairs to 80 base pairs, from 80 base pairs to 85 base pairs, from 85 base pairs to 90 base pairs, from 90 base pairs to 95 base pairs, from 95 base pairs to 100 base pairs, from 100 base pairs to 105 base pairs, from 105 base pairs to 110 base pairs, from 110 base pairs to 115 base pairs, from 115 base pairs to 120 base pairs, from 120 base pairs to 125 base pairs, from 125 base pairs to 130 base pairs, from 130 base pairs to 135 base pairs, from 135 base pairs to 140 base pairs, from 140 base pairs to 145 base pairs, from 145 base pairs to 150 base pairs, from 150 base pairs to 155 base pairs, from 155 base pairs to 160 base pairs, from 160 base pairs to 165 base pairs, from 165 base pairs to 170 base pairs, from 170 base pairs to 175 base pairs, from 175 base pairs to 180 base pairs, from 180 base pairs to 185 base pairs, from 185 base pairs to 190 base pairs, from 190 base pairs to 195 base pairs, or from 195 base pairs to 200 base pairs. In some embodiments, a double-stranded template DNA comprises a sense strand 5′ homology arm (e.g., a left homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs and a sense strand 3′ homology arm (e.g., a right homology arm) that has a length of about 200 base pairs to about 500 base pairs, e.g., from 200 base pairs to 210 base pairs, from 210 base pairs to 220 base pairs, from 220 base pairs to 230 base pairs, from 230 base pairs to 240 base pairs, from 240 base pairs to 250 base pairs, from 250 base pairs to 260 base pairs, from 260 base pairs to 270 base pairs, from 270 base pairs to 280 base pairs, from 280 base pairs to 290 base pairs, from 290 base pairs to 300 base pairs, from 300 base pairs to 310 base pairs, from 310 base pairs to 320 base pairs, from 320 base pairs to 330 base pairs, from 330 base pairs to 340 base pairs, from 340 base pairs to 350 base pairs, from 350 base pairs to 360 base pairs, from 360 base pairs to 370 base pairs, from 370 base pairs to 380 base pairs, from 380 base pairs to 390 base pairs, from 390 base pairs to 400 base pairs, from 400 base pairs to 410 base pairs, from 410 base pairs to 420 base pairs, from 420 base pairs to 430 base pairs, from 430 base pairs to 440 base pairs, from 440 base pairs to 450 base pairs, from 450 base pairs to 460 base pairs, from 460 base pairs to 470 base pairs, from 470 base pairs to 480 base pairs, from 480 base pairs to 490 base pairs, or from 490 base pairs to 500 base pairs. In some embodiments, a double-stranded template DNA comprises a sense strand 5′ homology arm (e.g., a left homology arm) that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb) and a sense strand 3′ homology arm (e.g., a right homology arm) that has a length of at least 500 base pairs, e.g., from 500 base pairs to 550 base pairs, from 550 base pairs to 600 base pairs, from 600 base pairs to 650 base pairs, from 650 base pairs to 700 base pairs, from 700 base pairs to 750 base pairs, from 750 base pairs to 800 base pairs, from 800 base pairs to 850 base pairs, from 850 base pairs to 900 base pairs, from 900 base pairs to 950 base pairs, or from 950 base pairs to 1,000 base pairs (1 kb).

In some embodiments, a template DNA comprises a 5′ homology arm (e.g., a left homology arm) and a 3′ homology arm (e.g., a right homology arm) that are identical in length. In some embodiments, a template DNA comprises a 5′ homology arm (e.g., a left homology arm) and a 3′ homology arm (e.g., a right homology arm) of different lengths. In some embodiments, the 5′ homology arm is about 10%, 15%, 20%, 30%, 35%, 40%, 45%, 50%, 55%, 60%, 65%, 70%, 75%, 80%, 85%, 90%, 95% 96%, 97%, 98%, or 99%, but not 100%, the length of the 3′ homology arm. In some embodiments, the 3′ homology arm is about 10%, 15%, 20%, 30%, 35%, 40%, 45%, 50%, 55%, 60%, 65%, 70%, 75%, 80%, 85%, 90%, 95% 96%, 97%, 98%, or 99%, but not 100%, the length of the 5′ homology arm.

In some embodiments, a template DNA comprises two homology arms that are similar in length (e.g., a right homology arm and a left homology arm of similar length). For example, in some embodiments, the 5′ homology arm (e.g., the left homology arm) is at least about 70% (e.g., about 70%, 71%, 72%, 73%, 74%, 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, or 99%) the length of the 3′ homology arm (e.g., the right homology arm). In some embodiments, the 5′ homology arm (e.g., the left homology arm) is at least about 75% (e.g., about 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, or 99%) the length of the 3′ homology arm (e.g., the right homology arm). In some embodiments, the 5′ homology arm (e.g., the left homology arm) is at least about 80% (e.g., about 80%, 81%, 82%, 83%, 84%, 85%, 86%, 87%, 88%, 89%, 90%, 91%, 92%, 93%, 94%, 95%, 96%, 97%, 98%, or 99%) the length of the 3′ homology arm (e.g., the right homology arm). In some embodiments, the 5′ homology arm (e.g., the left homology arm) is at least about 85% (e.g., about 85%, 86%, 87%, 88%, 89%, 90%, 91%, 92%, 93%, 94%, 95%, 96%, 97%, 98%, or 99%) the length of the 3′ homology arm (e.g., the right homology arm). In some embodiments, the 5′ homology arm (e.g., the left homology arm) is at least about 90% (e.g., about 90%, 91%, 92%, 93%, 94%, 95%, 96%, 97%, 98%, or 99%) the length of the 3′ homology arm (e.g., the right homology arm). In some embodiments, the 5′ homology arm (e.g., the left homology arm) is at least about 95% (e.g., about 95%, 96%, 97%, 98%, or 99%) the length of the 3′ homology arm (e.g., the right homology arm). In some embodiments, the 3′ homology arm (e.g., the right homology arm) is at least about 70% (e.g., about 70%, 71%, 72%, 73%, 74%, 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, or 99%) the length of the 5′ homology arm (e.g., the left homology arm). In some embodiments, the 3′ homology arm (e.g., the right homology arm) is at least about 75% (e.g., about 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, or 99%) the length of the 5′ homology arm (e.g., the left homology arm). In some embodiments, the 3′ homology arm (e.g., the right homology arm) is at least about 80% (e.g., about 80%, 81%, 82%, 83%, 84%, 85%, 86%, 87%, 88%, 89%, 90%, 91%, 92%, 93%, 94%, 95%, 96%, 97%, 98%, or 99%) the length of the 5′ homology arm (e.g., the left homology arm). In some embodiments, the 3′ homology arm (e.g., the right homology arm) is at least about 85% (e.g., about 85%, 86%, 87%, 88%, 89%, 90%, 91%, 92%, 93%, 94%, 95%, 96%, 97%, 98%, or 99%) the length of the 5′ homology arm (e.g., the left homology arm). In some embodiments, the 3′ homology arm (e.g., the right homology arm) is at least about 90% (e.g., about 90%, 91%, 92%, 93%, 94%, 95%, 96%, 97%, 98%, or 99%) the length of the 5′ homology arm (e.g., the left homology arm). In some embodiments, the 3′ homology arm (e.g., the right homology arm) is at least about 95% (e.g., about 95%, 96%, 97%, 98%, or 99%) the length of the 5′ homology arm (e.g., the left homology arm).

In some embodiments, a template DNA comprises a 5′ homology arm but does not comprise a 3′ homology arm. In some embodiments, a template DNA comprises a 3′ homology arm but does not comprise a 5′ homology arm.

In some embodiments, the insert sequence is situated within the corresponding region of the TTR target nucleic acid upstream of a 5′-NTTN-3′ sequence on the PAM strand. For example, the insert sequence is situated within about 20 nucleotides upstream of the 5′-NTTN-3′ sequence, e.g., 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides upstream of the 5′-NTTN-3′ sequence.

In some embodiments, the insert sequence is situated within the corresponding region of the TTR target nucleic acid downstream of a 5′-NTTN-3′ sequence on the PAM strand. For example, the insert sequence can be situated within about 60 nucleotides downstream of the 5′-NTTN-3′ sequence, e.g., 1 nucleotide, 2 nucleotides, 3 nucleotides, 4 nucleotides, 5 nucleotides. 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 55 nucleotides, or 60 nucleotides downstream of the 5′-NTTN-3′ sequence.

In some embodiments, a template DNA described herein is used to correct a TTR mutation associated with a disease. In some embodiments, the mutation is V30M, V122I, T60A, L58H, or 184S, and the disease is hATTR, FAP, or SSA. In some embodiments, mutations V30M, V122I, T60A, L58H, or 184S are in reference to the post-translationally cleaved TTR sequence of SEQ ID NO: 257. In some embodiments, these mutations are referred to as V50M, V1421, T80A, L78H, and 1104S with respect to the full-length TTR sequence of SEQ ID NO: 256.

In some embodiments, the target sequence encodes a V30M mutation, and the template DNA comprises an insert sequence encoding an M30V mutation. In some embodiments, the target sequence comprises a 148G>A mutation in the TTR coding sequence of SEQ ID NO: 258. In some embodiments, the template DNA comprises a 148A>G mutation in the corresponding region of the TTR coding sequence of SEQ ID NO: 258.

In some embodiments, the target sequence encodes a V122I mutation, and the template DNA comprises an insert sequence encoding an 1122V mutation. In some embodiments, the target sequence comprises a 424G>A mutation in the TTR coding sequence of SEQ ID NO: 258. In some embodiments, the template DNA comprises a 424A>G mutation in the corresponding region of the TTR coding sequence of SEQ ID NO: 258.

In some embodiments, the target sequence encodes a T60A mutation, and the template DNA comprises an insert sequence encoding an A60T mutation. In some embodiments, the target sequence comprises a 238A>G mutation in the TTR coding sequence of SEQ ID NO: 258. In some embodiments, the template DNA comprises a 238G>A mutation in the corresponding region of the TTR coding sequence of SEQ ID NO: 258.

In some embodiments, the target sequence encodes an L58H mutation, and the template DNA comprises an insert sequence encoding an H58L mutation. In some embodiments, the target sequence comprises a 233T>A mutation in the TTR coding sequence of SEQ ID NO: 258. In some embodiments, the template DNA comprises a 233A>T mutation in the corresponding region of the TTR coding sequence of SEQ ID NO: 258.

In some embodiments, the target sequence encodes an 184S mutation, and the template DNA comprises an insert sequence encoding an S841 mutation. In some embodiments, the target sequence comprises a 311T>G mutation in the TTR coding sequence of SEQ ID NO: 258. In some embodiments, the template DNA comprises a 311G>T mutation in the corresponding region of the TTR coding sequence of SEQ ID NO: 258.

In some embodiments, a template DNA described herein is used to introduce a protective mutation associated with a disease. In some embodiments, the mutation is TI 19M, and the disease is hATTR. In some embodiments, the mutation is T119M, and the disease is wild-type ATTR amyloidosis. In some embodiments, the TI 19M mutation is in reference to the post-translationally cleaved TTR sequence of SEQ ID NO: 257. In some embodiments, the T119M mutation is referred to as T139M with respect to the full-length TTR sequence of SEQ ID NO: 256. In some embodiments, a target sequence encodes a T119 residue, and the template DNA comprises an insert sequence encoding a TI 19M mutation. In some embodiments, the target sequence comprises a C in the 416 position of the TTR coding sequence of SEQ ID NO: 258. In some embodiments, the template DNA comprises a 416C>T mutation in the corresponding region of the TTR coding sequence of SEQ ID NO: 258.

In some embodiments, a template DNA has at least 90% (e.g., 90%, 91%, 92%, 93%, 94%, 95%, 96%, 97%, 98%, 99%, or 100%) identity to any one of SEQ ID NOs: 287-294 or SEQ ID NOs: 303-310 or a portion of any one of SEQ ID NOs: 287-294 or SEQ ID NOs: 303-310. In some embodiments, a template DNA has at least 95% (e.g., 95%, 96%, 97%, 98%, 99%, or 100%) identity to any one of SEQ ID NOs: 287-294 or SEQ ID NOs: 303-310 or a portion of any one of SEQ ID NOs: 287-294 or SEQ ID NOs: 303-310. In some embodiments, a template DNA comprises the sequence of any one of SEQ ID NOs: 287-294 or SEQ ID NOs: 303-310 or a portion of any one of SEQ ID NOs: 287-294 or SEQ ID NOs: 303-310. In some embodiments, a template DNA comprises the donor region of any one of SEQ ID NOs: 287-294 or SEQ ID NOs: 303-310 and further has a left homology arm. In some embodiments, a template DNA comprises the donor region of any one of SEQ ID NOs: 287-294 or SEQ ID NOs: 303-310 and further has a right homology arm. In some embodiments, a template DNA comprises the donor region of any one of SEQ ID NOs: 287-294 or SEQ ID NOs: 303-310 and further has a left homology arm and a right homology arm. In some embodiments, a template DNA comprises the donor region of any one of SEQ ID NOs: 287-294 or SEQ ID NOs: 303-310 and further comprises a homology arm of about 40 to about 50 nucleotides in length. In some embodiments, a template DNA comprises the donor region of any one of SEQ ID NOs: 287-294 or SEQ ID NOs: 303-310 and further comprises a homology arm shorter than about 40 to about 50 nucleotides in length. In some embodiments, a template DNA comprises the donor region of any one of SEQ ID NOs: 287-294 or SEQ ID NOs: 303-310 and further comprises a homology arm longer than about 40 to about 50 nucleotides in length.

C. Nucleic Acid Modifications

The RNA guide or template DNA may include one or more covalent modifications with respect to a reference sequence, in particular the parent polyribonucleotide, which are included within the scope of this disclosure.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide or template DNA may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210. Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone. Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In some embodiments, the template DNA comprises one or more internucleoside modifications (e.g., phosphorothioate modifications). In some embodiments, the left homology arm comprises one or more internucleoside modifications (e.g., phosphorothioate modifications). In some embodiments, the right homology arm comprises one or more internucleoside modifications (e.g., phosphorothioate modifications). In some embodiments, the left homology arm comprises one or more internucleoside modifications (e.g., phosphorothioate modifications) and the right homology arm comprises one or more internucleoside modifications (e.g., phosphorothioate modifications). In some embodiments, the left homology arm comprises two internucleoside modifications (e.g., phosphorothioate modifications) and the right homology arm comprises two internucleoside modifications (e.g., phosphorothioate modifications). In some embodiments, the phosphorothioate modifications are at the 5′ end of the left homology arm and the 3′ end of the right homology arm. In some embodiments, the left homology arm is 5′ of the right homology arm and the left homology arm comprises two phosphorothioate modifications at the 5′ end of the left homology arm, the right homology arm comprises two phosphorothioate modifications at the 3′ end of the right homology arm. In some embodiments, the template DNA is a double stranded DNA that comprises a first strand and a second strand, wherein the first strand comprises at least one (e.g., 2) phosphorothioate modifications at the 5′ end of the first strand or at least one (e.g., 2) phosphorothioate modifications at the 3′ end of the first strand, or both. In some embodiments, the second strand comprises at least one (e.g., 2) phosphorothioate modifications at the 5′ end of the second strand or at least one (e.g., 2) phosphorothioate modifications at the 3′ end of the second strand, or both.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(0.1-thiophosphate)-guanosine, 5′-O-(1-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J. Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine. 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR 1 marks dsRNA as “self”. Cell Res. 25, 1283-1284, which is incorporated by reference in its entirety.

When a gene editing system disclosed herein comprises nucleic acids encoding the Cas12i polypeptide disclosed herein, e.g., mRNA molecules, such nucleic acid molecules may contain any of the modifications disclosed herein, where applicable.

D. Cas12i Polypeptide

In some embodiments, the composition or system of the present disclosure includes a Cas12i polypeptide as described in WO/2019/178427, the relevant disclosures of which are incorporated by reference for the subject matter and purpose referenced herein.

In some embodiments, the genetic editing system of the present disclosure comprises a Cas12i2 polypeptide described herein (e.g., a polypeptide comprising SEQ ID NO: 222 and/or encoded by SEQ ID NO: 221 (or a version thereof in which T's are replaced with U's)). In some embodiments, the Cas12i2 polypeptide comprises at least one RuvC domain. In some embodiments, the genetic editing system of the present disclosure comprises a nucleic acid molecule (e.g., a DNA molecule or a polyribonucleotide molecule) encoding a Cas12i polypeptide.

A nucleic acid sequence encoding the Cas12i2 polypeptide described herein may be substantially identical to a reference nucleic acid sequence, e.g., SEQ ID NO: 221 (or a version thereof in which T's are replaced with U's). In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 221 (or a version thereof in which T's are replaced with U's). The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 221 (or a version thereof in which T's are replaced with U's).

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 222.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 222. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 222 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i2 polypeptide may contain one or more mutations relative to SEQ ID NO: 222, for example, at position D581, G624, F626, P868, I926, V1030, E1035, S1046, or any combination thereof. In some instances, the one or more mutations are amino acid substitutions, for example, D581R, G624R, F626R, P868T, I926R, V1030G, E1035R, S1046G, or a combination thereof.

In some embodiments, the Cas12i2 polypeptide comprises a polypeptide having a sequence of SEQ ID NO: 223, SEQ ID NO: 224, SEQ ID NO: 225, SEQ ID NO: 226, or SEQ ID NO: 227. In some examples, the Cas12i2 polypeptide contains mutations at positions D581, D911, I926, and V1030. Such a Cas12i2 polypeptide may contain amino acid substitutions of D581R, D91 IR, I926R, and V1030G (e.g., SEQ ID NO: 223). In some examples, the Cas12i2 polypeptide contains mutations at positions D581, I926, and V1030. Such a Cas12i2 polypeptide may contain amino acid substitutions of D581R, I926R, and V1030G (e.g., SEQ ID NO: 224). In some examples, the Cas12i2 polypeptide may contain mutations at positions D581, I926, V1030, and S1046. Such a Cas12i2 polypeptide may contain amino acid substitutions of D581R, I926R, V1030G, and S10460 (e.g., SEQ ID NO: 225). In some examples, the Cas12i2 polypeptide may contain mutations at positions D581, G624, F626, I926, V1030, E1035, and S1046. Such a Cas12i2 polypeptide may contain amino acid substitutions of D581R, G624R, F626R, I926R, V1030G, E1035R, and S1046G (e.g., SEQ ID NO: 226). In some examples, the Cas12i2 polypeptide may contain mutations at positions D581, G624, F626, P868, I926, V1030, E1035, and S1046. Such a Cas12i2 polypeptide may contain amino acid substitutions of D581R, G624R, F626R, P868T, I926R, V1030G, E1035R, and S1046G (e.g., SEQ ID NO: 227).

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 223, SEQ ID NO: 224, SEQ ID NO: 225, SEQ ID NO: 226, or SEQ ID NO: 227. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 223, SEQ ID NO: 224, SEQ ID NO: 225, SEQ ID NO: 226, or SEQ ID NO: 227 maintains the amino acid changes (or at least 1, 2, 3 etc. of these changes) that differentiate the polypeptide from its respective parent/reference sequence.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 223, SEQ ID NO: 224, SEQ ID NO: 225, SEQ ID NO: 226, or SEQ ID NO: 227. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 223, SEQ ID NO: 224, SEQ ID NO: 225, SEQ ID NO: 226, or SEQ ID NO: 227 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i4 polypeptide described herein (e.g., a polypeptide comprising SEQ ID NO: 253 and/or encoded by SEQ ID NO: 252 (or a version thereof in which T's are replaced with U's)). In some embodiments, the Cas12i4 polypeptide comprises at least one RuvC domain.

A nucleic acid sequence encoding the Cas12i4 polypeptide described herein may be substantially identical to a reference nucleic acid sequence, e.g., SEQ ID NO: 252 (or a version thereof in which T's are replaced with U's). In some embodiments, the Cas12i4 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 252 (or a version thereof in which T's are replaced with U's). The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas12i4 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 252 (or a version thereof in which T's are replaced with U's).

In some embodiments, the Cas12i4 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 253.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 253. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 253 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ ID NO: 254 or SEQ ID NO: 255.

In some embodiments, the Cas12i4 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 254 or SEQ ID NO: 255. In some embodiments, a Cas12i4 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 254 or SEQ ID NO: 255 maintains the amino acid changes (or at least 1, 2, 3 etc. of these changes) that differentiate it from its respective parent/reference sequence.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 254 or SEQ ID NO: 255. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 254 or SEQ 1D NO: 255 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i1 polypeptide described herein (e.g., a polypeptide comprising SEQ ID NO: 265). In some embodiments, the Cas12i4 polypeptide comprises at least one RuvC domain.

In some embodiments, the Cas12i1 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 265.

In some embodiments, the present disclosure describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 265. Homology or identity can be determined by amino acid sequence alignment, e.g., using a program such as BLAST, ALIGN, or CLUSTAL, as described herein.

Also provided is a Cas12i1 polypeptide of the present disclosure having enzymatic activity, e.g., nuclease or endonuclease activity, and comprising an amino acid sequence which differs from the amino acid sequences of SEQ ID NO: 265 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i3 polypeptide described herein (e.g., a polypeptide comprising SEQ ID NO: 266). In some embodiments, the Cas12i4 polypeptide comprises at least one RuvC domain.

In some embodiments, the Cas12i3 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 266.

In some embodiments, the present disclosure describes a Cas12i3 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 266. Homology or identity can be determined by amino acid sequence alignment, e.g., using a program such as BLAST, ALIGN, or CLUSTAL, as described herein.

Also provided is a Cas12i3 polypeptide of the present disclosure having enzymatic activity, e.g., nuclease or endonuclease activity, and comprising an amino acid sequence which differs from the amino acid sequences of SEQ ID NO: 266 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors.” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at the world wide web site of kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, Pa.). In some examples, the nucleic acid encoding the Cas12i polypeptides such as Cas12i2 polypeptides as disclosed herein can be an mRNA molecule, which can be codon optimized.

Exemplary Cas12i polypeptide sequences and corresponding nucleotide sequences are listed in Table 6.

TABLE 6
Cas12i and TTR Sequences

| SEQ ID | | |
|---|---|---|
| NO: | Sequence | Description |
| 221 | ATGAGCAGCGCGATCAAAAGCTACAAGAGCGTTCTGCGTCCGAACGAGCGTAAGA | Nucleotide |
| | ACCAACTGCTGAAAAGCACCATTCAGTGCCTGGAAGACGGTAGCGCGTTCTTTTT | sequence |
| | CAAGATGCTGCAAGGCCTGTTTGGTGGCATCACCCCGGAGATTGTTCGTTTCAGC | encoding |
| | ACCGAACAGGAGAAACAGCAACAGGATATCGCGCTGTGGTGCGCGGTTAACTGGT | Cas12i2 |
| | TCCGTCCGGTGAGCCAAGACAGCCTGACCCACACCATTGCGAGCGATAACCTGGT | |
| | GGAGAAGTTTGAGGAATACTATGGTGGCACCGCGAGCGACGCGATCAAACAGTAC | |
| | TTCAGCGCGAGCATTGGCGAAAGCTACTATTGGAACGACTGCCGTCAACACTACT | |
| | ATGATCTGTGCCGTGAGCTGGGTGTTGAGGTGAGCGACCTGACCCATGATCTGGA | |
| | GATCCTGTGCCGTGAAAAGTGCCTGGCGGTTGCGACCGAGAGCAACCAGAACAAC | |
| | AGCATCATTAGCGTTCTGTTTGGCACCGGCGAAAAAGAGGACCGTAGCGTGAAAC | |
| | TGCGTATCACCAAGAAAATTCTGGAGGCGATCAGCAACCTGAAAGAAATCCCGAA | |
| | GAACGTTGCGCCGATTCAAGAGATCATTCTGAACGTGGCGAAAGCGACCAAGGAA | |
| | ACCTTCCGTCAGGTGTATGCGGGTAACCTGGGTGCGCCGAGCACCCTGGAGAAAT | |
| | TTATCGCGAAGGACGGCCAAAAAGAGTTCGATCTGAAGAAACTGCAGACCGACCT | |
| | GAAGAAAGTTATTCGTGGTAAAAGCAAGGAGCGTGATTGGTGCTGCCAGGAAGAG | |
| | CTGCGTAGCTACGTGGAGCAAAACACCATCCAGTATGACCTGTGGGCGTGGGGCG | |
| | AAATGTTCAACAAAGCGCACACCGCGCTGAAAATCAAGAGCACCCGTAACTACAA | |
| | CTTTGCGAAGCAACGTCTGGAACAGTTCAAAGAGATTCAGAGCCTGAACAACCTG | |
| | CTGGTTGTGAAGAAGCTGAACGACTTTTTCGATAGOGAATTTTTCAGCGGCGAGG | |
| | AAACCTACACCATCTGCGTTCACCATCTGGGTGGCAAGGACCTGAGCAAACTGTA | |
| | TAAGGCGTGGGAGGATGATCCGGCGGACCCGGAAAACGCGATTGTGGTTCTGTGC | |
| | GACGATCTGAAAAACAACTTTAAGAAAGAGCCGATCCGTAACATTCTGCGTTACA | |
| | TCTTCACCATTCCTCAAGAATGCAGCGCGCAGGACATCCTGGCGGCGGCGAAGTA | |
| | CAACCAACAGCTGGATCGTTATAAAAGCCAAAAGGCGAACCCGAGCGTTCTGGGT | |
| | AACCAGGGCTTTACCTGGACCAACGCGGTGATCCTGCCGGAGAAGGCGCAGCGTA | |
| | ACGACCGTCCGAACAGCCTGGATCTGCGTATTTGGCTGTACCTGAAACTGCGTCA | |
| | CCCGGACGGTCGTTGGAAGAAACACCATATCCCGTTCTACGATACCCGTTTCTTC | |
| | CAAGAAATTTATGCGGCGGGCAACAGCCCGGTTGACACCTGCCAGTTTCGTACCC | |
| | CGCGTTTCGGTTATCACCTGCCGAAACTGACCGATCAGACCGCGATCCGTGTTAA | |
| | CAAGAAACATGTGAAAGCGGCGAAGACCGAGGCGCGTATTCGTCTGGCGATCCAA | |
| | CAGGGCACCCTGCCGGTGAGCAACCTGAAGATCACCGAAATTAGCGCGACCATCA | |
| | ACAGCAAAGGTCAAGTGCGTATTCCGGTTAAGTTTGACGTGGGTCGTCAAAAAGG | |
| | CACCCTGCAGATCGGTGACCGTTTCTGCGGCTACGATCAAAACCAGACCGCGAGC | |
| | CACGCGTATAGCCTGTGGGAAGTGGTTAAAGAGGGTCAATACCATAAAGAGCTGG | |
| | GCTGCTTTGTTCGTTTCATCAGCAGCGGTGACATCGTGAGCATTACCGAGAACCG | |
| | TGGCAACCAATTTGATCAGCTGAGCTATGAAGGTCTGGCGTACCCGCAATATGCG | |
| | GACTGGCGTAAGAAAGCGAGCAAGTTCGTGAGCCTGTGGCAGATCACCAAGAAAA | |
| | ACAAGAAAAAGGAAATCGTGACCGTTGAAGCGAAAGAGAAGTTTGACGCGATCTG | |
| | CAAGTACCAGCCGCGTCTGTATAAATTCAACAAGGAGTACGCGTATCTGCTGCGT | |
| | GATATTGTTCGTGGCAAAAGCCTGGTGGAACTGCAACAGATTCGTCAAGAGATCT | |
| | TTCGTTTCATTGAACAGGACTGCGGTGTTACCCGTCTGGGCAGCCTGAGCCTGAG | |
| | CACCCTGGAAACCGTGAAAGCGGTTAAGGGTATCATTTACAGCTATTTTAGCACC | |
| | GCGCTGAACGCGAGCAAGAACAACCCGATCAGCGACGAACAGCGTAAAGAGTTTG | |
| | ATCCGGAACTGTTCGCGCTGCTGGAAAAGCTGGAGCTGATTCGTACCCGTAAAAA | |
| | GAAACAAAAAGTGGAACGTATCGCGAACAGCCTGATTCAGACCTGCCTGGAGAAC | |
| | AACATCAAGTTCATTCGTGGTGAAGGCGACCTGAGCACCACCAACAACGCGACCA | |
| | AGAAAAAGGCGAACAGCCGTAGCATGGATTGGTTGGCGCGTGGTGTTTTTAACAA | |
| | AATCCGTCAACTGGCGCCGATGCACAACATTACCCTGTTCGGTTGCGGCAGCCTG | |
| | TACACCAGCCACCAGGACCCGCTGGTGCATCGTAACCCGGATAAAGCGATGAAGT | |
| | GCCGTTGGGCGGCCATCCCGGTTAAGGACATTGGCGATTGGGTGCTGCGTAAGCT | |
| | GAGCCAAAACCTGCGTGCGAAAAACATCGGCACCGGCGAGTACTATCACCAAGGT | |
| | GTTAAAGAGTTCCTGAGCCATTATGAACTGCAGGACCTGGAGGAAGAGCTGCTGA | |
| | AGTGGCGTAGCGATCGTAAAAGCAACATTCCGTGCTGGGTGCTGCAGAACCGTCT | |
| | GGCGGAGAAGCTGGGCAACAAAGAAGCGGTGGTTTACATCCCGGTTCGTGGTGGC | |
| | CGTATTTATTTTGCGACCCACAAGGTGGCGACCGGTGCGGTGAGCATCGTTTTCG | |
| | ACCAAAAACAAGTGTGGGTTTGCAACGCGGATCATGTTGCGGCGGCGAACATCGC | |
| | GCTGACCGTGAAGGGTATTGGCGAACAAAGCAGCGACGAAGAGAACCCGGATGGT | |
| | AGCCCTATCAAACTGCAGCTGACCAGC | |
| 222 | MSSAIKSYKSVLRPNERKNQLLKSTIQCLEDGSAFFFKMLQGLFGGITPEIVRFS | Cas12i2 |
| | TEQEKQQQDIALWCAVNWFRPVSQDSLTHTIASDNLVEKFEEYYGGTASDAIKQY | amino acid |
| | FSASIGESYYWNDCRQQYYDLCRELGVEVSDLTHDLEILCREKCLAVATESNQNN | sequence |
| | SIISVLFGTGEKEDRSVKLRITKKILEAISNLKEIPKNVAPIQEIILNVAKATKE | |
| | TFRQVYAGNLGAPSTLEKFIARDGQKEFDLKKLQTDLKRVIRGKSKERDWCCQEE | |
| | LRSYVEQNTTQYDLWAWGEMFNKAHTALKIRSTRNYNFAKQRLEQFKEIQSLNNL | |
| | LVVKRLNDFFDSEFFSGEETYTICVHHLGGKDLSKLYKAWEDDPADPENAIVVLC | |
| | DDLKNNFKKEPIRNILRYIFTIRQECSAQDILAAAKYNQQLDRYKSQKANPSVLG | |
| | NQGFTWTNAVILPEKAQRNDRPNSLDLRIWLYLKLRHPDGRWKKHHIPFYDTRFF | |
| | QEIYAAGNSPVDTCQFRTPRFGYHLPKLTDQTAIRVNKKHVKAAKTEARIRLAIQ | |
| | QGTLPVSNLKITEISATINSKGQVRIPVKFDVGRQKGTLQIGDRFCGYDQNQTAS | |
| | HAYSLWEVVKEGQYHKELGCFVRFISSGDIVSITENRGNQFDQLSYEGLAYPQYA | |
| | DWRKKASKFVSLWQITKKNKKKEIVTVEAKEKFDAICKYQPRLYKFNKEYAYLLR | |
| | DIVRGKSLVELQQIRQEIFRFIEQDCGVTRLGSLSLSTLETVKAVKGIIYSYFST | |
| | ALNASKNNPISDEQRKEFDPELFALLEKLELIRTRKKKQKVERIANSLIQTCLEN | |
| | NIKFIRGEGDLSTTNNATKKKANSRSMDWLARGVFNKIRQLAPMHNITLFGCGSL | |
| | YTSHQDPLVHRNPDKAMKCRWAAIPVKDIGDWVLRKLSQNLRAKNIGTGEYYHQG | |
| | VKEFLSHYELQDLEEELLKWRSDRKSNIPCWVLQNRLAEKLGNKEAVVYIPVRGG | |
| | RIYFATHKVATGAVSIVFDQKQVWVCNADHVAAANIALTVKGIGEQSSDEENPDG | |
| | SRIKLQLTS | |
| 223 | MSSAIKSYKS VLRPNERKNQ LLKSTIQCLE DGSAFFFKML QGLFGGITPE | Variant |
| | TVRFSTEQEK QQQDIALWCA VNWFRPVSQD SLTHTIASDN LVEKFEEYYG | Cas 1212 of |
| | GTASDAIKQY FSASIGESYY WNDCRQQYYD LCRELGVEVS DLTADLEILC | SEQ ID NO: |
| | REKCLAVATE SNQNNSIISV LFGTGEKEDR SVKLRITKKI LEAISNLKEI | 3 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMFNKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVRAAK TEARIRLATQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMR NITLFGCGSL YTSHQDPLVH RNPDKAMKCR | |
| | WAAIPVKDIG RWVLRKLSQN LRAKNRGTGE YYHQGVKEFL SHYELQDLEE | |
| | ELLRWRSDRK SNIPCWVLON RLAEKLGNKE AVVYIPVRGG RIYFATHKVA | |
| | TGAVSIVFDQ KQVWVCNADH VAAANTALTG KGIGEQSSDE ENPDGSRIKL | |
| | QLTS | |
| 224 | MSSAIKSYKS VLRPNERKNQ LLKSTIQCLE DGSAFFFKML QGLFGGITPE | Variant |
| | TVRFSTEQEK QQQDIALWCA VNWFRPVSQD SLTHTIASDN LVEKFEEYYG | Cas 1212 of |
| | GTASDAIKQY FSASIGESYY WNDCRQQYYD LCRELGVEVS DLTHDLEILC | SEQ ID NO: |
| | REKCLAVATE SNQNNSIISV LFGTGEKEDR SVKLRITKKI LEAISNLKEI | 4 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMFNKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLATQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANTALTG KGIGEQSSDE ENPDGSRIKL | |
| | QLTS | |
| 225 | MSSAIKSYKS VLRPNERKNQ LLKSTIQCLE DGSAFFFKML QGLFGGITPE | Variant |
| | TVRFSTEQEK QQQDIALWCA VNWFRPVSQD SLTHTIASDN LVEKFEEYYG | Cas 1212 of |
| | GTASDAIKQY FSASIGESYY WNDCRQQYYD LCRELGVEVS DLTHDLEILC | SEQ ID NO: |
| | REKCLAVATE SNQNNSIISV LFGTGEKEDR SVKLRITKKI LEAISNLKEI | 5 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMFNKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQELIW.NAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLATQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QSIFRFIEQD CGVTRLGSLS | |
| | LSTLETVKAV KGIIYSYFST ALNASKNNPI SDEQRKEFDP ELFALLEKLE | |
| | LTRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIFVRGG RIYFATHKVA | |
| | TGAVSIVFDQ KQVWVCNADH VAAANIALTG KGIGEQSSDE SNPDGGRIKL | |
| | QLTS | |
| 226 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFSSYYG | Cas12i2 of |
| | GTASDAIKQY FSASIGESYY WNDCRQQYYD LCRELGVEVS DLTHDLEILC | SEQ ID NO: |
| | REKCLAVATE SNQNNSIISV LFGTGEKEDR SVKLRITKKI LEAISNLKSI | 495 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMFNKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQS CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELRCRVRFI SSGDIVSITS NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFISQD CGVTRLGSLS | |
| | LSTLETVKAV KGIIYSYFST ALNASKNNPI SDEQRKEFDP ELFALLEKLE | |
| | LTRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLASKLGNKE AVVYIPVRGG RIYFATHKVA | |
| | TGAVSIVFDQ KQVWVCNADH VAAANIALTG KGIGRQSSDS SNPDGGRIKL | |
| | QLTS | |
| 227 | MSSAIKSYKS VLRPNERKNQ LLKSTIQCLE DGSAFFFKML QGLFGGITPE | |
| | IVRFSTEQEK QQQDIALWCA VNWFRPVSQD SLTHTIASDN LVEKFSSYYG | SEQ ID NO: |
| | GTASDAIKQY FSASIGESYY WNDCRQQYYD LCRELGVEVS DLTHDLEILC | 496 of |
| | REKCLAVATE SNQNNSIISV LFGTGEKEDR SVKLRITKKI LEAISNLKSI | PCT/US2021/ |
| | PKNVAPIQEI ILNVAKATKE TFRQVYAGNL GAPSTLEKFI AKDGQKEFDL | 025257 |
| | KKLQTDLKKV IRGKSKERDW CCQEELRSYV EQNTIQYDLW AWGEMFNKAH | Cas12i2 of |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQS CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIFVKF RVGRQKGTLQ IGDRFCGYDQ | |
| | NQTASHAYSL WEVVKEGQYH KELRCRVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QSIFRFIEQD CGVTRLGSLS | |
| | LSTLETVKAV KGIIYSYFST ALNASKNNPI SDEQRKEFDP ELFALLEKLE | |
| | LTRTRKKKQK VERIANSLIQ TCLENKIKFI RGEGDLSTTN NATKKKANSR | |
| | SMDWLARGVF NKIRQLATMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIFVRGG RIYFATHKVA | |
| | TGAVSIVFDQ KQVWVCNADH VAAANIALTG KGIGRQSSDS SNPDGGRIKL | |
| | QLTS | |
| 252 | ATGGCTTCCATCTCTAGGCCATACGGCACCAAGCTGCGACCGGACGCACGGAAGA | Nucleotide |
| | AGGAGATGCTCGATAAGTTCTTIAATACACTGACTAAGGGTCAGCGCGTGTTCGC | sequence |
| | AGACCTGGCCCTGTGCATCTATGGCTCCCTGACCCTGGAGATGGCCAAGTCTCTG | encoding |
| | GAGCCAGAAAGTGATTCAGAACTGGTGTGCGCTATTGGGTGGTTTCGGCTGGTGG | Cas12i4 |
| | ACAAGACCATCTGGTCCAAGGATGGCATCAAGCAGGAGAATCTGGTGAAACAGTA | |
| | CGAAGCCTATTCCGGAAAGGAGGCTTCTGAAGTGGTCAAAACATACCTGAACAGC | |
| | CCCAGCTCCGACAAGTACGTGTGGATCGATTGCAGGCAGAAATTCCTGAGGTTTC | |
| | AGCGCGAGCTCGGCACTCGCAACCTGTCCGAGGACTTCGAATGTATGCTCTTTGA | |
| | ACAGTACATTAGACTGACCAAGGGCGAGATCGAAGGGTATGCCGCTATTTCAAAT | |
| | ATGTTCGGAAACGGCGAGAAGGAAGACCGGAGCAAGAAAAGAATGTACGCTACAC | |
| | GGATGAAAGATTGGCTGGAGGCAAACGAAAATATCACTTGGGAGCAGTATAGAGA | |
| | GGCCCTGAAGAACCAGCTGAATGCTAAAAACCTGGAGCAGGTTGTGGCCAATTAC | |
| | AAGGGGAACGCTGGCGGGGCAGACCCCTTCTTTAAGTATAGCTTCTCCAAAGAGG | |
| | GAATGGTGAGCAAGAAAGAACATGCACAGCAGCTCGACAAGTTCAAAACCGTCCT | |
| | GAAGAACAAAGCCCGGGACCTGAATTTTCCAAACAAGGAGAAGCTGAAGCAGTAC | |
| | CTGGAGGCCGAAATCGGCATTCCGGTCGACGCTAACGTGTACTCCCAGATGTTCT | |
| | CTAACGGGGTGAGTGAGGTCCAGCCTAAGACCACACGGAATATGTCTTTTAGTAA | |
| | CGAGAAACTGGATCTGCTCACTGAACTGAAGGACCTGAACAAGGGCGATGGGTTC | |
| | GAGTACGCCAGAGAAGTGCTGAACGGGTTCTTTGACTCCGAGCTCCACACTACCG | |
| | AGGATAAGTTTAATATCACCTCTAGGTACCTGGGAGGCGACAAATCAAACCGCCT | |
| | GAGCAAACTCTATAAGATCTGGAAGAAAGAGGGTGTGGACTGCGAGGAAGGCATT | |
| | CAGCAGTTCTGTGAAGCCGTCAAAGATAAGATGGGCCAGATCCCCATTCGAAATG | |
| | TGCTGAAGTACCTGTGGCAGTTCCGGGAGACAGTCAGTGCCGAGGATTTTGAAGC | |
| | AGCCGCTAAGGCTAACCATCTGGAGGAAAAGATCAGCCGGGTGAAAGCCCACCCA | |
| | ATCGTGATTAGCAATAGGTACTGGGCTTTTGGGACTTCCGCACTGGTGGGAAACA | |
| | TTATGCCCGCAGACAAGAGGCATCAGGGAGAGTATGCCGGTCAGAATTTCAAAAT | |
| | GTGGCTGGAGGCTGAACTGCACTACGATGGCAAGAAAGCAAAGCACCATCTGCCT | |
| | TTTTATAACGCCCGCTTCTTTGAGGAAGTGTACTGCTATCACCCCTCTGTCGCCG | |
| | AGATCACTCCTTTCAAAACCAAGCAGTTTGGCTGTGAAATCGGGAAGGACATTCC | |
| | AGATTACGTGAGCGTCGCTCTGAAGGACAATCCGTATAAGAAAGCAACCAAACGA | |
| | ATCCTGCGTGCAATCTACAATCCCGTCGCCAACACAACTGGCGTTGATAAGACCA | |
| | CAAACTGCAGCTTCATGATCAAACGCGAGAATGACGAATATAAGCTGGTCATCAA | |
| | CCGAAAAATTTCCGTGGATCGGCCTAAGAGAATCGAAGTGGGCAGGACAATTATG | |
| | GGGTACGACCGCAATCAGACAGCTAGCGATACTTATTGGATTGGCCGGCTGGTGC | |
| | CACCTGGAACCCGGGGCGCATACCGCATCGGAGAGTGGAGCGTCCAGTATATTAA | |
| | GTCCGGGCCTGTCCTGTCTAGTACTCAGGGAGTTAACAATTCCACTACCGACCAG | |
| | CTGGTGTACAACGGCATGCCATCAAGCTCCGAGCGGTTCAAGGCCTGGAAGAAAG | |
| | CCAGAATGGCTTTTATCCGAAAACTCATTCGTCAGCTGAATGACGAGGGACTGGA | |
| | ATCTAAGGGTCAGGATTATATCCCCGAGAACCCTTCTAGTTTCGATGTGCGGGGC | |
| | GAAACCCTGTACGTCTTTAACAGTAATTATCTGAAGGCCCTGGTGAGCAAACACA | |
| | GAAAGGCCAAGAAACCTGTTGAGGGGATCCTGGACGAGATTGAAGCCTGGACATC | |
| | TAAAGACAAGGATTCATGCAGCCTGATGCGGCTGAGCAGCCTGAGCGATGCTTCC | |
| | ATGCAGGGAATCGCCAGCCTGAAGAGTCTGATTAACAGCTACTTCAACAAGAATG | |
| | GCTGTAAAACCATCGAGGACAAAGAAAAGTTTAATCCCGTGCTGTATGCCAAGCT | |
| | GGTTGAGGTGGAACAGCGGAGAACAAACAAGCGGTCTGAGAAAGTGGGAAGAATC | |
| | GCAGGTAGTCTGGAGCAGCTGGCCCTGCTGAACGGGGTTGAGGTGGTCATCGGCG | |
| | AAGCTGACCTGGGGGAGGTCGAAAAAGGAAAGAGTAAGAAACAGAATTCACGGAA | |
| | CATGGATTGGTGCGCAAAGCAGGTGGCACAGCGGCTGGAGTACAAACTGGCCTTC | |
| | CATGGAATCGGTTACTTTGGAGTGAACCCCATGTATACCAGCCACCAGGACCCTT | |
| | TCGAACATAGGCGCGTGGCTGATCACATCGTCATGCGAGCACGTTTTGAGGAAGT | |
| | CAACGTGGAGAACATTGCCGAATGGCACGTGCGAAATTTCTCAAACTACCTGCGT | |
| | GCAGACAGCGGCACTGGGCTGTACTATAAGCAGGCCACCATGGACTTCCTGAAAC | |
| | ATTACGGTCTGGAGGAACACGCTGAGGGCCTGGAAAATAAGAAAATCAAGTTCTA | |
| | TGACTTTAGAAAGATCCTGGAGGATAAAAACCTGACAAGCGTGATCATTCCAAAG | |
| | AGGGGCGGGCGCATCTACATGGCCACCAACCCAGTGACATCCGACTCTACCCCGA | |
| | TTACATACGCCGGCAAGACTTATAATAGGTGTAACGCTGATGAGGTGGCAGCCGC | |
| | TAATATCGTTATTTCTGTGCTGGCTCCCCGCAGTAAGAAAAACGAGGAACAGGAC | |
| | GATATCCCTCTGATTACCAAGAAAGCCGAGAGTAAGTCACCACCGAAAGACCGGA | |
| | AGAGATCAAAAACAAGCCAGCTGCCTCAGAAA | |
| 253 | MASISRPYGTKLRPDARKKEMLDKFFNTLTKGQRVFADLALCIYGSLTLEMAKSL | Cas12i4 |
| | EPESDSELVCAIGWFRLVDKTIWSKDGIKQENLVKQYEAYSGKEASEVVKTYLNS | amino acid |
| | PSSDKYVWIDCRQKFLRFQRELGTRNLSEDFECMLFEQYIRLTKGEIEGYAAISN | sequence |
| | MFGNGEKEDRSKKRMYATRMKDWLEANENITWEQYREALKNQLNAKNLEQVVANY | |
| | KGNAGGADPFFKYSFSKEGMVSKKEHAQQLDKFKTVLKNKARDLNFPNKERLKQY | |
| | LEAEIGIPVDANVYSQMFSNGVSEVQPKTTRNMSFSNEKLDLLTELKDLNKGDGE | |
| | EYAREVLNGFFDSELHTTEDKFNITSRYLGGDKSNRLSRLYKIWKKEGVDCEEGI | |
| | QQFCEAVKDKMGQIPIRNVLKYLWQFRETVSAEDFEAAAKANHLEEKISRVKAHP | |
| | IVISNRYWAFGTSALVGNIMPADKRHQGEYAGQNFKMWLEAELHYDGKKAKHHLP | |
| | FYNARFFEEVYCYHPSVAEITPFKTKQFGCEIGKDIPDYVSVALKDNPYKKATKR | |
| | ILRAIYNPVANTTGVDKTTNCSFMIKRENDEYKLVINRKISVDRPKRIEVGRTIM | |
| | GYDRNQTASDTYWIGRLVPPGTRGAYRIGEWSVQYIKSGPVLSSTQGVNNSTTDQ | |
| | LVYNGMPSSSERFKAWKKARMAFIRKLIRQLNDEGLESKGQDYIPENPSSFDVRG | |
| | ETLYVFNSNYLKALVSKHRKAKKPVEGILDEIEAWTSKDKDSCSLMRLSSLSDAS | |
| | MQGIASLKSLINSYFNKNGCKTTEDKEKFNPVLYAKLVEVEQRRTNKRSEKVGRI | |
| | AGSLEQLALLNGVEVVIGEADLGEVEKGKSKKQNSRNMDWCAKQVAQRLEYKLAF | |
| | HGIGYFGVNPMYTSHQDPFEHRRVADHIVMRARFEEVNVENIAEWHVRNFSNYLR | |
| | ADSGTGLYYKQATMDFLKHYGLEEHAEGLENKKIKFYDFRKILEDKNLTSVIIPK | |
| | RGGRIYMATNPVTSDSTPITYAGKTYNRCNADEVAAANIVISVLAPRSKKNEEQD | |
| | DIPLITKKAESKSPPKDRKRSKTSQLPQK | |
| 254 | MASISRPYGT KLRPDARKKE MLDKFFNTLT KGQRVFADLA LCIYGSLTLE | Variant |
| | MAKSLEPESD SELVCAIGWF RLVDKTIWSK DGIKQENLVK QYEAYSGKEA | Cas12i4 A |
| | SEVVKTYLNS PSSDKYVWID CRQKFLRFQR ELGTRNLSED FECMLFEQYI | |
| | RLTKGEIEGY AAISNMFGNG EKEDRSKKRM YATRMKDWLE ANENITWEQY | |
| | REALKNQLNA KNLEQVVANY KGNAGGADPF FKYSFSKEGM VSKKEHAQQL | |
| | DKFKTVLKNK ARDLNFPNKE KLKQYLEAEI GIPVDANVYS QMFSNGVSEV | |
| | QPKTTRNMSF SNEKLDLLTE LKDLNKGDGF EYAREVLNGF FDSELHTTED | |
| | KFNITSRYLG GDKSNRLSKL YKIWKKEGVD CEEGIQQFCE AVKDKMGQIP | |
| | IRNVLKYLWQ FRETVSAEDF EAAAKANHLE EKISRVKAHP IVISNRYWAF | |
| | GTSALVGNIM PADKRHQGEY AGQNFKMWLE AELHYDGKKA KHHLPFYNAR | |
| | FFEEVYCYHP SVAEITPFKT KQFGCEIGKD IPDYVSVALK DNPYKKATKR | |
| | ILRAIYNPVA NTTGVDKTTN CSFMIKREND EYKLVINRKI SRDRPKRIEV | |
| | GRTIMGYDRN QTASDTYWIG RLVPPGTRGA YRIGEWSVQY IKSGPVLSST | |
| | QGVNNSTTDQ LVYNGMPSSS ERFKAWKKAR MAFIRKLIRQ LNDEGLESKG | |
| | QDYIPENPSS FDVRGETLYV FNSNYLKALV SKHRKAKKPV EGILDEIEAW | |
| | TSKDKDSCSL MRLSSLSDAS MQGIASLKSL INSYFNKNGC KTIEDKEKFN | |
| | PVLYAKLVEV EQRRTNKRSE KVGRIAGSLE QLALLNGVEV VIGEADLGEV | |
| | EKGKSKKONS RNMDWCAKOV AQRLEYKLAF HGIGYFGVNP MYTSRQDPFE | |
| | HRRVADHIVM RARFEEVNVE NIAEWHVRNF SNYLRADSGT GLYYKQATMD | |
| | FLKHYGLEEH AEGLENKKIK FYDFRKILED KNLTSVIIPK RGGRIYMATN | |
| | PVTSDSTPIT YAGKTYNRCN ADEVAAANIV ISVLAPRSKK NREQDDIPLI | |
| | TKKAESKSPP KDRKRSKTSQ LPQK | |
| 255 | MASISRPYGT KLRPDARKKE MLDKFFNTLT KGQRVFADLA LCIYGSLTLE | Variant |
| | MAKSLEPESD SELVCAIGWF RLVDKTIWSK DGIKQENLVK QYEAYSGKEA | Cas1214 B |
| | SEVVKTYLNS PSSDKYVWID CRQKFLRFQR ELGTRNLSED FECMLFEQYI | |
| | RLTKGEIEGY AAISNMFGNG EKEDRSKKRM YATRMKDWLE ANENITWEQY | |
| | REALKNQLNA KNLEQVVANY KGNAGGADPF FKYSFSKEGM VSKKEHAQQL | |
| | DKFKTVLKNK ARDLNFPNKE KLKQYLEAEI GIPVDANVYS QMFSNGVSEV | |
| | QPKTTRNMSF SNEKLDLLTE LKDLNKGDGF EYAREVLNGF FDSELHTTED | |
| | KFNITSRYLG GDKSNRLSKL YKIWKKEGVD CEEGIQQFCE AVKDKMGQIP | |
| | IRNVLKYLWQ FRETVSAEDF EAAAKANHLE EKISRVKAHP IVISNRYWAF | |
| | GTSALVGNIM PADKRHQGEY AGQNFKMWLR AELHYDGKKA KHHLPFYNAR | |
| | FFEEVYCYHP SVAEITPFKT KQFGCEIGKD IPDYVSVALK DNPYKKATKR | |
| | ILRAIYNPVA NTTRVDKTTN CSFMIKREND EYKLVINRKI SRDRPKRIEV | |
| | GRTIMGYDRN QTASDTYWIG RLVPPGTRGA YRIGEWSVQY IKSGPVLSST | |
| | QGVNNSTTDQ LVYNGMPSSS ERFKAWKKAR MAFIRKLIRQ LNDEGLESKG | |
| | QDYIPENPSS FDVRGETLYV FNSNYLKALV SKHRKAKKPV EGILDEIEAW | |
| | TSKDKDSCSL MRLSSLSDAS MQGIASLKSL INSYFNKNGC KTIEDKEKFN | |
| | PVLYAKLVEV EQRRTNKRSE KVGRIAGSLE QLALLNGVEV VIGEADLGEV | |
| | EKGKSKKQNS RNMDWCAKQV AQRLEYKLAF HGIGYFGVNP MYTSHQDPFE | |
| | HRRVADHIVM RARFEEVNVE NIAEWHVRNF SNYLRADSGT GLYYKQATMD | |
| | FLKHYGLEEH AEGLENKKIK FYDFRKILED KNLTSVIIPK RGGRIYMATN | |
| | PVTSDSTPIT YAGKTYNRCN ADEVAAANIV ISVLAPRSKK NREQDDIPLI | |
| | TKKAESKSPP KDRKRSKTSQ LPQK | |
| 265 | MSNKEKNASETRRAYTTKMIPRSHDRMKLLGNFMDYLMDGTPIFFELWNQFGGGI | Cas12il |
| | DRDIISGTANKDKISDDLLLAVNWFKVMPINSKPQGVSPSNLANLFQQYSGSEPD | (SEQ ID NO: |
| | IQAQEYFASNFDTEKHQWKDMRVEYERLLAELQLSRSDMHHDLKLMYKEKCIGLS | 3 of U.S. |
| | LSTAHYITSVMFGTGAKNNRQTKHQFYSKVIQLLEESTQINSVEQLASIILKAGD | Patent No. |
| | CDSYRKLRIRCSRKGATPSILKIVQDYELGTNHDDEVNVPSLIANLKEKLGRFEY | 10,808,245) |
| | ECEWKCMEKIKAFLASKVGPYYLGSYSAMLENALSPIKGMTTKNCKFVLKQIDAK | |
| | NDIKYENEPFGKIVEGFFDSPYFESDTNVKWVLHPHHIGESNIKTLWEDLNAIHS | |
| | KYEEDIASLSEDKKEKRIKVYQGDVCQTINTYCEEVGKEAKTPLVQLLRYLYSRK | |
| | DDIAVDKIIDGITFLSKKHKVEKQKINPVIQKYPSFNFGNNSKLLGKIISPKDKL | |
| | KHNLKCNRNQVDNYIWIEIKVLNTKTMRWERHHYALSSTRFLEEVYYPATSENPP | |
| | DALAARFRTKTNGYEGKPALSAEQIEQIRSAPVGLRKVRRRQMRLEAARQQNLLP | |
| | RYTWGKDFNINICKRGNNFEVTLATKVKKKKEKNYKVVLGYDANIVRKNTYAAIE | |
| | AHANGDGVIDYNDLPVKPIESGFVTVESQVRDKSYDQLSYNGVKLLYCKPHVESR | |
| | RSFLEKYRNGTMKDNRGNNIQIDFMKDFEAIADDETSLYYFNMKYCKLLQSSIRN | |
| | HSSQAKEYREEIFELLRDGKLSVLKLSSLSNLSFVMFKVAKSLIGTYFGHLLKKP | |
| | KNSKSDVKAPPITDEDKQKADPEMFALRLALEEKRLNKVKSKKEVIANKIVAKAL | |
| | ELRDKYGPVLIKGENISDTTKKGKKSSTNSFLMDWLARGVANKVKEMVMMHQGLE | |
| | FVEVNPNFTSHQDPFVHKNPENTFRARYSRCTPSELTERNRKEILSFLSDKPSKR | |
| | PTNAYYNEGAMAFLATYGLKKNDVLGVSLERFRQIMANILHQRSEDQLLFPSRGG | |
| | MFYLATYKLDADATSVNWNGKQFWVCNADLVAAYNVGLVDIQKDFKKK | |
| 266 | MSISNNNILPYNPKLLPDDRKHKMLVDTFNQLDLIRNNLHDMIIALYGALKYDNI | Cas12i3 |
| | KQFASKEKPHISADALCSINWFRLVKTNERKPAIESNQIISKFIQYSGHTPDKYA | (SEQ ID NO: |
| | LSHITGNHEPSHKWIDCREYAINYARIMHLSFSQFQDLATACLNCKILILNGTLT | 14 of U.S. |
| | SSWAWGANSALFGGSDKENFSVKAKILNSFIENLKDEMNTTKFQVVERVCQQIGS | Patent No. |
| | SDAADLFDLYRSTVKDGNRGPATGRNPKVMNLFSQDGEISSEQREDFIESFQKVM | 10,808,245) |
| | QEKNSKQIIPHLDRLKYHLVKQSGLYDIYSWAAAIKNANSIIVASNSSNLNTILN | |
| | KTEKQQTFEELRKDEKIVACSKILLSVNDTLPEDLHYNPSTSNLGKNLDVFFDLL | |
| | NENSVHTIENKEEKNKIVKECVNQYMEECKGLNKPPMPVLLTFISDYAHKHQAQD | |
| | FLSAAKMNFIDLKIKSIKVVPTVHGSSPYTWISNLSKKNKDGKMIRTPNSSLIGW | |
| | IIPPEEIHDQKFAGQNPIIWAVLRVYCNNKWEMHHFPFSDSRFFTEVYAYKPNLP | |
| | YLPGGENRSKRFGYRHSTNLSNESRQILLDKSKYAKANKSVLRCMENMTHNVVFD | |
| | PKTSLNIRIKTDKNNSPVLDDKGRITFVMQINHRILEKYNNTKIEIGDRILAYDQ | |
| | NQSENHTYAILQRTEEGSHAHQFNGWYVRVLETGKVTSIVQGLSGPIDQLNYDGM | |
| | PVTSHKFNCWQADRSAFVSQFASLKISETETFDEAYQAINAQGAYTWNLFYLRIL | |
| | RKALRVCHMENINQFREEILAISKNRLSPMSLGSLSQNSLKMIRAFKSIINCYMS | |
| | RMSFVDELQKKEGDLELHTIMRLTDNKLNDKRVEKINRASSFLTNKAHSMGCKMI | |
| | VGESDLPVADSKTSKKQNVDRMDWCARALSHKVEYACKLMGLAYRGIPAYMSSHQ | |
| | DPLVHLVESKRSVLRPRFVVADKSDVKQHHLDNLRRMLNSKTKVGTAVYYREAVE | |
| | LMCEELGIHKTDMAKGKVSLSDFVDKFIGEKAIFPQRGGRFYMSTKRLTTGAKLI | |
| | CYSGSDVWLSDADEIAAINIGMFWCDQTGAFKKKKKEKLDDEECDILPFRPM | |
| 228 | ACAGAAGTCCACTCATTCTTGGCAGGATGGCTTCTCATCGTCTGCTCCTCCTCTG | TTR |
| | CCTTGCTGGACTGGTATTTGTGTCTGAGGCTGGCCCTACGGTGAGTGTTTCTGTG | |
| | ACATCCCATTCCTACATTTAAGATTCACGCTAAATGAAGTAGAAGTGACTCCTTC | |
| | CAGCTTTGCCAACCAGCTTTTATTACTAGGGCAAGGGTACCCAGCATCTATTTTT | |
| | AATATAATTAATTCAAACTTCAAAAAGAATGAAGTTCCACTGAGCTTACTGAGCT | |
| | GGGACTTGAACTCTGAGCATTCTACCTCATTGCTTTGGTGCATTAGGTTTGTAAT | |
| | ATCTGGTACCTCTGTTTCCTCAGATAGATGATAGAAATAAAGATATGATATTAAG | |
| | GAAGCTGTTAATACTGAATTTTCAGAAAAGTATCCCTCCATAAAATGTATTTGGG | |
| | GGACAAACTGCAGGAGATTATATTCTGGCCCTATAGTTATTCAAAACGTATTTAT | |
| | TGATTAATCTTTAAAAGGCTTAGTGAACAATATTCTAGTCAGATATCTAATTCTT | |
| | AAATCCTCTAGAAGAATTAACTAATACTATAAAATGGGTCTGGATGTAGTTCTGA | |
| | CATTATTTTATAACAACTGGTAAGAGGGAGTGACTATAGCAACAACTAAAATGAT | |
| | CTCAGGAAAACCTGTTTGGCCCTATGTATGGTACATTACATCTTTTCAGTAATTC | |
| | CACTCAAATGGAGACTTTTAACAAAGCAACTGTTCTCAGGGGACCTATTTTCTCC | |
| | CTTAAAATTCATTATACACATCCCTGGTTGATAGCAGTGTGTCTGGAGGCAGAAA | |
| | CCATTCTTGCTTTGGAAACAATTACGTCTGTGTTATACTGAGTAGGGAAGCTCAT | |
| | TAATTGTCGACACTTACGTTCCTGATAATGGGATCAGTGTGTAATTCTTGTTTCG | |
| | CTCCAGATTTCTAATACCACAAAGAATAAATCCTTTCACTCTGATCAATTTTGTT | |
| | AACTTCTCACGTGTCTTCTCTACACCCAGGGCACCGGTGAATCCAAGTGTCCTCT | |
| | GATGGTCAAAGTTCTAGATGCTGTCCGAGGCAGTCCTGCCATCAATGTGGCCGTG | |
| | CATGTGTTCAGAAAGGCTGCTGATGACACCTGGGAGCCATTTGCCTCTGGGTAAG | |
| | TTGCCAAAGAACCCTCCCACAGGACTTGGTTTTATCTTCCCGTTTGCCCGTCACT | |
| | TGGTAGAGAGAGGCTCACATCATCTGCTAAAGAATTTACAAGTAGATTGAAAAAC | |
| | GTAGGCAGAGGTCAAGTATGCCCTCTGAAGGATGCCCTCTTTTTGTTTTGCTTAG | |
| | CTAGGAAGTGACCAGGAACCTGAGCATCATTTAGGGGCAGACAGTAGAGAAAAGA | |
| | AGGAATCAGAACTCCTCTCCTCTAGCTGTGGTTTGCAACCCTTTTGGGTCACAGA | |
| | ACACTTTATGTAGGTGATGAAAAGTAAACATTCTATGCCCAGAAAAAATGCACAG | |
| | ATACACACACATACAAAATCATATATGTGATTTTAGGAGTTTCACAGATTCCC1G | |
| | GTGTCCCTGGGTAACACCAAAGCTAAGTGTCCTTGTCTTAGAATTTTAGGAAAAG | |
| | GTATAATGTGTATTAACCCATTAACAAAAGGAAAGGAATTCAGAAATATTATTAA | |
| | CCAGGCATCTGTCTGTAGTTAATATGGATCACCCAAAACCCAAGGCTTTTGCCTA | |
| | ATGAACACTTTGGGGCACCTACTGTGTGCAAGGCTGGGGGCTGTCAAGCTCAGTT | |
| | AAAAAAAAAAAGATAGAAGAGATGGATCCATGAGGCAAAGTACAGCCCCAGGCTA | |
| | ATCCCACGATCACCCGACTTCATGTCCAAGAGTGGCTTCTCACCTTCATTAGCCA | |
| | GTTCACAATTTTCATGGAGTTTTTCTACCTGCACTAGCAAAAACTTCAAGGAAAA | |
| | TACATATTAATAAATCTAAGCAAAGTGACCAGAAGACAGAGCAATCAGGAGACCC | |
| | TTTGCATCCAGCAGAAGAGGAACTGCTAAGTATTTACATCTCCACAGAGAAGAAT | |
| | TTCTGTTGGGTTTTAATTGAACCCCAAGAACCACATGATTCTTCAACCATTATTG | |
| | GGAAGATCATTTTCTTAGGTCTGGTTTTAACTGGCTTTTTATTTGGGAATTCATT | |
| | TATGTTTATATAAAATGCCAAGCATAACATGAAAAGTGGTTACAGGACTATTCTA | |
| | AGGGAGAGACAGAATGGACACCAAAAATATTCCAATGTTCTTGTGAATCTTTTCC | |
| | TTGCACCAGGACAAAAAAAAAAAGAAGTGAAAAGAAGAAAGGAGGAGGGGCATAA | |
| | TCAGAGTCAGTAAAGACAACTGCTATTTTTATCTATCGTAGCTGTTGCAGTCAAA | |
| | TGGGAAGCAATTTCCAACATTCAACTATGGAGCTGGTACTTACATGGAAATAGAA | |
| | GTTGCCTAGTGTTTGTTGCTGGCAAAGAGTTATCAGAGAGGTTAAATATATAAAA | |
| | GGGAAAAGAGTCAGATACAGGTTCTTCTTCCTACTTTAGGTTTTCCACTGTGTGT | |
| | GCAAATGATACTCCCTGGTGGTGTGCAGATGCCTCAAAGCTATCCTCACACCACA | |
| | AGGGAGAGGAGCGAGATCCTGCTGTCCTGGAGAAGTGCAGAGTTAGAACAGCTGT | |
| | GGCCACTTGCATCCAATCATCAATCTTGAATCACAGGGACTCTTTCTTAAGTAAA | |
| | CATTATACCTGGCCGGGCACGGTGGCTCACGCCTGTAATCCCAGCACTTTGGGAT | |
| | GCCAAAGTGGGCATATCATCTGAGGTCAGGAGTTCAAGACCAGCCTGGCCAACAT | |
| | GGCAAAACTCCGTCTTTATGAAAAATACAAAAATTAGCCAGGCATGGTGGCAGGC | |
| | GCCTGTAATCCCAGCTAATTGGGAGGCTGAGGCTGGAGAATCCCTTGAATCTAGG | |
| | AGGCAGAGGTTGCAGTGAGCTGAGATCGTGCCATTGCACTCCAGCCTGGGTGACA | |
| | AGAGTAAAACTCTGTCTCAAAAAAAAAAAATTATACCTACATTCTCTTCTTATCA | |
| | GAGAAAAAAATCTACAGTGAGCTTTTCAAAAAGTTTTTACAAACTTTTTGCCATT | |
| | TAATTTCAGTTAGGAGTTTTCCCTACTTCTGACTTAGTTGAGGGGAAATGTTCAT | |
| | AACATGTTTATAACATGTTTATGTGTGTTAGTTGGTGGGGGTGTATTACTTTGCC | |
| | ATGCCATTTGTTTCCTCCATGCGTAACTTAATCCAGACTTTCACACCTTATAGGA | |
| | AAACCAGTGAGTCTGGAGAGCTGCATGGGCTCACAACTGAGGAGGAATTTGTAGA | |
| | AGGGATATACAAAGTGGAAATAGACACCAAATCTTACTGGAAGGCACTTGGCATC | |
| | TCCCCATTCCATGAGCATGCAGAGGTGAGTATACAGACCTTCGAGGGTTGTTTTG | |
| | GTTTTGGTTTTTGCTTTTGGCATTCCAGGAAATGCACAGTTTTACTCAGTGTACC | |
| | ACAGAAATGTCCTAAGGAAGGTGATGAATGACCAAAGGTTCCCTTTCCTATTATA | |
| | CAAGAAAAAATTCACAACACTCTGAGAAGCAAATTTCTTTTTGACTTTGATGAAA | |
| | ATCCACTTAGTAACATGACTTGAACTTACATGAAACTACTCATAGTCTATTCATT | |
| | CCACTTTATATGAATATTGATGTATCTGCTGTTGAAATAATAGTTTATGAGGCAG | |
| | CCCTCCAGACCCCACGTAGAGTGTATGTAACAAGAGATGCACCATTTTATTTCTC | |
| | GAAAACCCGTAACATTCTTCATTCCAAAACACATCTGGCTTCTCGGAGGTCTGGA | |
| | CAAGTGATTCTTGGCAACACATACCTATAGAGACAATAAAATCAAAGTAATAATG | |
| | GCAACACAATAGATAACATTTACCAAGCATACACCATGTGGCAGACACAATTATA | |
| | AGTGTTTTCCATATTTAACCTACTTAATCC7CAGGAATAAGCCACTGAGGTCAGT | |
| | CCTATTATTATCCCCATCTTATAGATGAAGAAAATGAGGCACCAGGAAGTCAAAT | |
| | AACTTGTCAAAGGTCACAAGACTAGGAAATACACAAGTAGAAATGTTTACAATTA | |
| | AGGCCCAGGCTGGGTTTGCCCTCAGTTCTGCTATGCCTCGCATTATGCCCCAGGA | |
| | AACTTTTTCCCTTGTGAAAGCCAAGCTTAAAAAAAGAAAAGCCACATTTGTAACG | |
| | TGCTCTGTTCCCCTGCCTATGGTGAGGATCTTCAAACAGTTATACATGGACCCAG | |
| | TCCCCCTGCCTTCTCCTTAATTTCTTAAGTCATTTGAAACAGATGGCTGTCATGG | |
| | AAATAGAATCCAGACATGTTGGTCAGAGTTAAAGATCAACTAATTCCATCAAAAA | |
| | TAGCTCGGCATGAAAGGGAACTATTCTCTGGCTTAGTCATGGATGAGACTTTCAA | |
| | TTGCTATAAAGTGGTTCCTTTATTAGACAATGTTACCAGGGAAACAACAGGGGTT | |
| | TGTTTGACTTCTGGGGCCCACAAGTCAACAAGAGAGCCCCATCTACCAAGGAGCA | |
| | TGTCCCTGACTACCCCTCAGCCAGCAGGAAGACATGGACCCCAGTCAGGGCAGGA | |
| | GCAGGGTTTCGGCGGCGCCCAGCACAAGACATTGCCCCTAGAGTCTCAGCCCCTA | |
| | CCCTCGAGTAATAGATCTGCCTACCTGAGACTGTTGTTTGCCCAAGAGCTGGGTC | |
| | TCAGCCTGATGGGAACCATATAAAAAGGTTCACTGACATACTGCCCACATGTTGT | |
| | TCTCTTTCATTAGATCTTAGCTTCCTTGTCTGCTCTTCATTCTTGCAGTATTCAT | |
| | TCAACAAACATTAAAAAAAAAAAAAAGCATTCTATGTGTGGAACACTCTGCTAGA | |
| | TGCTGTGGATTTAGAAATGAAAATACATCCCGACCCTTGGAATGGAAGGGAAAGG | |
| | ACTGAAGTAAGACAGATTAAGCAGGACCGTCAGCCCAGCTTGAAGCCCAGATAAA | |
| | TACGGAGAACAAGAGAGAGCGAGTAGTGAGAGATGAGTCCCAATGCCTCACTTTG | |
| | GTGACGGGTGCGTGGTGGGCTTCATGCAGCTTCTTCTGATAAATGCCTCCTTCAG | |
| | AACTGGTCAACTCTACCTTGGCCAGTGACCCAGGTGGTCATAGTAGATTTACCAA | |
| | GGGAAAATGGAAACTTTTATTAGGAGCTCTTAGGCCTCTTCACTTCATGGATTTT | |
| | TTTTTCCTTTTTTTTTGAGATGGAGTTTTGCCCTGTCACCCAGGCTGGAATGCAG | |
| | TGGTGCAATCTCAGCTCACTGCAACCTCCGCCTCCCAGGTTCAAGCAATTCTCCT | |
| | GCCTCAGCCTCCCGAGTAGCTGGGACTACAGGTGTGCGCCACCACACCAGGCTAA | |
| | TTTTTGTATTTTTTGTAAAGACAGGTTTTCACCACGTTGGCCAGGCTGGTCTGAA | |
| | CTCCAGACCTCAGGTGATTCACCTGTCTCAGCCTCCCAAAGTGCTGGGATTACAG | |
| | GTGTGAGCCACCGTGCCCGGCTACTTCATGGATTTTTGATTACAGATTATGCCTC | |
| | TTACAATTTTTAAGAAGAATCAAGTGGGCTGAAGGTCAATGTCACCATAAGACAA | |
| | AAGACATTTTTAXTAGTTGATTCTAGGGAATTGGCCTTAAGGGGAGCCCTTTCTT | |
| | CCTAAGAGATTCTTAGGTGATTCTCACTTCCTCTTGCCCCAGTATTATTTTTGTT | |
| | TTTGGTATGGCTCACTCAGATCCTTTTTTCCTCCTATCCCTAAGTAATCCGGGTT | |
| | TCTTTTTCCCATATTTAGAACAAAATGTATTTATGCAGAGTGTGTCCAAACCTCA | |
| | ACCCAAGGCCTGTATACAAAATAAATCAAATTAAACACATCTTTACTGTCTTCTA | |
| | CCTCTTTCCTGACCTCAATATATCCCAACTTGCCTCACTCTGAGAACCAAGGCTG | |
| | TCCCAGCACCTGAGTCGCAGATATTCTACTGATTTGAGAGAACTGTGTGACTATC | |
| | TGGAACAGCATTTTGATCCACAATTTGCCCAGTTACAAAGCTTAAATGAGCTCTA | |
| | GTGCATGCATATATATTTCAAAATTCCACCATGATCTTCCACACTCTGTATTGTA | |
| | AATAGAGCCCTGTAATGCTTTTACTTCGTATTTCATTGCTTGTTATACATAAAAA | |
| | TATACTTTTCTTCTTCATGTTAGAAAATGCAAAGAATAGGAGGGTGGGGGAATCT | |
| | CTGGGCTTGGAGACAGGAGACTTGCCTTCCTACTATGGTTCCATCAGAATGTAGA | |
| | CTGGGACAATACAATAATTCAAGTCTGGTTTGCTCATCTGTAAATTGGGAAGAAT | |
| | GTTTCCAGCTCCAGAATGCTAAATCTCTAAGTCTGTGGTTGGCAGCCACTATTGC | |
| | AGCAGCTCTTCAATGACTCAATGCAGTTTTGCATTCTCCCTACCTTTTTTTTCTA | |
| | AAACCAATAAAATAGATACAGCCTTTAGGCTTTCTGGGATTTCCCTTAGTCAAGC | |
| | TAGGGTCATCCTGACTTTCGGCGTGAATTTGCAAAACAAGACCTGACTCTGTACT | |
| | CCTGCTCTAAGGACTGTGCATGGTTCCAAAGGCTTAGCTTGCCAGCATATTTGAG | |
| | CTTTTTCCTTCTGTTCAAACTGTTCCAAAATATAAAAGAATAAAATTAATTAAGT | |
| | TGGCACTGGACTTCCGGTGGTCAGTCATGTGTGTCATCTGTCACGTTTTTCGGGC | |
| | TCTGGTGGAAATGGATCTGTCTGTCTTCTCTCATAGGTGGTATTCACAGCCAACG | |
| | ACTCCGGCCCCCGCCGCTACACCATTGCCGCCCTGCTGAGCCCCTACTCCTATTC | |
| | CACCACGGCTGTCGTCACCAATCCCAAGGAATGAGGGACTTCTCCTCCAGTGGAC | |
| | CTGAAGGACGAGGGATGGGATTTCATGTAACCAAGAGTATTCCATTTTTACTAAA | |
| | GCAGTGTTTTCACCTCATATGCTATGTTAGAAGTCCAGGCAGAGACAATAAAACA | |
| | TTCCTGTGAAAGGCA | |
| 229 | ACAGAAGTCCACTCATTCTTGGCAGGATGGCTTCTCATCGTCTGCTCCTCCTCTG | TTR exon 1 |
| | CCTTGCTGGACTGGTATTTGTGTCTGAGGCTGGCCCTACGGTGAGTGTTTCTGTG | |
| | ACATCCCATTCCTACATTTAAGATTCACGCTAAAT | |
| 230 | TTCACTCTGATCAATTTTGTTAACTTCTCACGTGTCTTCTCTACACCCAGGGCAC | TTR exon 2 |
| | CGGTGAATCCAAGTGTCCTCTGATGGTCAAAGTTCTAGATGCTGTCCGAGGCAGT | |
| | CCTGCCATCAATGTGGCCGTGCATGTGTTCAGAAAGGCTGCTGATGACACCTGGG | |
| | AGCCATTTGCCTCTGGGTAAGTTGCCAAAGAACCCTCCCACAGGACTTGGTTTTA | |
| | TCTTCCCGTTT | |
| 231 | CCATTTGTTTCCTCCATGCGTAACTTAATCCAGACTTTCACACCTTATAGGAAAA | TTR exon 3 |
| | CCAGTGAGTCTGGAGAGCTGCATGGGCTCACAACTGAGGAGGAATTTGTAGAAGG | |
| | GATATACAAAGTGGAAATAGACACCAAATCTTACTGGAAGGCACTTGGCATCTCC | |
| | CCATTCCATGAGCATGCAGAGGTGAGTATACAGACCTTCGAGGGTTGTTTTGGTT | |
| | TTGGTTTTTGCTTTTG | |
| 232 | CACGTTTTTCGGGCTCTGGTGGAAATGGATCTGTCTGTCTTCTCTCATAGGTGGT | TTR exon 4 |
| | ATTCACAGCCAACGACTCCGGCCCCCGCCGCTACACCATTGCCGCCCTGCTGAGC | |
| | CCCTACTCCTATTCCACCACGGCTGTCGTCACCAATCCCAAGGAATGAGGGACTT | |
| | CTCCTCCAGTGGACCTGAAGGACGAGGGATGGGATTTCATGTAACCAAGAGTATT | |
| | CCATTTTTACTAAAGCAGTGTTTTCACCTCATATGCTATGTTAGAAGTCCAGGCA | |
| | GAGACAATAAAACATTCCTGTGAAAGGCA | |
| 256 | MASHRLLLLCLAGLVFVSEAGPTGTGESKCPLMVKVLDAVRGSPAINVAVHVFRK | TTR (full |
| | AADDTWEPEASGKTSESGELHGLTTEEEFVEGIYKVEIDTKSYWKALGISPFHEH | prolein |
| | AEVVFTANDSGFRRYTIAALLSPYSYSTTAVVTNPKE | sequence) |
| 257 | GPTGTGESKCPLMVKVLDAVRGSPAINVAVHVFRKAADDTWEPFASGKTSESGEL | TTR (post- |
| | HGLTTEEEEVEGlYKVEIDTKSYWKALGISPFHEHAEVVFTANDSGPRRYTIAAL | transla- |
| | LSPYSYSTTAVVTNPKE | tionally |
| | | cleaved |
| | | sequence) |
| | | V30,T119, |
| | | and VI22 |
| | | are |
| | | shown in |
| | | bold |
| 258 | ATGGCTTCTCATCGTCTGCTCCTCCTCTGCCTTGCTGGACTGGTATTTGTGTCTG | TTR coding |
| | AGGCTGGCCCTACGGGCACCGGTGAATCCAAGTGTCCTCTGATGGTCAAAGTTCT | sequence |
| | AGATGCTGTCCGAGGCAGTCCTGCCATCAATGTGGCCGTGCATGTGTTCAGAAAG | |
| | GCTGCTGATGACACCTGGGAGCCATTTGCCTCTGGGAAAACCAGTGAGTCTGGAG | |
| | AGCTGCATGGGCTCACAACTGAGGAGGAATTTGTAGAAGGGATATACAAAGTGGA | |
| | AATAGACACCAAATCTTACTGGAAGGCACTTGGCATCTCCCCATTCCATGAGCAT | |
| | GCAGAGGTGGTATTCACAGCCAACGACTCCGGCCCCCGCCGCTACACCATTGCCG | |
| | CCCTGCTGAGCCCCTACTCCTATTCCACCACGGCTGTCGTCACCAATCCCAAGGA | |

In some embodiments, the gene editing system disclosed herein may comprise a Cas12i polypeptide as disclosed herein. In other embodiments, the gene editing system may comprise a nucleic acid encoding the Cas12i polypeptide. For example, the gene editing system may comprise a vector (e.g., a viral vector such as an AAV vector, such as AAV1, AAV2, AAV3, AAV4, AAV5, AAV6, AAV7, AAV8, AAV9, AAV10, AAV1I and AAV12) encoding the Cas12i polypeptide. Alternatively, the gene editing system may comprise a mRNA molecule encoding the Cas12i polypeptide. In some instances, the mRNA molecule may be codon-optimized.

### II. Preparation of Gene Editing System Components

The present disclosure provides methods for production of components of the gene editing systems disclosed herein, e.g., the RNA guide, methods for production of the Cas12i polypeptide, and methods for complexing the RNA guide and Cas12i polypeptide.

A. RNA Guide

In some embodiments, the RNA guide is made by in vitro transcription of a DNA molecule. Thus, for example, in some embodiments, the RNA guide is generated by in vitro transcription of a DNA molecule encoding the RNA guide using an upstream promoter sequence (e.g., a T7 polymerase promoter sequence). In some embodiments, the DNA molecule encodes multiple RNA guides or the in vitro transcription reaction includes multiple different DNA molecules, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

B. Template DNA

In some embodiments, the template DNA is made using chemical synthetic methods. In some embodiments, the template DNA is made by oligonucleotide synthesis or annealing based connection of oligonucleotides. In some embodiments, the template DNA is synthesized as single-stranded oligo DNA nucleotides (ssODNs). In some embodiments, the template DNA is synthesized as double-stranded oligo DNA nucleotides (dsODNs). In some embodiments, the template DNA is made in cells transfected with a plasmid including the template DNA sequence. In some embodiments, the plasmid comprises different template DNA sequences. In some embodiments, multiple different plasmids, each comprising a different template DNA, are transfected into the cells. In some embodiments, a plasmid comprising the template DNA further encodes the RNA guide and/or a Cas12i polypeptide.

In some embodiments, the template DNA is purchased from a commercial vendor. In some embodiments, the template DNA is synthesized using one or more modified nucleotide, e.g., as described above.

C. Cas12i Polypeptide

In some embodiments, the Cas12i polypeptide of the present disclosure can be prepared by (a) culturing bacteria which produce the Cas12i polypeptide of the present disclosure, isolating the Cas12i polypeptide, optionally, purifying the Cas12i polypeptide, and complexing the Cas12i polypeptide with an RNA guide. The Cas12i polypeptide can be also prepared by (b) a known genetic engineering technique, specifically, by isolating a gene encoding the Cas12i polypeptide of the present disclosure from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

The present disclosure further provides methods of in vivo expression of a Cas12i polypeptide in a cell, comprising providing a polyribonucleotide encoding the Cas12i polypeptide to a host cell wherein the polyribonucleotide encodes the Cas12i polypeptide and expressing the Cas12i polypeptide in the cell. In some embodiments, the polyribonucleotide encoding the Cas12i polypeptide is delivered to the cell with an RNA guide and, once expressed in the cell, the Cas12i polypeptide and the RNA guide form a complex. In some embodiments, the polyribonucleotide encoding the Cas12i polypeptide and the RNA guide are delivered to the cell within a single composition. In some embodiments, the polyribonucleotide encoding the Cas12i polypeptide and the RNA guide are comprised within separate compositions. In some embodiments, the host cell is present in a subject, e.g., a human patient.

D. Complexes

In some embodiments, an RNA guide targeting TTR is complexed with a Cas12i polypeptide to form a ribonucleoprotein (RNP). In some embodiments, complexation of the RNA guide and Cas12i polypeptide occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously, separately, or sequentially from a single mRNA or DNA construct, such that the RNP complex is formed in the cell.

In some embodiments, the template DNA is bound to the Cas12i polypeptide. In some embodiments, the template DNA is bound covalently to the Cas12i polypeptide. In some embodiments, the template DNA is bound non-covalently to the Cas12i polypeptide. In some embodiments, the template DNA is bound to an RNP. In some embodiments, the template DNA is bound covalently to an RNP. In some embodiments, the template DNA is bound non-covalently to an RNP.

### III. Genetic Editing Methods

The present disclosure also provides methods of modifying a target sequence within the TTR gene or methods of introducing a mutation into the TTR gene. In some embodiments, the methods comprise introducing a TTR-targeting RNA guide, a Cas12i polypeptide, and a template DNA into a cell. The TTR-targeting RNA guide and Cas12i polypeptide can be introduced as a ribonucleoprotein complex into a cell. The TTR-targeting RNA guide, template DNA, and Cas12i polypeptide can be introduced on a nucleic acid vector. The Cas12i polypeptide can be introduced as an mRNA. The RNA guide and template DNA can be introduced directly into the cell.

Any of the gene editing systems disclosed herein may be used to genetically engineered a TTR gene. The gene editing system may comprise a guide RNA, a Cas12i2 polypeptide, and a template DNA. The guide RNA comprises a spacer sequence specific to a target sequence in the TTR gene, e.g., specific to a region in exon2, exon 3, or exon 4 of the TTR gene.

A. Target Sequences

In some embodiments, an RNA guide as disclosed herein is designed to be complementary to a target sequence that is adjacent to a 5′-TTN-3′ PAM sequence or 5′-NTTN-3′ PAM sequence.

In some embodiments, the target sequence is within a TTR gene or a locus of a TTR gene (e.g., exon 2, exon 3, or exon 4), to which the RNA guide can bind via base pairing. In some embodiments, a cell has only one copy of the target sequence. In some embodiments, a cell has more than one copy, such as at least about any one of 2, 3, 4, 5, 10, 100, or more copies of the target sequence.

In some embodiments, the TTR gene is a mammalian gene. In some embodiments, the TTR gene is a human gene. For example, in some embodiments, the target sequence is within the sequence of SEQ ID NO: 228, or the reverse complement thereof, or SEQ ID NO: 258, or the reverse complement thereof. In some embodiments, the target sequence is within an exon of the TTR gene set forth in SEQ ID NO: 228, or the reverse complement thereof, or SEQ ID NO: 258, or the reverse complement thereof, e.g., within a sequence of SEQ ID NO: 229, 230, 231, or 232 (or a reverse complement of any thereof). Target sequences within an exon region of the TTR gene of SEQ ID NO: 228 are set forth in Table 6. In some embodiments, the target sequence is within an intron of the TTR gene set forth in SEQ ID NO: 228, or the reverse complement thereof. In some embodiments, the target sequence is within a variant (e.g., a polymorphic variant) of the TTR gene sequence set forth in SEQ ID NO: 228, or the reverse complement thereof, or SEQ ID NO: 258, or the reverse complement thereof. In some embodiments, the TTR gene sequence is a homolog of the sequence set forth in SEQ ID NO: 228, or the reverse complement thereof, or SEQ ID NO: 258, or the reverse complement thereof. In some embodiments, the TTR gene sequence is a non-human TTR sequence.

In some embodiments, the target sequence is adjacent to a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′. 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTfN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′. 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CITT-3′, 5′-CTfG-3′, or 5′-CTTC-3′. The PAM sequence may be 5′ to the target sequence.

In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, the RNA guide binds to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR-3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TITC-3′, 5′-GTTA-3′, 5′-GTIT-3′, 5′-GTTG-3′, 5′-GTTC-3′, 5′-CTTA-3′, 5′-CTTT-3′, 5′-CTTG-3′, or 5′-CTC-3′. In some embodiments, the RNA guide is designed to bind to a first strand of a double-stranded target nucleic acid (i.e., the non-PAM strand), and the 5′-NTTN-3′ PAM sequence is present in the second, complementary strand (i.e., the PAM strand). In some embodiments, the RNA guide binds to a region on the non-PAM strand that is complementary to a target sequence on the PAM strand, which is adjacent to a 5′-NAAN-3′ sequence.

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc.

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene.

B. Gene Editing

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TITA-3′, 5′-TTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTIT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the TTR gene. In some embodiments, the deletion alters function of the TTR gene. In some embodiments, the deletion inactivates the TTR gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TITA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTF-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTIT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CITG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence.

In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTIT-3′, 5′-GTTG-3′, 5′-GTTC-3′, 5′-CTTA-3′, 5′-CTfT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTT-3′, 5′-TTTG-3′, 5′-TMTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TITT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5‘-TT’IA-3′, 5′-TTT-3′, 5′-TTTG-3′, 5′-TTC-3′, 5′-GTTA-3′, 5′-GTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.
In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TITT-3′, 5′-TITG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTT-3′, 5′-TITG-3′, 5′-TTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATT-3′, 5′-ATTG-3′, 5′-ATTC-3′, 5′-TTTA-3′, 5′-TITT-3′, 5′-TTTG-3′, 5′-TITC-3′, 5′-GTTA-3′, 5′-GTTT-3′, 5′-GTTG-3′, 5′-GTTC-3′, 5′-CITA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATT-3′, 5′-ATTG-3′, 5′-ATTC-3′, 5′-ITTA-3′, 5′-TlTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTF-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TITT-3′, 5′-TTTG-3′, 5′-TITC-3′, 5′-GTTA-3′, 5′-GTTT-3′, 5′-GTTG-3′, 5′-GTTC-3′, 5′-CITA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CITT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATT-3′, 5′-ATTG-3′, 5′-ATTG-3′, 5′-TTTA-3′, 5′-TTrT-3′, 5′-TTTG-3′, 5′-TITC-3′, 5′-GTTA-3′, 5′-GTTT-3′, 5′-GTTG-3′, 5′-GTTC-3′, 5′-CITA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATIT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TITT-3′, 5′-TITG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′. 5′-TITT-3′, 5′-TTTG-3′, 5′-TTTC-3′, 5′-GTTA-3′, 5′-GTTF-3′, 5′-GTTG-3′, 5′-GTTC-3′, 5′-CTTA-3′, 5′-CIT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′. 5′-ATTG-3′, 5′-ATTC-3′, 5′-TTTA-3′. 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′. 5′-CTTG-3′, or 5′-CTTC-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5‘-A’TTG-3′, 5′-ATTC-3′, 5′-TTA-3′, 5′-TITT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence.

In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TrTA-3′, 5′-TTTF-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CITf-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTIT-3′, 5′-ITG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CITG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTIT-3′, 5′-TTTG-3′, 5′-1TTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TFTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CITC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TITT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5‘-A’TTC-3′, 5′-TTTA-3′, 5′-TTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TITT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 50 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 nucleotides). In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, two or more RNA guides described herein are used to introduce a deletion that has a length of greater than 40 nucleotides. In some embodiments, two or more RNA guides described herein are used to introduce a deletion of at least about 41, 42, 43, 44, 45, 46, 47, 48, 49, 50, 55, 60, 65, 70, 75, 80, 85, 90, 95, 100, 110, 120, 130, 140, 150, 16, 170, 180, 190, 200, 210, 220, 230, 240, 250, 260, 270, 280, 290, 300, 310, 320, 330, 340, 350, 360, 370, 380, 390, or 400 nucleotides. In some embodiments, two or more RNA guides described herein are used delete all or a portion of the TTR gene or SEQ ID NO: 228. In some embodiments, two or more RNA guides are used to delete all or a portion of the TTR coding sequence of SEQ ID NO: 258.

In some embodiments, the methods described herein are used to engineer a cell comprising a deletion as described herein in a TTR gene. In some embodiments, the methods are carried out using a complex comprising a Cas12i enzyme as described herein and an RNA guide comprising a direct repeat sequence and a spacer sequence as described herein. In some embodiments, the sequence of the RNA guide has at least 90% identity (e.g., at least 90%, 91%, 92%, 93%, 94%, 95%, 96%, 97%, 98%, or 99% identity) to a sequence of any one of SEQ ID NOs: 345-362. In some embodiments, an RNA guide has a sequence of any one of SEQ ID NOs: 345-362.

In some embodiments, the RNA guide targeting TTR is encoded in a plasmid. In some embodiments, the RNA guide targeting TTR is synthetic or purified RNA. In some embodiments, the Cas12i polypeptide is encoded in a plasmid. In some embodiments, the Cas12i polypeptide is encoded by an RNA that is synthetic or purified.

In some embodiments, a template DNA described herein is used to correct a mutation associated with a disease. In some embodiments, the mutation is V30M, V122I, T60A, L58H, or 184S, and the disease is hATTR, FAP, or SSA. In some embodiments, mutations V30M, V122I, T60A, L58H, or 184S are in reference to the post-translationally cleaved TTR sequence of SEQ ID NO: 257. When the full length TTR sequence (SEQ ID NO: 256) is used as the reference sequence, these mutations are referred to as V50M, V1421, T80A, L78H, and I104S with respect to the full-length TTR sequence of SEQ ID NO: 256.

In some embodiments, a template DNA described herein is used to introduce a protective mutation associated with a disease. In some embodiments, the mutation is T119M, and the disease is hATTR. In some embodiments, the mutation is T119M, and the disease is wild-type ATTR amyloidosis. In some embodiment, the TI 19M mutation is in reference to the post-translationally cleaved TTR sequence of SEQ ID NO: 257. In some embodiments, the T119M mutation is referred to as T139M with respect to the full-length TTR sequence of SEQ ID NO: 256.

In some embodiments, a template DNA described herein is used to correct a mutation associated with a disease and to introduce a protective mutation. In some embodiments, the mutation associated with disease is V30M, V122I, T60A, L58H, or I84S and the disease is (hATTR). In some embodiments, the protective mutation is TI 19M. In some embodiments, the disease is hATTR or wild-type ATTR amyloidosis. For example, in some embodiments, a template DNA described herein is used to correct a V30M mutation and to introduce a TI 19M mutation. In some embodiments, a template DNA described herein is used to correct a V122I mutation and to introduce a T119M mutation. In some embodiments, a template DNA described herein is used to correct a T60A mutation and to introduce a T119M mutation. In some embodiments, a template DNA described herein is used to correct an L58H mutation and to introduce a T119M mutation. In some embodiments, a template DNA described herein is used to correct an 184S mutation and to introduce a T119M mutation.

In some embodiments, the insert sequence (e.g., the donor region comprising a sequence difference relative to the target nucleic acid) is incorporated into the target nucleic acid within the target region (e.g., the region of the target nucleic acid to which the RNA guide binds). In some embodiments, the insert sequence is incorporated into the target nucleic acid outside of the target region. In some embodiments, the insert sequence is incorporated into the non-target strand of the target nucleic acid by HDR using a single-stranded template DNA.

In some embodiments, the mutation is incorporated into the target nucleic acid upstream of a 5′-NTTN-3′ sequence. For example, the mutation is incorporated within about 20 nucleotides upstream of the 5′-NTTN-3′ sequence, e.g., 1 nucleotide, 2 nucleotides, 3 nucleotides. 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides upstream of the 5′-NTTN-3′ sequence.

In some embodiments, the mutation is incorporated into the target nucleic acid downstream of a 5′-NTTN-3′ sequence. For example, the mutation can be incorporated within about 60 nucleotides downstream of the 5′-NTTN-3′ sequence, e.g., 1 nucleotide, 2 nucleotides. 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 55 nucleotides, or 60 nucleotides downstream of the 5′-NTTN-3′ sequence.

In some embodiments, a composition described herein is introduced into a plurality of cells. In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of the cells comprise a wild-type TTR gene (e.g., one of more of the following mutations were corrected: V30M, V122I, T60A, L58H, and 184S).

In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of the cells comprise a T119M mutation.

In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of the cells comprise a wild-type TTR gene (e.g., one of more of the following mutations were corrected: V30M, V122I, T60A, L58H, and 184S) as well as a TI 19M mutation.

In some embodiments, the RNA guide targeting TTR is encoded in a plasmid. In some embodiments, the RNA guide targeting TTR is synthetic or purified RNA. In some embodiments, the Cas12i polypeptide is encoded in a plasmid. In some embodiments, the Cas12i polypeptide is encoded by an RNA that is synthetic or purified.

C. Delivery

Components of any of the gene editing systems disclosed herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, adeno-associated virus (AAV)), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, template DNA, etc.), one or more transcripts thereof, and/or a pre-formed RNA guide/Cas12i polypeptide complex to a cell, where a ternary complex is formed. In some embodiments, an RNA guide and an RNA encoding a Cas12i polypeptide are delivered together in a single composition. In some embodiments, an RNA guide and an RNA encoding a Cas12i polypeptide are delivered in separate compositions. In some embodiments, an RNA guide and an RNA encoding a Cas12i polypeptide delivered in separate compositions are delivered using the same delivery technology. In some embodiments, an RNA guide and an RNA encoding a Cas12i polypeptide delivered in separate compositions are delivered using different delivery technologies.

In some embodiments, the Cas12i component and the RNA guide component are delivered together. For example, the Cas12i component and the RNA guide component are packaged together in a single AAV particle. In another example, the Cas12i component and the RNA guide component are delivered together via lipid nanoparticles (LNPs). In some embodiments, the Cas12i component and the RNA guide component are delivered separately. For example, the Cas12i component and the RNA guide are packaged into separate AAV particles. In another example, the Cas12i component is delivered by a first delivery mechanism and the RNA guide is delivered by a second delivery mechanism.

Exemplary intracellular delivery methods, include, but are not limited to: viruses, such as AAV, or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, a lipid nanoparticle comprises an mRNA encoding a Cas12i polypeptide, an RNA guide, or an mRNA encoding a Cas12i polypeptide and an RNA guide. In some embodiments, the mRNA encoding the Cas12i polypeptide is a transcript of the nucleotide sequence set forth in SEQ ID NO: 221 or SEQ ID NO: 255 or a variant thereof. In some embodiments, the present application further provides cells produced by such methods, and organisms (such as animals, plants, or fungi) comprising or produced from such cells.

D. Genetically Modified Cells

Any of the gene editing systems disclosed herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

Any of the genetically modified cells produced using any of the gene editing system disclosed herein is also within the scope of the present disclosure. Such modified cells may comprise a disrupted TTR gene.

Any of the gene editing systems, compositions comprising such, vectors, nucleic acids, RNA guides and cells disclosed herein may be used in therapy. Gene editing systems, compositions, vectors, nucleic acids, RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a TTR sequence as disclosed herein. In some embodiments, a cell engineered using an RNA guide disclosed herein is used for ex vivo gene therapy.

### IV. Therapeutic Applications

Any of the gene editing systems or modified cells generated using such a gene editing system as disclosed herein may be used for treating a disease that is associated with the TTR gene, for example, amyloidogenic transthyretin (ATTR). In some instances, the ATTR is hereditary ATTR (hATTR) or wild-type ATTR amyloidosis.

hATTR amyloidosis (also referred to as transthyretin familial amyloid polyneuropathy (TTR-FAP) or familial amyloid cardiomyopathy (TTR-FAC) is a systemic disorder characterized by the extracellular deposition of misfolded transthyretin (TTR) protein. Normally, TTR is a tetramer made up of 4 single-chain monomers. TTR gene mutations are thought to destabilize the protein and cause tetramer dissociation into monomers, which aggregate into amyloid fibrils. These amyloid fibrils then accumulate in multiple organs throughout the body.

hATTR amyloidosis is an autosomal dominant disease with variable penetrance. Amyloid deposition or symptomatic disease typically occurs in adults ranging from 30 to 70 years of age, depending on mutation. Over 120 amyloidogenic TTR mutations have been identified. Some hATTR disease causing/associated SNPs are shown below in Table 7. The positions of the DNA mutations are relative to the TTR coding sequence of SEQ ID NO: 258. The positions of the amino acid mutations are relative to the post-translationally cleaved TTR protein sequence of SEQ ID NO: 257 (top mutation) or the full-length TTR protein sequence of SEQ ID NO: 256 (bottom mutation in parentheses).

TABLE 7
hATTR Disease Causing/Associated SNPs

| | Amino | | | | |
|---|---|---|---|---|---|
| DNA | acid | Age of | | | Cases |
| mutation | mutation | onset | Disease course | Location | published |
| None | WT | 80 | Late-onset | Worldwide | ~25% of |
| | | | cardiac | | people |
| | | | involvement | | over |
| | | | | | age 80 |
| c. 148G > A | V30M | 30-60 | Presents with | Worldwide | >400 |
| | (V50M) | | polyneuropathy | | |
| | | | and autonomic | | |
| | | | dysfunction, | | |
| | | | then systemic | | |
| | | | involvement | | |
| c. 424G > A | V122I | 60 s | Late-onset | African- | Most |
| | (V142I) | | cardiac | American | common |
| | | | amyloidosis | | variant |
| c. 238G > A | T60A | 50 s | Late-onset | Appalachian, | >40 |
| | (T80A) | | cardiac | Ireland | |
| | | | amyloidosis | | |
| | | | with some | | |
| | | | neurologic | | |
| | | | involvement | | |
| c. 233T > A | L58H | 40 s | Usually | Maryland, | >50 |
| | (L78H) | | presents with | German | |
| | | | CTS and is | | |
| | | | slowly | | |
| | | | progressive | | |
| | | | over two | | |
| | | | decades | | |
| c. 311T > G | I84S | 20-30 | CTS at 30, | Indiana, | >30 |
| | (I104S) | | vitreous | Switzerland | |
| | | | opacites 40- | | |
| | | | 50 s, then heart | | |
| | | | involvement | | |

The T119M mutation is considered to be non-amyloidogenic and stabilize the TTR tetramer in patients with hATTR. See, e.g., Batista et al., Gene Therapy 21: 1041-50 (2014) and Yee et al., Nature Communications 10: 925 (2019). Therefore, T119M is considered a protective mutation. The T119M mutation can be introduced to treat hATTR in subjects having an amyloidogenic TTR mutation or to treat patients having wild-type ATTR amyloidosis.

In some embodiments, provided herein is a method for treating a target disease as disclosed herein (e.g., amyloidogenic transthyretin (ATTR) such as hATTR) comprising administering to a subject (e.g., a human patient) in need of the treatment any of the gene editing systems disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by a CRISPR nuclease, reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edit in the TTR gene. In some examples, the cells can be suspended in a suitable media.

In some embodiments, provided herein is a composition comprising the gene editing system or components thereof. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition (e.g., the gene editing system or components thereof), which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits that can be used, for example, to carry out a method described herein for genetical modification of the TTR gene. In some embodiments, the kits include an RNA guide and a Cas12i polypeptide. In some embodiments, the kits include an RNA guide, a template DNA, and a Cas12i polypeptide. In some embodiments, the kits include a polynucleotide that encodes such a Cas12i polypeptide, and optionally the polynucleotide is comprised within a vector, e.g., as described herein. In some embodiments, the kits include a polynucleotide that encodes an RNA guide disclosed herein. The Cas12i polypeptide (or polynucleotide encoding the Cas12i polypeptide) and the RNA guide (e.g., as a ribonucleoprotein) can be packaged within the same or other vessel within a kit or can be packaged in separate vials or other vessels, the contents of which can be mixed prior to use.

The Cas12i polypeptide, the RNA guide, and the template DNA can be packaged within the same or other vessel within a kit or can be packaged in separate vials or other vessels, the contents of which can be mixed prior to use. The kits can additionally include, optionally, a buffer and/or instructions for use of the RNA guide, template DNA, and Cas12i polypeptide.

All references and publications cited herein are hereby incorporated by reference.

### ADDITIONAL EMBODIMENTS

Provided below are additional embodiments, which are also within the scope of the present disclosure.

Embodiment 1: A composition comprising an RNA guide, wherein the RNA guide comprises (i) a spacer sequence that is substantially complementary or completely complementary to a target sequence of a target nucleic acid within a TTR gene and (ii) a direct repeat sequence; wherein the target sequence is adjacent to a protospacer adjacent motif (PAM) comprising the sequence 5′-NTTN-3′.

In Embodiment 1, the TTR gene may comprise the sequence of SEQ ID NO: 228, the reverse complement of SEQ ID NO: 228, a variant of SEQ ID NO: 228, the reverse complement of a variant of SEQ ID NO: 228, the sequence of SEQ ID NO: 258, the reverse complement of SEQ ID NO: 258, a variant of SEQ ID NO: 258, or the reverse complement of a variant of SEQ ID NO: 258. In some examples, the target sequence is within exon 1, exon 2, exon 3, or exon 4 of the TTR gene.

In Embodiment 1, the spacer sequence comprises:
a. nucleotide 1 through nucleotide 16 of a sequence that is at least 90% identical to a sequence of any one of SEQ ID NOs: 116-220;
b. nucleotide 1 through nucleotide 17 of a sequence that is at least 90% identical to a sequence of any one of SEQ ID NOs: 116-220;
c. nucleotide 1 through nucleotide 18 of a sequence that is at least 90% identical to a sequence of any one of SEQ ID NOs: 116-220;
d. nucleotide 1 through nucleotide 19 of a sequence that is at least 90% identical to a sequence of any one of SEQ ID NOs: 116-220;
e. nucleotide 1 through nucleotide 20 of a sequence that is at least 90% identical to a sequence of any one of SEQ ID NOs: 116-220;
f. nucleotide 1 through nucleotide 21 of a sequence that is at least 90% identical to a sequence of any one of SEQ ID NOs: 116-220;
g. nucleotide 1 through nucleotide 22 of a sequence that is at least 90% identical to a sequence of any one of SEQ ID NOs: 116-220;
h. nucleotide 1 through nucleotide 23 of a sequence that is at least 90% identical to a sequence of any one of SEQ ID NOs: 116-220;
i. nucleotide 1 through nucleotide 24 of a sequence that is at least 90% identical to a sequence of any one of SEQ ID NOs: 116-220;
j. nucleotide 1 through nucleotide 25 of a sequence that is at least 90% identical to a sequence of any one of SEQ ID NOs: 116-220;
k. nucleotide 1 through nucleotide 26 of a sequence that is at least 90% identical to a sequence of any one of SEQ ID NOs: 116-220;
l. nucleotide 1 through nucleotide 27 of a sequence that is at least 90% identical to a sequence of any one of SEQ ID NOs: 116-220;
m. nucleotide 1 through nucleotide 28 of a sequence that is at least 90% identical to a sequence of any one of SEQ ID NOs: 116-220;
n. nucleotide 1 through nucleotide 29 of a sequence that is at least 90% identical to a sequence of any one of SEQ ID NOs: 116-220; or
o. nucleotide 1 through nucleotide 30 of a sequence that is at least 90% identical to a sequence of any one of SEQ ID NOs: 116-220.

In some examples, the spacer sequence may comprise:
a. nucleotide 1 through nucleotide 16 of any one of SEQ ID NOs: 116-220;
b. nucleotide 1 through nucleotide 17 of any one of SEQ ID NOs: 116-220;
c. nucleotide 1 through nucleotide 18 of any one of SEQ ID NOs: 116-220;
d. nucleotide 1 through nucleotide 19 of any one of SEQ ID NOs: 116-220;
e. nucleotide 1 through nucleotide 20 of any one of SEQ ID NOs: 116-220;
f. nucleotide 1 through nucleotide 21 of any one of SEQ ID NOs: 116-220;
g. nucleotide 1 through nucleotide 22 of any one of SEQ ID NOs: 116-220;
h. nucleotide 1 through nucleotide 23 of any one of SEQ ID NOs: 116-220;
i. nucleotide 1 through nucleotide 24 of any one of SEQ ID NOs: 116-220;
j. nucleotide 1 through nucleotide 25 of any one of SEQ ID NOs: 116-220;
k. nucleotide 1 through nucleotide 26 of any one of SEQ ID NOs: 116-220;
l. nucleotide 1 through nucleotide 27 of any one of SEQ ID NOs: 116-220;
m. nucleotide 1 through nucleotide 28 of any one of SEQ ID NOs: 116-220;
n. nucleotide 1 through nucleotide 29 of any one of SEQ ID NOs: 116-220; or
o. nucleotide 1 through nucleotide 30 of any one of SEQ ID NOs: 116-220.

In Embodiment 1, the direct repeat may comprises:

a. nucleotide 1 through nucleotide 36 of a sequence that is at least 90% identical to a sequence of any one of SEQ ID NOs: 1-8;

b. nucleotide 2 through nucleotide 36 of a sequence that is at least 90% identical to a sequence of any one of SEQ ID NOs: 1-8:

c. nucleotide 3 through nucleotide 36 of a sequence that is at least 90% identical to a sequence of any one of SEQ ID NOs: 1-8;

d. nucleotide 4 through nucleotide 36 of a sequence that is at least 90% identical to a sequence of any one of SEQ ID NOs: 1-8;

e. nucleotide 5 through nucleotide 36 of a sequence that is at least 90% identical to a sequence of any one of SEQ ID NOs: 1-8;

f. nucleotide 6 through nucleotide 36 of a sequence that is at least 90% identical to a sequence of any one of SEQ ID NOs: 1-8;

g. nucleotide 7 through nucleotide 36 of a sequence that is at least 90% identical to a sequence of any one of SEQ ID NOs: 1-8;

h. nucleotide 8 through nucleotide 36 of a sequence that is at least 90% identical to a sequence of any one of SEQ ID NOs: 1-8;

i. nucleotide 9 through nucleotide 36 of a sequence that is at least 90% identical to a sequence of any one of SEQ ID NOs: 1-8;

j. nucleotide 10 through nucleotide 36 of a sequence that is at least 90% identical to a sequence of any one of SEQ ID NOs: 1-8;

k. nucleotide 11 through nucleotide 36 of a sequence that is at least 90% identical to a sequence of any one of SEQ ID NOs: 1-8;

l. nucleotide 12 through nucleotide 36 of a sequence that is at least 90% identical to a sequence of any one of SEQ ID NOs: 1-8;

m. nucleotide 13 through nucleotide 36 of a sequence that is at least 90% identical to a sequence of any one of SEQ ID NOs: 1-8;

n. nucleotide 14 through nucleotide 36 of a sequence that is at least 90% identical to a sequence of any one of SEQ ID NOs: 1-8;

o. nucleotide 1 through nucleotide 34 of a sequence that is at least 90% identical to a sequence of SEQ ID NO: 9;

p. nucleotide 2 through nucleotide 34 of a sequence that is at least 90% identical to a sequence of SEQ ID NO: 9;

q. nucleotide 3 through nucleotide 34 of a sequence that is at least 90% identical to a sequence of SEQ ID NO: 9;

r. nucleotide 4 through nucleotide 34 of a sequence that is at least 90% identical to a sequence of SEQ ID NO: 9;

s. nucleotide 5 through nucleotide 34 of a sequence that is at least 90% identical to a sequence of SEQ ID NO: 9;

t. nucleotide 6 through nucleotide 34 of a sequence that is at least 90% identical to a sequence of SEQ ID NO: 9;

u. nucleotide 7 through nucleotide 34 of a sequence that is at least 90% identical to a sequence of SEQ ID NO: 9;

v. nucleotide 8 through nucleotide 34 of a sequence that is at least 90% identical to a sequence of SEQ ID NO: 9;

w. nucleotide 9 through nucleotide 34 of a sequence that is at least 90% identical to a sequence of SEQ ID NO: 9;

x. nucleotide 10 through nucleotide 34 of a sequence that is at least 90% identical to a sequence of SEQ ID NO: 9;

y. nucleotide 11 through nucleotide 34 of a sequence that is at least 90% identical to a sequence of SEQ ID NO: 9;

z. nucleotide 12 through nucleotide 34 of a sequence that is at least 90% identical to a sequence of SEQ ID NO: 9; or

aa. a sequence that is at least 90% identical to a sequence of SEQ ID NO: 10 or a portion thereof.

In some examples, the direct repeat may comprise:

a. nucleotide 1 through nucleotide 36 of any one of SEQ ID NOs: 1-8;

b. nucleotide 2 through nucleotide 36 of any one of SEQ ID NOs: 1-8;

c. nucleotide 3 through nucleotide 36 of any one of SEQ ID NOs: 1-8;

d. nucleotide 4 through nucleotide 36 of any one of SEQ ID NOs: 1-8;

e. nucleotide 5 through nucleotide 36 of any one of SEQ ID NOs: 1-8;

f. nucleotide 6 through nucleotide 36 of any one of SEQ ID NOs: 1-8;

g. nucleotide 7 through nucleotide 36 of any one of SEQ ID NOs: 1-8;

h. nucleotide 8 through nucleotide 36 of any one of SEQ ID NOs: 1-8;

i. nucleotide 9 through nucleotide 36 of any one of SEQ ID NOs: 1-8;

j. nucleotide 10 through nucleotide 36 of any one of SEQ ID NOs: 1-8;

k. nucleotide 11 through nucleotide 36 of any one of SEQ ID NOs: 1-8;

l. nucleotide 12 through nucleotide 36 of any one of SEQ ID NOs: 1-8;

m. nucleotide 13 through nucleotide 36 of any one of SEQ ID NOs: 1-8;

n. nucleotide 14 through nucleotide 36 of any one of SEQ ID NOs: 1-8;

o. nucleotide 1 through nucleotide 34 of SEQ ID NO: 9;

p. nucleotide 2 through nucleotide 34 of SEQ ID NO: 9;

q. nucleotide 3 through nucleotide 34 of SEQ ID NO: 9;

r. nucleotide 4 through nucleotide 34 of SEQ ID NO: 9:

s. nucleotide 5 through nucleotide 34 of SEQ ID NO: 9;

L nucleotide 6 through nucleotide 34 of SEQ ID NO: 9;

u. nucleotide 7 through nucleotide 34 of SEQ ID NO: 9;

v. nucleotide 8 through nucleotide 34 of SEQ ID NO: 9;

w. nucleotide 9 through nucleotide 34 of SEQ ID NO: 9;

x. nucleotide 10 through nucleotide 34 of SEQ ID NO: 9;

y. nucleotide 1 I through nucleotide 34 of SEQ ID NO: 9;

z. nucleotide 12 through nucleotide 34 of SEQ ID NO: 9; or

aa. SEQ ID NO: 10 or a portion thereof.

In any of the composition of Embodiment 1, the direct repeat may comprise:

a. nucleotide 1 through nucleotide 36 of a sequence that is at least 90% identical to a sequence of any one of SEQ ID NOs: 233-250;

b. nucleotide 2 through nucleotide 36 of a sequence that is at least 90% identical to a sequence of any one of SEQ ID NOs: 233-250;

c. nucleotide 3 through nucleotide 36 of a sequence that is at least 90% identical to a sequence of any one of SEQ ID NOs: 233-250;

d. nucleotide 4 through nucleotide 36 of a sequence that is at least 90% identical to a sequence of any one of SEQ ID NOs: 233-250;

e. nucleotide 5 through nucleotide 36 of a sequence that is at least 90% identical to a sequence of any one of SEQ ID NOs: 233-250;

f. nucleotide 6 through nucleotide 36 of a sequence that is at least 90% identical to a sequence of any one of SEQ ID NOs: 233-250;

g. nucleotide 7 through nucleotide 36 of a sequence that is at least 90% identical to a sequence of any one of SEQ ID NOs: 233-250;

h. nucleotide 8 through nucleotide 36 of a sequence that is at least 90% identical to a sequence of any one of SEQ ID NOs: 233-250;

i. nucleotide 9 through nucleotide 36 of a sequence that is at least 90% identical to a sequence of any one of SEQ ID NOs: 233-250;

j. nucleotide 10 through nucleotide 36 of a sequence that is at least 90% identical to a sequence of any one of SEQ ID NOs: 233-250;

k. nucleotide 11 through nucleotide 36 of a sequence that is at least 90% identical to a sequence of any one of SEQ ID NOs: 233-250;

l. nucleotide 12 through nucleotide 36 of a sequence that is at least 90% identical to a sequence of any one of SEQ ID NOs: 233-250;

m. nucleotide 13 through nucleotide 36 of a sequence that is at least 90% identical to a sequence of any one of SEQ ID NOs: 233-250;

n. nucleotide 14 through nucleotide 36 of a sequence that is at least 90% identical to a sequence of any one of SEQ ID NOs: 233-250; or

o. a sequence that is at least 90% identical to a sequence of SEQ ID NO: 251 or a portion thereof.

In some examples, the direct repeat may comprise:

a. nucleotide 1 through nucleotide 36 of any one of SEQ ID NOs: 233-250;

b. nucleotide 2 through nucleotide 36 of any one of SEQ ID NOs: 233-250;

c. nucleotide 3 through nucleotide 36 of any one of SEQ ID NOs: 233-250;

d. nucleotide 4 through nucleotide 36 of any one of SEQ ID NOs: 233-250;

e. nucleotide 5 through nucleotide 36 of any one of SEQ ID NOs: 233-250;

f. nucleotide 6 through nucleotide 36 of any one of SEQ ID NOs: 233-250;

g. nucleotide 7 through nucleotide 36 of any one of SEQ ID NOs: 233-250;

h. nucleotide 8 through nucleotide 36 of any one of SEQ ID NOs: 233-250;

i. nucleotide 9 through nucleotide 36 of any one of SEQ ID NOs: 233-250;

j. nucleotide 10 through nucleotide 36 of any one of SEQ ID NOs: 233-250;

k. nucleotide 11 through nucleotide 36 of any one of SEQ ID NOs: 233-250;

l. nucleotide 12 through nucleotide 36 of any one of SEQ ID NOs: 233-250;

m. nucleotide 13 through nucleotide 36 of any one of SEQ ID NOs: 233-250;

n. nucleotide 14 through nucleotide 36 of any one of SEQ ID NOs: 233-250; or

o. SEQ ID NO: 251 or a portion thereof.

In other examples, the direct repeat may comprise:

a. nucleotide 1 through nucleotide 36 of a sequence that is at least 90% identical to SEQ ID NO: 259;

b. nucleotide 2 through nucleotide 36 of a sequence that is at least 90% identical to SEQ ID NO: 259;

c. nucleotide 3 through nucleotide 36 of a sequence that is at least 90% identical to SEQ ID NO: 259;

d. nucleotide 4 through nucleotide 36 of a sequence that is at least 90% identical to SEQ ID NO: 259;

e. nucleotide 5 through nucleotide 36 of a sequence that is at least 90% identical to SEQ ID NO: 259;

f. nucleotide 6 through nucleotide 36 of a sequence that is at least 90% identical to SEQ ID NO: 259;

g. nucleotide 7 through nucleotide 36 of a sequence that is at least 90% identical to SEQ ID NO: 259;

h. nucleotide 8 through nucleotide 36 of a sequence that is at least 90% identical to SEQ ID NO: 259;

i. nucleotide 9 through nucleotide 36 of a sequence that is at least 90% identical to SEQ ID NO: 259;

j. nucleotide 10 through nucleotide 36 of a sequence that is at least 90% identical to SEQ ID NO: 259;

k. nucleotide 11 through nucleotide 36 of a sequence that is at least 90% identical to SEQ ID NO: 259;

l. nucleotide 12 through nucleotide 36 of a sequence that is at least 90% identical to SEQ ID NO: 259;

m. nucleotide 13 through nucleotide 36 of a sequence that is at least 90% identical to SEQ ID NO: 259;

n. nucleotide 14 through nucleotide 36 of a sequence that is at least 90% identical to SEQ ID NO: 259; or

o. a sequence that is at least 90% identical to a sequence of SEQ ID NO: 260 or SEQ ID NO: 261 or a portion thereof.

In still other examples, the direct repeat may comprise:

a. nucleotide 1 through nucleotide 36 of SEQ ID NO: 259;

b. nucleotide 2 through nucleotide 36 of SEQ ID NO: 259;

c. nucleotide 3 through nucleotide 36 of SEQ ID NO: 259;

d. nucleotide 4 through nucleotide 36 of SEQ ID NO: 259;

e. nucleotide 5 through nucleotide 36 of SEQ ID NO: 259;

f. nucleotide 6 through nucleotide 36 of SEQ ID NO: 259;

g. nucleotide 7 through nucleotide 36 of SEQ ID NO: 259;

h. nucleotide 8 through nucleotide 36 of SEQ ID NO: 259;

i. nucleotide 9 through nucleotide 36 of SEQ ID NO: 259;

j. nucleotide 10 through nucleotide 36 of SEQ ID NO: 259;

k. nucleotide 11 through nucleotide 36 of SEQ ID NO: 259;

l. nucleotide 12 through nucleotide 36 of SEQ ID NO: 259;

m. nucleotide 13 through nucleotide 36 of SEQ ID NO: 259;

n. nucleotide 14 through nucleotide 36 of SEQ ID NO: 259; or

o. SEQ ID NO: 260 or SEQ ID NO: 261 or a portion thereof.

In other examples, the direct repeat may comprise:

a. nucleotide 1 through nucleotide 36 of a sequence that is at least 90% identical to a sequence of SEQ ID NO: 262 or SEQ ID NO: 263;

b. nucleotide 2 through nucleotide 36 of a sequence that is at least 90% identical to a sequence of SEQ ID NO: 262 or SEQ ID NO: 263;

c. nucleotide 3 through nucleotide 36 of a sequence that is at least 90% identical to a sequence of SEQ ID NO: 262 or SEQ ID NO: 263;

d. nucleotide 4 through nucleotide 36 of a sequence that is at least 90% identical to a sequence of SEQ ID NO: 262 or SEQ ID NO: 263;

e. nucleotide 5 through nucleotide 36 of a sequence that is at least 90% identical to a sequence of SEQ ID NO: 262 or SEQ ID NO: 263;

f. nucleotide 6 through nucleotide 36 of a sequence that is at least 90% identical to a sequence of SEQ ID NO: 262 or SEQ ID NO: 263;

g. nucleotide 7 through nucleotide 36 of a sequence that is at least 90% identical to a sequence of SEQ ID NO: 262 or SEQ ID NO: 263;

h. nucleotide 8 through nucleotide 36 of a sequence that is at least 90% identical to a sequence of SEQ ID NO: 262 or SEQ ID NO: 263;

i. nucleotide 9 through nucleotide 36 of a sequence that is at least 90% identical to a sequence of SEQ ID NO: 262 or SEQ ID NO: 263;

j. nucleotide 10 through nucleotide 36 of a sequence that is at least 90% identical to a sequence of SEQ ID NO: 262 or SEQ ID NO: 263;

k. nucleotide II through nucleotide 36 of a sequence that is at least 90% identical to a sequence of SEQ ID NO: 262 or SEQ ID NO: 263;

l. nucleotide 12 through nucleotide 36 of a sequence that is at least 90% identical to a sequence of SEQ ID NO: 262 or SEQ ID NO: 263;

m. nucleotide 13 through nucleotide 36 of a sequence that is at least 90% identical to a sequence of SEQ ID NO: 262 or SEQ ID NO: 263;

n. nucleotide 14 through nucleotide 36 of a sequence that is at least 90% identical to a sequence of SEQ ID NO: 262 or SEQ ID NO: 263;

o. nucleotide 15 through nucleotide 36 of a sequence that is at least 90% identical to a sequence of SEQ ID NO: 262 or SEQ ID NO: 263; or

p. a sequence that is at least 90% identical to a sequence of SEQ ID NO: 264 or a portion thereof.

In some examples, the direct repeat may comprise:

a. nucleotide 1 through nucleotide 36 of SEQ ID NO: 262 or SEQ ID NO: 263;

b. nucleotide 2 through nucleotide 36 of SEQ ID NO: 262 or SEQ ID NO: 263;

c. nucleotide 3 through nucleotide 36 of SEQ ID NO: 262 or SEQ ID NO: 263;

d. nucleotide 4 through nucleotide 36 of SEQ ID NO: 262 or SEQ ID NO: 263;

e. nucleotide 5 through nucleotide 36 of SEQ ID NO: 262 or SEQ ID NO: 263;

f. nucleotide 6 through nucleotide 36 of SEQ ID NO: 262 or SEQ ID NO: 263;

g. nucleotide 7 through nucleotide 36 of SEQ ID NO: 262 or SEQ ID NO: 263;

h. nucleotide 8 through nucleotide 36 of SEQ ID NO: 262 or SEQ ID NO: 263;

i. nucleotide 9 through nucleotide 36 of SEQ ID NO: 262 or SEQ ID NO: 263;

j. nucleotide 10 through nucleotide 36 of SEQ ID NO: 262 or SEQ ID NO: 263;

k. nucleotide 11 through nucleotide 36 of SEQ ID NO: 262 or SEQ ID NO: 263;

l. nucleotide 12 through nucleotide 36 of SEQ ID NO: 262 or SEQ ID NO: 263;

m. nucleotide 13 through nucleotide 36 of SEQ ID NO: 262 or SEQ ID NO: 263:

n. nucleotide 14 through nucleotide 36 of SEQ ID NO: 262 or SEQ ID NO: 263;

o. nucleotide 15 through nucleotide 36 of SEQ ID NO: 262 or SEQ ID NO: 263; or p. SEQ ID NO: 264 or a portion thereof.

In any of the compositions of Embodiment 1, the spacer sequence is substantially complementary or completely complementary to the complement of a sequence of any one of SEQ ID NOs: 11-115.

In any of the compositions of Embodiment 1, the PAM comprises the sequence 5′-ATTA-3′, 5′-ATTT-3′, 5′-ATTG-3′, 5′-ATTC-3′, 5′-TTA-3′, 5′-TTIT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence.

In any of the compositions of Embodiment 1, the RNA guide has a sequence that is at least 90% identical to a sequence of any one of SEQ ID NOs: 273-278 or 345-362. In some examples, the RNA guide has the sequence of any one of SEQ ID NOs: 273-278 or 345-362.

Embodiment 2: The composition of Embodiment 1 may further comprise a Cas12i polypeptide or a polyribonucleotide encoding a Cas12i polypeptide. In some examples, the Cas12i polypeptide can be:

a. a Cas12i2 polypeptide comprising a sequence that is at least 90% identical to the sequence of SEQ ID NO: 222, SEQ ID NO: 223, SEQ ID NO: 224. SEQ ID NO: 225, SEQ ID NO: 226, or SEQ ID NO: 227;

b. a Cas12i4 polypeptide comprising a sequence that is at least 90% identical to the sequence of SEQ ID NO: 253, SEQ ID NO: 254, or SEQ ID NO: 255:

c. a Cas12i1 polypeptide comprising a sequence that is at least 90% identical to the sequence of SEQ ID NO: 265; or

d. a Cas12i3 polypeptide comprising a sequence that is at least 90% identical to the sequence of SEQ ID NO: 266.

In some examples, the Cas12i polypeptide is:
a. a Cas12i2 polypeptide comprising a sequence of SEQ ID NO: 222, SEQ ID NO: 223, SEQ ID NO: 224. SEQ ID NO: 225, SEQ ID NO: 226, or SEQ ID NO: 227;
b. a Cas12i4 polypeptide comprising a sequence of SEQ ID NO: 253, SEQ ID NO: 254, or SEQ ID NO: 255;
c. a Cas12i1 polypeptide comprising a sequence of SEQ ID NO: 265; or
d. a Cas12i3 polypeptide comprising a sequence of SEQ ID NO: 266.

In some examples, the RNA guide and the Cas12i polypeptide form a ribonucleoprotein complex. For example, the ribonucleoprotein complex binds the target nucleic acid.

Embodiment 3: The composition of Embodiment 1 or Embodiment 2 may further comprise a template DNA, which may comprise a donor region comprising a first sequence difference relative to the target nucleic acid. In some instances, the template DNA comprises a homology arm (e.g., a left homology arm and/or a right homology arm). In some examples, the template DNA is human DNA.

In some examples, the template DNA is double stranded. For example, the two strands of the double stranded template DNA are substantially complementary or perfectly complementary to each other. In other examples, the template DNA is single stranded. In some instances, the template DNA is single stranded and the left homology arm or the right homology arm, each independently, has at least 80%, 85%, 90%, 95%, 99%, or 100% identity to the corresponding region of the non-target strand (the PAM strand).

In some specific examples, the template DNA is single stranded and has reverse complementarity relative to the target strand. In some instances, the template DNA is single stranded and the left homology arm or the right homology arm, each independently, has at least 80%, 85%, 90%, 95%, 99%, or 100% identity to the corresponding region of the target strand. In other instances, the template DNA is single stranded and has reverse complementarity relative to the non-target strand.

Embodiment 4: in any of the compositions of any one of Embodiments 1-3, the target nucleic acid comprises a mutation associated with a disease. For example, the target nucleic acid comprises a TTR gene having a mutation associated with a disease, and the template DNA comprises a wild-type allele of the TTR gene, or a portion thereof corresponding to the mutation.

In some examples, the template DNA comprises a wild-type sequence corresponding to a mutant sequence in the target nucleic acid. In some examples, the template DNA does not comprise a mutation associated with a disease. In some examples, the template DNA comprises a mutation associated with a disease. In specific examples, the mutation associated with disease is V30M, V122I, T60A, L58H, or 184S relative to the TTR sequence of SEQ ID NO: 257.

In some examples, the template DNA comprises a protective mutation associated with a disease. For example, the template DNA comprises a mutation associated with a disease and a protective mutation. In specific examples, the protective mutation is T119M relative to the TTR sequence of SEQ ID NO: 257.

Embodiment 5: in any of the compositions of any one of Embodiments 1-4, the disease is hereditary ATTR (hATTR) or wild-type ATTR amyloidosis.

In some examples, the template DNA comprises a left homology arm and does not comprise a right homology arm. In other examples, the template DNA comprises a right homology arm and does not comprise a left homology arm. Alternatively, the template DNA comprises a right homology arm and a left homology arm.

In some examples, the left homology arm is the same length as or is about the same length as the right homology arm. In some examples, the left homology arm is about 10-30, 20-40, 30-50, 40-60, 50-80, 70-100, 90-150, 140-200, 190-250, 240-300, 290-350, 340-400, 390-450, or 440-500 nucleotides in length. In other examples, the left homology arm is about 20-200 (e.g., about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, or 195-200 nucleotides), about 200-500 (e.g., about 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, or 490-500) nucleotides in length.

Alternatively or in addition, the right homology arm is about 10-30, 20-40, 30-50, 40-60, 60-80, 80-100, 100-150, 150-200, 200-250, 250-300, 300-350, 350-400, 400-450, or 450-500 nucleotides in length. For example, the right homology arm is about 20-200 (e.g., about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, or 195-200 nucleotides), about 200-500 (e.g., about 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, or 490-500) nucleotides in length.

Embodiment 6: in the composition of any one of Embodiments 1-5, the first sequence difference comprised by the donor region is situated within the corresponding region of the target sequence.

In some examples, the first sequence difference comprised by the donor region is situated within the corresponding region 10 nucleotides upstream or downstream of the target sequence. In some examples, the first sequence difference comprised by the donor region is situated within the corresponding region 5 nucleotides upstream or downstream of the target sequence. In some examples, the first sequence difference comprises a substitution relative to the target nucleic acid. In some examples, the first sequence difference comprises a silent mutation (e.g., a mutation to the third position of a codon that does not change the amino acid encoded by that codon). In some examples, the first sequence difference comprises a polymorphism relative to the target nucleic acid.

Embodiment 7: in the composition of any one of Embodiments 1-6, the donor sequence further comprises a second sequence difference relative to the target nucleic acid.

Embodiment 8: in the composition of any one of Embodiments 1-7, the second sequence difference comprised by the donor region is situated within the region corresponding to the target sequence.

In some examples, the second sequence difference comprised by the donor region is situated within the corresponding region 10 nucleotides upstream or downstream of the target sequence. In some examples, the second sequence difference comprised by the donor region is situated within the corresponding region 5 nucleotides upstream or downstream of the target sequence. In some examples, the second sequence difference is a substitution.

Embodiment 9: in the composition of any one of Embodiments 1-8, the donor region further comprises a third sequence difference, and optionally a fourth sequence difference, relative to the target nucleic acid.

Embodiment 10: in the composition of any one of Embodiments 1-9, the template DNA comprises one or more internucleoside modifications (e.g., phosphorothioate modifications).

In some examples, the left homology arm comprises one or more internucleoside modifications (e.g., phosphorothioate modifications). In some examples, the right homology arm comprises one or more internucleoside modifications (e.g., phosphorothioate modifications). Alternatively, the left homology arm comprises one or more internucleoside modifications (e.g., phosphorothioate modifications) and the right homology arm comprises one or more internucleoside modifications (e.g., phosphorothioate modifications). In some examples, the left homology arm comprises two internucleoside modifications (e.g., phosphorothioate modifications) and the right homology arm comprises two internucleoside modifications (e.g., phosphorothioate modifications).

In some specific examples, the phosphorothioate modifications are at the 5′ end of the left homology arm and the 3′ end of the right homology arm. In other specific examples, the left homology arm is 5′ of the right homology arm and the left homology arm comprises two phosphorothioate modifications at the 5′ end of the left homology arm, the right homology arm comprises two phosphorothioate modifications at the 3′ end of the right homology arm.

Embodiment 11: in the composition of any one of Embodiments 1-10, the template DNA is a double stranded DNA that comprises a first strand and a second strand, wherein the first strand comprises at least one (e.g., 2) phosphorothioate modifications at the 5′ end of the first strand or at least one (e.g., 2) phosphorothioate modifications at the 3′ end of the first strand, or both.

In some examples, the second strand comprises at least one (e.g., 2) phosphorothioate modifications at the 5′ end of the second strand or at least one (e.g., 2) phosphorothioate modifications at the 3′ end of the second strand, or both.

Embodiment 12: the composition of any one of Embodiments 1-11 is present within a cell.

Embodiment 13: in the composition of any one of Embodiments 1-12, the RNA guide and the Cas12i polypeptide are encoded in a vector, e.g., expression vector. In some examples, the RNA guide and the Cas12i polypeptide are encoded in a single vector or the RNA guide is encoded in a first vector and the Cas12i polypeptide is encoded in a second vector.

Embodiment 14: in the composition of any one of Embodiments 1-13, the template DNA is in a vector.

Embodiment 15, An RNA guide comprising (i) a spacer sequence that is substantially complementary or completely complementary to a target sequence within a TTR gene and (ii) a direct repeat sequence. In some examples, the target sequence is within exon 1, exon 2, exon 3, or exon 4 of the TTR gene. In some examples, the TTR gene comprises the sequence of SEQ ID NO: 228, the reverse complement of SEQ ID NO: 228, a variant of SEQ ID NO: 228, the reverse complement of a variant of SEQ ID NO: 228, the sequence of SEQ ID NO: 258, the reverse complement of SEQ ID NO: 258, a variant of SEQ ID NO 258, or the reverse complement of a variant of SEQ ID NO: 258.

In Embodiment 15, the spacer sequence may comprise: a. nucleotide 1 through nucleotide 16 of a sequence that is at least 90% identical to a sequence of any one of SEQ ID NOs: 116-220; b. nucleotide 1 through nucleotide 17 of a sequence that is at least 90% identical to a sequence of any one of SEQ ID NOs: 116-220; c. nucleotide 1 through nucleotide 18 of a sequence that is at least 90% identical to a sequence of any one of SEQ ID NOs: 116-220; d. nucleotide 1 through nucleotide 19 of a sequence that is at least 90% identical to a sequence of any one of SEQ ID NOs: 116-220; e. nucleotide 1 through nucleotide 20 of a sequence that is at least 90% identical to a sequence of any one of SEQ ID NOs: 116-220; f. nucleotide 1 through nucleotide 21 of a sequence that is at least 90% identical to a sequence of any one of SEQ ID NOs: 116-220; g. nucleotide 1 through nucleotide 22 of a sequence that is at least 90% identical to a sequence of any one of SEQ ID NOs: 116-220; h. nucleotide 1 through nucleotide 23 of a sequence that is at least 90% identical to a sequence of any one of SEQ ID NOs: 116-220; i. nucleotide 1 through nucleotide 24 of a sequence that is at least 90% identical to a sequence of any one of SEQ ID NOs: 116-220; j. nucleotide 1 through nucleotide 25 of a sequence that is at least 90% identical to a sequence of any one of SEQ ID NOs: 116-220; k. nucleotide 1 through nucleotide 26 of a sequence that is at least 90% identical to a sequence of any one of SEQ ID NOs: 116-220; l. nucleotide 1 through nucleotide 27 of a sequence that is at least 90% identical to a sequence of any one of SEQ ID NOs: 116-220; m. nucleotide 1 through nucleotide 28 of a sequence that is at least 90% identical to a sequence of any one of SEQ ID NOs: 116-220; n. nucleotide 1 through nucleotide 29 of a sequence that is at least 90% identical to a sequence of any one of SEQ ID NOs: 116-220; or o. nucleotide 1 through nucleotide 30 of a sequence that is at least 90% identical to a sequence of any one of SEQ ID NOs: 116-220.

In some examples, the spacer sequence may comprise: a. nucleotide 1 through nucleotide 16 of any one of SEQ ID NOs: 116-220; b. nucleotide 1 through nucleotide 17 of any one of SEQ ID NOs: 116-220; c. nucleotide 1 through nucleotide 18 of any one of SEQ ID NOs: 116-220; d. nucleotide 1 through nucleotide 19 of any one of SEQ ID NOs: 116-220; e. nucleotide 1 through nucleotide 20 of any one of SEQ ID NOs: 116-220; f. nucleotide 1 through nucleotide 21 of any one of SEQ ID NOs: 116-220; g. nucleotide 1 through nucleotide 22 of any one of SEQ ID NOs: 116-220; h. nucleotide 1 through nucleotide 23 of any one of SEQ ID NOs: 116-220; i. nucleotide 1 through nucleotide 24 of any one of SEQ ID NOs: 116-220; j. nucleotide 1 through nucleotide 25 of any one of SEQ ID NOs: 116-220; k. nucleotide 1 through nucleotide 26 of any one of SEQ ID NOs: 116-220; l. nucleotide 1 through nucleotide 27 of any one of SEQ ID NOs: 116-220; m. nucleotide 1 through nucleotide 28 of any one of SEQ ID NOs: 116-220; n. nucleotide 1 through nucleotide 29 of any one of SEQ ID NOs: 116-220; or o. nucleotide 1 through nucleotide 30 of any one of SEQ ID NOs: 116-220.

In Embodiment 15, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In some examples, the direct repeat may comprise: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In some examples, the direct repeat comprises: a. nucleotide 1 through nucleotide 36 of a sequence that is at least 90% identical to a sequence of any one of SEQ ID NOs: 233-250; b. nucleotide 2 through nucleotide 36 of a sequence that is at least 90% identical to a sequence of any one of SEQ ID NOs: 233-250; c. nucleotide 3 through nucleotide 36 of a sequence that is at least 90% identical to a sequence of any one of SEQ ID NOs: 233-250; d. nucleotide 4 through nucleotide 36 of a sequence that is at least 90% identical to a sequence of any one of SEQ ID NOs: 233-250; e. nucleotide 5 through nucleotide 36 of a sequence that is at least 90% identical to a sequence of any one of SEQ ID NOs: 233-250; f. nucleotide 6 through nucleotide 36 of a sequence that is at least 90% identical to a sequence of any one of SEQ ID NOs: 233-250; g. nucleotide 7 through nucleotide 36 of a sequence that is at least 90% identical to a sequence of any one of SEQ ID NOs: 233-250; h. nucleotide 8 through nucleotide 36 of a sequence that is at least 90% identical to a sequence of any one of SEQ ID NOs: 233-250; i. nucleotide 9 through nucleotide 36 of a sequence that is at least 90% identical to a sequence of any one of SEQ ID NOs: 233-250; j. nucleotide 10 through nucleotide 36 of a sequence that is at least 90% identical to a sequence of any one of SEQ ID NOs: 233-250; k. nucleotide 11 through nucleotide 36 of a sequence that is at least 90% identical to a sequence of any one of SEQ ID NOs: 233-250; 1. nucleotide 12 through nucleotide 36 of a sequence that is at least 90% identical to a sequence of any one of SEQ ID NOs: 233-250; m. nucleotide 13 through nucleotide 36 of a sequence that is at least 90% identical to a sequence of any one of SEQ ID NOs: 233-250; n. nucleotide 14 through nucleotide 36 of a sequence that is at least 90% identical to a sequence of any one of SEQ ID NOs: 233-250; or o. a sequence that is at least 90% identical to a sequence of SEQ ID NO: 251 or a portion thereof.

In some examples, the direct repeat comprises: a. nucleotide 1 through nucleotide 36 of any one of SEQ ID NOs: 233-250; b. nucleotide 2 through nucleotide 36 of any one of SEQ ID NOs: 233-250; c. nucleotide 3 through nucleotide 36 of any one of SEQ ID NOs: 233-250; d. nucleotide 4 through nucleotide 36 of any one of SEQ ID NOs: 233-250; e. nucleotide 5 through nucleotide 36 of any one of SEQ ID NOs: 233-250; f. nucleotide 6 through nucleotide 36 of any one of SEQ ID NOs: 233-250; g. nucleotide 7 through nucleotide 36 of any one of SEQ ID NOs: 233-250; h. nucleotide 8 through nucleotide 36 of any one of SEQ ID NOs: 233-250; i. nucleotide 9 through nucleotide 36 of any one of SEQ ID NOs: 233-250; j. nucleotide 10 through nucleotide 36 of any one of SEQ ID NOs: 233-250; k. nucleotide 11 through nucleotide 36 of any one of SEQ ID NOs: 233-250; l. nucleotide 12 through nucleotide 36 of any one of SEQ ID NOs: 233-250; m. nucleotide 13 through nucleotide 36 of any one of SEQ ID NOs: 233-250; n. nucleotide 14 through nucleotide 36 of any one of SEQ ID NOs: 233-250; or o. SEQ ID NO: 251 or a portion thereof.

In some examples, the direct repeat comprises: a. nucleotide 1 through nucleotide 36 of a sequence that is at least 90% identical to SEQ ID NO: 259; b. nucleotide 2 through nucleotide 36 of a sequence that is at least 90% identical to SEQ ID NO: 259; c. nucleotide 3 through nucleotide 36 of a sequence that is at least 90% identical to SEQ ID NO: 259; d. nucleotide 4 through nucleotide 36 of a sequence that is at least 90% identical to SEQ ID NO: 259; e. nucleotide 5 through nucleotide 36 of a sequence that is at least 90% identical to SEQ ID NO: 259; f. nucleotide 6 through nucleotide 36 of a sequence that is at least 90% identical to SEQ ID NO: 259; g. nucleotide 7 through nucleotide 36 of a sequence that is at least 90% identical to SEQ ID NO: 259; h. nucleotide 8 through nucleotide 36 of a sequence that is at least 90% identical to SEQ ID NO: 259; i. nucleotide 9 through nucleotide 36 of a sequence that is at least 90% identical to SEQ ID NO: 259; j. nucleotide 10 through nucleotide 36 of a sequence that is at least 90% identical to SEQ ID NO: 259; k. nucleotide 11 through nucleotide 36 of a sequence that is at least 90% identical to SEQ ID NO: 259; l. nucleotide 12 through nucleotide 36 of a sequence that is at least 90% identical to SEQ ID NO: 259; m. nucleotide 13 through nucleotide 36 of a sequence that is at least 90% identical to SEQ ID NO: 259; n. nucleotide 14 through nucleotide 36 of a sequence that is at least 90% identical to SEQ ID NO: 259; or o. a sequence that is at least 90% identical to a sequence of SEQ ID NO: 260 or SEQ ID NO: 261 or a portion thereof.

In some examples, the direct repeat comprises: a. nucleotide 1 through nucleotide 36 of SEQ ID NO: 259; b. nucleotide 2 through nucleotide 36 of SEQ ID NO: 259; c. nucleotide 3 through nucleotide 36 of SEQ ID NO: 259; d. nucleotide 4 through nucleotide 36 of SEQ ID NO: 259; e. nucleotide 5 through nucleotide 36 of SEQ ID NO: 259; f. nucleotide 6 through nucleotide 36 of SEQ ID NO: 259; g. nucleotide 7 through nucleotide 36 of SEQ ID NO: 259; h. nucleotide 8 through nucleotide 36 of SEQ ID NO: 259; i. nucleotide 9 through nucleotide 36 of SEQ ID NO: 259; j. nucleotide 10 through nucleotide 36 of SEQ ID NO: 259; k. nucleotide 11 through nucleotide 36 of SEQ ID NO: 259; l. nucleotide 12 through nucleotide 36 of SEQ ID NO: 259; m. nucleotide 13 through nucleotide 36 of SEQ ID NO: 259; n. nucleotide 14 through nucleotide 36 of SEQ ID NO: 259; or o. SEQ ID NO: 260 or SEQ ID NO: 261 or a portion thereof.

In some examples, the direct repeat comprises: a. nucleotide 1 through nucleotide 36 of a sequence that is at least 90% identical to a sequence of SEQ ID NO: 262 or SEQ ID NO: 263; b. nucleotide 2 through nucleotide 36 of a sequence that is at least 90% identical to a sequence of SEQ ID NO: 262 or SEQ ID NO: 263; c. nucleotide 3 through nucleotide 36 of a sequence that is at least 90% identical to a sequence of SEQ ID NO: 262 or SEQ ID NO: 263; d. nucleotide 4 through nucleotide 36 of a sequence that is at least 90% identical to a sequence of SEQ ID NO: 262 or SEQ ID NO: 263; e. nucleotide 5 through nucleotide 36 of a sequence that is at least 90% identical to a sequence of SEQ ID NO: 262 or SEQ ID NO: 263; f. nucleotide 6 through nucleotide 36 of a sequence that is at least 90% identical to a sequence of SEQ ID NO: 262 or SEQ ID NO: 263; g. nucleotide 7 through nucleotide 36 of a sequence that is at least 90% identical to a sequence of SEQ ID NO: 262 or SEQ ID NO: 263; h. nucleotide 8 through nucleotide 36 of a sequence that is at least 90% identical to a sequence of SEQ ID NO: 262 or SEQ ID NO: 263; i. nucleotide 9 through nucleotide 36 of a sequence that is at least 90% identical to a sequence of SEQ ID NO: 262 or SEQ ID NO: 263; j. nucleotide 10 through nucleotide 36 of a sequence that is at least 90% identical to a sequence of SEQ ID NO: 262 or SEQ ID NO: 263; k. nucleotide 1I through nucleotide 36 of a sequence that is at least 90% identical to a sequence of SEQ ID NO: 262 or SEQ ID NO: 263; l. nucleotide 12 through nucleotide 36 of a sequence that is at least 90% identical to a sequence of SEQ ID NO: 262 or SEQ ID NO: 263; m. nucleotide 13 through nucleotide 36 of a sequence that is at least 90% identical to a sequence of SEQ ID NO: 262 or SEQ ID NO: 263; n. nucleotide 14 through nucleotide 36 of a sequence that is at least 90% identical to a sequence of SEQ ID NO: 262 or SEQ ID NO: 263; o. nucleotide 15 through nucleotide 36 of a sequence that is at least 90% identical to a sequence of SEQ ID NO: 262 or SEQ ID NO: 263; or p. a sequence that is at least 90% identical to a sequence of SEQ ID NO: 264 or a portion thereof.

In some examples, the direct repeat comprises: a. nucleotide 1 through nucleotide 36 of SEQ ID NO: 262 or SEQ ID NO: 263; b. nucleotide 2 through nucleotide 36 of SEQ ID NO: 262 or SEQ ID NO: 263; c. nucleotide 3 through nucleotide 36 of SEQ ID NO: 262 or SEQ ID NO: 263; d. nucleotide 4 through nucleotide 36 of SEQ ID NO: 262 or SEQ ID NO: 263; e. nucleotide 5 through nucleotide 36 of SEQ ID NO: 262 or SEQ ID NO: 263; f. nucleotide 6 through nucleotide 36 of SEQ ID NO: 262 or SEQ ID NO: 263; g. nucleotide 7 through nucleotide 36 of SEQ ID NO: 262 or SEQ ID NO: 263; h. nucleotide 8 through nucleotide 36 of SEQ ID NO: 262 or SEQ ID NO: 263; i. nucleotide 9 through nucleotide 36 of SEQ ID NO: 262 or SEQ ID NO: 263; j. nucleotide 10 through nucleotide 36 of SEQ ID NO: 262 or SEQ ID NO: 263; k. nucleotide 11 through nucleotide 36 of SEQ ID NO: 262 or SEQ ID NO: 263; l. nucleotide 12 through nucleotide 36 of SEQ ID NO: 262 or SEQ ID NO: 263; m. nucleotide 13 through nucleotide 36 of SEQ ID NO: 262 or SEQ ID NO: 263; n. nucleotide 14 through nucleotide 36 of SEQ ID NO: 262 or SEQ ID NO: 263; o. nucleotide 15 through nucleotide 36 of SEQ ID NO: 262 or SEQ ID NO: 263; or p. SEQ ID NO: 264 or a portion thereof.

In Embodiment 15, the spacer sequence is substantially complementary or completely complementary to the complement of a sequence of any one of SEQ ID NOs: 11-115.

In Embodiment 15, the target sequence is adjacent to a protospacer adjacent motif (PAM) comprising the sequence 5′-NTTN-3′, wherein N is any nucleotide. In some examples, the PAM comprises the sequence 5′-ATTA-3′, 5′-ATTT-3′, 5′-ATTG-3′, 5′-ATTC-3′, 5′-TTTA-3′, 5′-TITT-3′, 5′-TTG-3′, 5′-TTTC-3′, 5′-GTTA-3′, 5′-GTTT-3′, 5′-GTTG-3′, 5′-GTTC-3′, 5′-CTTA-3′, 5′-CT7T-3′, 5′-CTTG-3′, or 5′-CTTC-3′.

In some examples, the target sequence is immediately adjacent to the PAM sequence.

In specific examples, the RNA guide has a sequence that is at least 90% identical to a sequence of any one of SEQ ID NOs: 273-278 or 345-362. For example, the RNA guide has the sequence of any one of SEQ ID NOs: 273-278 or 345-362.

Embodiment 16: A nucleic acid encoding an RNA guide as described herein (e.g., set forth in Embodiment 15).

Embodiment 17: A vector comprising a nucleic acid described herein (e.g., set forth in Embodiment 16).

Embodiment 18: A vector system comprising one or more vectors encoding (i) an RNA guide as described herein and (ii) a Cas12i polypeptide, optionally wherein the vector system comprises a first vector encoding the RNA guide and a second vector encoding the Cas12i polypeptide.

Embodiment 19: A cell comprising a composition, an RNA guide, a nucleic acid, a vector, or vector system as described herein. In some examples, the cell is a eukaryotic cell, an animal cell, a mammalian cell, a human cell, a primary cell, a cell line, a stem cell, or a T cell. In specific examples, the cell is a liver cell (e.g., a hepatocyte).

Embodiment 20: A kit comprising a composition, RNA guide, nucleic acid, vector, or vector system described herein.

Embodiment 21: A method of editing a TTR sequence, the method comprising contacting a TTR sequence with a composition or an RNA guide as described herein, wherein optionally the method is carried out in vivo, in vitro, or ex rivo. In some examples, the TTR sequence is in a cell. In some examples, the composition or the RNA guide induces a deletion in the TTR sequence.

In some examples, the deletion is adjacent to a 5′-NTTN-3′ sequence, wherein N is any nucleotide. For example, the deletion is downstream of the 5′-NTTN-3′ sequence. In some instances, the deletion is up to about 50 nucleotides in length. In some instances, the deletion is up to about 40 nucleotides in length. In some instances, the deletion is from about 4 nucleotides to 40 nucleotides in length. In some instances, the deletion is from about 4 nucleotides to 25 nucleotides in length. In some specific examples, the deletion is from about 10 nucleotides to 25 nucleotides in length. In other specific examples, the deletion is from about 10 nucleotides to 15 nucleotides in length.

In some examples, the deletion starts within about 5 nucleotides to about 15 nucleotides of the 5′-NTTN-3′ sequence. In some instances, the deletion starts within about 5 nucleotides to about 10 nucleotides of the 5′-NTTN-3′ sequence. In some instances, the deletion starts within about 10 nucleotides to about 15 nucleotides of the 5′-NTTN-3′ sequence. In some instances, the deletion starts within about 5 nucleotides to about 15 nucleotides downstream of the 5′-NTTN-3′ sequence. In some instances, the deletion starts within about 5 nucleotides to about 10 nucleotides downstream of the 5′-NTTN-3′ sequence. in some instances, the deletion starts within about 10 nucleotides to about 15 nucleotides downstream of the 5′-NTTN-3′ sequence.

In some examples, the deletion ends within about 20 nucleotides to about 30 nucleotides of the 5′-NTTN-3′ sequence. In some instances, the deletion ends within about 20 nucleotides to about 25 nucleotides of the 5′-NTTN-3′ sequence. In some instances, the deletion ends within about 25 nucleotides to about 30 nucleotides of the 5′-NTTN-3′ sequence. In some instances, the deletion ends within about 20 nucleotides to about 30 nucleotides downstream of the 5′-NTTN-3′ sequence. In some instances, the deletion ends within about 20 nucleotides to about 25 nucleotides downstream of the 5′-NTTN-3′ sequence. In some instances, the deletion ends within about 25 nucleotides to about 30 nucleotides downstream of the 5′-NTTN-3′ sequence.

In some specific examples, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence. In other specific examples, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence. In yet other specific examples, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence. Alternatively, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence. In still other examples, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence. In other instances, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence. In other instances, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence. In yet other instances, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence. In yet other instances, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In the method of Embodiment 21, the 5′-NTTN-3′ sequence is 5′-CTTT-3′, 5′-CTTC-3′, 5′-GTTT-3′, 5′-GTTC-3′, 5′-TITC-3′, 5′-GTTA-3′, or 5′-GTTG-3′.

In some examples, the deletion overlaps with a mutation in the gene. In some examples, the deletion disrupts one or both alleles of the gene.

In the method of Embodiment 22, the method introduces a first sequence difference into the target nucleic acid. In some examples, the first sequence difference comprised by the donor region is situated within the target sequence. In some examples, the first sequence difference is situated within 10 nucleotides upstream or downstream of the target sequence.

In some examples, the first sequence difference is situated within 5 nucleotides upstream or downstream of the target sequence. In some examples, the first sequence difference comprises a substitution relative to the target nucleic acid.

In some examples, the first sequence difference comprises a silent mutation (e.g., a mutation to the third position of a codon that does not change the amino acid encoded by that codon). In some examples, the first sequence difference comprises a polymorphism relative to the target nucleic acid.

In the method of Embodiment 22, the method introduces a second sequence difference into the target nucleic acid. In some examples, the second sequence difference is situated within the target sequence. In some examples, the second sequence difference is situated within 10 nucleotides upstream or downstream of the target sequence. In some examples, the second sequence difference is situated within 5 nucleotides upstream or downstream of the target sequence. In some examples, the second sequence difference is a substitution.

In the method of Embodiment 22, the method further introduces a third sequence difference, and optionally a fourth sequence difference, into to the target nucleic acid.

Any of the methods of Embodiment 22 corrects a mutation associated with a disease. In some examples, the method introduces a protective mutation. In some examples, the method corrects a mutation associated with a disease and introduces a protective mutation. In some instances, the disease is hATTR or wild-type ATTR amyloidosis. In some examples, the mutation associated with disease is V30M, V122I, T60A, L58H, or 184S relative to the TTR sequence of SEQ ID NO: 257. In some examples, the protective mutation is TI 19M relative to the TIR sequence of SEQ ID NO: 257.

Any of the methods of Embodiment 22 may reduce TTR expression in a cell. Alternatively, the method may stabilize TTR in a cell. In some examples, the method reduces aggregation of amyloid fibrils in a cell.

In any of the methods of Embodiment 22, the cell is a liver cell (e.g., a hepatocyte). In some examples, the cell resides in a patient with hATTR or wild-type ATTR amyloidosis.

Embodiment 23: A composition, RNA guide, nucleic acid, vector, vector system, cell, kit, or method described herein, the RNA guide comprises the sequence of any one of SEQ ID NOs: 273-278 or 345-362.

Embodiment 24: A method of treating amyloid disease, senile systemic amyloidosis, familial amyloid polyneuropathy, or familial amyloid cardiomyopathy in a subject, the method comprising administering a composition, RNA guide, or cell as described herein to the subject.

Embodiment 25: A composition, cell, kit, or method described herein, the RNA guide and/or the polyribonucleotide encoding the Cas12i polypeptide are comprised within a lipid nanoparticle.

Embodiment 26: A composition, cell, kit, or method described herein, the RNA guide and the polyribonucleotide encoding the Cas12i polypeptide are comprised within the same lipid nanoparticle.

Embodiment 27: A composition, cell, kit, or method described herein, the RNA guide and the polyribonucleotide encoding the Cas12i polypeptide are comprised within separate lipid nanoparticles.

Embodiment 28: An RNA guide comprising (i) a spacer sequence that is complementary to a target sequence within a TTR gene and (ii) a direct repeat sequence, wherein the target sequence is a sequence of any one of SEQ ID NOs: 267-272 or 327-344 or the reverse complement thereof. In some examples, the RNA guide comprises the direct repeat sequence, which can be any direct repeat sequences disclosed herein. In any of the RNA guides of Embodiment 28, each of the first three nucleotides of the RNA guide comprises a 2′-O-methyl phosphorothioate modification. Alternatively or in addition, each of the last four nucleotides of the RNA guide comprises a 2′-O-methyl phosphorothioate modification. In specific examples, each of the first to last, second to last, and third to last nucleotides of the RNA guide comprises a 2′-O-methyl phosphorothioate modification, and wherein the last nucleotide of the RNA guide is unmodified.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press. 1999); The Antibodies (M. Zanetti and J. D. Capra, eds. Harwood Academic Publishers, 1995); DNA Cloning: A practical Approach, Volumes I and 1l (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the present disclosure in any way whatsoever. All publications cited herein are incorporated by reference for the purposes or subject matter referenced herein.

### EXAMPLES

The following examples are provided to further illustrate some embodiments of the present disclosure but are not intended to limit the scope of the present disclosure; it will be understood by their exemplary nature that other procedures, methodologies, or techniques known to those skilled in the art may alternatively be used.

### Example 1—Editing of Transthyretin (TTR) Target Sites in Mammalian Cells

This Example describes introduction of indels into TTR using Cas12i2 introduced into HEK293T cells.

Cas12i2 RNA guides (crRNAs) were designed and ordered from integrated DNA Technologies (IDT). crRNAs were resuspended to a final concentration of 1 mM in 250 mM NaCl. The sequences are shown in Table 8.

TABLE 8
Target and erRNA Sequences

| | | | Target | |
|---|---|---|---|---|
| | | | sequence | |
| | | | (on | |
| guide | | target | non-PAM | |
| identifier | PAM* | strand | Strand) | crRNA sequence |
| Cas122_TTR_ | TTTC | TS | TGAACACAT | rArGrArArArUrCrCrGrUrCrUrUrUr |
| V30M_1 | | | GCACGGCCA | CrArUrUrGrArCrGrGrUrGrArArCr |
| | | | CA (SEQ ID | ArCrArUrGrCrArCrGrGrCrCrArCr |
| | | | NO: 267) | A (SEQ ID NO: 273) |
| Cas122_TTR_ | CTTT | TS | CTGAACACA | rArGrArArArUrCrCrGrUCrUrUrUr |
| V30M_2 | | | TGCACGGCC | CrArUrUrGrArCrGrGrCrUrGrArAr |
| | | | AC (SEQ ID | CrArCrArUrGrCrArCrGrGrCrCrAr |
| | | | NO: 268) | C (SEQ ID NO: 274) |
| Cas122_TTR_ | ATTC | BS | CACCACGGC | rArGrArArArUrCrCrGrUrCrUrUrUr |
| V122I_1 | | | TGTCGTCAC | CrArUrUrGrArCrGrGrCrArCrCrAr |
| | | | CA | CrGrGrCrUrGrUrCrGrUrCrArCrCr |
| | | | (SEQ ID | A (SEQ ID NO: 275) |
| | | | NO: 269) | |
| Cas12i2_TTR_ | CTTG | TS | GGATTGGTG | rArGrArArArUrCrCrGrUrCrUrUrUr |
| V122I_3 | | | ACGACAGCC | CrArUrUrGrArCrGrGrGrGrArUrUr |
| | | | GT (SEQ ID | GrGrUrGrArCrGrArCrArGrCrCrGr |
| | | | NO: 270) | U (SEQ ID NO: 276) |
| Cas1212_ | ATTC | BS | CACCACGGC | rArGrArAArUrCrCrGrUrCrUrUrUr |
| TTR_T119M_1 | | | TGTCGTCACC | CrArUrUrGrArCrGrGrCrArCrCrAr |
| | | | A (SEQ ID | CrGrGrCrUrGrUrCrGrUrCrArCrCr |
| | | | NO: 271) | A (SEQ ID NO: 277) |
| Cas12i2_TTR_ | CTTG | TS | GGATTGGTG | rArGrArArArUrCrCrGrUrCrUrUrUr |
| T119M_2 | | | ACGACAGCC | CrArUrUrGrArCrGrGrGrGrArUrUr |
| | | | GT (SEQ ID | GrGrUrGrArCrGrArCrArGrCrCrGr |
| | | | NO: 272) | U (SEQ ID NO: 278) |

*the TTN 3-nt PAM motif is in boldface.
TS refers to “top strand” and BS refers to “bottom strand”

Cas12i2 RNP complexation reactions were made by mixing purified Cas12i2 polypeptide (400 μM) with crRNA (1 mM in 250 mM NaCl) at a 1:1 (Cas12i2 polypeptide:crRNA) volume ratio (2.5:1 crRNA:Cas12i2 molar ratio). Complexations were incubated on ice for 30-60 min.

During incubation, HEK293T cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 16,480 cells/μL. Resuspended cells were dispensed at 3e5 cells/reaction into Lonza 16-well NUCLEOCUVETTE® strips. Complexed Cas12i2 RNP was added to each reaction at a final concentration of 10 μM. Non-targeting guides were used as negative controls.

The strips were electroporated using an electroporation device (program CM-130, Lonza 4D-NUCLEOFECTOR™). Immediately following electroporation, 80 μL of pre-warmed DMEM+10% FBS was added to each well and mixed gently by pipetting. For each technical replicate plate, plated 10 μL (30,000 cells) of diluted nucleofected cells into pre-warmed 96-well plate with wells containing 100 μL DMEM+10% FBS. Editing plates were incubated for 3 days at 37° C. with 5% C02.

After 3 days, wells were harvested using TRYPLE™ (recombinant cell-dissociation enzymes; ThermoFisher) and transferred to 96-well TWIN.TEC® PCR plates (Eppendorf) PCR plates. Media was flicked off and cells were resuspended in 20 μL QUICKEXTRACT™ (DNA extraction buffer; Lucigen). Samples were then cycled in PCR machine at 65° C. for 15 min, 68° C. for 15 min, 98° C. for 10 min. Samples were then frozen at −20° C.

Samples for Next Generation Sequencing (NOS) were prepared by rounds of PCR. The first round (PCR I) was used to amplify the genomic regions flanking the target site and add NGS adapters. The second round (PCR 11) was used to add NGS indexes. Reactions were then pooled, purified by column purification, and quantified on a fluorometer (Qubit). Sequencing runs were done using a 150 cycle NGS instrument (NEXTSEQ™ v2.5) mid or high output kit (Illumina) and run on an NGS instrument (NEXTSEQ™ 550; Illumina).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides are mismatches were filtered out as well. Up to 50,000 reads passing those filters were used for analysis, and reads were counted as an indel read if they contained an insertion or deletion. The % indels was calculated as the number of indel-containing reads divided by the number of reads analyzed (reads passing filters up to 50,000). The QC standard for the minimum number of reads passing filters was 10,000.

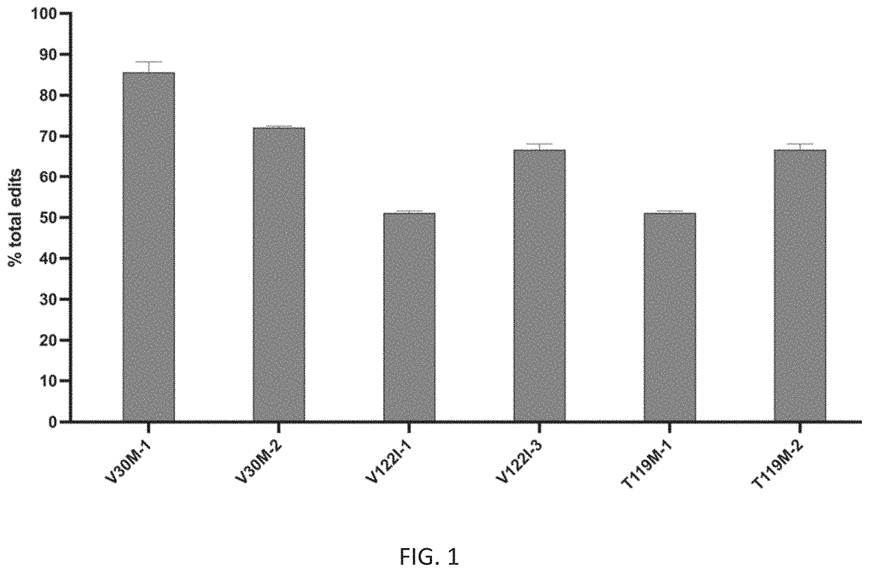

FIG. 1 shows that indel activity was observed for each of the RNA guides used. Cas12i2 was thus able to edit multiple regions of the TTR target nucleic acid in regions known to carry mutations associated with disease.

### Example 2—HDR-Mediated Editing of TTR Target Sites

This Example describes introduction of substitutions within TTR target sites via HDR using Cas12i2 introduced into HEK293T cells.

Cas12i2 RNA guides (crRNAs) were designed and ordered from Integrated DNA Technologies (IDT). crRNAs were resuspended to a final concentration of 1 mM in 250 mM NaCl. The sequences are shown in Table 8 above.

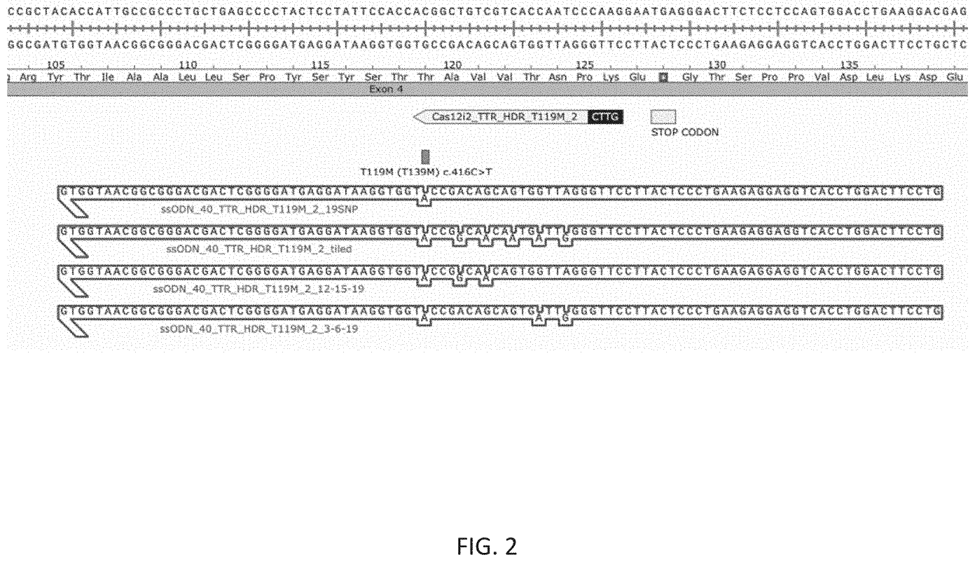

Single-stranded oligo donors (ssODNs) were also designed and ordered from IDT and resuspended to a final concentration of 100 μM in water. The sequences are shown in Table 9 below. ssODNs were designed with either 40 bp or 50 bp homology arms on both sides of the target/guide sequence and comprised at least one SNP modification compared to the target DNA sequence. The first two bases were phosphorothioate modified, and the last 2 of 3 bases were phosphorothioate modified; the last base was unmodified. Three missense mutations were introduced (V30M, T119M, V122I) with or without additional silent mutations inserted to prevent re-targeting of the RNA guides. Each missense mutation was numbered according to the amino acid residue following post-translational protein cleavage of the first 20 residues of TTR. Therefore, as an example, V50M is referred to as V30M since the first 20 amino acids are cleaved post-translationally. FIG. 2 is a schematic depicting ssODNs with 40-bp homology arms and various SNPs introduced within the Cas12i2_TTR_HDR_T119M_2 target sequence. Introduced SNPs within each ssODN are indicated below than the corresponding nucleotide sequence.

Cas12i2 RNP complexation reactions were made by mixing purified Cas12i2 polypeptide (400 μM) with crRNA (1 mM in 250 mM NaCl) at a 1:1 (Cas12i2 polypeptide:crRNA) volume ratio (2.5:1 crRNA:Cas12i2 polypeptide molar ratio). Complexations were incubated on ice for 30-60 min.

During incubation, HEK293T cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 16,480 cells/μL. Resuspended cells were dispensed at 3e5 cells/reaction into Lonza 16-well NUCLEOCUVETTE™ strips. Complexed Cas12i2 RNP was added to each reaction at a final concentration of 10 μM, and ssODN donor sequences were then added at a final concentration of 4 μM. The final volume of each electroporated reaction was 20 μL. Non-targeting guides were used as negative controls.

The strips were electroporated using an electroporation device (program CM-130, Lonza 4D-NUCLEOFECTOR™). Immediately following electroporation, 80 μL of pre-warmed DMEM+10% FBS was added to each well and mixed gently by pipetting. For each technical replicate plate, plated 10 μL (30,000 cells) of diluted nucleofected cells into pre-warmed 96-well plate with wells containing 100 μL DMEM+10% FBS. Editing plates were incubated for 3 days at 37° C. with 5% CO₂.

After 3 days, wells were harvested using TRYPLE™ (recombinant cell-dissociation enzymes; ThermoFisher) and transferred to 96-well TWIN.TEC® PCR plates (Eppendorf) PCR plates. Media was flicked off and cells were resuspended in 20 μL DNA QUICKEXTRACT™ (DNA extraction buffer; Lucigen). Samples were then cycled in PCR machine at 65° C. for 15 min, 68° C. for 15 min, 98° C. for 10 min. Samples were then frozen at −20° C.

Samples for Next Generation Sequencing (NOS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NEXTSEQ™ v2.5) mid or high output kit (Illumina) and run on an NGS instrument (NEXTSEQ™ 550; Illumina).

TABLE 9
SSODN Sequences

| | | | posi- | posi- | number | | | |
|---|---|---|---|---|---|---|---|---|
| | | | tion | tion | of | homol- | | |
| ssODN | guide | | of SNP | of | total | ogy | | |
| identi- | identi- | SNP to | within | silent | mis- | arm | ssODN | |
| fier | fier | correct | target | mutations | matches | length | strand | ssODN sequence |
| ssODN_40_ | Cas12i2_T | V30M | 13 | 14, 17 | 3 | 40 | BS | CCAGAGGCAAATGGCTCCCAGGTGTCATCAGC |
| TTR_HDR_ | TR_HDR_ | (V50M) | | | | | | AGCCTTTCTGAACACATGCATAGCTACATTGAT |
| V30M_1_13 | V30M_1 | c.148 | | | | | | GGCAGGACTGCCTCGGACAGCATCTAGAACTT |
| -14-17 | | G > A | | | | | | TGA (SEQ ID NO: 279) |
| ssODN_40_ | Cas12i2_T | V30M | 13 | 14, 17, | 4 | 40 | BS | CCAGAGGCAAATGGCTCCCAGGTGTCATCAGC |
| TTR_HDR_ | TR_HDR_ | (V50M) | | 20 | | | | AGCCTTTCTGAACACATGCATAGCTACGTTGAT |
| V30M_1_13 | V30M_1 | c.148 | | | | | | GGCAGGACTGCCTCGGACAGCATCTAGAACTT |
| -14-17-20 | | G > A | | | | | | TGA (SEQ ID NO: 280) |
| ssODN_40_ | Cas12i2_T | V30M | 13 | N/A | 1 | 40 | BS | CCAGAGGCAAATGGCTCCCAGGTGTCATCAGC |
| TTR_HDR_ | TR_HDR_ | (V50M) | | | | | | AGCCTTTCTGAACACATGCATGGCCACATTGAT |
| V30M_1_13 | V30M_1 | c.148 | | | | | | GGCAGGACTGCCTCGGACAGCATCTAGAACTT |
| SNP | | G > A | | | | | | TGA (SEQ ID NO: 281) |
| ssODN_40_ | Cas12i2_T | V30M | 13 | 1, 2, | 8 | 40 | BS | CCAGAGGCAAATGGCTCCCAGGTGTCATCAGC |
| TTR_HDR_ | TR_HDR_ | (V50M) | | 5, 8, | | | | AGCCTTTCGAAATACGTGCATAGCTACGTTGAT |
| V30M_1_ | V30M_1 | c.148 | | 14, 17, | | | | GGCAGGACTGCCTCGGACAGCATCTAGAACTT |
| tiled | | G > A | | 20 | | | | TGA (SEQ ID NO: 282) |
| ssODN_40_ | Cas12i2_T | V30M | 14 | 2, 3, | 7 | 40 | BS | CCCAGAGGCAAATGGCTCCCAGGTGTCATCAG |
| TTR_HDR_ | TR_HDR_ | (V50M) | | 6, 9, | | | | CAGCCTTTCGAAATACGTGCATAGCTACATTGA |
| V30M_2_2- | V30M_2 | c.148 | | 15, 18 | | | | TGGCAGGACTGCCTCGGACAGCATCTAGAACT |
| 3-6-9-14- | | G > A | | | | | | TTG (SEQ ID NO: 283) |
| 15-18 | | | | | | | | |
| ssODN_40_ | Cas12i2_T | V30M | 14 | 15, 18 | 3 | 40 | BS | CCCAGAGGCAAATGGCTCCCAGGTGTCATCAG |
| TTR_HDR_ | TR_HDR_ | (V50M) | | | | | | CAGCCTTOCTGAACACATGCATAGCTACATTGA |
| V30M_2_14 | V30M_2 | c.148 | | | | | | TGGCAGGACTGCCTCGGACAGCATCTAGAACT |
| -15-18 | | G > A | | | | | | TTG (SEQ ID NO: 284) |
| ssODN_40_ | Cas12i2_T | V30M | 14 | N/A | 1 | 40 | BS | CCCAGAGGCAAATGGCTCCCAGGTGTCATCAG |
| TTR_HDR_ | TR_HDR_ | (V50M) | | | | | | CAGCCTTTCTGAACACATGCATGGCCACATTGA |
| V30M_2_14 | V30M_2 | c.148 | | | | | | TGGCAGGACTGCCTCGGACAGCATCTAGAACT |
| SNP | | G > A | | | | | | TTG (SEQ ID NO: 285) |
| ssODN_40_ | Cas12i2_T | V30M | 14 | 1, 2, | 8 | 40 | BS | CCCAGAGGCAAATGGCTCCCAGGTGTCATCAG |
| TTR_HDR | TR_HDR_ | (V50M) | | 3, 6, 9, | | | | CAGCCTTCCGAAATACGTGCATAGCTACATTGA |
| V30M_2_ | V30M_2 | c.148 | | 15, 18 | | | | TGGCAGGACTGCCTCGGACAGCATCTAGAACT |
| tiled | | G > A | | | | | | TTG (SEQ ID NO: 286) |
| ssODN_40_ | Cas12i2_T | T119M | 6 | −2, −1, | 5 | 40 | TS | GCTACACCATTGCCGCCCTGCTGAGCCCCTACT |
| TTR_HDR_ | TR_HDR_ | (T139M) | | 1, 4 | | | | CCTATAGTACTATGGCTGTCGTCACCAATCCCA |
| T119M- | T119M_1 | c.416 | | | | | | AGGAATGAGGGACTTCTCCTCCAGTGGACCTG |
| 2-1-1-4-6 | | C > T | | | | | | AA (SEQ ID NO: 287) |
| ssODN_40_ | Cas12i2_T | T119M | 6 | 13, 16, | 4 | 40 | TS | GCTACACCATTGCCGCCCTGCTGAGCCCCTACT |
| TTR_HDR_ | TR_HDR_ | (T139M) | | 19 | | | | CCTATTCCACCATGGCTGTTGTTACTAATCCCA |
| T119M_1_6 | T119M_1 | c.416 | | | | | | AGGAATGAGGGACTTCTCCTCCAG1GGACCTG |
| -13-16-19 | | C > T | | | | | | AA (SEQ ID NO: 288) |
| ssODN_40_ | Cas12i2_T | T119M | 6 | N/A | 1 | 40 | TS | GCTACACCATTGCCGCCCTGCTGAGCCCCTACT |
| TTR_HDR_ | TR_HDR_ | (T139M) | | | | | | CCTATTCCACCATGGCTGTCGTCACCAATCCCA |
| T119M_1_6 | T119M_1 | c.416 | | | | | | AGGAATGAGGGACTTCTCCTCCAGTGGACCTG |
| SNP | | C > T | | | | | | AA (SEQ ID NO: 289) |
| SsODN_40_ | Cas12i2_T | T119M | 6 | −2, −1, | 9 | 40 | TS | GCTACACCATTGCCGCCCTGCTGAGCCCCTACT |
| TTR_HDR | TR_HDR_ | (T139M) | | 1, 4, | | | | CCTATAGTACTATGGCCGTTGTTACTAATCCCA |
| T119M_I_ | T119M_1 | c.416 | | 10, 13, | | | | AGGAATGAGGGACTTCTCCTCCAGTGGACCTG |
| tiled | | C > T | | 16, 19 | | | | AA (SEQ ID NO: 290) |
| ssODN_40_ | CasT2i2_T | T119M | 19 | 3, 6 | 3 | 40 | BS | GTCCTTCAGGTCCACTGGAGGAGAAGTCCCTC |
| TTR_HDR | TR_HDR_ | (T139M) | | | | | | ATTCCTTGGGGTTAGTGACGACAGCCATGGTGG |
| T119M_2_3 | T119M_2 | c.416 | | | | | | AATAGGAGTAGGGGCTCAGCAGGGCGGCAAlG |
| -6-19 | | C > T | | | | | | GTG (SEQ ID NO: 291) |
| ssODN_40_ | Cas12i2_T | T119M | 19 | 12, 15 | 3 | 40 | BS | GTCCTTCAGGTCCACTGGAGGAGAAGTCCCTC |
| TTR_HDR_ | TR_HDR_ | (T139M) | | | | | | ATTCCTTGGGATTGGTGACAACGGCCATGGTGG |
| T119M_2_1 | T119M_2 | c.416 | | | | | | AATAGGAGTAGGGGCTCAGCAGGGCGGCAATG |
| 2-15-19 | | C > T | | | | | | GTG (SEQ ID NO: 292) |
| ssODN_40_ | Cas12i2_T | T119M | 19 | N/A | 1 | 40 | BS | GTCCTTCAGGTCCACTGGAGGAGAAGTCCCTC |
| TTR_HDR_ | TR_HDR_ | (T139M) | | | | | | ATTCCTTGGGATTGGTGACGACAGCCATGGTGG |
| T119M_2_1 | T119M_2 | c.416 | | | | | | AATAGGAGTAGGGGCTCAGCAGGGCGGCAATG |
| 9SNP | | C > T | | | | | | GTG (SEQ ID NO: 293) |
| ssODN_40_ | Cas12i2_T | T119M | 19 | 3, 6, | 6 | 40 | BS | GTCCTTCAGGTCCACTGGAGGAGAAGTCCCTC |
| TTR_HDR | TR_HDR_ | (T139M) | | 9, 12, | | | | ATTCCTTGGGGTTAGTAACAACGGCCATGGTGG |
| T119M_2_ti | T119M_2 | c.416 | | 15 | | | | AATAGGAGTAGGGGCTCAGCAGGGCGGCAATG |
| led | | C > T | | | | | | GTG (SEQ ID NO: 294) |
| ssODN_40_ | Cas12i2_T | V122I | 14 | 1, 4, | 7 | 40 | TS | GCTACACCATTGCCGCCCTGCTGAGCCCCTACT |
| TTR_HDR_ | TR_HDR_ | (V142I) | | 7, 10, | | | | CCTATTCTACTACAGCCGTTATCACTAATCCCA |
| V122I_1_1- | V122I1 | c.424 | | 13, 19 | | | | AGGAATGAGGGACTTCTCCTCCAGTGGACCTG |
| 4-7-10-13- | | G > A | | | | | | AA (SEQ ID NO: 295) |
| 14-19 | | | | | | | | |
| ssODN_40_ | Cas12i2_T | V122I | 14 | 13, 19 | 3 | 40 | TS | GCTACACCATTGCCGCCCTGCTGAGCCCCTACT |
| TTR_HDR_ | TR_HDR_ | (V142I) | | | | | | CCTATTCCACCACGGCTGTTATCACTAATCCCA |
| V122I_1_13 | V122I_1 | c.424 | | | | | | AGGAATGAGGGACTTCTCCTCCAGTGGACCTG |
| -14-19 | | G > A | | | | | | AA (SEQ ID NO: 296) |
| ssODN_40_ | Cas12i2_T | V122I | 14 | N/A | 1 | 40 | TS | GCTACACCATTGCCGCCCTGCTGAGCCCCTACT |
| TTR_HDR_ | TR_HDR_ | (V142I) | | | | | | CCTATTCCACCACGGCTGTCATCACCAATCCCA |
| V122I_1_14 | V122IJ | c.424 | | | | | | AGGAATGAGGGACTTCTCCTCCAGTGGACCTG |
| SNP | | G > A | | | | | | AA (SEQ ID NO: 297) |
| ssODN_40_ | Cas12i2_T | V122I | 14 | −2, −1, | 9 | 40 | TS | GCTACACCATTGCCGCCCTGCTGAGCCCCTACT |
| TTR_HDR_ | TR_HDR_ | (V142I) | | 1, 4, 7, | | | | CCTATAGTACTACAGCCGTTATCACTAATCCCA |
| V122I_1_ | V122L1 | c.424 | | 10, 13, | | | | AGGAATGAGGGACTTCTCCTCCAGTGGACCTG |
| tiled | | G > A | | 19 | | | | AA (SEQ ID NO: 298) |
| ssODN_40_ | Cas12i2_T | V122I | 11 | 12, 15 | 3 | 40 | BS | GTCCTTCAGGTCCACTGGAGGAGAAGTCCCTC |
| TTR_HDR_ | TR_HDR_ | (V142I) | | | | | | ATTCCTTGGGATTGGTGATAACGGCCGTGGTGG |
| V122I3_11 | V122I3 | c.424 | | | | | | AATAGGAGTAGGGGCTCAGCAGGGCGGCAATG |
| -12-15 | | G > A | | | | | | GTG (SEQ ID NO: 299) |
| ssODN_40_ | Cas12i2_T | V122I | 11 | 12, 15, | 4 | 40 | BS | GTCCTTCAGGTCCACTGGAGGAGAAGTCCCTC |
| TTR_HDR_ | TR_HDR_ | (V142I) | | 18 | | | | ATTCCTTGGGATTGGTGATAACGGCTGTGGTGG |
| V122I3_11 | V122I_3 | c.424 | | | | | | AATAGGAGTAGGGGCTCAGCAGGGCGGCAATG |
| -12-15-18 | | G > A | | | | | | GTG (SEQ ID NO: 300) |
| SsODN_40_ | Cas12i2_T | V122I | 11 | N/A | 1 | 40 | BS | GTCCTTCAGGTCCACTGGAGGAGAAGTCCCTC |
| TTR_HDR_ | TR_HDR_ | (V142I) | | | | | | ATTCCTTGGGATTGGTGATGACAGCCGTGGTGG |
| V122I3_11 | V122I_3 | c.424 | | | | | | AATAGGAGTAGGGGCTCAGCAGGGCGGCAATG |
| SNP | | G > A | | | | | | GTG (SEQ ID NO: 301) |
| ssODN_40_ | Cas12i2_T | V122I | 11 | 3, 6, 12, | 6 | 40 | BS | GTCCTTCAGGTCCACTOGAGGAGAAGTCCCTC |
| TTR_HDR | TR_HDR_ | (V142I) | | 15, 18 | | | | ATTCCTTGGGGTTAGEGATAACGGCTGTGGTGG |
| V122I3_ | V122I3 | c.424 | | | | | | AATAGGAGTAGGGGCTCAGCAGGGCGGCAATG |
| tiled | | G > A | | | | | | GTG (SEQ ID NO: 302) |
| ssODN_50_ | Cas12i2_T | T119M | 6 | −2, −1, | 5 | 50 | TS | GGCCCCCGCCGCTACACCATTGCCGCCCTGCTG |
| TTR_HDR_ | TR_HDR_ | (T139M) | | 1, 4 | | | | AGCCCCTACTCCTATAGTACTATGGCTGTCGTC |
| T119M_I_- | T119MJ | c.416 | | | | | | ACCAATCCCAAGGAATGAGGGACTTCTCCTCC |
| 2-1-1-4-6 | | C > T | | | | | | AGTGGACCTGAAGGACGAGGGA (SEQ ID NO: |
| | | | | | | | | 303) |
| ssODN_50_ | Cas12i2_T | T119M | 6 | 13, 16, | 4 | 50 | TS | GGCCCCCGCCGCTACACCATTGCCGCCCTGCTG |
| TTR_HDR_ | TR_HDR_ | (T139M) | | 19 | | | | AGCCCCTACTCCTATTCCACCATGGCTGTTGTT |
| T119M-16 | T119M_1 | c.416 | | | | | | ACTAATCCCAAGGAATGAGGGACTTCTCCTCC |
| -13-16-19 | | C > T | | | | | | AGTGGACCTGAAGGACGAGGGA (SEQ ID NO: |
| | | | | | | | | 304) |
| SsODN_50_ | Cas12i2_T | T119M | 6 | N/A | 1 | 50 | TS | GGCCCCCGCCGCTACACCATTGCCGCCCTGCTG |
| TTR_HDR_ | TR_HDR_ | (T139M) | | | | | | AGCCCCTACTCCTATTCCACCATGGCTGTCGTC |
| IT19M_1_6 | T119M_1 | c.416 | | | | | | ACCAATCCCAAGGAATGAGGGACTTCTCCTCC |
| SNP | | C > T | | | | | | AGTGGACCTGAAGGACGAGGGA (SEQ ID NO: |
| | | | | | | | | 305) |
| ssODN_50_ | Cas12i2_T | TH9M | 6 | −2, −1, | 9 | 50 | TS | GGCCCCCGCCGCTACACCATTGCCGCCCTGCTG |
| TTR_HDR_ | TR_HDR_ | (T139M) | | 1, 4, | | | | AGCCCCTACTCCTATAGTACTATGGCCGTTGTT |
| T119M_1_ | T119M_1 | c.416 | | 10, 13, | | | | ACTAATCCCAAGGAATGAGGGACTTCTCCTCC |
| tiled | | C > T | | 16, 19 | | | | AGTGGACCTGAAGGACGAGGGA (SEQ ID NO: |
| | | | | | | | | 306) |
| ssODN_50_ | Cas12i2_T | T119M | 19 | 3, 6 | 3 | 50 | BS | CCCATCCCTCGTCCTTCAGGTCCACTGGAGGAG |
| TTR_HDR_ | TR_HDR_ | (T139M) | | | | | | AAGTCCCTCATTCCTTGGGGTTAGTGACGACAG |
| T119M_2_3 | T119M_2 | c.416 | | | | | | CCATGGTGGAATAGGAGTAGGGGCTCAGCAGG |
| -6-19 | | C > T | | | | | | GCGGCAATGGTGTAGCGGCGGG (SEQ ID NO: |
| | | | | | | | | 307) |
| ssODN_50_ | Cas12i2_T | T119M | 19 | 12, 15 | 3 | 50 | BS | CCCATCCCTCGTCCTTCAGGTCCACTGGAGGAG |
| TTR_HDR_ | TR_HDR_ | (T139M) | | | | | | AAGTCCC1CATTCCTTGGGATTGGTGACAACGG |
| T119M2_1 | T119M_2 | c.416 | | | | | | CCATGGTGGAATAGGAGTAGGGGCTCAGCAGG |
| 2-15-19 | | C > T | | | | | | GCGGCAATGGTGTAGCGGCGGG (SEQ ID NO: |
| | | | | | | | | 308) |
| ssODN_50_ | Cas12i2_T | T119M | 19 | NZA | 1 | 50 | BS | CCCATCCCTCGTCCTTCAGGTCCACTGGAGGAG |
| TTR_HDR_ | TR_HDR_ | (T139M) | | | | | | AAGTCCCTCATTCCTTGGGATTGGTGACGACAG |
| T119M_2_1 | T119M_2 | c.416 | | | | | | CCATGGTGGAATAGGAGTAGGGGCTCAGCAGG |
| 9SNP | | C > T | | | | | | GCGGCAATGGTGTAGCGGCGGG (SEQ ID NO: |
| | | | | | | | | 309) |
| ssODN_50_ | Cas12i2_T | T119M | 19 | 3, 6, 9, | 6 | 50 | BS | CCCATCCCTCGTCCTTCAGGTCCACTGGAGGAG |
| TTR_HDR | TR_HDR_ | (T139M) | | 12, 15 | | | | AAGTCCCTCATTCCTTGGGGTTAGTAACAACGG |
| T119M_2_ | T119M_2 | c.416 | | | | | | CCATGGTGGAATAGGAGTAGGGGCTCAGCAGG |
| tiled | | C > T | | | | | | GCGGCAATGGTGTAGCGGCGGG (SEQ ID NO: |
| | | | | | | | | 310) |
| SsODN_50_ | Cas1212_T | V30M | 13 | 14, 17 | 3 | 50 | BS | GGCAACTTACCCAGAGGCAAATGGCTCCCAGG |
| TTR_HDR | TR_HDR_ | (V50M) | | | | | | TGTCATCAGCAGCCTTTCTGAACACATGCATAG |
| V30M_1_13 | V30M_1 | c.148 | | | | | | CTACATTGATGGCAGGACTGCCTCGGACAGCA |
| -14-17 | | G > A | | | | | | TCTAGAACTTTGACCATCAGAGG (SEQ ID NO: |
| | | | | | | | | 311) |
| ssODN_50_ | Cas1212_T | V30M | 13 | 14, 17, | 4 | 50 | BS | GGCAACTTACCCAGAGGCAAATGGCTCCCAGG |
| TTR_HDR | TR_HDR_ | (V50M) | | 20 | | | | TGTCATCAGCAGCCTTTCTGAACACATGCATAG |
| V30M_1_13 | V30MJ | c.148 | | | | | | CTACG1TGATGGCAGGACTGCCTCGGACAGCA |
| -14-17-20 | | G > A | | | | | | TCTAGAACTTTGACCATCAGAGG (SEQ ID NO: |
| | | | | | | | | 312) |
| ssODN_50_ | Cas12i2_T | V30M | 13 | N/A | 1 | 50 | BS | GGCAACTTACCCAGAGGCAAATGGCTCCCAGG |
| TTR_HDR_ | TR_HDR_ | (V50M) | | | | | | TGTCATCAGCAGCCTTTCTGAACACATGCATGG |
| V30M_1_13 | V30M_1 | c.148 | | | | | | CCACATTGATGGCAGGACTGCCTCGGACAGCA |
| SNP | | G > A | | | | | | TCTAGAACTTTGACCATCAGAGG (SEQ ID NO: |
| | | | | | | | | 313) |
| ssODN_50_ | Cas12i2_T | V30M | 13 | 1, | g | 50 | BS | GGCAACTTACCCAGAGGCAAATGGCTCCCAGG |
| TTR_HDR_ | TR_HDR_ | (V50M) | | 2, 5, | | | | TGTCATCAGCAGCCTTTCGAAATACGTGCATAG |
| V30M_1_ | V30M_1 | c.148 | | 8, 14, | | | | CTACGTTGATGGCAGGACTGCCTCGGACAGCA |
| tiled | | G > A | | 17, 20 | | | | TCTAGAACTTTGACCATCAGAGG (SEQ ID NO: |
| | | | | | | | | 314) |
| ssODN_50_ | Cas12i2_T | V30M | 14 | 2, 3, | 7 | 50 | BS | TGGCAACTTACCCAGAGGCAAATGGCTCCCAG |
| TTR_HDR_ | TR_HDR_ | (V50M) | | 6, 9, | | | | GTGTCATCAGCAGCCTTTCGAAATACGTGCATA |
| V30M_2_2- | V30M_2 | c.148 | | 15, 18 | | | | GCTACATTGATGGCAGGACTGCCTCGGACAGC |
| 3-6-9-14- | | G > A | | | | | | ATCTAGAACTTTGACCATCAGAG (SEQ ID NO: |
| 15-18 | | | | | | | | 315) |
| ssODN_50_ | Cas12i2_T | V30M | 14 | 15, 18 | 3 | 50 | BS | TGGCAACTTACCCAGAGGCAAATGGCTCCCAG |
| TTR_HDR_ | TR_HDR_ | (V50M) | | | | | | GTGTCATCAGCAGCCTTTCTGAACACATGCATA |
| V30M_2_14 | V30M_2 | c.148 | | | | | | GCTACATTGATGGCAGGACTGCCTCGGACAGC |
| -15-18 | | G > A | | | | | | ATCTAGAACTTTGACCATCAGAG (SEQ ID NO: |
| | | | | | | | | 316) |
| ssODN_50_ | Cas12i2_T | V30M | 14 | N/A | 1 | 50 | BS | TGGCAACTTACCCAGAGGCAAATGGCTCCCAG |
| TTR_HDR | TR_HDR_ | (V50M) | | | | | | GTGTCATCAGCAGCCTTTCTGAACACATGCATG |
| V30M_2_14 | V30M_2 | c.148 | | | | | | GCCACATTGATGGCAGGACTGCCTCGGACAGC |
| SNP | | G > A | | | | | | ATCTAGAACTTTGACCATCAGAG (SEQ ID NO: |
| | | | | | | | | 317) |
| SsODN_50_ | Cas1212_T | V30M | 14 | 1, 2, 3, | 8 | 50 | BS | TGGCAACTTACCCAGAGGCAAATGGCTCCCAG |
| TTR_HDR | TR_HDR_ | (V50M) | | 6, 9, | | | | GTGTCATCAGCAGCCTTCCGAAATACGTGCATA |
| V30M_2_til | V30M_2 | c.148 | | 15, 18 | | | | GCTACATTGATGGCAGGACTGCCTCGGACAGC |
| ed | | G > A | | | | | | ATCTAGAACTTTGACCATCAGAG (SEQ ID NO: |
| | | | | | | | | 318) |
| ssODN_50_ | Cas12i2_T | V122I | 14 | 1, 4, | 7 | 50 | TS | GGCCCCCGCCGCTACACCATTGCCGCCCTGCTG |
| TTR_HDR_ | TR_HDR_ | (V142I) | | 7, 10, | | | | AGCCCCTACTCCTATTCTACTACAGCCGTTATC |
| V122I_1_1- | V122I_1 | c.424 | | 13, 19 | | | | ACTAATCCCAAGGAATGAGGGACTTCTCCTCC |
| 4-7-10-13- | | G > A | | | | | | AGTGGACCTGAAGGACGAGGGA (SEQ ID NO: |
| 14-19 | | | | | | | | 319) |
| ssODN_50_ | Cas12i2_T | V122I | 14 | 13, 19 | 3 | 50 | TS | GGCCCCCGCCGCTACACCATTGCCGCCCTGCTG |
| TTR_HDR | TR_HDR_ | (V142I) | | | | | | AGCCCCTACTCCTATTCCACCACGGCTGTTATC |
| V122I_1_13 | V122I_1 | c.424 | | | | | | ACTAATCCCAAGGAATGAGGGACTTCTCCTCC |
| -14-19 | | G > A | | | | | | AGTGGACCTGAAGGACGAGGGA (SEQ ID NO: |
| | | | | | | | | 320) |
| ssODN_50_ | Cas12i2_T | V122I | 14 | N/A | 1 | 50 | TS | GGCCCCCGCCGCTACACCATTGCCGCCCTGCTG |
| TTR_HDR_ | TR_HDR_ | (V142I) | | | | | | AGCCCCTACTCCTATTCCACCACGGCTGTCATC |
| V122I_1_14 | V122I_1 | c.424 | | | | | | ACCAATCCCAAGGAATGAGGGACTTCTCCTCC |
| SNP | | G > A | | | | | | AGTGGACCTGAAGGACGAGGGA (SEQ ID NO: |
| | | | | | | | | 321) |
| ssODN_50_ | Cas12i2_T | V122I | | | | | | |
| TTR_HDR_ | TR_HDR_ | (V142I) | | 10, 13, | | | | AGCCCCTACTCCTATAGTACTACAGCCGTTATC |
| V122L_1_til | V122I_1 | c.424 | | 19 | | | | ACTAATCCCAAGGAATGAGGGACTTCTCCTCC |
| ed | | G > A | | | | | | AGTGGACCTGAAGGACGAGGGA (SEQ ID NO: |
| | | | | | | | | 322) |
| ssODN_50_ | Cas12i2_T | V122I | 11 | 12, 15 | 3 | 50 | BS | CCCATCCCTCGTCCTTCAGGTCCACTGGAGGAG |
| TTR_HDR | TR_HDR_ | (V142I) | | | | | | AAGTCCCTCATTCCTTGGGATTGGTGATAACGG |
| V122I3_11 | V122I_3 | c.424 | | | | | | CCGTGGTGGAATAGGAGTAGGGGCTCAGCAGG |
| -12-15 | | G > A | | | | | | GCGGCAATGGTGTAGCGGCGGG (SEQ ID NO: |
| | | | | | | | | 323) |
| SsODN_50_ | Cas1212_T | V122I | 11 | 12, 15, | 4 | 50 | BS | CCCATCCCTCGTCCTTCAGGTCCACTGGAGGAG |
| TTR_HDR | TR_HDR_ | (V142I) | | 18 | | | | AAGTCCCTCATTCCTTGGGATTGGTGATAACGG |
| V122I3_11 | V122I_3 | c.424 | | | | | | CTGTGGTGGAATAGGAGTAGGGGCTCAGCAGG |
| -12-15-18 | | G > A | | | | | | GCGGCAATGGTGTAGCGGCGGG (SEQ ID NO: |
| | | | | | | | | 324) |
| ssODN_50_ | Cas1212_T | V122I | 11 | N/A | 1 | 50 | BS | CCCATCCCTCGTCCTTCAGGTCCACTGGAGGAG |
| TTR_HDR | TR_HDR_ | (VI421) | | | | | | AAGTCCCTCATTCCTTGGGATTGGTGATGACAG |
| V122I3_11 | V122I_3 | c.424 | | | | | | CCGTGGGGAAAGGAGTAGGGGCTCAGCAGG |
| SNP | | G > A | | | | | | GCGGCAATGGTGTAGCGGCGGG (SEQ ID NO: |
| | | | | | | | | 325) |
| ssODN_50_ | Cas12i2_T | V122I | 11 | 3, 6, | 6 | 50 | BS | CCCATCCCTCGTCCTTCAGGTCCACTGGAGGAG |
| TTR_HDR | TR_HDR_ | (V142I) | | 12, 15, | | | | AAGTCCCTCATTCCTTGGGGTTAGTGATAACGG |
| V122I_3_ | V122I_3 | C.424 | | 18 | | | | CTGTGGTGGAATAGGAGTAGGGGCTCAGCAGG |
| tiled | | G > A | | | | | | GCGGCAATGGTGTAGCGGCGGG (SEQ ID NO: |
| | | | | | | | | 326) |

For NGS analysis, the indel mapping function used a sample's fastq file, the amplicon reference sequence, and the forward primer sequence. For each read, a kmer-scanning algorithm was used to calculate the edit operations (match, mismatch. insertion, deletion) between the read and the reference sequence. In order to remove small amounts of primer dimer present in some samples, the first 30 nt of each read was required to match the reference and reads where over half of the mapping nucleotides are mismatches were filtered out as well. Up to 50.000 reads passing those filters were used for analysis. and reads were counted as an indel read if they contained an insertion or deletion. The % indels was calculated as the number of indel-containing reads divided by the number of reads analyzed (reads passing filters up to 50,000). The % full HDR integration was calculated as the number of reads containing the entire integrated ssODN sequence divided by the number of reads analyzed (reads passing filter up to 50,000). The % partial HDR integration was calculated as the number of reads containing the 20 bp ssODN sequence that overlapped with the guide sequence divided by the number of reads analyzed (reads passing filter up to 50,000). The % total edits was calculated by adding the % indels to the % full HDR integration. To summarize, the % full HDR integration represents the number of reads where the entire ssODN donor was integrated into the correct genomic position, while the % partial HDR integration represents any reads (with or without indels) that contain the 20 bp ssODN sequence that overlapped with the guide sequence (i.e. the region containing SNPs). The QC standard for the minimum number of reads passing filters was 10,000.

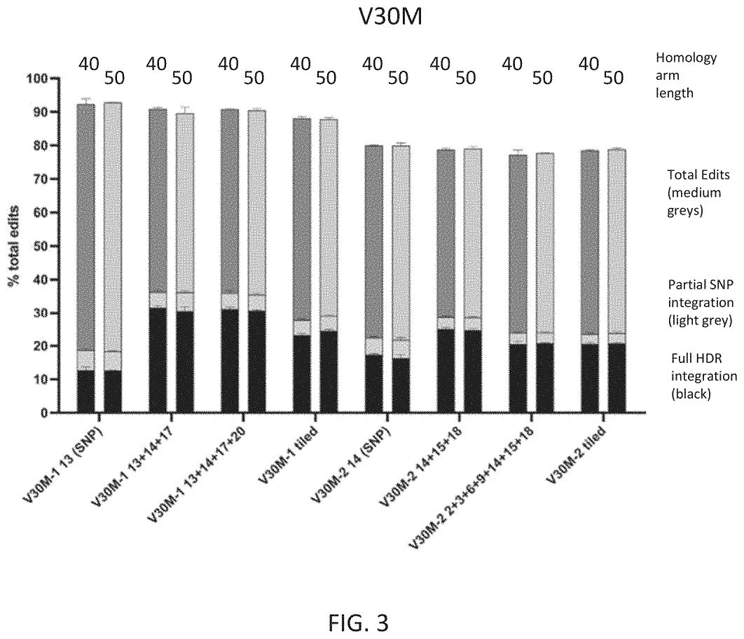

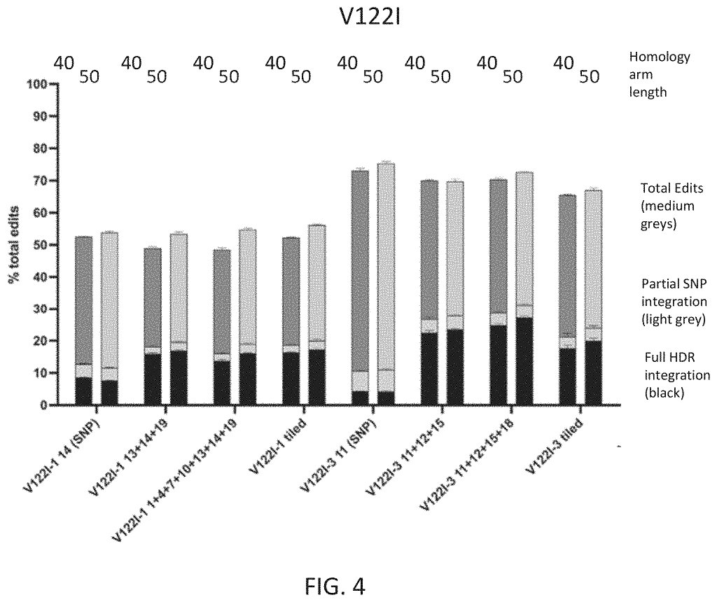

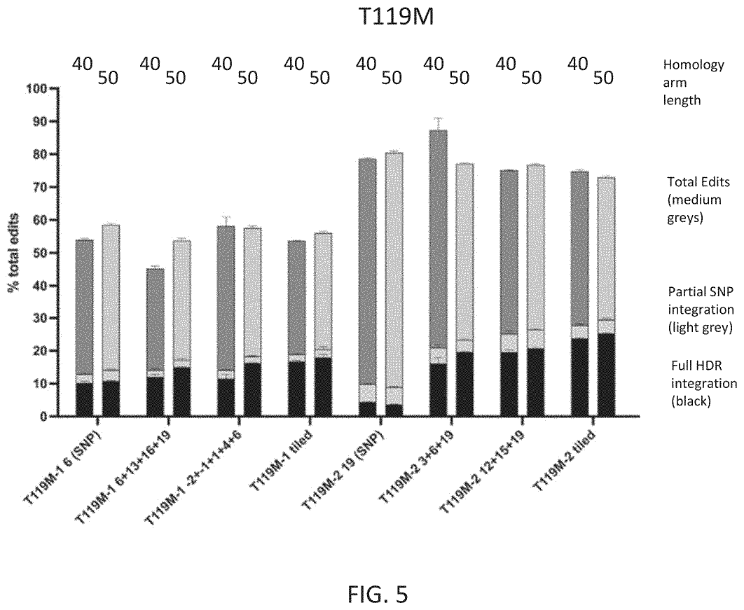

FIG. 2 shows efficiency of HDR-templated substitutions at the V30M locus in HEK293T cells following RNP transfection with Cas12i2. FIG. 3 shows efficiency of HDR-templated substitutions at the V122I locus in HEK293T cells following RNP transfection with Cas12i2. FIG. 4 shows efficiency of HDR-templated substitutions at the T119M locus in HEK293T cells following RNP transfection with Cas12i2. The black bars reflect the mean full % HDR integration edits measured in one bioreplicate. the light grey bars reflect the mean partial % SNP integration edits, and the medium grey bars represent % total edits (described above). Error bars represent the average of three technical replicates.

As shown in FIG. 2, FIG. 3. and FIG. 4. significant levels of full integration of ssODN donors that comprised at least three SNPs were observed. Lower full % HDR integration in the single SNP ssODN donor samples was likely due to higher levels of re-targeting by the RNA guides.

This Example shows that SNPs can be introduced into TTR target sites via HDR with Cas12i2. For example, protective SNPs associated with disease (e.g., T119M) can be introduced into the TTR gene. Furthermore, SNPs associated with disease (e.g., V30M and V122I) can be corrected using this method.

### Example 3—Cas12i2-Mediated Editing of TTR Target Sites in HEK293T Cells

This Example describes the genomic editing of the TTR gene using Cas12i2 introduced into HEK293T cells by RNP.

Cas12i2 RNA guides (crRNAs) were designed and ordered from Integrated DNA Technologies (IDT). crRNAs were resuspended to a final concentration of 1 mM in 250 mM NaCl. The target and RNA guide sequences are shown in Table 10.

TABLE 10
TTR Target and RNA Guide Sequences

| | PAM | | |
|---|---|---|---|
| Target | sequence* | Target sequence | crRNA sequence |
| E1T1 | CTTC | TCATCGTCTGCTCCTCCTC | AGAAAUCCGUCUUUCAUUGACGGUCA |
| | | T (SEQ ID NO: 327) | UCGUCUGCUCCUCCUCU (SEQ ID NO: |
| | | | 345) |
| E2T1 | CTTC | TCTACACCCAGGGCACCG | AGAAAUCCGUCUUUCAUUGACGGUCU |
| | | GT (SEQ ID NO: 328) | ACACCCAGGGCACCGGU (SEQ ID NO: |
| | | | 346) |
| E2T2 | CTTT | GACCATCAGAGGACACTT | AGAAAUCCGUCUUUCAUUGACGGGAC |
| | | GG (SEQ ID NO: 329) | CAUCAGAGGACACUUGG (SEQ ID NO: |
| | | | 347) |
| E2T3 | GTTC | TAGATGCTGTCCGAGGCA | AGAAAUCCGUCUUUCAUUGACGGUAG |
| | | GT (SEQ ID NO: 330) | AUGCUGUCCGAGGCAGU (SEQ ID NO: |
| | | | 348) |
| E2T4 | GTTC | AGAAAGGCTGCTGATGAC | AGAAAUCCGUCUUUCAUUGACGGAGA |
| | | AC (SEQ ID NO: 331) | AAGGCUGCUGAUGACAC (SEQ ID NO: |
| | | | 349) |
| E2T5 | CTTT | CTGAACACATGCACGGCC | AGAAAUCCGUCUUUCAUUGACGGCUG |
| | | AC (SEQ ID NO: 332) | AACACAUGCACGGCCAC (SEQ ID NO: |
| | | | 350) |
| E2T6 | CTTT | GGCAACTTACCCAGAGGC | AGAAAUCCGUCUUUCAUUGACGGGGC |
| | | AA (SEQ ID NO: 333) | AACUUACCCAGAGGCAA (SEQ ID NO: |
| | | | 351) |
| E2T7 | GTTC | TTTGGCAACTTACCCAGAG | AGAAAUCCGUCUUUCAUUGACGGUUU |
| | | G (SEQ ID NO: 334) | GGCAACUUACCCAGAGG (SEQ ID NO: |
| | | | 352) |
| E3TI | CTTT | CACACCTTATAGGAAAAC | AGAAAUCCGUCUUUCAUUGACGGGAC |
| | | CA (SEQ ID NO: 335) | ACCUUAUAGGAAAACCA (SEQ ID NO: |
| | | | 353) |
| E3T2 | CTTC | TACAAATTCCTCCTCAGTT | AGAAAUCCGUCUUUCAUUGACGGUAC |
| | | G (SEQ ID NO: 336) | AAAUUCCUCCUCAGUUG (SEQ ID NO: |
| | | | 354) |
| E3T3 | CTTT | GTATATCCCTTCTACAAAT | AGAAAUCCGUCUUUCAUUGACGGGUA |
| | | T (SEQ ID NO: 337) | UAUCCCUUCUACAAAUU (SEQ ID NO: |
| | | | 355) |
| E3T4 | CTTC | CAGTAAGATTTGGTGTCTA | AGAAAUCCGUCUUUCAUUGACGGCAG |
| | | T (SEQ ID NO: 338) | UAAGAUUUGGUGUCUAU (SEQ ID NO: |
| | | | 356) |
| E4T1 | ATTG | CCGCCCTGCTGAGCCCCTA | AGAAAUCCGUCUUUCAUUGACGGCCG |
| | | C (SEQ ID NO: 339) | CCCUGCUGAGCCCCUAC (SEQ ID NO: |
| | | | 357) |
| E4T2 | ATTC | CACCACGGCTGTCGTCACC | AGAAAUCCGUCUUUCAUUGACGGCAC |
| | | A (SEQ ID NO: 340) | CACGGCUGUCOUCACCA (SEQ ID NO: |
| | | | 358) |
| E4T3 | ATTG | GTGACGACAGCCGTGGTG | AGAAAUCCGUCUUUCAUUGACGGGUG |
| | | GA (SEQ ID NO: 341) | ACGACAGCCGUGGUGGA (SEQ ID NO: |
| | | | 359) |
| E4T4 | CTTG | GGATTGGTGACGACAGCC | AGAAAUCCGUCUUUCAUUGACGGGGA |
| | | GT (SEQ ID NO: 342) | UUGGUGACGACAGCCGU (SEQ ID NO: |
| | | | 360) |
| E4T5 | ATTC | CTTGGGATTGGTGACGAC | AGAAAUCCGUCUUUCAUUGACGGCUU |
| | | AG (SEQ ID NO: 343) | GGGAUUGGLIGACGACAG (SEQ ID NO: |
| | | | 361) |
| E4T6 | CTTC | AGGTCCACTGGAGGAGAA | AGAAAUCCGUCUUUCAUUGACGGAGG |
| | | GT (SEQ ID NO: 344) | UCCACUGGAGGAGAAGU (SEQ ID NO: |
| | | | 362) |

*the TTN 3-nt PAM motif is in boldface.

Cas12i2 RNP complexation reactions were made by mixing purified Cas12i2 polypeptide (400 μM) with crRNA (1 mM in 250 mM NaCl) at a 1:1 (Cas12i2 polypeptide:crRNA) volume ratio (2.5:1 crRNA:Cas12i2 polypeptide molar ratio). Complexations were incubated on ice for 30-60 min.

During incubation. HEK293T cells were harvested using TRYPLE™ (recombinant cell-dissociation enzymes: ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 16.480 cells/μL. Resuspended cells were dispensed at 3e5 cells/reaction into Lonza 16-well NUCLEOCUVETTE™ strips. Complexed Cas12i2 RNP was added to each reaction at a final concentration of 10 μM. Non-targeting guides were used as negative controls.

The strips were electroporated using an electroporation device (program CM-130, Lonza 4D-NUCLEOFECTOR™). Immediately following electroporation, 80 μL of pre-warmed DMEM+10% FBS was added to each well and mixed gently by pipetting. For each technical replicate plate. plated 10 μL (30.000 cells) of diluted nucleofected cells into pre-warmed 96-well plate with wells containing 100 μL DMEM+10% FBS. Editing plates were incubated for 3 days at 37° C. with 5% C02.

After 3 days, wells were harvested using TRYPLE™ (recombinant cell-dissociation enzymes: ThermoFisher) and transferred to 96-well TWIN.TEC® PCR plates (Eppendorf) PCR plates. Media was flicked off and cells were resuspended in 20 μL QUICKEXTRACT™ (DNA extraction buffer; Lucigen). Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification. and quantified on a fluorometer (Qubit). Sequencing runs were done using a 150 cycle NGS instrument (NEXTSEQr v2.5) mid or high output kit (Illumina) and run on an NGS instrument (NEXTSEQ™ 550; Illumina).

For NGS analysis. the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples. the first 30 nt of each read was required to match the reference and reads where over half of the mapping nucleotides are mismatches were filtered out as well. Up to 50.000 reads passing those filters were used for analysis, and reads were counted as an indel read if they contained an insertion or deletion. The % indels was calculated as the number of indel-containing reads divided by the number of reads analyzed (reads passing filters up to 50,000). The QC standard for the minimum number of reads passing filters was 10.000.

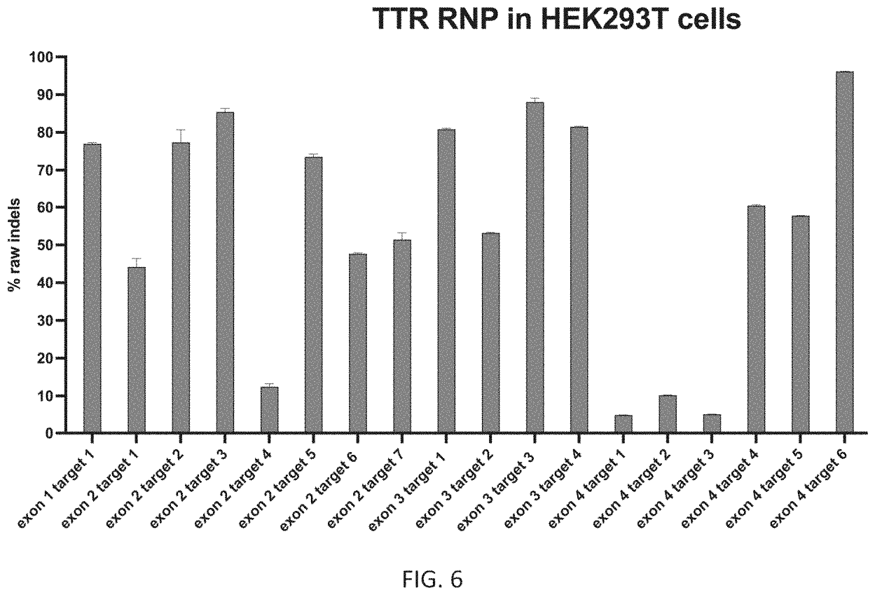

FIG. 6 shows indels in TTR in HEK293T cells following RNP delivery. Error bars represent the average of three technical replicates across one biological replicate. Following delivery, indels were detected in each of the 18 tested TTR target sequences. Indels were detected in at least 40% of NGS reads for 14 of the RNA guides (E2T3, E3T3, and E4T6). Indels were detected in at least 80% of NGS reads for 3 of the RNA guides (E2T3, E3T3, and E4T6).

This Example thus shows that exon 1, exon 2, exon 3, and exon 4 of TTR can be targeted by Cas12i2 RNPs in mammalian cells such as HEK293T cells.

### Example 4—Cas12i2-Mediated Editing of TTR Target Sites in HepG2 Cells

This Example describes the genomic editing of the TTR gene using Cas12i2 introduced into HepG2 cells by RNP.

Cas12i2 RNP complexation reactions were made by mixing purified Cas12i2 polypeptide with a TTR-targeting crRNA of Table 8 at a 2.5:1 crRNA:Cas12i2 molar ratio. Complexations were incubated on ice for 30-60 min.

HepG2 cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 13.889 cells/μL. Resuspended cells were dispensed at 2.5e5 cells/reaction into Lonza 16-well NUCLEOCUVETTE™ strips. Complexed Cas12i2 RNP was added to each reaction at a final concentration of 20 μM (Cas12i2), with no transfection enhancer oligo. The final volume of each electroporated reaction was 20 μL. Non-targeting guides were used as negative controls.

The strips were electroporated using an electroporation device (program DJ-100, Lonza 4D-NUCLEOFECTOR™). Immediately following electroporation, 80 μL of pre-warmed EMEM+10% FBS was added to each well and mixed gently by pipetting. For each technical replicate plate, plated 10 μL (25.000 cells) of diluted nucleofected cells into pre-warmed 96-well plate with wells containing 100 μL EMEM+10% FBS. Editing plates were incubated for 3 days at 37° C. with 5% CO₂.

After 3 days, wells were harvested using TRYPLE™ (recombinant cell-dissociation enzymes; ThermoFisher) and transferred to 96-well TWIN.TEC® PCR plates (Eppendorf). Media was flicked off and cells were resuspended in 20 μL QUICKEXTRACT™ (DNA extraction buffer; Lucigen). Samples were then cycled in a PCR machine at 65° C. for 15 min, 68° C. for 15 min. 98° C. for 10 min. Samples were then frozen at −20° C. and analyzed by NGS as described in Example 1.

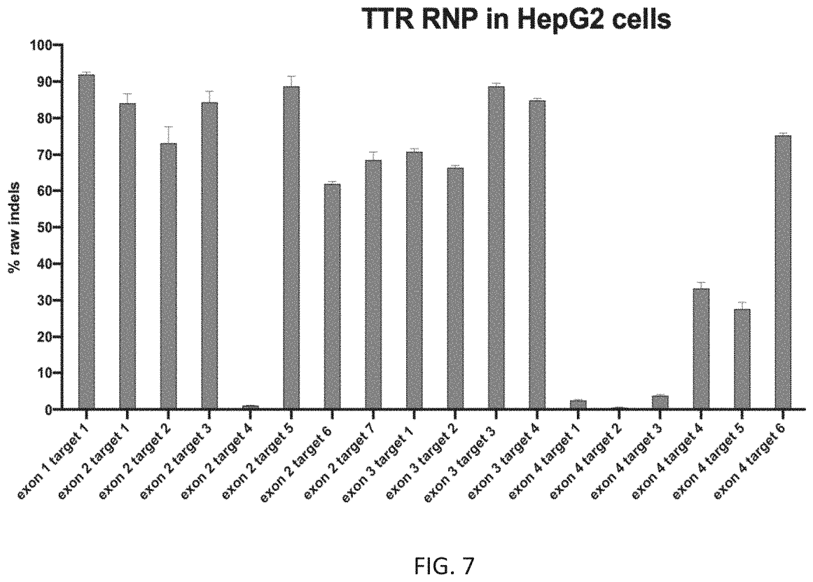

FIG. 7 shows percentage of NGS reads comprising indels in TTR target sites in HepG2 cells following RNP delivery. Error bars represent the average of three technical replicates across one biological replicate. Following delivery, indels were detected in 17 of the 18 tested TTR target sites. Indels were detected in at least 80% of NGS reads for 6 of the RNA guides (E1T1. E2T1, E2T3, E2T5, E3T3, and E3T4).

This Example thus shows that exon 1, exon 2, exon 3, and exon 4 of TTR can be targeted by Cas12i2 RNPs in mammalian cells such as HepG2 cells.

### Example 5—Cas12i2-Mediated Editing of TTR Target Sites in Primary Hepatocytes

This Example describes the genomic editing of the TTR using Cas12i2 introduced into primary hepatocytes cells by RNP.

Cas12i2 RNP complexation reactions were made as described in Example 3 with the 14 RNA guides identified as being active in HepG2 cells.

Primary hepatocyte cells from human donors were thawed from liquid nitrogen very quickly in a 37° C. water bath. The cells were added to pre-warmed hepatocyte recovery media (Thermofisher, CM7000) and centrifuged at 100 g for 10 minutes. The cell pellet was resuspended in appropriate volume of hepatocyte plating medium (Williams' Medium E. Thermofisher A1217601 supplemented with Hepatocyte Plating Supplement Pack (serum-containing). Thermofisher CM3000). The cells were subjected to trypan blue viability count with an INCUCYTE® disposable hemocytometer (Fisher Scientific, 22-600-100). The cells were then washed in PBS and resuspended in P3 buffer+supplement (P3 PRIMARY CELL 4D-NUCLEOFECTOR™ X Kit S; Lonza. VXP-3032) at a concentration of ˜7.500 cells/μL. Resuspended cells were dispensed at 150,000 cells/reaction into Lonza 16-well NUCLEOCUVETTE® strips. Complexed Cas12i2 RNP was added to each reaction at a final concentration of 20 μM (Cas12i2), and transfection enhancer oligos were then added at a final concentration of 4 μM. The final volume of each electroporated reaction was 20 μL. Non-targeting guides were used as negative controls.

The strips were electroporated using an electroporation device (program DS-150, Lonza 4D-NUCLEOFECTOR™). Immediately following electroporation. 45 μL of pre-warmed hepatocyte plating medium was added to each well and mixed very gently by pipetting. For each technical replicate plate, plated 65 μL (150.000 cells) of diluted nucleofected cells into a pre-warmed collagen-coated 96-well plate (Thermofisher) with wells containing 60 μL Hepatocyte plating medium. The cells were then incubated in a 37° C. incubator. The media was changed to hepatocyte maintenance media (Williams' Medium E. Thermofisher A1217601 supplemented with William's E medium Cell Maintenance Cocktail, Thermofisher CM 4000) after the cells attached after 4 hours. Fresh hepatocyte maintenance media was replaced 1 day, 3 days and 5 days post RNP electroporation.

The cells were harvested either 3 days or 7 days post RNP electroporation, by collecting the media and detaching the cells by shaking at (500 rpm) in a 37° C. incubator, with 2 mg/ml collagenase IV (Thermofisher, 17104019) dissolved in PBS containing Ca/Mg (Thermofisher). After cells were dissociated from the plate and transferred to 96-well TWIN.TEC® PCR plates (Eppendorf) and centrifuged. Media was flicked off and cells were resuspended in 20 μL QUICKEXTRACT™ (DNA extraction buffer; Lucigen). Samples were then cycled in a PCR machine at 65° C. for 15 min, 68° C. for 15 min, 98° C. for 10 min. Samples were then frozen at ˜20° C. and analyzed by NGS as described in Example 1.

At the time of harvesting the cells 7 days post electroporation, the media from each well was centrifuged at 100 g for 10 minutes and the supernatant was stored at ˜80C for subsequent ELISA assay. The cell culture supernatant from each condition was diluted to an optimal dilution (1:20) and subjected to TTR ELISA (Human) (Aviva Systems Biology. OKBB01176). following manufacturer's instructions.

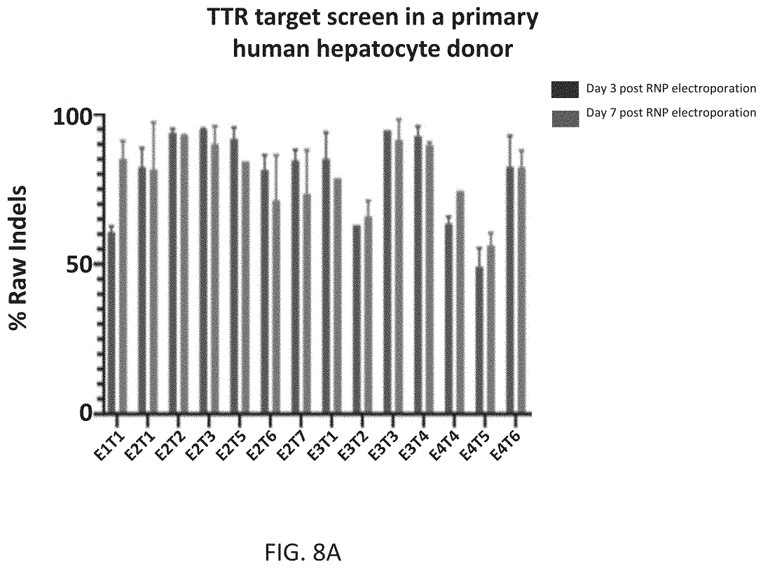

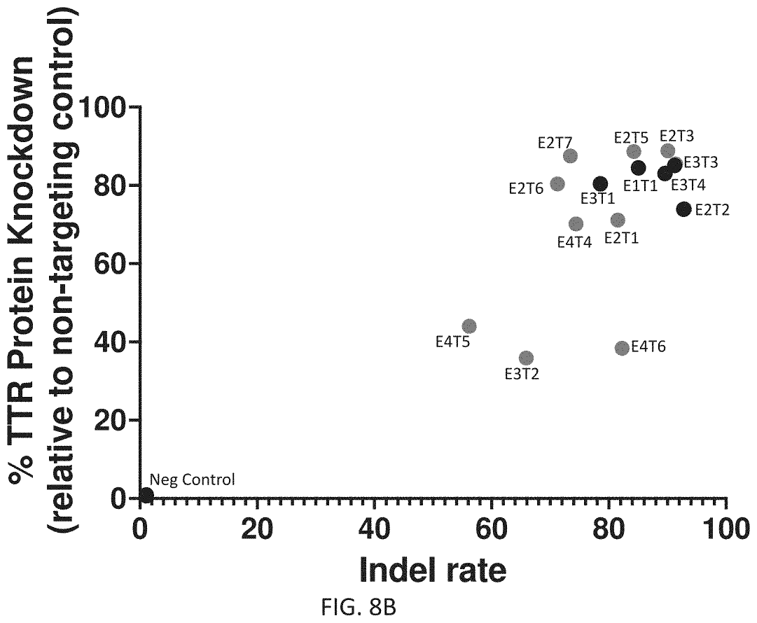

As shown in FIG. 8A, each tested TTR-targeting RNA guide induced indels in the TTR target sites. Indels were not induced with the non-targeting control. Indels were measured in over 90% of the NGS reads for 5 of the 14 tested TTR targets. Indels were retained by primary human hepatocytes up to 7 days post RNP electroporation.

As shown in FIG. 8B, each of the tested RNPs induced indels and reduced TTR protein levels in primary human hepatocytes. The black datapoints correspond to TTR targets with homology to TTR sequences in non-human primates; the grey datapoints correspond to TTR targets that do not have homology to TTR sequences in non-human primates. 11 of the 14 RNA guides induced indels in over 60% of the NGS reads and reduced TTR protein levels by at least 60% relative to the non-targeting control. RNA guides E1T1. E2T3. E2T5. E3T3. and E3T4 induced indels in over 80% of the NGS reads and reduced TTR protein levels by at least 80% relative to the non-targeting control.

This Example thus shows that TTR can be targeted by Cas12i2 RNPs in mammalian cells such as primary human hepatocytcs.

### Example 6—Targeting of TTR Target Sequences in HeuG2 Cells with Cas12i2 and Cas2i4

This Example describes del assessment on multiple TTR targets using variants introduced into HepG2 cells by transient transfection.

The Cas12i2 variants of SEQ ID NO: 224 and SEQ ID NO: 227 and the Cas12i4 variant of SEQ ID NO: 255 were individually cloned into a pcda3.1 backbone (Invitrogen). Nucleic acids encoding RNA guides were cloned into a pUC 19 backbone (New England Biolabs). The plasmids were then maxi-prepped and diluted. The RNA guide and target sequences are shown in Table 11.

TABLE 11
TTR Target Sequences and Corresponding crRNAs

| Target | | Cas12i2 | Cas12i4 |
|---|---|---|---|
| Identi- | | crRNA | crRNA |
| fier | Target Sequence | Sequence | Sequence |
| E1T1 | TCATCGTCTGCTCCT | AGAAAUCCGUCUUUC | AGACAUGUGUCCUCAGU |
| | CCTCT (SEQ ID NO: | AUUGACGGUCAUCGU | GACACUCAUCGUCUGCU |
| | 327) | CUGCUCCUCCUCU | CCUCCUCU (SEQ ID NO: |
| | | (SEQ ID NO: 345) | 386) |
| E1T2 | TTGGCAGGATGGCT | AGAAAUCCGUCUUUC | AGACAUGUGUCCUCAGU |
| | TCTCAT (SEQ ID NO: | AUUGACGGUUGGCAG | GACACUUGGCAGGAUG |
| | 363) | GAUGGCUUCUCAU | GCUUCUCAU (SEQ ID NO: |
| | | (SEQ ID NO: 375) | 387) |
| E1T3 | GCAGGATGGCTTCT | AGAAAUCCGUCUUUC | AGACAUGUGUCCUCAGU |
| | CATCGT (SEQ ID NO: | AU UGACGGGCAGGAU | GACACGC AGGAUGGCUU |
| | 364) | GGCUUCUCAUCGU | CUCAUCGU (SEQ ID NO: |
| | | (SEQ ID NO: 376) | 388) |
| E2T1 | TCTACACCCAGGGC | AGAAAUCCGUCUUUC | AGACAUGUGUCCUCAGU |
| | ACCGGT (SEQ ID NO: | AUUGACGGUCUACACC | GACACUCUACACCCAGG |
| | 328) | CAGGGCACCGGU (SEQ | GCACCGGU (SEQ ID NO: |
| | | ID NO: 346) | 389) |
| E2T2 | GACCATCAGAGGAC | AGAAAUCCGUCUUUC | AGACAUGUGUCCUCAGU |
| | ACTTGG (SEQ ID NO: | AUUGACGGGACCAUC | GACACGACCAUCAGAGG |
| | 329) | AGAGGACACUUGG | ACACUUGG (SEQ ID NO: |
| | | (SEQ ID NO: 347) | 390) |
| E2T3 | TAGATGCTGTCCCA | AGAAAUCCGUCUUUC | AGACAUGUGUCCUCAGU |
| | GGCAGT (SEQ ID NO: | AUUGACGGUAGAUGC | G ACACUAGAUGCUGUCC |
| | 330) | UGUCCGAGGCAGU | GAGGCAGU (SEQ ID NO: |
| | | (SEQ ID NO: 348) | 391) |
| E2T8 | ACCATCAGAGGACA | AGAAAUCCGUCUUUC | AGACAUGUGUCCUCAGU |
| | CTTGGA (SEQ ID NO: | AUUGACGGACCAUCA | GACACACCAUCAGAGGA |
| | 365) | GAGGACACUUGGA | CACUUGGA (SEQ ID NO: |
| | | (SEQ ID NO: 377) | 392) |
| E3T1 | CACACCTTATAGGA | AGAAAUCCGUCUUUC | AGACAUGUGUCCUCAGU |
| | AAACCA (SEQ ID NO: | AUUGACGGCACACCUU | GACACCACACCUUAUAG |
| | 335) | AUAGGAAAACCA (SEQ | GAAAACCA (SEQ ID NO: |
| | | ID NO: 353) | 393) |
| E3T2 | TACAAATTCCTCCTC | AGAAAUCCGUCUUUC | AGACAUGUGUCCUCAGU |
| | AGTTG (SEQ ID NO: | AUUGACGGUACAAAU | GACACUACAAAUUCCUC |
| | 336) | UCCUCCUCAGUUG | CUCAGUUG (SEQ ID NO: |
| | | (SEQ ID NO: 354) | 394) |
| E3T3 | GTATATCCCTTCTAC | AGAAAUCCGUCUUUC | AGACAUGUGUCCUCAGU |
| | AAATT (SEQ ID NO: | AUUGACGGGUAUAUC | GACACGUAUAUCCCUUC |
| | 337) | CCUUCUACAAAUU | UACAAAUU (SEQ ID NO: |
| | | (SEQ ID NO: 355) | 395) |
| E3T4 | CAGTAAGATTTGGT | AGAAAUCCGUCUUUC | AGACAUGUGUCCUCAGU |
| | GTCTAT (SEQ ID NO: | AUUGACGGCAGUAAG | GACACCAGUAAGAUUU |
| | 338) | AUUUGGUGUCUAU | GGUGUCU AU (SEQ ID |
| | | (SEQ ID NO: 356) | NO: 396) |
| E3T5 | ACACCTTATAGGAA | AGAAAUCCGUCUUUC | AGACAUGUGUCCUCAGU |
| | AACCAG (SEQ ID NO: | AUUGACGGACACCUU | GACACACACCUUAUAGG |
| | 366) | AUAGGAAAACCAG | AAAACCAG (SEQ ID NO: |
| | | (SEQ ID NO: 378) | 397) |
| E3T6 | TAGGAAAACCAGTG | AGAAAUCCGUCUUUC | AGACAUGUGUCCUCAGU |
| | AGTCTG (SEQ ID NO: | AUUGACGGUAGGAAA | GACACUAGGAAAACCAG |
| | 367) | ACCAGUGAGUCUG | UGAGUCUG (SEQ ID NO: |
| | | (SEQ ID NO: 379) | 398) |
| E3T7 | GTAGAAGGGATATA | AGAAAUCCGUCUUUC | AGACAUGUGUCCUCAGU |
| | CAAAGT (SEQ ID NO: | AUUGACGGGUAGAAG | GACACCUAGAAGGGAU |
| | 368) | GGAUAUACAAAGU | AUACAAAGU (SEQ ID |
| | | (SEQ ID NO: 380) | NO: 399) |
| E3T8 | TAGAAGGGATATAC | N.A. | AGACAUGUGUCCUCAGU |
| | AAAGTG (SEQ ID NO: | | GACACUAGAAGGGAUA |
| | 369) | | UACAAAGUG (SEQ ID |
| | | | NO: 400) |
| E3T9 | CACTITGTATATCCC | AGAAAUCCGUCUUUC | AGACAUGUGUCCUCAGU |
| | TTCTA (SEQ ID NO: | AUUGACGGCACUUUG | GACACCACUUUGUAUAU |
| | 370) | UAUAUCCCUUCUA | CCCUUCUA (SEQ ID NO: |
| | | (SEQ ID NO: 381) | 401) |
| EAT10 | CCACTTTGTATATCC | AGAAAUCCGUCUUUC | AGACAUGUGUCCUCAGU |
| | CTTCT (SEQ ID NO: | AUUGACGGCCACUUU | GACACCCACUUUGUAUA |
| | 371) | GUAUAUCCCUUCU | UCCCUUCU (SEQ ID NG: |
| | | (SEQ ID NO: 382) | 402) |
| E3T11 | GTGTCTATTTCCACT | AGAAAUCCGUCUUUC | AGACAUGUGUCCUCAGU |
| | TTGTA (SEQ ID NO: | AUUGACGGGUGUCUA | GACACGUGUCUAUUUCC |
| | 372) | UUUCCACUUUGUA | ACUUUGUA (SEQ ID NO: |
| | | (SEQ ID NO: 383) | 403) |
| E3T12 | GGTGTCTATTTCCAC | AGAAAUCCGUCUUUC | AGACAUGUGUCCUCAGU |
| | TTTGT (SEQ ID NO: | AUUGACGGGGUGUCU | GACACGGUGUCUAUUUC |
| | 373) | AUUUCCACUUUGU | CACUUUGU (SEQ ID NO: |
| | | (SEQ ID NO: 384) | 404) |
| E4T2 | CACCACGGCTGTCG | AGAAAUCCGUCUUUC | AGACAUGUGUCCUCAGU |
| | TCACCA (SEQ ID NO: | AUUGACGGCACCACGG | GACACCACCACOGCUGU |
| | 340) | CUGUCGUCACCA (SEQ | CGUCACCA (SEQ ID NO: |
| | | ID NO: 358) | 405) |
| E4T3 | GTGACGACAGCCGT | AGAAAUCCGUCUUUC | AGACAUGUGUCCUCAGU |
| | GGTGGA (SEQ ID NO: | AUUGACGGGUGACGA | GACACGUGACGACAGCC |
| | 341) | CAGCCGUGGUGGA | GUGGUGGA (SEQ ID NO: |
| | | (SEQ ID NO: 359) | 406) |
| E4T4 | GGATTGGTGACGAC | AGAAAUCCGUCUUUC | AGACAUGUGUCCUCAGU |
| | AGCCGT (SEQ ID NO: | AUUGACGGGGAUUGG | GACACGGAUUGGUGAC |
| | 342) | UGACGACAGCCGU | GACAGCCGU (SEQ ID NO: |
| | | (SEQ ID NO: 360) | 407) |
| E4T5 | CTTGGGATTGGTGA | AGAAAUCCGUCUUUC | AGACAUGUGUCCUCAGU |
| | CGAOAG (SEQ ID NO: | AUUGACGGCUUGGGA | GACACCUUGGGAUUGG |
| | 343) | UUGGUGACGACAG | UGACGACAG (SEQ ID |
| | | (SEQ ID NO: 361) | NO: 408) |
| E4T7 | TCTCATAGGTGGTAT | AGAAAUCCGUCUUUC | AGACAUGUGUCCUCAGU |
| | TCACA (SEQ ID NO: | AUUGACGGUCUCAUA | GACACUCUCAUAGGUGG |
| | 374) | GGUGGUAUUCACA | UAUUCACA (SEQ ID NO: |
| | | (SEQ ID NO: 385) | 409) |

HepG2 cells were harvested using TRYPLE™ (recombinant cell-dissociation enzymes; ThermoFisher) and counted. Cells were washed once with PBS and resuspended in SF buffer+supplement (SF CELL LINE 41D-NUCLEOFECTOR™ X KIT S; Lonra #V4XC-2032).

Approximately 16 hours prior to transfection, 25.000 HepG2 cells in EMEM/10% FBS were plated into each well of a 96-well plate. On the day of transfection, the cells were 70-90% confluent. For each well to be transfected, a mixture of Lipofectamine™ 3000 and Opti-MEM® was prepared and then incubated at room temperature for 5 minutes (Solution 1). After incubation, the Lipofectamine™:OptiMEM® mixture was added to a separate mixture containing nuclease plasmid and RNA guide plasmid and P3000 reagent (Solution 2). In the case of negative controls, the crRNA was not included in Solution 2. The Solution 1 and Solution 2 were mixed by pipetting up and down and then incubated at room temperature for 15 minutes. Following incubation, the Solution I and Solution 2 mixture was added dropwise to each well of a 96 well plate containing the cells.

After 3 days. wells were harvested using TRYPLE™ (recombinant cell-dissociation enzymes; ThermoFisher) and transferred to 96-well TWIN.TEC® PCR plates (Eppendorf). Media was flicked off and cells were resuspended in 20 μL QUICKEXTRACT™ (DNA extraction buffer; Lucigen). Samples were then cycled in a PCR machine at 65° C. for 15 min. 68° C. for 15 min, 98° C. for 10 min. Samples were then frozen at −20° C. and analyzed by NGS as described in Example 1.

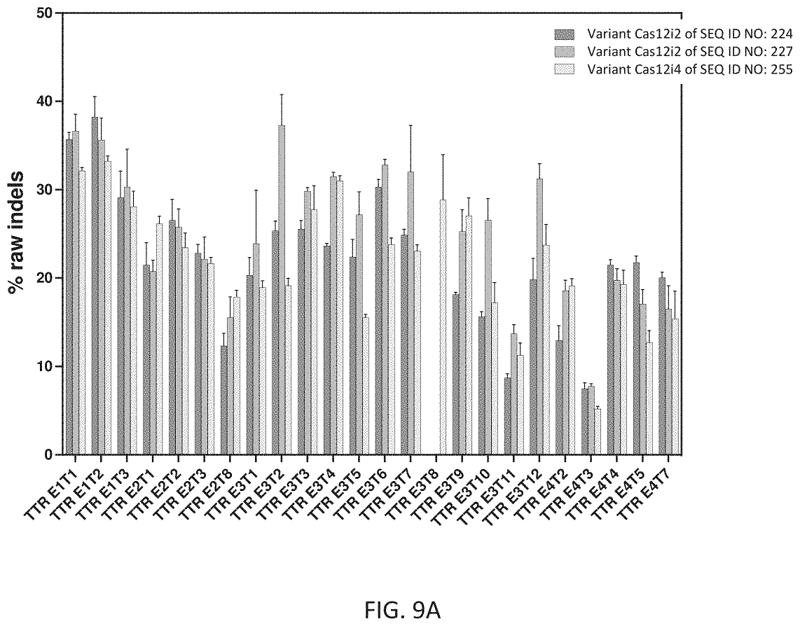

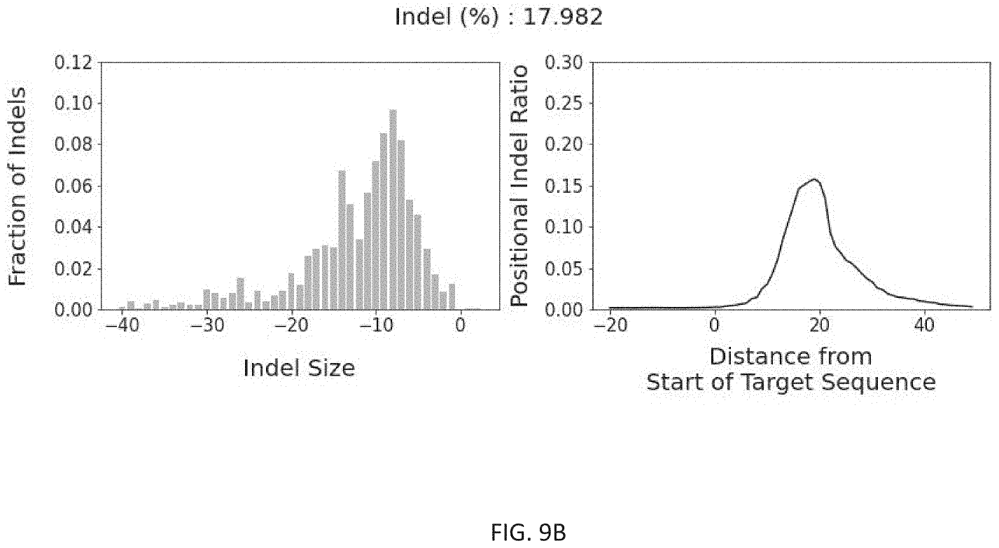

As shown in FIG. 9A, nine guides demonstrated significantly higher activity with variant Cas12i2 of SEQ ID NO: 227 compared to variant Cas12i2 of SEQ ID NO: 224. while thirteen guides showed no significant difference variant Cas12i2 of SEQ ID NO: 227 and variant Cas12i2 of SEQ ID NO: 224. Comparable editing was observed between variant Cas12i4 and the Cas12i2 variants. FIG. 9B shows the indel size frequency (left) and indel start position relative to the PAM for E2T3 and the variant Cas12i2 of SEQ ID NO: 224. As shown on the left, deletions ranged in size from 1 nucleotide to about 40 nucleotides. The majority of the deletions were about 4 nucleotides to about 18 nucleotides in length. As shown on the right. the target sequence is represented as starting at position 0 and ending at position 20. Indels started within about 5 nucleotides and about 35 nucleotides downstream of the PAM sequence. The majority of indels started near the end of the target sequence, e.g., about 15 nucleotides to about 25 nucleotides downstream of the PAM sequence.

Thus, this Example shows that TTR is capable of being targeted by both Cas12i2 and Cas12i4 variants.

### Example 7—Off-Target Analysis of Cas12i2 and TTR-Targeting RNA Guides

This Example describes on-target versus off-target assessment of a Cas12i2 variant and a TTR-targeting RNA guide.

HEK293T cells were transfected with a plasmid encoding the variant Cas12i2 of SEQ ID NO: 224 and a plasmid encoding E3T1 (SEQ ID NO: 353). E2T2 (SEQ ID NO: 347). E3T4 (SEQ ID NO: 356), E3T3 (SEQ ID NO: 355), E4T2 (SEQ ID NO: 358), E1T2 (SEQ ID NO: 363), or E1T1 (SEQ ID NO: 345) according the method described in Example 16 of PCT/US21/25257. The tagmentation-based tag integration site sequencing (TTISS) method described in Example 16 of PCT/US21/25257 was then carried out.

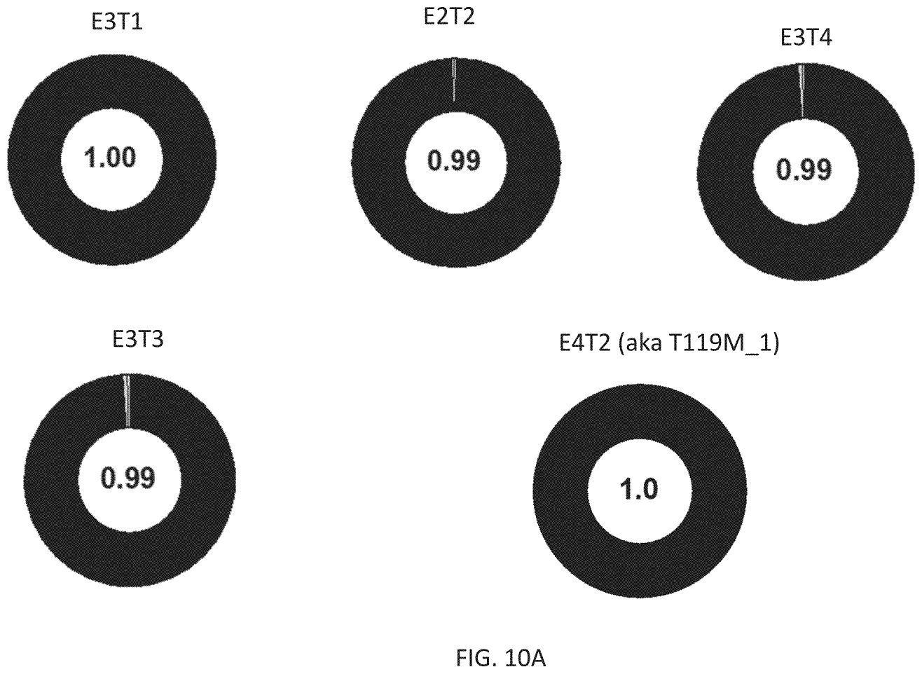

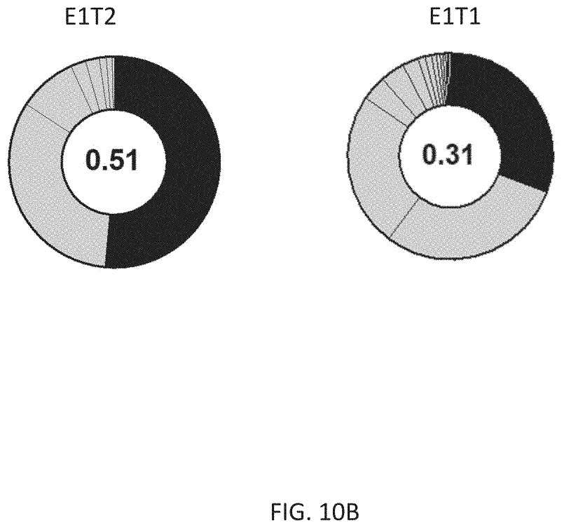

FIGS. 10A and 10B show plots depicting on-target and off-target TTISS reads. The black wedge and centered number represent the fraction of on-target TTISS reads. Each grey wedge represents a unique off-target site identified by TTISS. The size of each grey wedge represents the fraction of TTISS reads mapping to a given off-target site.

As shown in FIG. 10A, variant Cas12i2 of SEQ ID NO: 224 paired with E3T1 or E4T2 demonstrated a low likelihood of off-target editing, as 100% of TTISS reads mapped to the on-target. No TTISS reads mapped to potential off-target sites. E2T2, E3T4, and E3T3 also showed a low likelihood of off-target editing. For E2T2, E3T4, and E3T3, 99% of TTISS reads mapped to the on-target; off-target sites represented a combined 1% of TTISS reads for each RNA guide. However, as also shown in FIG. 10B, E1T2 and E1T1 demonstrated a higher likelihood of off-target editing using the TTISS method. For E1T2, only 51% of TTISS reads mapped to the on-target, and 7 potential off-target sites represented the remaining 49% of TTISS reads. For E1T1, only 31% of TTISS reads mapped to the on-target, and 14 potential off-target sites represented the remaining 69% of TTISS reads.

Therefore, this Example shows that compositions comprising Cas12i2 and TTR-targeting RNA guides comprise different off-target activity profiles.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof. can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize. or be able to ascertain using no more than routine experimentation, many equivalents to the specific inventive embodiments described herein. It is. therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature. system, article, material, kit, and/or method described herein. In addition, any combination of two or more such features, systems, articles, materials, kits, and/or methods, if such features. systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an.” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive. i.e., the inclusion of at least one, but also including more than one, of a number or list of elements, and, optionally, additional unlisted items. Only terms clearly indicated to the contrary. such as “only one of” or “exactly one of,” or, when used in the claims. “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one. A, with no B present (and optionally including elements other than B); in another embodiment, to at least one, optionally including more than one, B, with no A present (and optionally including elements other than A); in yet another embodiment, to at least one, optionally including more than one, A. and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A gene editing system for genetic editing of a transthyretin (TTR) gene, comprising
(i) a Cas12i2 polypeptide or a first nucleic acid encoding the Cas12i2 polypeptide, wherein the Cas12i2 polypeptide comprises an amino acid sequence at least 95% identical to SEQ ID NO: 222 and comprises one or more mutations relative to SEQ ID NO: 222;
(ii) an RNA guide or a second nucleic acid encoding the RNA guide, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence.

2. The gene editing system of claim 1, wherein the one or more mutations in the Cas12i2 polypeptide are at positions D581, G624, F626, P868, I926, V1030, E1035, and/or S1046 of SEQ ID NO: 222.

3. The gene editing system of claim 1 or claim 2, wherein the one or more mutations are amino acid substitutions, which optionally is D581R, G624R, F626R, P868T, I926R, V1030G, E1035R, S1046G, or a combination thereof.

4. The gene editing gene editing system of claim 3, wherein the Cas12i2 polypeptide comprises:
(i) mutations at positions D581, D911, I926, and V1030, which optionally are amino acid substitutions of D581R, D911R, I926R, and V1030G;
(ii) mutations at positions D581, I926, and V1030, which optionally are amino acid substitutions of D581R, I926R, and V1030G;
(iii) mutations at positions D581, I926, V1030, and S1046, which optionally are amino acid substitutions of D581R, I926R, V1030G, and S1046G;
(iv) mutations at positions D581, G624, F626, I926, V1030, E1035, and S1046, which optionally are amino acid substitutions of D581R, G624R, F626R, I926R, V1030G, E1035R, and S1046G; or
(v) mutations at positions D581, G624, F626, P868, I926, V1030, E1035, and S1046, which optionally are amino acid substitutions of D581R, G624R, F626R, P868T, I926R, V1030G, E1035R, and S1046G.

5. The gene editing system of claim 1, wherein the Cas12i2 polypeptide comprises the amino acid sequence of SEQ ID NO: 223-227, optionally wherein the Cas12i2 polypeptide comprises the amino acid sequence of SEQ ID NO: 224 or SEQ ID NO: 227.

6. The gene editing system of any one of claims 1-5, which comprises the first nucleic acid encoding the Cas12i2 polypeptide.

7. The gene editing system of claim 6, wherein the first nucleic acid is a messenger RNA (mRNA).

8. The gene editing system of claim 6, wherein the first nucleic acid is included in a viral vector, which optionally is an adeno-associated viral (AAV) vector.

9. The gene editing system of any one of claims 1-8, wherein the target sequence is within exon 2, exon 3, or exon 4 of the TTR gene.

10. The gene editing system of claim 9, wherein the target sequence comprises:
(i)
(SEQ ID NO: 329)
GACCATCAGAGGACACTTGG,
(ii)
(SEQ ID NO: 330)
TAGATGCTGTCCGAGGCAGT,
(iii)
(SEQ ID NO: 332)
CTGAACACATGCACGGCCAC,
(iv)
(SEQ ID NO: 333)
GGCAACTTACCCAGAGGCAA,
(v)
(SEQ ID NO: 334)
TTTGGCAACTTACCCAGAGG,
(vi)
(SEQ ID NO: 335)
CACACCTTATAGGAAAACCA,
(vii)
(SEQ ID NO: 337)
GTATATCCCTTCTACAAATT,
(viii)
(SEQ ID NO: 338)
CAGTAAGATTTGGTGTCTAT,
or
(ix)
(SEQ ID NO: 271)
CACCACGGCTGTCGTCACCA.

11. The gene editing system of claim 10, wherein the spacer sequence is set forth as:
(i)
(SEQ ID NO: 410)
GACCAUCAGAGGACACUUGG,
(ii)
(SEQ ID NO: 411)
UAGAUGCUGUCCGAGGCAGU,
(iii)
(SEQ ID NO: 412)
CUGAACACAUGCACGGCCAC,
(iv)
(SEQ ID NO: 413)
GGCAACUUACCCAGAGGCAA,
(v)
(SEQ ID NO: 414)
UUUGGCAACUU ACCCAGAGG,
(vi)
(SEQ ID NO: 415)
CACACCUUAUAGGAAAACCA,
(vii)
(SEQ ID NO: 416)
GUAUAUCCCUUCUACAAAUU,
(viii)
(SEQ ID NO: 417)
CAGUAAGAUUUGGUGUCUAU,
or
(xi)
(SEQ ID NO: 418)
CACCACGGCUGUCGUCACCA.

12. The gene editing system of any one of claims 1-11, wherein the spacer sequence is 20-30-nucleotide in length, optionally wherein the spacer sequence is 20-nucleotide in length.

13. The gene editing system of any one of claims 1-12, wherein the RNA guide comprises the spacer sequence and a direct repeat sequence.

14. The gene editing system of claim 13, wherein the direct repeat sequence is 23-36-nucleotide in length.

15. The gene editing system of claim 14, wherein the direct repeat sequence is at least 90% identical to any one of SEQ ID NOs: 1-10 or a fragment thereof that is at least 23-nucleotide in length.

16. The gene editing system of claim 13, wherein the direct repeat sequence is any one of SEQ ID NOs: 1-10, or a fragment thereof that is at least 23-nucleotide in length.

17. The gene editing system of claim 16, wherein the direct repeat sequence is 5′-AGAAAUCCGUCUUUCAUUGACGG-3′ (SEQ ID NO: 10).

18. The gene editing system of claim 1, wherein the RNA guide comprises the nucleotide sequence of:
(i)
(SEQ ID NO: 347)
AGAAAUCCGUCUUUCAUUGACGGGACCAUCAGAGGACACUUGG,
(ii)
(SEQ ID NO: 348)
AGAAAUCCGUCUUUCAUUGACGGUAGAUGCUGUCCGAGGCAGU,
(iii)
(SEQ ID NO: 350)
AGAAAUCCGUCUUUCAUUGACGGCUGAACACAUGCACGGCCAC,
(iv)
(SEQ ID NO: 351)
AGAAAUCCGUCUUUCAUUGACGGGGCAACUUACCCAGAGGCAA,
(v)
(SEQ ID NO: 352)
AGAAAUCCGUCUUUCAUUGACGGUUUGGCAACUUACCCAGAGG,
(vi)
(SEQ ID NO: 353)
AGAAAUCCGUCUUUCAUUGACGGCACACCUUAUAGGAAAACCA,
(vii)
(SEQ ID NO: 355)
AGAAAUCCGUCUUUCAUUGACGGGUAUAUCCCUUCUACAAAUU,
(viii)
(SEQ ID NO: 356)
AGAAAUCCGUCUUUCAUUGACGGCAGUAAGAUUUGGUGUCUAU,
or
(ix)
(SEQ ID NO: 358)
AGAAAUCCGUCUUUCAUUGACGGCACCACGGCUGUCGUCACCA.

19. The gene editing system of any one of claims 1-18, wherein the system comprises the second nucleic acid encoding the RNA guide.

20. The gene editing system of claim 19, wherein the nucleic acid encoding the RNA guide is located in a viral vector.

21. The gene editing system of any one of claims 8-20, wherein the viral vector comprises both the first nucleic acid encoding the Cas12i2 polypeptide and the second nucleic acid encoding the RNA guide.

22. The gene editing system of any one of claims 1-20, wherein the system comprises the first nucleic acid encoding the Cas12i2 polypeptide, which is located in a first vector, and wherein the system comprises the second nucleic acid encoding the RNA guide, which is located in a second vector.

23. The gene editing system of any one of claims 1-22, wherein the system further comprises (iii) a template DNA, which comprising (a) a first segment homologous to a first site in the TTR gene that is upstream to a TTR gene target site for genetic editing, (b) a second segment homologous to a second site that is downstream to the TTR gene target site for genetic editing, and (c) a donor region, which is homologous to the TTR gene target site for genetic editing and comprises at least one nucleotide variation relative to the TTR gene target site for genetic editing; and wherein the donor region is flanked by the first and second segments.

24. The gene editing system of claim 23, wherein the TTR gene target site for genetic editing comprises the target sequence, the PAM, or a combination thereof.

25. The gene editing system of claim 23 or claim 24, wherein the TTR gene target site for genetic editing comprises a mutation associated with a disease, and wherein the donor region comprises a sequence that fixes the mutation.

26. The gene editing system of claim 25, wherein the mutation associated with the disease leads to the amino acid residue substitution of V30M, V122I, T60A, L58H, or I84S relative to the TTR sequence of SEQ ID NO: 257.

27. The gene editing system of claim 23 or claim 24, wherein the donor region comprises a protective mutation relative to the TTR gene target site for genetic editing.

28. The gene editing system of claim 27, wherein the protective mutation leads to the amino acid residue substitution of T119M relative to the TTR sequence of SEQ ID NO: 257.

29. The gene editing system of any one of claims 23-28, wherein the templated DNA is located in a viral vector, which optionally is an AAV vector.

30. The gene editing system of any one of claims 1-29, wherein the system comprises one or more lipid nanoparticles (LNPs), which encompass (i), (ii), or both, and optionally (iii).

31. The gene editing system of claim 30, wherein the system comprises the LNP, which encompass (i), and wherein the system comprises a viral vector comprising the second nucleic acid encoding the RNA guide; optionally wherein the viral vector is an AAV vector.

32. The gene editing system of claim 30, wherein the system comprises the LNP, which encompass (ii), and wherein the system comprises a viral vector comprising the first nucleic acid encoding Cas12i2 polypeptide; optionally wherein the viral vector is an AAV vector.

33. A gene editing system for genetic editing of a transthyretin (TTR) gene, comprising
(i) a Cas12i polypeptide or a first nucleic acid encoding the Cas12i polypeptide, optionally wherein the Cas12i polypeptide is a Cas12i2 polypeptide;
(ii) an RNA guide or a second nucleic acid encoding the RNA guide, wherein the RNA guide comprises a spacer sequence specific to a target sequence within exon 2, exon 3, or exon 4 of a TTR gene, the target sequence being adjacent to a protospacer adjacent motif (PAM) comprising the motif of 5′-TTN-3′, which is located 5′ to the target sequence.

34. The gene editing system of claim 33, wherein the target sequence comprises:
(i)
(SEQ ID NO: 329)
GACCATCAGAGGACACTTGG,
(ii)
(SEQ ID NO: 330)
TAGATGCTGTCCGAGGCAGT,
(iii)
(SEQ ID NO: 332)
CTGAACACATGCACGGCCAC,
(iv)
(SEQ ID NO: 333)
GGCAACTTACCCAGAGGCAA,
(v)
(SEQ ID NO: 334)
TTTGGCAACTTACCCAGAGG,
(vi)
(SEQ ID NO: 335)
CACACCTTATAGGAAAACCA,
(vii)
(SEQ ID NO: 337)
GTATATCCCTTCTACAAATT,
(viii)
(SEQ ID NO: 338)
CAGTAAGATTTGGTGTCTAT,
or
(ix)
(SEQ ID NO: 271)
CACCACGGCTGTCGTCACCA.

35. The gene editing system of claim 34, wherein the spacer sequence is set forth as:
(i)
(SEQ ID NO: 410)
GACCAUCAGAGGACACUUGG,
(ii)
(SEQ ID NO: 411)
UAGAUGCUGUCCGAGGCAGU,
(iii)
(SEQ ID NO: 412)
CUGAACACAUGCACGGCCAC,
(iv)
(SEQ ID NO: 413)
GGCAACUUACCCAGAGGCAA,
(v)
(SEQ ID NO: 414)
UUUGGCAACUUACCCAGAGG,
(vi)
(SEQ ID NO: 415)
CACACCUUAUAGGAAAACCA,
(vii)
(SEQ ID NO: 416)
GUAUAUCCCUUCUACAAAUU,
(viii)
(SEQ ID NO: 417)
CAGUAAGAUUUGGUGUCUAU,
or
(xi)
(SEQ ID NO: 418)
CACCACGGCUGUCGUCACCA,

36. The gene editing system of any one of claims 33-35, which comprises the first nucleic acid encoding the Cas12i2 polypeptide.

37. The gene editing system of claim 36, wherein the first nucleic acid is a messenger RNA (mRNA).

38. The gene editing system of claim 37, wherein the first nucleic acid is included in a viral vector, which optionally is an adeno-associated viral (AAV) vector.

39. The gene editing system of any one of claims 33-38, wherein the spacer sequence is 20-30-nucleotide in length, optionally wherein the spacer sequence is 20-nucleotide in length.

40. The gene editing system of any one of claims 33-39, wherein the RNA guide comprises the spacer sequence and a direct repeat sequence.

41. The gene editing system of claim 40, wherein the direct repeat sequence is 23-36-nucleotide in length.

42. The gene editing system of claim 41, wherein the direct repeat sequence is at least 90% identical to any one of SEQ ID NOs: 1-10 or a fragment thereof that is at least 23-nucleotide in length.

43. The gene editing system of claim 42, wherein the direct repeat sequence is any one of SEQ ID NOs: 1-10, or a fragment thereof that is at least 23-nucleotide in length.

44. The gene editing system of claim 43, wherein the direct repeat sequence is 5′-AGAAAUCCGUCUUUCAUUGACGG-3′ (SEQ ID NO: 10).

45. The gene editing system of claim 43, wherein the RNA guide comprises the nucleotide sequence of:
(i)
(SEQ ID NO: 347)
AGAAAUCCGUCUUUCAUUGACGGGACCAUCAGAGGACACUUGG
(ii)
(SEQ ID NO: 348)
AGAAAUCCGUCUUUCAUUGACGGUAGAUGCUGUCCGAGGCAGU
(iii)
(SEQ ID NO: 350)
AGAAAUCCGUCUUUCAUUGACGGCUGAACACAUGCACGGCCAC
(iv)
(SEQ ID NO: 351)
AGAAAUCCGUCUUUCAUUGACGGGGCAACUUACCCAGAGGCAA
(v)
(SEQ ID NO: 352)
AGAAAUCCGUCUUUCAUUGACGGUUUGGCAACUUACCCAGAGG
(vi)
(SEQ ID NO: 353)
AGAAAUCCGUCUUUCAUUGACGGCACACCUUAUAGGAAAACCA
(vii)
(SEQ ID NO: 355)
AGAAAUCCGUCUUUCAUUGACGGGUAUAUCCCUUCUACAAAUU
(viii)
(SEQ ID NO: 356)
AGAAAUCCGUCUUUCAUUGACGGCAGUAAGAUUUGGUGUCUAU
or
(ix)
(SEQ ID NO: 358)
AGAAAUCCGUCUUUCAUUGACGGCACCACGGCUGUCGUCACCA

46. The gene editing system of any one of claims 33-45, wherein the system comprises the second nucleic acid encoding the RNA guide.

47. The gene editing system of claim 46, wherein the nucleic acid encoding the RNA guide is located in a viral vector.

48. The gene editing system of any one of claims 38-47, wherein the viral vector comprises the both the first nucleic acid encoding the Cas12i2 polypeptide and the second nucleic acid encoding the RNA guide.

49. The gene editing system of any one of claims 33-48, wherein the system comprises the first nucleic acid encoding the Cas12i2 polypeptide, which is located on a first vector, and wherein the system comprises the second nucleic acid encoding the RNA guide, which is located on a second vector.

50. The gene editing system of any one of claims 33-49, wherein the system further comprises (iii) a template DNA, which comprising (a) a first segment homologous to a first site in the TTR gene that is upstream to a TTR gene target site for genetic editing, (b) a second segment homologous to a second site that is downstream to the TTR gene target site for genetic editing, and (c) a donor region, which is homologous to the TTR gene target site for genetic editing and comprises at least one nucleotide variation relative to the TTR gene target site for genetic editing; and wherein the donor region is flanked by the first and second segments.

51. The gene editing system of claim 50, wherein the TTR gene target site for genetic editing comprises the target sequence, the PAM, or a combination thereof.

52. The gene editing system of claim 50 or claim 51, wherein the TTR gene target site for genetic editing comprises a mutation associated with a disease, and wherein the donor region comprises a sequence that fixes the mutation.

53. The gene editing system of claim 52, wherein the mutation associated with the disease leads to the amino acid residue substitution of V30M, V122I, T60A, L58H, or 184S relative to the TTR sequence of SEQ ID NO: 257.

54. The gene editing system of claim 50 or claim 53, wherein the donor region comprises a protective mutation relative to the TTR gene target site for genetic editing.

55. The gene editing system of claim 54, wherein the protective mutation leads to the amino acid residue substitution of T119M relative to the TTR sequence of SEQ ID NO: 257.

56. The gene editing system of any one of claims 50-55, wherein the templated DNA is located in a viral vector, which optionally is an AAV vector.

57. The gene editing system of any one of claims 33-56, wherein the system comprises one or more lipid nanoparticles (LNPs), which encompass (i), (ii), or both, or optionally (iii).

58. The gene editing system of claim 57, wherein the system comprises the LNP, which encompass (i), and wherein the system comprises a viral vector comprising the second nucleic acid encoding the RNA guide; optionally wherein the viral vector is an AAV vector.

59. The gene editing system of claim 57, wherein the system comprises the LNP, which encompass (ii), and wherein the system comprises a viral vector comprising the first nucleic acid encoding Cas12i2 polypeptide; optionally wherein the viral vector is an AAV vector.

60. A gene editing system for genetic editing of a transthyretin (TTR) gene, comprising
(i) a Cas12i polypeptide or a first nucleic acid encoding the Cas12i polypeptide, optionally wherein the Cas12i polypeptide is a Cas12i2 polypeptide;
(ii) an RNA guide or a second nucleic acid encoding the RNA guide, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence; and
(iii) a template DNA, which comprising (a) a first segment homologous to a first site in the TTR gene that is upstream to a TTR gene target site for genetic editing, (b) a second segment homologous to a second site that is downstream to the TTR gene target site for genetic editing, and (c) a donor region, which is homologous to the TTR gene target site for genetic editing and comprises at least one nucleotide variation relative to the TTR gene target site for genetic editing; and wherein the donor region is flanked by the first and second segments.

61. The gene editing system of claim 60, wherein the donor region in the template DNA comprises a sequence that fixes a mutation in the TTR gene target site associated with a disease, comprises a protective mutation, or a combination thereof.

62. A pharmaceutical composition comprising the gene editing system set forth in any one of claims 1-61.

63. A kit comprising the elements (i) and (ii), and optionally (iii) of the gene editing system set forth in any one of claims 1-61.

64. A method for editing a transthyretin (TTR) gene in a cell, the method comprising contacting a host cell with the gene editing system for editing the TTR gene set forth in any one of claims 1-61 to genetically edit the TTR gene in the host cell.

65. The method of claim 64, wherein the host cell is cultured in vitro.

66. The method of claim 65, wherein the contacting step is performed by administering the system for editing the TTR gene to a subject comprising the host cell.

67. A cell comprising a mutated transthyretin (TTR) gene, wherein the cell optionally is produced by contacting a host cell with the gene editing system of any one of claims 1-61 to genetically edit the TTR gene in the host cell, thereby mutating the TTR gene.

68. The cell of claim 67, wherein the cell comprises a disrupted TTR gene.

69. The cell of claim 67, wherein the cell comprises a modified TTR gene, which expresses a mutated TTR relative to a wild-type counterpart cell.

70. A method for treating amyloidogenic transthyretin (ATTR) in a subject, comprising administering to a subject in need thereof a gene editing system for editing a transthyretin (TTR) gene set forth in any one of claims 1-61 or the cell of any one of claims 67-69.

71. The method of claim 70, wherein the subject is a human patient having hereditary ATTR (hATTR) or wild-type ATTR amyloidosis.

72. An RNA guide, comprising (i) a spacer sequence that is specific to a target sequence in a transthyretin (TTR) gene, wherein the target sequence is adjacent to a protospacer adjacent motif (PAM) comprising the motif of 5′-TTN-3′, which is located 5′ to the target sequence; and (ii) a direct repeat sequence.

73. The RNA guide of claim 72, wherein the spacer sequence is 20-30-nucleotide in length, optionally 20-nucleotide in length.

74. The RNA guide of claim 72 or claim 73, wherein the direct repeat sequence is 23-36-nucleotide in length, optionally 23-nucleotide in length.

75. The RNA guide of any one of claims 72-74, wherein the target sequence is within exon 2, exon 3, or exon 4 of the TTR gene.

76. The RNA guide of claim 75, wherein the target sequence comprises:
(i)
(SEQ ID NO: 329)
GACCATCAGAGGACACTTGG,
(ii)
(SEQ ID NO: 330)
TAGATGCTGTCCGAGGCAGT,
(iii)
(SEQ ID NO: 332)
CTGAACACATGCACGGCCAC,
(iv)
(SEQ ID NO: 333)
GGCAACTTACCCAGAGGCAA,
(v)
(SEQ ID NO: 334)
TTTGGCAACTTACCCAGAGG,
(vi)
(SEQ ID NO: 335)
CACACCTTATAGGAAAACCA,
(vii)
(SEQ ID NO: 337)
GTATATCCCTTCTACAAATT,
(viii)
(SEQ ID NO: 338)
CAGTAAGATTTGGTGTCTAT,
or
(ix)
(SEQ ID NO: 271)
CACCACGGCTGTCGTCACCA.

77. The RNA guide of claim 76, wherein the spacer sequence is set forth as:
(i)
(SEQ ID NO: 410)
GACCAUCAGAGGACACUUGG,
(ii)
(SEQ ID NO: 411)
UAGAUGCUGUCCGAGGCAGU,
(iii)
(SEQ ID NO: 412)
CUGAACACAUGCACGGCCAC,
(iv)
(SEQ ID NO: 413)
GGCAACUUACCCAGAGGCAA,
(v)
(SEQ ID NO: 414)
UUUGGCAACUUACCCAGAGG,
(vi)
(SEQ ID NO: 415)
CACACCUUAUAGGAAAACCA,
(vii)
(SEQ ID NO: 416)
GUAUAUCCCUUCUACAAAUU,
(viii)
(SEQ ID NO: 417)
CAGUAAGAUUUGGUGUCUAU,
or
(xi)
(SEQ ID NO: 418)
CACCACGGCUGUCGUCACCA.

78. The RNA guide of any one of claims 72-77, wherein the direct repeat sequence is at least 90% identical to any one of SEQ ID NOs: 1-10 or a fragment thereof that is at least 23-nucleotide in length.

79. The RNA guide of claim 78, wherein the direct repeat sequence is any one of SEQ ID NOs: 1-10, or a fragment thereof that is at least 23-nucleotide in length.

80. The RNA guide of claim 79, wherein the direct repeat sequence is 5′-AGAAAUCCGUCUUUCAUUGACGG-3′ (SEQ ID NO: 10).

81. The RNA guide of claim 78, which comprises the nucleotide sequence of:
(i)
(SEQ ID NO: 347)
AGAAAUCCGUCUUUCAUUGACGGGACCAUCAGAGGACACUUGG,
(ii)
(SEQ ID NO: 348)
AGAAAUCCGUCUUUCAUUGACGGUAGAUGCUGUCCGAGGCAGU,
(iii)
(SEQ ID NO: 350)
AGAAAUCCGUCUUUCAUUGACGGCUGAACACAUGCACGGCCAC,
(iv)
(SEQ ID NO: 351)
AGAAAUCCGUCUUUCAUUGACGGGGCAACUUACCCAGAGGCAA,
(v)
(SEQ ID NO: 352)
AGAAAUCCGUCUUUCAUUGACGGUUUGGCAACUUACCCAGAGG,
(vi)
(SEQ ID NO: 353)
AGAAAUCCGUCUUUCAUUGACGGCACACCUUAUAGGAAAACCA,
(vii)
(SEQ ID NO: 355)
AGAAAUCCGUCUUUCAUUGACGGGUAUAUCCCUUCUACAAAUU,
(viii)
(SEQ ID NO: 356)
AGAAAUCCGUCUUUCAUUGACGGCAGUAAGAUUUGGUGUCUAU,
or
(ix)
(SEQ ID NO: 358)
AGAAAUCCGUCUUUCAUUGACGGCACCACGGCUGUCGUCACCA.
